(19) United States
(12) Patent Application Publication
(10) Pub. No.: US 2026/0048137 A1
(43) Pub. Date: Feb. 19, 2026

(54) ANTI-MESOTHELIN ANTIBODY CONJUGATES AND METHODS OF USE THEREOF

(71) Applicant: Ardeagen Corporation, Woodinville, WA (US)

(72) Inventors: Maria Leia Smith, Seattle, WA (US); May Kung Sutherland, Bothell, WA (US); Yaohua HU, Shanghai (CN); Xia WANG, Shanghai (CN); Siwei NIE, Shanghai (CN); Jijie GU, Shanghai (CN)

(21) Appl. No.: 19/300,071

(22) Filed: Aug. 14, 2025

(30) Foreign Application Priority Data
Aug. 16, 2024 (WO) PCT/CN2024/112762

(51) Int. Cl. A61K 47/68; A61P 35/00; C07K 16/30

(52) U.S. Cl. CPC A61K 47/6851; A61K 47/68031; A61P 35/00; C07K 16/30; C07K 2317/24; C07K 2317/33; C07K 2317/77; C07K 2317/92

(57) ABSTRACT

The present disclosure relates to anti-MSLN conjugates that include a binding agent, at least one linker attached to the binding agent; and at least one cytotoxic agent attached to the at least one linker. In some embodiments, the binding agent includes a heavy chain variable (VH) region and a light chain variable (VL) region, wherein the VH region includes a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region includes a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6. In some embodiments, the anti-MSLN conjugates may be used in methods of treating a MSLN+ cancer.

### CROSS-REFERENCE TO RELATED APPLICATION

This application claims the benefit of International Patent Application No. PCT/CN2024/112762, filed Aug. 16, 2024, which application is incorporated herein by reference in its entirety.

### REFERENCE TO AN ELECTRONIC SEQUENCE LISTING

The contents of the electronic sequence listing (120301_406WO_seqListing.xml; Size: 4,928,909 bytes; and Date of Creation: Jul. 26, 2024) are herein incorporated by reference in their entirety.

### BACKGROUND

Effective and tumor-targeted treatment for various types of cancer remains an important need to improve the survival rate of patients. Worldwide in 2022, there were 2.5 million cases of lung cancer with 1.8 million deaths (Global Cancer Observatory, WHO). In the same year, 1.6 million cases of gastric/esophageal cancer, 1.9 million colorectal cancer, 0.6 million cervical/uterine cancers, 0.4 million ovarian cancer, 0.4 million triple-negative breast cancer, and 0.5 million cases of pancreatic cancer accounted for over 4 million deaths (Global Cancer Observatory, WHO). Currently, there are limited therapeutic options for at least pancreatic and ovarian cancers.

### BRIEF SUMMARY

In some aspects, the present disclosure provides an anti-mesothelin (anti-MSLN) conjugate including a binding agent, at least one linker attached to the binding agent; and at least one cytotoxic agent attached to the at least one linker.

Another embodiment of the present disclosure is an anti-MSLN conjugate including a binding agent that includes a heavy chain variable (VH) region and a light chain variable (VL) region, wherein the VH region comprises a complementarity determining region (HCDR)1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region includes a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6; at least one linker attached to the binding agent; and at least one cytotoxic agent attached to the at least one linker.

Another embodiment of the present disclosure is a method of treating a MSLN+ cancer, including administering to a subject in need thereof a therapeutically effective amount of an anti-MSLN conjugate, wherein the conjugate includes a binding agent, at least one linker attached to the binding agent; and at least one cytotoxic agent attached to the at least one linker.

Another embodiment of the present disclosure is a method of improving treatment outcome in a subject receiving immunotherapy and/or chemotherapy for a MSLN+ cancer, including administering an effective amount of an immunotherapy or chemotherapy to the subject; and administering a therapeutically effective amount of a conjugate, wherein the conjugate includes a binding agent, at least one linker attached to the binding agent; and at least one cytotoxic agent attached to the at least one linker.

### BRIEF DESCRIPTION OF THE DRAWINGS

FIGS. 1A-1B show FACS binding of different antibodies to human MSLN engineered cell in the absence (FIG. 1A) or presence (FIG. 1B) of soluble MSLN. “Amgen mAb” refers to anti-MSLN parental antibody of Amgen MSLN-BiTE and “Harpoon mAb” refers to anti-MSLN parental antibody of Harpoon HPN536.

FIGS. 2A-2B show ELISA binding of IgG conversion antibodies to extracellular domain of human MSLN (FIG. 2A) and MSLN region III proteins (FIG. 2B).

FIGS. 3A-3D show FACS binding of benchmark antibodies and PTM removed antibodies to a human MSLN expressing cell line with or without soluble MSLN.

FIG. 4 shows the internalization activity of PTM removed mAbs to human MSLN expressing engineered cells.

FIG. 5 shows the SDS-PAGE result of Ab-1 (also referred to as W305044-1.100.1-p3-uIgG1KV320 and W305044 mAb).

FIGS. 6A-6C show the purity (FIG. 6A), thermal stability (FIG. 6B) and retention time (FIG. 6C) of Ab-1.

FIGS. 7A-7C show the FACS binding of anti-MSLN mAbs (including Ab-1/W305044) to human MSLN (FIG. 7A), cyno MSLN (FIG. 7B) and mouse MSLN (FIG. 7C) expressing engineered cells.

FIGS. 8A-8E show the FACS binding of anti-MSLN mAbs (including Ab-1/W305044) to five selected human tumor cell lines.

FIGS. 9A-9C show the ELISA binding of antibody to human MSLN (FIG. 9A) and MSLN region III (FIG. 9B and FIG. 9C) proteins.

FIG. 10 shows the result of Fab-ZAP internalization assay on CHO cells expressing human MSLN (W3xx044-CHOK1.hPro1.G8) cells.

FIGS. 11A-11C show the result of FACS binding to human MSLN engineered cells with and without soluble MSLN.

FIGS. 12A-12C show the result of FACS binding to NCI-N87 human tumor cells with and without soluble MSLN.

FIGS. 13A-13C show the result of FACS binding to OVCAR3 human tumor cells with and without soluble MSLN.

FIGS. 14A-14E show the affinity result of anti-MSLN mAbs to MSLN by SPR analysis.

FIGS. 15A-15F show the affinity result of anti-MSLN mAbs to human tumor cell surface MSLN by FACS analysis.

FIG. 16 shows the radius of Ab-1 (also referred to as W305044-1.100.1-p3-uIgG1KV320 and W305044 mAb) measured by DLS (Dynamic Light Scattering).

FIG. 17 shows the determination of diffusion interaction parameter (kD) of Ab-1 (also referred to as W305044-1.100.1-p3-uIgG1KV320 and W305044 mAb) by DLS.

FIG. 18 shows the structure of ADC-1. ADC-1 is Ab-1 (also referred to as W305044-1.100.1-p3-uIgG1KV320 and W305044 mAb) with an enzyme-cleavable linker and a topoisomerase I inhibitor deruxtecan (Dxd) payload. Drug loading (DAR) as assessed by LC-MS was an average of about 7-8.

FIG. 19 shows the structure of ADC-2. ADC-2 is Ab-1 (also known as W305044-1.100.1-p3-uIgG1KV320 and W305044 mAb) with an acid-labile and enzyme-cleavable linker and a topoisomerase I inhibitor SN38 payload. Drug loading (DAR) as assessed by LC-MS was an average of about 7-8.

FIG. 20 shows the structure of ADC-3. ADC-3 is Ab-1 (also known as W305044-1.100.1-p3-uIgG1KV320 and W305044 mAb) with an enzyme-cleavable linker and an exatecan topoisomerase I inhibitor payload. Drug loading (DAR) as assessed by LC-MS would be expected to be an average of about 8.

FIG. 21 shows the structure of ADC-4. ADC-4 is Ab-1 (also referred to as W305044-1.100.1-p3-uIgG1KV320 and W305044 mAb) with an enzyme-cleavable linker and auristatin (MMAE) payload. Drug loading (DAR) as assessed by LC-MS was an average of about 4.

FIGS. 22A-22B show a comparison of Flow Cytometry (FACS) binding of Ab-1 and competitor mAb to target-expressing cell lines in the absence or presence of soluble MSLN. The competitor mAb is the Harpoon mAb, which is the anti-MSLN parental antibody of Harpoon HPN536 (HRPN-536 parent Ab). FIGS. 22A-22B demonstrate that the binding of Ab-1 is not competed by soluble antigen.

FIGS. 23A-23C show flow cytometry (FACS) binding of Ab-1, Ab-1-vcMMAE (ADC-4) and Ab-1-Dxd (ADC-1) ADCs to target-positive and target-negative cell lines. FIGS. 23A-23C demonstrate specific binding of Ab-1 and Ab-1-based ADCs on the 2 target-expressing cancer cell lines.

FIG. 24 shows the antitumor effect of Ab-1-vcMMAE (ADC-4) and Ab-1-Dxd (ADC-1) ADCs in the NCI-N87 gastric carcinoma xenograft model. ADCs were given intravenously once every 4 days for 3 doses (arrows). Ab-1-Dxd and Ab-1-vcMMAE ADCs (ADC-1 and ADC-4, respectively) demonstrate significant anti-tumor activity in NCI-N87 gastric carcinoma model.

FIG. 25 shows the antitumor effect of Ab-1-vcMMAE (ADC-4) and Ab-1-Dxd (ADC-1) ADCs in the OVCAR3 ovarian carcinoma xenograft model. ADCs were given intravenously once every 5 days for 2 doses (arrows). Both Ab-1-Dxd (ADC-1) and Ab-1-vcMMAE (ADC-4) demonstrate a significant anti-tumor activity in the OVCAR3 ovarian carcinoma model.

### DETAILED DESCRIPTION

The present disclosure relates to anti-mesothelin (anti-MSLN) antibody drug conjugates (ADCs), and methods for the treatment of cancer using the same.

In some aspects, the present disclosure provides anti-MSLN antibody conjugates with cytotoxic agents and pharmaceutical compositions that include such conjugates. The benefits provided by the present disclosure are broadly applicable in the field of antibody therapeutics. In particular, the conjugates, and pharmaceutical compositions of the disclosure may be useful in treating a MSLN+ cancer, alone or in combination with other cancer therapeutic agents.

Mesothelin (MSLN) is a 40 kD glycosylphosphatidylinositol (GPI)-anchored membrane protein that is expressed on some normal mesothelial cells including those of the pleura, peritoneum and pericardium (see, for example, Chang et al. Int J Cancer 50: 373, 1992). While the function of MSLN in normal cells is unknown, the protein has been reported to be a tumor-differentiation antigen (see, for example, Chang et al. PNAS 93: 136, 1996) that is overexpressed on multiple solid tumors including ovarian carcinoma (see, for example, Chang et al. Int J Cancer 50: 373, 1992), mesothelioma (see, for example, Chang et al. Int J Cancer 50: 373, 1992), colon carcinoma (see, for example, Ordonez, Am J Surg Path 27: 1418, 2003), pancreatic carcinoma (see, for example, Hassan et al. Am J Clin Path 124: 838, 2005), lung adenocarcinoma (see, for example, Thomas et al. Oncotarg 6: 11694, 2015), extrahepatic bile duct cancer (see, for example, Ordonez, Am J Surg Path 27: 1418, 2003), gastric cancer (see, for example, Ito et al. Oncol Rep 31: 27, 2014; Einama et al. WJGP 107: 137, 2016) and triple-negative breast cancer (see, for example, Tozbikian et al. PLOS One 9: e114900, 2015; Byelogu et al. Biomed Pharm 70: 190, 2015). High expression of MSLN was recently found on blasts from newly diagnosed and relapsed AML patients (see, for example, Kaeding et al. Blood Adv 5: 2350, 2021). Elevated expression of MSLN has been correlated with worse prognosis for patients with ovarian cancer, cholangiocarcinoma, lung adenocarcinoma, triple-negative breast cancer, and resectable pancreatic adenocarcinoma (see, for example, Hassan, et al. JCO 34: 4171, 2016). The limited expression of MSLN on normal human tissues and its high expression in many cancers make it an attractive candidate for cancer therapy (see, for example, Pastan et al. Cancer Res 74: 2907, 2014).

In preclinical studies, MSLN has been shown to play a role in promoting the survival, growth, adhesion, and migration of tumor cells as well as in chemoresistance (see, for example, Faust, Cancers (Basel) 14: 1550, 2022) through activation of multiple signaling pathways such as STAT3/MAPK/ERK/JNK, increased production of IL-6 cytokine, and decreased signaling of apoptotic pathways (see, for example, Bharawaj et al. Carcinogenesis 32: 1013, 2011; Tang et al. Anticancer Agents Med Chem 13: 276, 2013). MSLN mediates these activities through interactions with the glycoprotein CA125/MUC16 (see, for example, Rump et al. JBC 279: 9190, 2004) or with cell surface MSLN molecules on adjacent cells (see, for example, Chang et al. Biochem J 442: 292, 2012).

The MSLN gene encodes a precursor protein of 71-kDa that is processed to yield a 31-kDa shed protein named megakaryocyte-potentiating factor (MPF) and the 40-kDa cell bound fragment MSLN. The GPI-linked mature MSLN can also be shed from the cell through the action of the tumor necrosis factor α-converting enzyme protease (TACE/ADAM17) (see, for example, Zhang, Yujian, et al. Cancer Res 71: 5915, 2011) or members of the MMP or BACE family of proteases (Liu et al. Commun Biol 3: 728, 2022), leaving behind a small fragment at the membrane. Shed MSLN, also called soluble MSLN, creates a pool of free antigen in blood, ascites fluid or pleural fluid that can serve as a prognosis biomarker (see, for example, Hassan et al. Clin Can Res 12: 447, 2006; Inami et al. Onco Rep 20: 1375, 2008; Burt et al. Ann Thor Surg 104: 1679, 2017; and Grosso et al. Diagnostics (Basel) 11: 2015, 2021). Studies have shown that soluble MSLN can interact with cell surface MSLN to trigger the activation of signaling pathways to promote tumor cell proliferation and invasion (see, for example, Chang et al. Biochem J 442: 292, 2012). Serum MSLN has been approved by the US Food and Drug Administration as a diagnostic biomarker in malignant mesothelioma (see, for example, Chopra, A. ¹¹¹In-Labeled CHX-A”-DTPA-conjugated MORAb-009, a chimeric monoclonal antibody directed against mesothelin (2012) In: Molecular Imaging and Contrast Agent Database (MICAD)). It has been reported that the normal range of MSLN is up to 9 ng/mL, while the serum MSLN levels in patients with mesothelioma can reach over 200 ng/mL (see, for example, Hassan, R., Clin Cancer Res 12: 447, 2006). The serum MSLN in patients with ovarian cancer can be 100 ng/mL or more (see, for example, Hassan, R., Clin Cancer Res 12: 447, 2006).

A number of MSLN-targeting therapeutic approaches have been, and currently are, in development (see, for example, Faust, Cancers (Basel) 14: 1550, 2022), with limited success to date. One possible explanation is that many of the antibodies and corresponding antibody-based therapies bind the distal region of MSLN which recognizes CA125/MUC1. The activities of such agents have been shown to be impacted by circulating levels of soluble MSLN (see, for example, Zhang et al. Cancer Res 71: 5915, 2011; Awuah et al. Mol Can Ther 15: 1648, 2016; Hatterer et al. mAbs 12: e1739408, 2020; and Le et al. Clin Cancer Res 27:5718, 2021).

To circumvent this, one strategy is to develop and evaluate antibodies that bind to the more proximal membrane region of MSLN. Targeting of MSLN using such an antibody armed with a cytotoxic agent (for example, an antibody drug conjugate (ADC)) may provide a means of selectively attacking the cancer cells and sparing normal tissues. In some embodiments, the present disclosure provides ADCs generated from a fully human antibody (for example, Ab-1) whose binding is minimally affected by soluble MSLN and which binds specifically to and kills MSLN-expressing tumor cells. In some embodiments, Ab-1 conjugated to an auristatin or topoisomerase I payload may demonstrate significant antitumor activities in mouse xenograft models.

The present disclosure, in part, provides conjugates that include chimeric and/or human monoclonal antibodies against MSLN. In some embodiments, the anti-MSLN antibody, or antigen-binding portion thereof, comprises the antibody Ab-1 (also referred to as W305044-1.100.1-p3-uIgG1KV320 and W305044 mAb), or an antigen-binding portion thereof, as described, for example, by sequences in Table 1. In some embodiments, methods for validating the function of anti-MSLN antibodies in vitro and in vivo are provided. In some embodiments, methods of treating a subject having cancer by administering the anti-MSLN conjugates as disclosed herein are provided. In some embodiments, methods of improving treatment outcome in a subject receiving immunotherapy and/or chemotherapy for a MSLN+ cancer by administering the anti-MSLN conjugates as disclosed herein are provided.

In some aspects, the present disclosure provides an anti-MSLN conjugate that includes an isolated antibody or an antigen-binding portion thereof that binds MSLN, such as human MSLN and/or cynomolgus monkey (cyno) MSLN.

In some embodiments, the isolated antibody, or the antigen-binding portion thereof, or anti-MSLN conjugate, may exhibit at least one of the following properties:
(a) specific binding to cell surface expressed human MSLN protein and/or cyno MSLN protein;
(b) minimal or low interference with, or minimally affected by, circulating soluble MSLN proteins;
(c) binding to MSLN that involves MSLN region III;
(d) good target internalization and cytotoxic activity;
(e) good thermal stability; and
(f) containing little oligomeric form and having low aggregation propensity.

The anti-MSLN conjugates and methods of the present disclosure may, among other benefits, include anti-MSLN antibodies that bind more selectively to the proximal membrane region of MSLN, as compared to other anti-MSLN antibodies and anti-MSLN conjugates. In some embodiments, the anti-MSLN antibodies, or antigen-binding portion thereof, and anti-MSLN conjugates of the present disclosure, may bind an epitope on MSLN different from known anti-MSLN antibodies. In turn, this may provide for improved binding to MSLN, for example, binding that is minimally affected by soluble MSLN, as compared to other anti-MSLN conjugates. In some embodiments, targeting of MSLN using the anti-MSLN conjugates and methods of the present disclosure (for example, that include an anti-MSLN antibody armed with a cytotoxic agent), may provide for improved selective attacking of cancer cells and sparing of normal tissues, as compared to other anti-MSLN conjugates. In some embodiments, the anti-MSLN conjugates of the present disclosure, may bind specifically to and kill MSLN-expressing tumor cells. In some embodiments, the anti-MSLN conjugates of the present disclosure may demonstrate improved antitumor activities (for example, when conjugated to an auristatin or topoisomerase I payloads), as compared to other anti-MSLN conjugates.

The following sections provide a detailed description of conjugates, for example ADCs, that include a binding agent, at least one linker attached to the binding agent; and at least one cytotoxic agent attached to the linker, and methods related to such conjugates. In some embodiments, the binding agent is an antibody (for example, an anti-MSLN antibody), or an antigen-binding portion thereof. Prior to setting forth this disclosure in more detail, it may be helpful to an understanding thereof to provide definitions of certain terms to be used herein. Additional definitions are set forth throughout this disclosure.

Unless stated otherwise, or implicit from context, the following terms and phrases have the meanings provided below. The definitions are provided to aid in describing particular embodiments and are not intended to limit the claimed invention. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

The use of the alternative (for example, “or”) should be understood to mean either one, both, or any combination thereof of the alternatives. As used throughout the disclosure, the terms “have”, “include” and “comprise” are used synonymously.

“Optional” or “optionally” means that the subsequently described element, component, event, or circumstance may or may not occur, and that the description includes instances in which the element, component, event, or circumstance occurs and instances in which they do not.

The phrase “at least one of” when followed by a list of items or elements refers to an open-ended set of one or more of the elements in the list, which may, but does not necessarily, include more than one of the elements.

In the present description, the term “about” means ±20% of the indicated range, value, or structure, unless otherwise indicated.

Throughout the disclosure, any concentration range, percentage range, ratio range, or integer range is to be understood to include any value (including integers or fractions) or subrange within the recited range unless otherwise indicated.

The term “comprise” (and similar terms such as “comprising of” and “comprised of”) means the presence of the stated features, integers, steps, or components as referred to in the claims, but that it does not preclude the presence or addition of one or more other features, integers, steps, components, or groups thereof. The term “consisting essentially of” limits the scope of a claim to the specified materials or steps and those that do not materially affect the basic and novel characteristics of the claimed invention.

The term “consisting of” refers to compositions, methods, and respective components thereof as described herein, which are exclusive of any element not recited in that description of the embodiment.

The term “statistically significant” or “significantly” refers to statistical significance and generally means a two standard deviation (2SD) difference, above or below a reference value.

The terms “decrease,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

The word “substantially” does not exclude “completely”; for example, a composition which is “substantially free” from Y may be completely free from Y. Where necessary, the word “substantially” may be omitted from definitions provided herein.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “isolated” or “partially purified” as used herein refer in the case of a nucleic acid, polypeptide or protein, to a nucleic acid, polypeptide or protein separated from at least one other component (for example, nucleic acid or polypeptide or protein) that is present with the nucleic acid, polypeptide or protein as found in its natural source and/or that would be present with the nucleic acid, polypeptide or protein when expressed by a cell, or secreted in the case of secreted polypeptides and proteins. A chemically synthesized nucleic acid, polypeptide or protein, or one synthesized using in vitro transcription/translation, is considered “isolated.” The terms “purified” or “substantially purified” refer to an isolated nucleic acid, polypeptide or protein that is at least 95% by weight the subject nucleic acid, polypeptide or protein, including, for example, at least 96%, at least 97%, at least 98%, or at least 99% or more. The term “isolated,” as used herein, may also refer to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (for example, an isolated antibody that specifically binds a MSLN protein is substantially free of antibodies that specifically bind antigens other than MSLN proteins). An isolated antibody that specifically binds a human MSLN protein may, however, have cross-reactivity to other antigens, such as MSLN proteins from other species. Moreover, an isolated antibody can be substantially free of other cellular material and/or chemicals.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of protein amino acids, including modified amino acids (for example, phosphorylated, glycated, glycosylated, and the like) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

The term “amino acid,” as used herein, generally refers to a naturally occurring or non-naturally occurring amino acid (for example, an amino acid analogue). The non-naturally occurring (or unnatural) amino acid may be an engineered or synthesized amino acid. An amino acid may contain a “side chain”, which may differentiate amino acid types from one another.

The terms “amino acid sequence,” “peptide sequence,” and “polypeptide sequence,” as used herein, generally refer to a sequence of at least two amino acids or amino acid analogs that are covalently linked (for example, by a peptide (amide) bond or an analog of a peptide bond). A peptide sequence may refer to a complete sequence or a portion of a sequence. For example, a peptide sequence may contain gaps, positions with unknown identities, or positions that can accommodate distinct species.

As used herein, the term “side chain” generally refers to a structure attached to an alpha carbon (attaching an amine and a carboxylic acid group of an amino acid) that may be unique to each type of amino acid. A side chain may have a certain shape, size, charge, reactivity, or a combination thereof. A side chain may contain a basic moiety (for example, the guanidino group in arginine), an acidic moiety (for example, the carboxylic acid in aspartic acid), a polar moiety (for example, the hydroxyl groups in serine, threonine, and tyrosine), a hydrophobic moiety (for example, the alkyl groups in leucine, isoleucine, alanine, and valine), or any combination thereof. In some cases, an amino acid contains more than one side chain. The side chain may be or include hydrogen, an alkyl group, a hydroxyl group, an aryl group, a heteroaryl group, a carboxylic acid, an amide, an amine, a guanidine, a thiol, a thioether, a selenol, or any combination thereof. In some instances, the side chain is a hydrogen (an amino acid with a hydrogen side chain may be, for example, glycine).

As used herein, the term “vector,” refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

As used herein, the term “host cell,” refers to a cellular system which can be engineered to generate proteins, protein fragments, or peptides of interest. Host cells include, without limitation, cultured cells, for example, mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell but are still included within the scope of the term “host cell.”

As used herein, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “sequence variant” refers to any sequence having one or more alterations in comparison to a reference sequence, whereby a reference sequence is any of the sequences listed in the sequence listing, for example, SEQ ID NO:1 to SEQ ID NO:27. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, for example, any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, for example, serine and threonine, with another; substitution of one acidic residue, for example, glutamic acid or aspartic acid, with another; replacement of one amide-containing residue, for example, asparagine and glutamine, with another; replacement of one aromatic residue, for example, phenylalanine and tyrosine, with another; replacement of one basic residue, for example, lysine, arginine, and histidine, with another; and replacement of one small amino acid, for example, alanine, serine, threonine, methionine, and glycine, with another.

As used herein, the term “immunogenicity”, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity may be the most important property of an antigen. Whether an antigen can successfully induce the generation of an immune response in a host depends on three factors, properties of an antigen, reactivity of a host, and immunization means.

As used herein, the term “transfection,” refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, for example, Graham et al., 1973, Virology 52:456; Sambrook J. & Russell D. Molecular Cloning: A Laboratory Manual, 3rd ed., Cold Spring Harbor Laboratory Press, Cold Spring Harbor, N.Y. (2000); Davis et al., 1986, Basic Methods in Molecular Biology, Elsevier; Chu et al, 1981, Gene 13:197. In some embodiments of the invention, human MSLN gene may be transfected into CHO cells.

As used herein, the term “SPR” or “surface plasmon resonance” refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. Ann Biol Clin 51:19-26, 1993; Jansson, U., et al. Biotechniques 11:620-627, 1991; Johnsson, B., et al. J Mol Recognit 8:125-131, 1995; and Johnnson, B., et al. Anal Biochem 198:268-277, 1991.

As used herein, the terms “fluorescence-activated cell sorting” or “FACS” refer to a specialized type of flow cytometry. This provides a method for sorting a heterogeneous mixture of biological cells into two or more containers, one cell at a time, based upon the specific light scattering and fluorescent characteristics of each cell (see, for example, FlowMetric. “Sorting Out Fluorescence Activated Cell Sorting”. Retrieved 2017 Nov. 9.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The terms “subject” and “patient” may be used interchangeably and include mammals such as humans and non-human primates, as well as rabbits, rats, mice, goats, pigs, and other mammalian species. The term does not necessarily indicate that the subject has been diagnosed with a particular disease, but typically refers to an individual under medical supervision.

As used herein, the term “conjugated” generally refers to at least two molecules, or moieties, being linked together. The molecules or moieties may be linked together by a chemical bond.

As used herein, an “epitope” refers to the amino acids typically bound by an immunoglobulin VH/VL pair, such as the antibodies and binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody or other binding agent, and thus represent the target of specificity of an antibody, antigen binding portion thereof or other immunoglobulin-based binding agent. In the case of a single domain antibody, an epitope represents the unit of structure bound by a variable domain in isolation.

As used herein, “MSLN”, or “mesothelin”, is a 40 kD glycosylphosphatidylinositol (GPI)-anchored membrane protein that is expressed in normal mesothelial cells. Shed or soluble MSLN may also be referred to as SMRP. It is reported to be overexpressed on multiple solid tumors including ovarian carcinoma, mesothelioma, colon carcinoma, pancreatic carcinoma, lung adenocarcinoma, extrahepatic bile duct cancer, gastric cancer and triple-negative breast cancer, among other cancers. MSLN polypeptides include, but are not limited to, those having the amino acid sequence set forth in NCBI Ref Seq. NP_001170826, (SEQ ID NO:15), NP_005814 (SEQ ID NO:16), and NP_037536 (SEQ ID NO:17); these sequences are incorporated by reference herein. An example of the amino acid sequence of human mesothelin may also be found at UniProtKB-Q13421-3. The term “mesothelin” or “MSLN” as used herein may also refer to any native MSLN from any vertebrate source, including mammals such as primates (for example, humans) and rodents (for example, mice and rats), as well as variants, homologs and fragments (for example, extracellular domain) thereof. The term also encompasses naturally occurring variants (for example, splice variants or allelic variants), precursors and mature forms of MSLN. In cell-surface mature mesothelin, 3 distinct domains have been proposed, Regions I (residues 296-390), II (residues 391-486), and III (residue 487-598). Region I at the N-terminal end of cell-surface mesothelin is presumed to be located far from the cell membrane and more accessible to antibodies, while Region III is considered as a membrane proximal region.

As used herein, “specifically binds” refers to the ability of a binding agent (for example, an antibody, or antigen binding portion thereof) described herein to bind to a target, such as MSLN, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody or other binding agent and the concentration of target polypeptide. The person of ordinary skill in the art can determine appropriate conditions under which the antibodies and other binding agents described herein selectively bind to MSLN using any suitable methods, such as titration of a binding agent in a suitable cell binding assay. A binding agent specifically bound to MSLN is not displaced by a non-similar competitor. In certain embodiments, an anti-MSLN antibody, or antigen-binding portion thereof, is said to specifically bind to MSLN when it preferentially recognizes its target antigen, MSLN, in a complex mixture of proteins and/or macromolecules.

In some embodiments, an anti-MSLN antibody, or antigen-binding portion thereof, or other binding agent or conjugate, as described herein, specifically binds to a MSLN polypeptide with a dissociation constant (K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, an anti-MSLN antibody, or antigen-binding portion thereof, or other binding agent or conjugate, as described herein, specifically binds to a MSLN polypeptide with a dissociation constant (K_{D}) of from about 10⁻⁵ M to 10⁻⁶ M. In some embodiments, an anti-MSLN antibody, or antigen-binding portion thereof, or other binding agent or conjugate, as described herein, specifically binds to a MSLN polypeptide with a dissociation constant (K_{D}) of from about 10⁻⁶ M to 10⁻⁷ M. In some embodiments, an anti-MSLN antibody, or antigen-binding portion thereof, or other binding agent or conjugate, as described herein, specifically binds to a MSLN polypeptide with a dissociation constant (K_{D}) of from about 10⁻⁷ M to 10⁻⁸ M. In some embodiments, an anti-MSLN antibody, or antigen-binding portion thereof, or other binding agent or conjugate, as described herein specifically binds to a MSLN polypeptide with a dissociation constant (K_{D}) of from about 10⁻⁸ M to 10⁻⁹ M. In some embodiments, an anti-MSLN antibody, or antigen-binding portion thereof, or other binding agent or conjugate, as described herein specifically binds to a MSLN polypeptide with a dissociation constant (K_{D}) of from about 10⁻⁹ M to 10⁻¹⁰ M. In some embodiments, an anti-MSLN antibody, or antigen-binding portion thereof, or other binding agent or conjugate, as described herein specifically binds to a MSLN polypeptide with a dissociation constant (K_{D}) of from about 10⁻¹⁰ M to 10⁻¹¹ M. In some embodiments, an anti-MSLN antibody, or antigen-binding portion thereof, or other binding agent or conjugate, as described herein specifically binds to a MSLN polypeptide with a dissociation constant (K_{D}) of from about 10⁻¹¹ M to 10⁻¹² M. In some embodiments, an anti-MSLN antibody, or antigen-binding portion thereof, or other binding agent or conjugate, as described herein, specifically binds to a MSLN polypeptide with a dissociation constant (K_{D}) of less than 10⁻¹² M.

### I. Antibodies

Provided herein are MSLN-binding antibodies (also referred to as anti-MSLN antibodies), and antigen binding portions thereof, that specifically bind to mesothelin (MSLN). Also provided herein are conjugates of anti-MSLN antibodies, and antigen binding portions, and cytotoxic agents (also referred to as antibody drug conjugates (ADCs) or anti-MSLN conjugates). In some embodiments, the anti-MSLN conjugates may reduce the number of MSLN+ cancer cells in a subject.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, for example, Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

As used herein, the term “antibody” refers to immunoglobulin molecules and immunologically active portions of immunoglobulin molecules, i.e., molecules that contain an antigen binding site that specifically binds to an antigen. The term generally refers to antibodies comprised of two immunoglobulin heavy chain variable regions and two immunoglobulin light chain variable regions including full length antibodies (having heavy and light chain constant regions) and antigen-binding portions thereof; including, for example, an intact monoclonal antibody, a Fab, a Fab′, a F(ab′)², a Fv, a disulfide linked Fv, a scFv, a scFab, a single domain antibody (dAb), a diabody, a multi-specific antibody, a dual specific antibody, a bi-specific antibody, and single chain antibodies (see, for example, Huston et al., Proc. Natl. Acad. Sci. U.S.A., 85, 5879-5883 (1988) and Bird et al., Science 242, 423-426 (1988), which are incorporated herein by reference). An antibody can include, for example, polyclonal, monoclonal, and genetically engineered antibodies, and antigen binding fragments thereof. An antibody can be, for example, murine, chimeric, humanized, heteroconjugate, bi-specific, diabody, tribody, or tetrabody.

In certain embodiments, an antibody or antigen-binding fragment of the present disclosure may be monospecific (e.g., binds to a single epitope) or may be multispecific (e.g., binds to multiple epitopes and/or target molecules). Antibodies and antigen binding fragments may be constructed in various formats. Exemplary antibody formats disclosed in Spiess et al., Mol. Immunol. 67(2):95, 2015, and in Brinkmann and Kontermann, mAbs 9(2):182-212, 2017, which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent Hcabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, Kk-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

Each heavy chain is typically composed of a variable region (abbreviated as VH) and a constant region. The heavy chain constant region may include three domains CH1, CH2 and CH3 and optionally a fourth domain, CH4. Each light chain is typically composed of a variable region (abbreviated as VL) and a constant region. The light chain constant region is a CL domain, which may be, for example, a kappa CL domain or a lambda CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged in N terminal to C terminal direction in the following order: FR1, CDR1, FR2, CDR2, FR3, CDR3, and FR4. This structure is well known to those skilled in the art. CDR and FR sequences may be determined by any of several different numbering schemes, including Kabat, Chothia, AbM, Contact, IMGT, North, Martin (also known as Enhanced Chothia) and/or Aho, or by a combination of two or more of the foregoing. In some embodiments, the CDRs and FRs are defined by Kabat.

In some embodiments, an antigen binding portion comprises a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3).

The amino acid sequences of the VH CDRs and VL CDRs, VH and VL, and constant regions of exemplary anti-MSLN antibodies of the present disclosure are set forth in Table 1. The phrase “wherein the CDRs of the heavy or light chain variable regions are not modified” refers to these VH and VL CDRs (for example, SEQ ID NOs:1-6), which do not have amino acid substitutions, deletions or insertions.

As used herein, an “antigen-binding portion” or “antigen-binding fragment” of an anti-MSLN antibody refers a region of an antibody molecule that specifically binds to an antigen. In some embodiments, the antigen-binding portion refers to the portions of an anti-MSLN antibody as described herein having the VH and VL sequences of the anti-MSLN antibody (for example, set forth in SEQ ID NOs:7 and 8, optionally modified as described herein). In accordance with the term “antigen-binding portion” of an antibody, examples of antigen binding portions include a Fab, a Fab′, a F(ab′)², a Fv, a disulfide linked Fv, a scFv, a single domain antibody (dAb), a diabody, heavy chain antibody (hcAb), VHH, VNAR, nanobody, and single chain antibodies. As used herein, the terms Fab, F(ab′)2 and Fv refer to the following: (i) an Fab fragment, for example, a monovalent fragment composed of the VL, VH, CL and CH1 domains; (ii) an F(ab′)2 fragment, for example, a bivalent fragment comprising two Fab fragments linked to one another in the hinge region via a disulfide bridge; and (iii) an Fv fragment composed of the VL and VH domains of an anti-MSLN antibody. Although the two domains of the Fv fragment, namely VL and VH, are encoded by separate coding regions, they may further be linked to one another using a synthetic linker, for example, a poly-G₄S amino acid sequence (‘(G₄S)_{n’} wherein n=1 to 5; SEQ ID NOs:18-22), making it possible to prepare them as a single protein chain in which the VL and VH regions combine in order to form monovalent molecules (known as single chain Fv (ScFv)). The term “antigen-binding portion” of an antibody is also intended to include such single chain antibodies. Other forms of single chain antibodies such as “diabodies” are likewise included here. Diabodies are bivalent, bi-specific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. Proc Natl Acad Sci USA 90:6444-6448, 1993; Poljak, R. J, et al. Structure 2:1121-1123), 1994.

The term “monoclonal antibody” or “mAb”, as used herein, refer to a preparation of antibody molecules of single molecular composition. A monoclonal antibody displays a single binding specificity and affinity for a particular epitope.

The term “chimeric antibody”, as used herein, refers to an antibody in which the variable region sequences are derived from one species and the constant region sequences are derived from another species, such as an antibody in which the variable region sequences are derived from human germline (for example, by immunizing human germline engineered rats) and the constant region sequences are derived from rat germline, or an antibody in which the variable region sequences are derived from non-human germline and the constant region sequences are derived from human. The chimeric antibodies as disclosed herein with non-human IgG constant regions may be IgG converted to obtain human antibodies.

The term “human antibody”, as used herein, is intended to include antibodies whose variable regions (including both the framework and CDR regions) and constant regions are derived from human germline immunoglobulin sequences. The human antibodies can include amino acid residues not encoded by human germline immunoglobulin sequences (for example, mutations introduced by random or site-specific mutagenesis in vitro or by somatic mutation in vivo).

The term “PTM” or “post-translational modification”, as used herein, refers to a process that occurs to one or more amino acids on a protein (for example, an antibody) after the protein has been translated. Proteins are usually produced by ribosomes that translate mRNA into polypeptide chains and then form mature protein products via PTM. PTM process includes phosphorylation, glycosylation, ubiquitination, S-nitrosylation, methylation, N-acetylation and lipidation. Preferably, potential PTM sites are removed during antibody optimization to avoid structural and functional heterogeneity brought by PTM process.

The term “binding affinity” is herein used as a measure of the strength of a non-covalent interaction between two molecules, for example, an antibody or antigen-portion thereof, and an antigen. Binding affinity between two molecules may be quantified by determination of the dissociation constant (K_{D}). In turn, K_{D} can be determined by measurement of the kinetics of complex formation and dissociation using, as a nonlimiting example, the surface plasmon resonance (SPR) method (Biacore™). The rate constants corresponding to the association and the dissociation of a monovalent complex are referred to as the association rate constants kₐ (or kₒₙ) and dissociation rate constant k_{d} (or k_{off}), respectively. The term kₐ (or kₒₙ) refers to the association rate of a particular antibody-antigen interaction, whereas the term k_{d} (or k_{off}) refers to the dissociation rate of a particular antibody-antigen interaction. K_{D} is related to kₐ and k_{d} through the equation K_{D}=k_{d}/kₐ or k_{off}/kₒₙ. The value of the dissociation constant can be determined directly by well-known methods, and can be computed even for complex mixtures by methods such as those, for example, set forth in Caceci et al. (1984, Byte 9: 340-362). The binding kinetics and binding affinity of the antibody also can be assessed by standard assays known in the art or as described in the Example section below.

The term “EC₅₀” as used herein, which is also termed as “half maximal effective concentration” refers to the concentration of a drug, antibody or toxicant which induces a response halfway between the baseline and maximum after a specified exposure time. In the context of the disclosure, EC₅₀ may be expressed in the unit of “nM” or “M”.

An immunoglobulin “constant region” refers to a heavy or light chain constant region. The constant region provide the general framework of the antibody and may not be involved directly in binding the antibody to an antigen, but can be involved in various effector functions, such as participation of the antibody in antibody-dependent cellular cytotoxicity (ADCC), ADCP (antibody-dependent cellular phagocytosis), CDC (complement-dependent cytotoxicity) and complement fixation, binding to Fc receptors (e.g., CD16, CD32, FcRn), greater in vivo half-life relative to a polypeptide lacking an Fc region, protein A binding, and perhaps even placental transfer (see, for example, Capon et al., Nature 337:525, 1989). As used throughout the disclosure, “Fc region” refers to the heavy chain constant region segment of the Fc fragment (the “fragment crystallizable” region or Fc region) from an antibody, which may include one or more constant domains, such as CH2, CH3, CH4, or any combination thereof. In some embodiments, an Fc region may include the CH2 and CH3 domains of an IgG, IgA, or IgD antibody, or the CH3 and CH4 domains of an IgM or IgE antibody.

Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAQ1, and IgA2. The heavy-chain constant regions (Fc) that corresponds to the different classes of immunoglobulins can be α, δ, ε, γ, and μ, respectively. The light chains can be one of either kappa (or κ) and lambda (or λ). Allotypic variants of immunoglobulin constant regions also exist, e.g., for IgG1, IgG2, IgG3, and IgA heavy chains, and Ig kappa light chain.

In some embodiments, a constant region may have an IgG1 isotype. In some embodiments, a constant region may have an IgG2 isotype. In some embodiments, a constant region may have an IgG3 isotype. In some embodiments, a constant region may have an IgG4 isotype. In some embodiments, an Fc region may have a hybrid isotype comprising constant domains from two or more isotypes. In some embodiments, an immunoglobulin constant region may be an IgG1 or IgG4 constant region. In some embodiments, a constant region may be an IgG1 allotypic variant (e.g., G1m1 or nG1m1).

Furthermore, an anti-MSLN antibody or an antigen-binding portion thereof may be part of a larger binding agent formed by covalent or noncovalent association of the antibody or antibody portion with one or more other proteins or peptides. Relevant to such binding agents are the use of the streptavidin core region in order to prepare a tetrameric scFv molecule (see, for example, Kipriyanov, S. M., et al., Human Antibodies and Hybridomas 6:93-101, 1995) and the use of a cysteine residue, a marker peptide and a C-terminal polyhistidinyl peptide, for example, a hexahistidinyl tag in order to produce bivalent and biotinylated scFv molecules (see, for example, Kipriyanov, S. M., et al. Mol Immunol 31:10471058, 1994).

As to the VH and VL amino acid sequences, one of skill in the art will recognize that individual substitutions, deletions or additions (insertions) to a nucleic acid encoding the VH or VL, or amino acids in polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to MSLN.

In some aspects, the disclosure provides an antibody or antigen-binding portion thereof capable of binding MSLN (such as human, mouse or cyno MSLN) with sufficient affinity such that it substantially or completely inhibits the biological activity of MSLN.

In some embodiments, the anti-MSLN antibody as disclosed herein is an antibody produced in human germline engineered rats (for example, OMT rats) immunized with a MSLN protein. In some embodiments, the anti-MSLN antibody as disclosed herein is a chimeric antibody. In some embodiments, the anti-MSLN antibody as disclosed herein is a fully human antibody. The antigen-binding portion of the antibody may be and not limited to, a Fab, a Fab′, a F(ab′)2, a single chain variable fragment (scFv), or a diabody.

The antibodies, antigen-binding portions thereof, binding agents or conjugates of the present disclosure may be characterized by particular functional features or properties. In some embodiments, the isolated antibody, or the antigen-binding portion thereof, binding agents or conjugates, may have one or more of the following properties:
(a) specific binding to cell surface expressed human MSLN protein and/or cyno MSLN protein, for example, with an EC₅₀ of about 1 nM as measured by FACS, while not binding to rodent MSLN protein;
(b) binding to cell surface expressed MSLN protein may be minimally interfered with or affected by circulating soluble MSLN proteins;
(c) binding to MSLN involves MSLN region III;
(d) good target internalization and cytotoxic activity, for example, the antibody shows an IC₅₀ of no more than 1 nM, no more than 0.5 nM, no more than 0.3 nM, or no more than 0.1 nM as measured in an internalization assay;
(e) good thermal stability; and
(f) containing little oligomeric form and having low aggregation propensity.

### Binding Affinity of Anti-MSLN Antibodies

The antibodies, antigen-binding portions thereof, binding agents, or conjugates as disclosed herein may bind to human and cynomolgus monkey MSLN with sufficiently high affinity. The binding of an antibody, antigen-binding portion thereof, binding agent, or conjugate of the disclosure to MSLN may be assessed using one or more techniques well established in the art, for instance, ELISA. The binding specificity of an antibody of the disclosure may also be determined by monitoring binding of the antibody to cells expressing an MSLN protein, for example, flow cytometry. For example, an antibody can be tested by a flow cytometry assay in which the antibody is reacted with a cell line that expresses human MSLN, such as CHOK1 cells that have been transfected to express human MSLN on their cell surface. Cells or cell lines that naturally express MSLN protein, such as OVCAR3 or NCI-N87 cells, may be used. Additionally or alternatively, the binding of the antibody, including the binding kinetics (for example, K_{D} value) can be tested in BIAcore binding assays or FACS affinity tests.

In some embodiments, the antibody, antigen-binding portion thereof, binding agent, or conjugate, may bind to cell surface expressing human MSLN with an EC₅₀ of no more than 3 nM, no more than 2 nM, no more than 1.5 nM, or no more than 1.1 nM, as measured by FACS. In some embodiments, the antibody, antigen-binding portion thereof, binding agent, or conjugate, may bind to cell surface expressing cyno MSLN with an EC₅₀ of no more than 2.5 nM, no more than 2.4 nM, no more than 2.3 nM, or no more than 2.2 nM, as measured by FACS. In some embodiments, the antibody, antigen-binding portion thereof, binding agent, or conjugate, thereof may bind to human MSLN with an EC₅₀ of no more than 2 nM, no more than 1.5 nM, no more than 1 nM, or no more than 0.9 nM, as measured by ELISA. In some further embodiments, the antibody, antigen-binding portion thereof, binding agent, or conjugate, may bind to human MSLN Region III with an EC₅₀ of less than 1 nM, as measured by ELISA.

In some embodiments, the antibody, antigen-binding portion thereof, binding agent, or conjugate, can bind to NCI-N87 cells with an EC50 of no more than 2 nM, no more than 1.5 nM, or no more than 1.1 nM, as measured by FACS. In some embodiments, the antibody or antigen-binding portion thereof can bind to OVCAR3 cells with an EC50 of no more than 2 nM, no more than 1.5 nM, or no more than 1.2 nM, as measured by FACS.

The binding between the antibody, antigen-binding portion thereof, binding agent, or conjugate, to cell surface MSLN may be minimally interfered with or affected by soluble MSLN. In some embodiments, at a concentration of 0.5 μg/mL soluble MSLN, the antibody, antigen-binding portion thereof, binding agent, or conjugate, can bind to human MSLN engineered cells with an EC₅₀ of no more than about 1.5 nM, as measured by FACS. In some embodiments, at a concentration of 0.5 μg/mL soluble MSLN, the antibody, antigen-binding portion thereof, binding agent, or conjugate, can bind to NCI-N87 cells with an EC₅₀ of no more than about 1.1 nM, as measured by FACS. In some embodiments, at a concentration of 0.5 μg/mL soluble MSLN, the antibody, antigen-binding portion thereof, binding agent, or conjugate, can bind to OVCAR3 cells with an EC50 of no more than about 1 nM, no more than about 0.6 nM, or no more than about 0.4 nM, as measured by FACS.

In some embodiments, the antibodies, antigen-binding portion thereof, binding agent, or conjugate, may bind an epitope on MSLN different from known anti-MSLN antibodies. In some embodiments, the antibody, antigen-binding portion thereof, binding agent, or conjugate, can bind to MSLN region III located proximally to cell membrane. In some embodiments, the antibody, antigen-binding portion thereof, binding agent, or conjugate, of the disclosure binds to cell surface expressed human MSLN with a sufficient affinity while minimally interfered by the presence of surrounding soluble MSLNs. Without wishing to be bound by theory, it is believed that by binding to region III which is closer to the cell surface than region I, the antibody, antigen-binding portion thereof, binding agent, or conjugate, may be less affected by circulating soluble MSLN.

In some embodiments, the antibody, antigen-binding portion thereof, binding agent, or conjugate, may be capable of specifically binding to human MSLN and cynomolgus monkey MSLN.

### Thermal Stability

Each antibody, antigen-binding portion thereof, binding agent, or conjugate, will have a characteristic melting temperature. In some embodiments, a higher melting temperature may indicate a greater overall stability in vivo (see, for example, Krishnamurthy R and Manning M C Curr Pharm Biotechnol 3:361-71, 2002). Generally, it is preferred that the Tm1 (the temperature of initial unfolding) be greater than 60° C., preferably greater than 65° C.

The melting point of an antibody can be measured using differential scanning calorimetry (see, for example, Chen et al Pharm Res 20:1952-60, 2003; Ghirlando et al Immunol Lett 68:47-52, 1999) or circular dichroism (see, for example, Murray et al. J. Chromatogr Sci 40:343-9, 2002). In some embodiments, the antibody, antigen-binding portion thereof, binding agent, or conjugate, as disclosed herein has minimal or low aggregation effects. Aggregation effects may lead to the triggering of an unwanted immune response and/or altered or unfavorable pharmacokinetic properties. Aggregation can be measured by several techniques, including size-exclusion column (SEC), high performance liquid chromatography (HPLC), and light scattering.

### Anti-MSLN Antibodies Comprising CDRs

In some embodiments, the present disclosure provides an isolated antibody or the antigen-binding portion thereof comprising:
A) one or more heavy chain CDRs (HCDRs) selected from the group consisting of:
a HCDR1 as set forth in SEQ ID NO: 1 or an amino acid sequence that differs from SEQ ID NO: 1 by an amino acid addition, deletion or substitution of not more than 2 amino acids; a HCDR2 as set forth in SEQ ID NO: 2 or an amino acid sequence that differs from SEQ ID NO: 2 by an amino acid addition, deletion or substitution of not more than 2 amino acids; and a HCDR3 as set forth in SEQ ID NO: 3 or an amino acid sequence that differs from SEQ ID NO: 3 by an amino acid addition, deletion or substitution of not more than 2 amino acids;
B) one or more light chain CDRs (LCDRs) selected from the group consisting of:
a LCDR1 as set forth in SEQ ID NO: 4 or an amino acid sequence that differs from SEQ ID NO: 4 by an amino acid addition, deletion or substitution of not more than 2 amino acids; a LCDR2 as set forth in SEQ ID NO: 5 or an amino acid sequence that differs from SEQ ID NO: 5 by an amino acid addition, deletion or substitution of not more than 2 amino acids; and a LCDR3 as set forth in SEQ ID NO: 6 or an amino acid sequence that differs from SEQ ID NO: 6 by an amino acid addition, deletion or substitution of not more than 2 amino acids; or
C) one or more HCDRs of A) and one or more LCDRs of B).

In some embodiments, the substitution is a conservative substitution. In some embodiments, the CDR identification is according to IMGT and Kabat definition.

In some embodiments, the isolated antibody, or the antigen-binding portion thereof, comprises: a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; a LCDR1 comprising the amino acid sequence of SEQ ID NO: 4; a LCDR2 comprising the amino acid sequence of SEQ ID NO: 5; and a LCDR3 comprising the amino acid sequence of SEQ ID NO: 6.

The extent of the framework region and CDRs can be precisely identified using methodology known in the art, for example, by the Kabat definition, the Chothia definition, the AbM definition, the Contact definition, the IMGT definition (all of which are well known in the art) and any combinations thereof (see, for example, Kabat, E. A., et al. (1991) Sequences of Proteins of Immunological Interest, Fifth Edition, U.S. Department of Health and Human Services, NIH Publication No. 91-3242, Chothia et al., Nature 342:877, 1989; Chothia, C. et al. J Mol Biol 196:901-917 (1987), Al-lazikani et al J Mol Biol 273:927-948 (1997); Edelman et al., Proc Natl Acad Sci USA. 63(1):78-85, 1969; and Martin and Allen, in “Handbook of Therapeutic Antibodies”, chapter 5, 2007; see, for example, also hgmp.mrc.ac.uk and bioinf.org.uk/abs). Correspondence or alignments between numberings according to different definitions can for example be found at www.imgt.org/(see also Giudicelli V et al. IMGT, the international ImMunoGeneTics database. Nucleic Acids Res 25:206-11, 1997; and Lefranc M P et al., IMGT unique numbering for immunoglobulin and T cell receptor variable domains and Ig superfamily V-like domains. Dev Comp Immunol. 27:55-77, 2003).

As will be appreciated by those in the art, the exact numbering and placement of the CDRs can be different among different numbering systems. However, it should be understood that the disclosure of a variable heavy sequence and/or a variable light sequence includes the disclosure of the associated (inherent) CDRs, regardless of which numbering approach is adopted. Accordingly, the disclosure of each variable region is a disclosure of the CDRs (for example, HCDR1, HCDR2 and HCDR3). Two antibodies having the same VH and VL means that their CDRs are identical when determined by the same approach (for example, the Kabat, AbM, Chothia, Contact, and IMGT numbering approaches as known in the art). The same antibody as disclosed herein may have a different set of CDRs when determined by a different numbering approach.

Variable regions and CDRs in an antibody sequence may be identified according to general rules that have been developed in the art (for example, the Kabat, AbM, Chothia, Contact, and IMGT numbering system) or by aligning the sequences against a database of known variable regions. Methods for identifying these regions are described in Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinforg.uk/abs (maintained by A.C. Martin in the Department of Biochemistry & Molecular Biology University College London, London, England) and the VBASE2 website at www.vbase2.org, as described in Retter et al., Nucl Acids Res, 33 (Database issue): D671-D674, 2005. Sequences may be analyzed using the Abysis database, which integrates sequence data from Kabat, IMGT and the Protein Data Bank (PDB) with structural data from the PDB (see, for example, Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs)). The Abysis database website further includes general rules that have been developed for identifying CDRs which can be used in accordance with the teachings herein.

In some embodiments, provided herein is an anti-MSLN antibody, or antigen-binding portion thereof, comprising at least one of the HCDR1, HCDR2 and HCDR3 of the VH region as set forth in SEQ ID NO: 7, and at least one of the LCDR1, LCDR2 and LCDR3 of the VL region as set forth in SEQ ID NO: 8.

In some embodiments, the anti-MSLN antibodies, or antigen-binding portions thereof, as disclosed herein, include a VH region and a VL region, wherein the VH region comprises FRW1-HCDR1-FRW2-HCDR2-FRW3-HCDR3-FRW4, and wherein HCDR1 has an amino acid sequence as set forth in SEQ ID NO: 1, HCDR2 has an amino acid sequence as set forth in SEQ ID NO: 2, and HCDR3 has an amino acid sequence as set forth in SEQ ID NO: 3, and/or wherein the VL region comprises FRW1-LCDR1-FRW2-LCDR2-FRW3-LCDR3-FRW4, and wherein LCDR1 has an amino acid sequence as set forth in SEQ ID NO: 4, LCDR2 has an amino acid sequence as set forth in SEQ ID NO: 5, and LCDR3 has an amino acid sequence as set forth in SEQ ID NO: 6.

In some embodiments, the framework (FR) regions are derived from human germline, for example, a human immunoglobulin. In some embodiments, the FR regions may include one or more individual FR residue modifications that improve antibody performance, such as stability, binding affinity, isomerization, immunogenicity, and the like. For example, the FR regions may comprise a PTM-removal modification to avoid post-translational modification (PTM). PTMs mainly include isomerization, deamination, glycosylation and oxidation in antibody discovery, all of them have a typical amino acid site, e.g. “DG” for isomerization, “NG” for deamination, “N*T/S” (* stand for other amino acid except P or D) for glycosylation and “M” or “C” for oxidation. Once the PTM sites are found in antibody sequence, especially in key regions like CDR3, PTM removal may be needed to avoid the potential risk of PTM modification while minimally affecting the binding compared to the parental antibody.

In some embodiments, the antibody, or antigen-binding portion thereof, may include an amino acid sequence of “NIS” at positions 70-72 of the amino acid sequence of the VH region. In some embodiments, the antibody, or antigen-binding portion thereof, may include an amino acid modification at any of positions 70-72 of the amino acid sequence of the VH region to remove the “NIS” sequence. In some embodiments, the antibody, or antigen-binding portion thereof, may include a substitution at position 70, 71 and/or 72 of the amino acid sequence of the VH region. In some embodiments, the antibody, or antigen-binding portion thereof, may include a N70Q substitution in the VH region compared to the parental antibody. In some embodiments, the antibody, or antigen-binding portion thereof, may include a I71P substitution in the VH region compared to the parental antibody. In some embodiments, the antibody, or antigen-binding portion thereof, may include a S72P substitution in the VH region compared to the parental antibody. The FRW1 and FRW4 at the N and C terminal of the VH and/or VL region may be truncated such that it includes only a partial FRW1 and/or FRW4.

In some embodiments, the antibody, antigen-binding portion thereof, binding agent, or conjugate, as disclosed herein includes at least one of the heavy chain FRW1, FRW2, FRW3 and FRW4 of the VH region as set forth in SEQ ID NO: 7, and at least one of the light chain FRW1, FRW2, FRW3 and FRW4 of the VL region as set forth in SEQ ID NO: 8.

### Anti-MSLN Antibodies Comprising a Heavy Chain Variable Region and a Light Chain Variable Region

In some embodiments, the isolated antibody, antigen-binding portion thereof, binding agent, or conjugate, may include:
(A) a heavy chain variable region (VH):
(i) comprising the amino acid sequence of SEQ ID NOs: 7;
(ii) comprising an amino acid sequence having at least 85%, 90%, or 95% identity with one of SEQ ID NO: 7; or
(iii) comprising an amino acid sequence with addition, deletion and/or substitution of one or more (e.g. 10, 9, 8, 7, 6, 5, 4, 3, 2) amino acids in the framework regions compared with the amino acid sequence of one of SEQ ID NO: 7; and/or
(B) a light chain variable region (VL):
(i) comprising the amino acid sequence of SEQ ID NO: 8;
(ii) comprising an amino acid sequence having at least 85%, 90%, or 95% identity with SEQ ID NO: 8; or
(iii) comprising an amino acid sequence with addition, deletion and/or substitution of one or more (e.g. 10, 9, 8, 7, 6, 5, 4, 3, 2) amino acids in the framework regions compared with the amino acid sequence of SEQ ID NO: 8.

In some embodiments, the amino acid sequences of the heavy chain variable region and/or the light chain variable region can be at least 85%, 86%, 87%, 88%, 89%, 90%, 91%, 92%, 93%, 94%, 95%, 96%, 97%, 98% or 99% identical to the respective sequences set forth above.

In some embodiments, the VH and VL as described above have the same set of CDRs as SEQ ID NO:7 and SEQ ID NO:8, respectively, and with the sequence having at least 85%, 90%, or 95% identity in the framework regions.

In some embodiments, the percent identity between two amino acid sequences may be determined using the algorithm of E. Meyers and W. Miller (Comput Appl Biosci, 4:11-17, 1988) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J Mol Biol 48:444-453, 1970) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure may further be used as a “query sequence” to perform a search against public databases to, for example, identify related sequences. Such searches can be performed using the XBLAST program (version 2.0) of Altschul, et al. J Mol Biol 215:403-10, 1990. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, Nucleic Acids Res 25(17):3389-3402, 1997. When utilizing BLAST and Gapped BLAST programs, the default parameters of the respective programs (e.g., XBLAST and NBLAST) can be used. See www.ncbi.nlm.nih.gov.

In some further embodiments, the isolated antibody, antigen-binding portion thereof, binding agent, or conjugate, may contain conservative substitution or modification of amino acids in the variable regions of the heavy chain and/or light chain. It is understood in the art that certain conservative sequence modification can be made which do not remove antigen binding. See, for example, Brummell et al. Biochem 32:1180-8, 1993; de Wildt et al. Prot. Eng. 10:835-41, 1997; Komissarov et al. J. Biol. Chem. 272:26864-26870, 1997; Hall et al. J. Immunol. 149:1605-12, 1992; Kelley and O'Connell Biochem. 32:6862-35, 1993; Adib-Conquy et al. Int. Immunol 10:341-6, 1998; and Beers et al. Clin. Can. Res. 6:2835-43, 2000.

The term “conservative substitution,” as used herein, may refer to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, and the like) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem 32: 1180-1187, 1993; Kobayashi et al., Protein Eng. 12(10): 879-884, 1999; and Burks et al., Proc. Natl. Acad. Sci. USA 94: 412-417, 1997, which are incorporated herein by reference).

In some embodiments, a conservatively modified variant of an anti-MSLN antibody, or antigen binding portion thereof, binding agent, or conjugate, may have alterations in the framework regions (FR), for example, other than in the CDRs. For example, a conservatively modified variant of an anti-MSLN antibody may have the amino acid sequences of the VH and VL CDRs (for example, as set forth in SEQ ID NOs:1-6) and at least one conservative amino acid substitution in the FR. In some embodiments, the VH and VL amino acid sequences (for example, as set forth in SEQ ID NOs:7 and 8, respectively) collectively may have no more than 8 or 6 or 4 or 2 or 1 conservative amino acid substitutions in the FR, as compared to the amino acid sequences of the VH and VL (as set forth in SEQ ID NOs:7 and 8, respectively). In some embodiments, the VH and VL amino acid sequences (for example, as set forth in SEQ ID NOs:7 and 8, respectively) have 8 to 1, 6 to 1, 4 to 1 or 2 to 1 conservative amino acid substitutions in the FR, as compared to the amino acid sequences of the VH and VL (as set forth in SEQ ID NOs:7 and 8, respectively). In further aspects of any of these embodiments, a conservatively modified variant of the anti-MSLN antibody, antigen binding portion thereof or other binding agent exhibits specific binding to MSLN.

For conservative amino acid substitutions, a given amino acid can be replaced by a residue having similar physiochemical characteristics, for example, substituting one aliphatic residue for another (such as Ile, Val, Leu, or Ala for one another), or substitution of one polar residue for another (such as between Lys and Arg; Glu and Asp; or Gln and Asn). Other such conservative amino acid substitutions, for example, substitutions of entire regions having similar hydrophobicity characteristics, are well known. Polypeptides comprising conservative amino acid substitutions can be tested in any one of the assays described herein to confirm that a desired activity, for example, antigen-binding activity and specificity of a native or reference polypeptide is retained, for example, to MSLN.

For conservative substitutions, amino acids can be grouped according to similarities in the properties of their side chains (see, for example, A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions may include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of an anti-MSLN antibody, or antigen binding portion thereof, may be at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs (SEQ ID NOs:1-6) are not modified.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

In some embodiments, the anti-MSLN antibody, of antigen binding portion thereof, binding agent, or anti-MSLN conjugate, includes a heavy chain variable (VH) region and a light chain variable (VL) region, wherein the VH region includes a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region includes a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6.

In some embodiments, the VH may include one, two, three, or four human framework regions and the VL may include one, two, three, or four human framework regions. In some embodiments, the VH may include human framework regions 1, 2, and 4, and in a framework region 3 may include one or two substitutions mutations relative to a human framework region 3, and the VL may include four human framework regions. In some embodiments, framework region 3 may include the mutation N70Q and/or S72A.

In some embodiments, the anti-MSLN antibody, or antigen binding portion thereof, binding agent, or anti-MSLN conjugate, includes a heavy chain variable (VH) region that includes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:7 and/or a light chain variable (VL) region that includes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:8.

In some embodiments, the anti-MSLN antibody, or antigen binding portion thereof, binding agent, or anti-MSLN conjugate, includes a heavy chain variable (VH) region having the amino acid sequence set forth in SEQ ID NO:7, and/or a light chain variable (VL) region having the amino acid sequence set forth in SEQ ID NO:8.

In some embodiments, the anti-MSLN antibody, or antigen binding portion thereof, binding agent, or anti-MSLN conjugate, includes a heavy chain variable (VH) region having the amino acid sequence set forth in SEQ ID NO:7, and a light chain variable (VL) region having the amino acid sequence set forth in SEQ ID NO:8.

In some embodiments, the isolated antibody or the antigen-binding portion thereof, binding agent, or anti-MSLN conjugate, includes: a heavy chain variable region comprising or consisting of the amino acid sequence of SEQ ID NO: 7, and a light chain variable region comprising or consisting of the amino acid sequence of SEQ ID NO: 8.

In some embodiments, the isolated antibody or the antigen-binding portion thereof, binding agent, or anti-MSLN conjugate, includes: a heavy chain variable region consisting of the amino acid sequence of SEQ ID NO: 7, and a light chain variable region consisting of the amino acid sequence of SEQ ID NO: 8.

In some embodiments, the heavy (VH) and light (VL) chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, provided herein is a binding agent including a heavy chain variable (VH) region having the amino acid sequence set forth in SEQ ID NO:7, and/or a light chain variable (VL) region having the amino acid sequence set forth in SEQ ID NO:8.

In some embodiments, provided herein is a binding agent including a heavy chain variable (VH) region having the amino acid sequence set forth in SEQ ID NO:7, and a light chain variable (VL) region having the amino acid sequence set forth in SEQ ID NO:8.

In some embodiments, the binding agent specifically binds to MSLN.

In some embodiments, the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified, and wherein the binding agent specifically binds to MSLN.

In some embodiments, the isolated antibody or the antigen-binding portion thereof, binding agent, or anti-MSLN conjugate, includes an antibody or an antigen-binding portion thereof that is a monoclonal antibody, a Fab, a Fab′, an F(ab′), an Fv, a disulfide linked FvFc, a scFv, a scFab, a single domain antibody, a diabody, a bi-specific antibody, or a multi-specific antibody.

In some embodiments, the isolated antibody or the antigen-binding portion thereof, binding agent, or anti-MSLN conjugate, includes a bi-specific antibody or multi-specific antibody that is a Bispecific T cell Engager (BiTE); a DART; a Knobs-Into-Holes (KIH) assembly; a scFv-CH3-KIH assembly; a KIH Common Light-Chain antibody; a TandAb; a Triple Body; a TriBi Minibody; a Fab-scFv; a scFv-CH-CL-scFv; a F(ab′)2-scFv2; a tetravalent Hcab; an intrabody; a CrossMab; a Dual Action Fab (DAF) (two-in-one or four-in-one); a DutaMab; a DT-IgG, a charge paired antibody; a Fab-arm Exchange antibody, a SEEDbody; a Triomab; a LUZ-Y assembly, an Fcab; a K-body; an orthogonal Fabs antibody; a DVD-Ig; am IgG(H)-scFv; an scFv-(H)IgG; an IgG(L)-scFv; an scFv-(L)IgG; an IgG(L,H)-Fv; an IgG(H)-V; a V(H)-IgG; an IgG(L)-V; a V(L)-IgG; a KIH IgG-scFab; a 2scFv-IgG; a IgG-2scFv; a scFv4-Ig; a Zybody; a DVI-IgG (four-in-one), a FIT-Ig; a WuxiBody; or an In-Elbow-Insert Ig.

In some embodiments, the heavy and/or light chain CDRs of an antibody, or antigen binding fragment thereof, may be identified by using any one of the following methods: Kabat, Chothia, AbM, Contact, IMGT, and/or Aho. In some embodiments, the CDRs may be defined by Kabat and IMGT.

In some embodiments, provided is a binding agent including a heavy chain variable (VH) region and a light chain variable (VL) region, wherein the VH region includes a complementarity determining region HCDR1 sequence having the amino acid sequence set forth in SEQ ID NO: 1, a HCDR2 having the amino acid sequence set forth in SEQ ID NO:2, and a HCDR3 having the amino acid sequence set forth in SEQ ID NO:3; and wherein the VL region includes a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6.

In some embodiments, each VH and VL includes a humanized framework region and the binding agent specifically binds to MSLN.

### Fc Region

Anti-MSLN antibodies, and antigen-binding portions thereof, provided herein may further comprise an immunoglobulin constant region comprising a Fc region, such as a human IgG1, IgG2, IgG3 or IgG4 Fc region (native or variant thereof), and optionally a hinge region. In some embodiments, the Fc region is a human IgG1 Fc region, such as a wild-type Fc region or an Fc variant. An Fc variant may possess at least about 80% homology with a native sequence Fc region, or at least about 90% homology therewith, for example, at least about 95% homology therewith. In some embodiments, the Fc region is a human IgG4 Fc region, such as a wild-type Fc region or a Fc variant comprising a S228P substitution. In some embodiments, the anti-MSLN antibodies, or antigen-binding portions thereof, disclosed herein may include wild-type human IgG1 Fc region. The variant Fc region may include one or more amino acid changes (for example, insertions, deletions or substitutions) that alters the antibody-dependent cellular cytotoxicity (ADCC) or other effector functions, or modifying the binding interaction between Fc and FcRn or FcγR, including but not limited to, Leu234Ala/Leu235Ala (LALA), S298A, E333A, K334A, M252Y/S254T/T256E (“YTE”), M428L/N434S (“LS”), as well as other conventionally adopted substitutions. S298A/E333A/K334A has been shown to result in enhanced ADCC compared to WT IgG when introduced into the humanized IgG1, “YTE” has been shown to increase the binding affinity of the antibody Fc to the MHC Class I neonatal FcR (FcRn) thereby permitting more efficient recycling of administered IgG1 antibody.

In certain embodiments, the Fc region may be an IgG4 Fc region including a S228P mutation (according to EU numbering as in Kabat et al., supra) that prevents Fab arm exchange and stabilizes IgG4 molecule. In certain embodiments, the Fc region may be a IgG1 Fc region and include a LALA mutation, for example, mutations of L234A and L235A. LALA mutation is perhaps the most commonly used mutation for disrupting antibody effector function, for example, to eliminate Fc binding to specific FcTRs, and/or reduce ADCC activity mediated by PBMCs and monocytes. The “EU numbering system” or “EU index” is generally used when referring to a residue in an immunoglobulin heavy chain constant region (for example, the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

Monoclonal antibodies can be prepared using a wide variety of techniques known in the art including hybridoma techniques, recombinant techniques, phage display technologies, transgenic animals (for example, a XenoMouse®) or some combination thereof. For example, monoclonal antibodies can be produced using hybridoma and art-recognized biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981) each of which is incorporated herein in its entirety by reference. It should be understood that a selected binding sequence may be further altered, for example, to improve affinity for the target, to humanize the target binding sequence, to improve its production in cell culture, to reduce its immunogenicity in vivo, to create a multispecific antibody, and the like, and that an antibody including the altered target binding sequence is also an antibody contemplated by this disclosure. In some embodiments, the anti-human MSLN monoclonal antibody is prepared by using hybridoma techniques. Generation of hybridomas is well-known in the art (see, for example, Harlow and Lane (1988) Antibodies, A Laboratory Manual, Cold Spring Harbor Publications, New York).

In some embodiments, an anti-MSLN antibody, antigen binding portion thereof or other binding agent, has an IgG1 heavy chain constant region. In some embodiments, the anti-MSLN antibody, antigen binding portion thereof or other binding agent, has a heavy chain constant region that has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:9. In some embodiments, the anti-MSLN antibody, antigen binding portion thereof or other binding agent, has a heavy chain constant region that has the amino acid sequence as set forth in SEQ ID NO:9.

In some embodiments, the anti-MSLN antibody, antigen binding portion thereof or other binding agent, has a light chain constant region. In some embodiments, the anti-MSLN antibody, antigen binding portion thereof or other binding agent, has a the light chain constant region that has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:10. In some embodiments, the anti-MSLN antibody, antigen binding portion thereof or other binding agent, has a light chain constant region that has the amino acid sequence as set forth in SEQ ID NO:10.

In some embodiments, an anti-MSLN antibody or antigen-binding portion thereof, or other binding agent, has fully human constant regions. In some embodiments, an anti-MSLN antibody or antigen-binding portion thereof, or other binding agent, has non-human constant regions. In some embodiments, an anti-MSLN antibody heavy chain has the amino acid sequence set forth in SEQ ID NO:11; and/or an anti-MSLN antibody light chain has the amino acid sequence set forth in SEQ ID NO:12. In some embodiments, an anti-MSLN antibody or antigen-binding portion thereof, or other binding agent, includes a heavy chain that has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:11. In some embodiments, an anti-MSLN antibody or antigen-binding portion thereof, or other binding agent, includes a light chain that has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:12. In some embodiments, an anti-MSLN antibody heavy chain has the amino acid sequence set forth in SEQ ID NO:11 and an anti-MSLN antibody light chain has the amino acid sequence set forth in SEQ ID NO:12. In some embodiments, a binding agent comprises the amino acid sequence set forth in SEQ ID NO:11 and the amino acid sequence set forth in SEQ ID NO:12.

In some embodiments, an anti-MSLN antibody or antigen-binding portion thereof, or other binding agent, includes two heavy chains and two light chains, wherein each heavy chain comprises or consists of SEQ ID NO:11 and each light chain comprises or consists of SEQ ID NO:12.

In various embodiments, anti-MSLN antibodies, antigen binding portions thereof and other binding agents may be produced in human, murine or other animal-derived cells lines. Recombinant DNA expression may be used to produce anti-MSLN antibodies, antigen binding portions thereof, and other binding agents. This may allow the production of anti-MSLN antibodies as well as a spectrum of MSLN antigen binding portions and other binding agents (including fusion proteins) in a host species of choice. The production of anti-MSLN antibodies, antigen binding portions thereof, and other binding agents, may be in bacteria, yeast, transgenic animals and chicken eggs, and/or cell-based production systems. The main advantages of transgenic animals may be potential high yields from renewable sources.

### Nucleic Acid Molecules Encoding Antibodies of the Disclosure

In some aspects, the disclosure is directed to an isolated nucleic acid molecule, comprising a nucleic acid sequence encoding the heavy chain variable region and/or the light chain variable region of the isolated anti-MSLN antibody, or antigen-binding portion thereof, as disclosed herein.

Nucleic acids of the disclosure can be obtained using standard molecular biology techniques. For antibodies expressed by hybridomas (for example, hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (for example, using phage display techniques), a nucleic acid encoding such antibodies can be recovered from the gene library.

The isolated nucleic acid encoding the VH region may be converted to a full-length heavy chain gene by operatively linking the VH-encoding nucleic acid to another DNA molecule encoding heavy chain constant regions (CH1, CH2 and CH3). The sequences of human heavy chain constant region genes are known in the art (see, for example, Kabat et al. (1991), supra) and DNA fragments encompassing these regions can be obtained by standard PCR amplification. The heavy chain constant region can be an IgG1, IgG2, IgG3, IgG4, IgA, IgE, IgM or IgD constant region, but more preferably is an IgG1 or IgG4 constant region.

The isolated nucleic acid encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see, for example, Kabat et al., supra) and DNA fragments encompassing these regions can be obtained by standard PCR amplification. In preferred embodiments, the light chain constant region can be a kappa or lambda constant region.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In some embodiments, the disclosure is directed to an isolated nucleic acid molecule, comprising a nucleic acid sequence encoding the heavy chain variable region of the isolated antibody, or antigen-binding portion thereof, as disclosed herein.

In some specific embodiments, the isolated nucleic acid molecule encodes the heavy chain variable region of the isolated antibody, antigen-binding portion thereof, and comprises a nucleic acid sequence selected from the group consisting of:
(A) a nucleic acid sequence that encodes a heavy chain variable region as set forth in any one of SEQ ID NOs: 7;
(B) a nucleic acid sequence having at least 85%, 90%, or 95% identity to the nucleic acid sequence of (A); or
(C) a nucleic acid sequence that hybridizes under high stringency conditions to the complementary strand of the nucleic acid sequence of (A).

In some embodiments, the disclosure is directed to an isolated nucleic acid molecule, comprising a nucleic acid sequence encoding the light chain variable region of the isolated antibody as disclosed herein.

In some specific embodiments, the isolated nucleic acid molecule encodes the light chain variable region of the isolated antibody comprises a nucleic acid sequence selected from the group consisting of:
(A) a nucleic acid sequence that encodes a light chain variable region as set forth in SEQ ID NO: 8;
(B) a nucleic acid sequence having at least 85%, 90%, or 95% identity to the nucleic acid sequence of (A); or
(C) a nucleic acid sequence that hybridizes under high stringency conditions to the complementary strand of the nucleic acid sequence of (A).

In some embodiments, the percentage of identity is derived from the degeneracy of the genetic code, and the encoded protein sequences remain unchanged.

Exemplary high stringency conditions include hybridization at 45° C. in 5×SSPE and 45% formamide, and a final wash at 65° C. in 0.1×SSC. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al, (Eds.), Molecular Cloning: A laboratory Manual. Cold Spring Harbor Laboratory Press: Cold Spring Harbor, New York (1989), pp. 9.47 to 9.51.

In some embodiments, an anti-MSLN heavy chain polypeptide with the amino acid sequence set forth in SEQ ID NO:11 is encoded by a nucleic acid. In some embodiments, an anti-MSLN light chain polypeptide with the amino acid sequence set forth in SEQ ID NO:12 is encoded by a nucleic acid. In some embodiments, the anti-MSLN heavy chain polypeptide with the amino acid sequence set forth in SEQ ID NO:11 is encoded by a nucleic acid having the sequence set forth in SEQ ID NO:13. In some embodiments, the anti-MSLN light chain polypeptide with the amino acid sequence set forth in SEQ ID NO:12 is encoded by a nucleic acid having the sequence set forth in SEQ ID NO:14.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. If single stranded, a nucleic acid may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing. In some embodiments, the nucleic acid can be a cDNA, for example, a nucleic acid lacking introns.

Nucleic acid molecules encoding the amino acid sequence of an anti-MSLN antibody, antigen binding portion thereof, as well as other binding agents, may be prepared by a variety of methods known in the art. These methods include, but are not limited to, preparation of synthetic nucleotide sequences encoding of an anti-MSLN antibody, antigen binding portion, or other binding agent(s). In addition, oligonucleotide-mediated (or site-directed) mutagenesis, PCR-mediated mutagenesis, and cassette mutagenesis may be used to prepare nucleotide sequences encoding an anti-MSLN antibody, or antigen binding portion, as well as other binding agents. A nucleic acid sequence encoding at least an anti-MSLN antibody, antigen binding portion thereof, binding agent, or a polypeptide thereof, as described herein, may be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, for example, by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and may be used to construct nucleic acid sequences and vectors that encode, for example, an anti-MSLN antibody, or antigen binding portion thereof, or a VH or VL polypeptide thereof.

A nucleic acid molecule, such as DNA, is said to be “capable of expressing” a polypeptide if it includes nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (for example, an anti-MSLN antibody, or antigen binding portion thereof, or other binding agent) are connected in such a way as to permit gene expression of a polypeptide(s) or antigen binding portions in recoverable amounts. The precise nature of the regulatory regions needed for gene expression may vary from organism to organism, as is well known in the analogous art (see, for example, Sambrook et al., 1989; Ausubel et al., 1987-1993).

### Host Cells

Host cells as disclosed in the present disclosure may be any cell which is suitable for expressing the antibodies, or antigen-binding portions thereof, or other binding agents, of the present disclosure, for example, yeast, bacterial, plant and mammalian cells. Mammalian host cells for expressing the antibodies of the present disclosure include Chinese Hamster Ovary (CHO cells) (including dhfr CHO cells, described in Urlaub and Chasin, Proc Natl Acad Sci USA 77:4216-4220, 1980, used with a DHFR selectable marker, for example, as described in R. J. Kaufman and P. A. Sharp J Mol Biol 159:601-621, 1982), 293F cells, NSO myeloma cells, COS cells and SP2 cells. In particular, for use with NSO myeloma cells, another expression system is the GS gene expression system disclosed in WO 87/04462, WO 89/01036 and EP 338,841. Also included are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc Natl Acad Sci USA 77:4216, 1980); mouse sertoli cells (TM4, Mather, Biol Reprod 23:243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68, 1982); MRC 5 cells; FS4 cells; mouse myeloma cells, such as NSO (e.g. RCB0213, Bio/Technology 10:169, 1992) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); PER.C6 cells; and a human hepatoma line (Hep G2). CHO cells are one of the cell lines that can be used herein, with CHO-K1, DUK-B11, CHO-DP12, CHO-DG44 (Somatic Cell and Molecular Genetics 12:555, 1986), and Lec13 being exemplary host cell lines. In the case of CHO-K1, DUK-B11, DG44 or CHO-DP12 host cells, these may be altered such that they are deficient in their ability to fucosylate proteins expressed therein. In some embodiments, the host cells herein are selected from CHO, CHO-S, HEK, HEK293, HEK-293F, Expi293F, PER.C6 or NSO cells or lymphocytic cells.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are also suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, for example, K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

When recombinant expression vectors encoding an antibody, or antigen-binding portion thereof, are introduced into mammalian host cells, the antibody, antigen-binding portion thereof, is produced by culturing the host cells for a period of time sufficient to allow for expression of the antibody in the host cells or, secretion of the antibody into the culture medium in which the host cells are grown. Antibodies, antigen-binding portion thereof, can be recovered from the culture medium using standard protein purification methods.

The expression of an anti-MSLN antibody, or antigen-binding portion thereof, or other binding agent, as described herein, may occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue may be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins may be accomplished. The fusion proteins so produced may be processed in vivo or purified and processed in vitro, allowing synthesis of an anti-MSLN antibody, or antigen binding portion thereof, as described herein, with a specified amino terminus sequence. Moreover, problems associated with retention of initiation codon-derived methionine residues in direct yeast (or bacterial) expression maybe avoided (see, for example, Sabin et al., 7 Bio/Technol. 705, 1989; Miller et al., 7 Bio/Technol. 698, 1989.) Any of a series of yeast gene expression systems incorporating promoter and termination elements from the actively expressed genes coding for glycolytic enzymes produced in large quantities when yeast are grown in medium rich in glucose may be utilized to obtain recombinant anti-MSLN antibodies, or antigen-binding portions thereof. Known glycolytic genes may also provide very efficient transcriptional control signals. For example, the promoter and terminator signals of the phosphoglycerate kinase gene may be utilized.

Production of anti-MSLN antibodies, or antigen-binding portions thereof, in insects may be achieved, for example, by infecting an insect host with a baculovirus engineered to express a polypeptide by methods known to those of ordinary skill in the art (see, for example, Ausubel et al., 1987-1993).

In some embodiments, the introduced nucleic acid sequence (encoding an anti-MSLN antibody or antigen binding portion thereof or a polypeptide thereof) is incorporated into a plasmid or viral vector capable of autonomous replication in a recipient host cell. Any of a wide variety of vectors may be employed for this purpose and are known and available to those of ordinary skill in the art (see, for example, Ausubel et al., 1987-1993). Factors of importance in selecting a particular plasmid or viral vector may include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary viral vectors include retrovirus, adenovirus, parvovirus (for example, adeno-associated viruses), coronavirus, negative strand RNA viruses such as ortho-myxovirus (for example, influenza virus), rhabdovirus (for example, rabies and vesicular stomatitis virus), paramyxovirus (for example, measles and Sendai), positive strand RNA viruses such as picornavirus and alphavirus, and double-stranded DNA viruses including adenovirus, herpesvirus (for example, Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (see, for example, Coffin, J. M., Retroviridae: The viruses and their replication, In Fundamental Virology, Third Edition, B. N. Fields et al., Eds., Lippincott-Raven Publishers, Philadelphia, 1996). In some such embodiments, the viral vector may be a lentiviral vector or a 7-retroviral vector.

Exemplary prokaryotic vectors known in the art include plasmids such as those capable of replication in E. coli. Other gene expression elements useful for the expression of DNA encoding anti-MSLN antibodies, or antigen-binding portions thereof, include, but are not limited to (a) viral transcription promoters and their enhancer elements, such as the SV40 early promoter (see, for example, Okayama et al., Mol. Cell. Biol., 3: 280-289 (1983)), Rous sarcoma virus LTR (see, for example, Gorman et al., PNAS, 79:6777, 1982), and Moloney murine leukemia virus LTR (see, for example, Grosschedl et al., Cell, 41:885, 1985); (b) splice regions and polyadenylation sites such as those derived from the SV40 late region (see, for example, Okayama et al., Mol. Cell. Biol., 3: 280-289, 1983), and (c) polyadenylation sites such as in SV40 (see, for example, Okayama et al., Mol. Cell. Biol., 3: 280-289, 1983). Immunoglobulin-encoding DNA genes can be expressed as described by Weidle et al., Gene, 51:21, 1987, using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter may be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion may be assembled in, or inserted into, an expression vector. Recipient cells capable of expressing the anti-MSLN variable region(s), or antigen binding portions thereof (for example, a VH having the amino acid sequence set forth in SEQ ID NO:7 and/or a VL having the amino acid sequence set forth in SEQ ID NO:8; or a variant thereof as described herein), are then transfected singly with nucleotides encoding an anti-MSLN antibody, or an antibody polypeptide or antigen-binding portion thereof, or are co-transfected with a polynucleotide(s) encoding VH and a VL chain coding regions. The transfected recipient cells may be cultured under conditions that permit expression of the incorporated coding regions and the expressed antibody chains, or intact antibodies, or antigen binding portions, are recovered from the culture.

In some embodiments, the nucleic acids containing the coding regions encoding an anti-MSLN antibody or antigen-binding portion thereof (for example, a VH having the amino acid sequence set forth in SEQ ID NO:7 and/or a VL having the amino acid sequence set forth in SEQ ID NO:8; or a variant thereof as described herein) are assembled in separate expression vectors that are then used to co-transfect a recipient host cell. Each vector may include one or more selectable genes. For example, in some embodiments, two selectable genes may be used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (for example, containing anti-MSLN antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively, the fused nucleotide sequences encoding VH and VL chains may be assembled on the same expression vector.

For transfection of the expression vectors and production of the anti-MSLN antibodies or antigen binding portions thereof, the recipient cell line may be a Chinese Hamster ovary cell line (for example, DG44) or a myeloma cell. Myeloma cells may synthesize, assemble and secrete immunoglobulins encoded by transfected immunoglobulin genes and possess the mechanism for glycosylation of the immunoglobulin. For example, in some embodiments, the recipient cell may be the recombinant Ig-producing myeloma cell SP2/0. SP2/0 cells only produce immunoglobulins encoded by the transfected genes. Myeloma cells may be grown in culture or in the peritoneal cavity of a mouse, where secreted immunoglobulin can be obtained from ascites fluid.

An expression vector encoding an anti-MSLN antibody, or antigen-binding portion thereof (for example, a VH having the amino acid sequence set forth in SEQ ID NO:7 and/or a VL having the amino acid sequence set forth in SEQ ID NO:8; or a variant thereof as described herein) may be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment, as known to one of ordinary skill in the art (see, for example, Johnston et al., 240 Science 1538, 1988).

Yeast may provide certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which may be used for production of the desired proteins in yeast. Yeast recognizes leader sequences of cloned mammalian gene products and secretes polypeptides bearing leader sequences (in some cases, known as pre-polypeptides) (see, for example, Hitzman et al., 11th Intl. Conf. Yeast, Genetics & Molec. Biol. (Montpelier, France, 1982)).

Yeast gene expression systems may be routinely evaluated for the levels of production, secretion and the stability of antibodies, and assembled anti-MSLN antibodies and antigen binding portions thereof. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter. A number of approaches can be taken for evaluating optimal expression plasmids for the expression of immunoglobulins in yeast. See II DNA Cloning 45, (Glover, ed., IRL Press, 1985) and e.g., U.S. Publication No. 2006/0270045 A1.

Bacterial strains can also be utilized as hosts for the production of the antibody molecules, or antigen binding portions thereof, described herein. For example, E. coli K12 strains such as E. coli W3110, Bacillus species, enterobacteria such as Salmonella typhimurium or Serratia marcescens, and various Pseudomonas species may be used. Plasmid vectors containing replicon and control sequences which are derived from species compatible with a host cell may be used in connection with these bacterial hosts. The vector may carry a replication site, as well as specific genes which are capable of providing phenotypic selection in transformed cells. A number of approaches may be taken for evaluating the expression plasmids for the production of anti-MSLN antibodies, and antigen binding portions thereof, in bacteria (see, for example, Glover, ed., IRL Press, 1985; and Ausubel et al., Current Protocols in Molecular Biology (John Wiley & Sons), 1987-1993).

Host mammalian cells may be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that may be used to express immunoglobulin polypeptides include, but are not limited to, COS cells, including COS 7 cells; 293 cells, including 293-6E cells; CHO cells, including CHO-S and DG44 cells; PERC6™ cells (Crucell); and NSO cells. In some embodiments, a particular eukaryotic host cell may be selected based on its ability to make desired post-translational modifications to the heavy chains and/or light chains. For example, in some embodiments, CHO cells may produce polypeptides that have a higher level of sialylation than the same polypeptide produced in 293 cells.

In some embodiments, one or more anti-MSLN antibodies, or antigen-binding portions thereof, or other binding agents (for example, a VH having the amino acid sequence set forth in SEQ ID NO:7 and/or a VL having the amino acid sequence set forth in SEQ ID NO:8; or a variant thereof as described herein) may be produced in vivo in an animal that has been engineered or transfected with one or more nucleic acid molecules encoding the polypeptides, according to any suitable method.

In some embodiments, an antibody, or antigen-binding portion thereof, or other binding agent (for example, a VH having the amino acid sequence set forth in SEQ ID NO:7 and/or a VL having the amino acid sequence set forth in SEQ ID NO:8; or a variant thereof as described herein) may be produced in a cell-free system. Non-limiting exemplary cell-free systems are described, for example, in Sitaraman et al., Methods Mol. Biol. 498: 229-44, 2009; Spirin, Trends Biotechnol. 22: 538-45, 2004; and Endo et al., Biotechnol. Adv. 21: 695-713, 2003.

Many vector systems are available for the expression of the VH and VL chains (for example, a VH having the amino acid sequence set forth in SEQ ID NO:7 and/or a VL having the amino acid sequence set forth in SEQ ID NO:8; or a variant thereof as described herein) in mammalian cells (see, for example, Glover, ed., IRL Press, 1985). Various approaches may be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof (for example, a VH having the amino acid sequence set forth in SEQ ID NO:7 and/or a VL having the amino acid sequence set forth in SEQ ID NO:8; or a variant thereof as described herein) may be placed into the same plasmid, which is then transfected into cells, thereby selecting directly for cells that express both chains. Alternatively, cells may be transfected first with a plasmid encoding one chain, for example the VL chain, followed by transfection of the resulting cell line with a VH chain plasmid containing a second selectable marker. Cell lines producing antibodies, or antigen-binding portions thereof, via either route could be transfected with plasmids encoding additional copies of peptides, VH, VL, or VH plus VL chains (for example, a VH having the amino acid sequence set forth in SEQ ID NO:7 and/or a VL having the amino acid sequence set forth in SEQ ID NO:8; or a variant thereof as described herein) in conjunction with additional selectable markers to generate cell lines with enhanced properties, such as higher production of assembled anti-MSLN antibodies or antigen binding portions thereof or enhanced stability of the transfected cell lines.

Additionally, plants may provide a convenient, safe and economical alternative expression system for recombinant antibody production, which are based on large scale culture of microbes or animal cells. Anti-MSLN antibodies or antigen binding portions can be expressed in plant cell culture, or plants grown conventionally. The expression in plants may be systemic, limited to sub-cellular plastids, or limited to seeds (endosperms) (see, for example, U.S. Patent Pub. No. 2003/0167531; U.S. Pat. Nos. 6,080,560; 6,512,162; PCT Publication No. WO 0129242). Several plant-derived antibodies have reached advanced stages of development, including clinical trials (see, for example, Biolex, N.C.).

For intact antibodies, the variable regions (VH and VL) of the anti-MSLN antibodies (for example, a VH having the amino acid sequence set forth in SEQ ID NO:7 and/or a VL having the amino acid sequence set forth in SEQ ID NO:8; or a variant thereof as described herein) may be linked to at least a portion of an immunoglobulin constant region (Fc), typically that of a human immunoglobulin. Human constant region DNA sequences can be isolated in accordance with well-known procedures from a variety of human cells, such as immortalized B-cells (see, for example, PCT Publication No. WO 87/02671; which is incorporated by reference herein in its entirety). An anti-MSLN antibody can contain both light chain and heavy chain constant regions. The heavy chain constant region can include CH1, hinge, CH2, CH3, and, sometimes, CH4 regions. In some embodiments, the CH2 domain may be deleted or omitted.

Alternatively, techniques described for the production of single chain antibodies (see, for example, U.S. Pat. No. 4,946,778; Bird, Science 242:423-42, 1988; Huston et al., Proc. Natl. Acad. Sci. USA 85:5879-5883, 1988; and Ward et al., Nature 334:544-54, 1989; which are incorporated by reference herein in their entireties) can be adapted to produce single chain antibodies that specifically bind to MSLN. Single chain antibodies may be formed by linking the heavy and light chain variable regions (for example, having the amino acid sequences set forth in SEQ ID NOs:7 and 8, or a variant thereof as described herein (for example, optionally modified with from 1 to 8 amino acid substitutions, deletions and/or insertions)) of the Fv region via an amino acid bridge, resulting in a single chain polypeptide. Techniques for the assembly of functional Fv fragments in E. coli may also be used (see, for example, Skerra et al., Science 242:1038-1041, 1988; which is incorporated by reference herein in its entirety).

Intact (for example, whole) antibodies, their dimers, individual light and heavy chains, or antigen binding portions thereof can be recovered and purified by known techniques, for example, immunoadsorption or immunoaffinity chromatography, chromatographic methods such as HPLC (high performance liquid chromatography), ammonium sulfate precipitation, gel electrophoresis, or any combination of these (see, for example, Scopes, Protein Purification (Springer-Verlag, N.Y., 1982)). Substantially pure anti-MSLN antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact anti-MSLN antibody, or antigen binding portion thereof, or other binding agent, as described herein, may then be used therapeutically or in developing and performing assay procedures, immunofluorescent staining, and the like (see, for example, Vols. I & II Immunol. Meth. (Lefkovits & Pernis, eds., Acad. Press, NY, 1979 and 1981)).

Additionally, and as described herein, an anti-MSLN antibody, or antigen binding portion thereof, or other binding agent, may be further optimized to decrease potential immunogenicity, while maintaining functional activity, for therapy in humans.

In some embodiments, an optimized MSLN binding antibody or antigen binding portion thereof, or other binding agent, or conjugate, as described herein, may be derived from an anti-MSLN antibody comprising (i) a heavy chain variable region having the amino acid sequence set forth in SEQ ID NO:7 and (ii) a light chain variable region having the amino acid sequence set forth in SEQ ID NO:8, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, an optimized MSLN binding antibody or antigen binding portion thereof is derived from a MSLN binding antibody comprising (i) a heavy chain variable region having the amino acid sequence set forth in SEQ ID NO:7 and (ii) a light chain variable region having the amino acid sequence set forth in SEQ ID NO:8, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In this regard, functional activity means an anti-MSLN antibody or antigen binding portion thereof capable of displaying one or more known functional activities associated with a MSLN binding antibody or antigen binding portion thereof comprising (i) a heavy chain variable region having the amino acid sequence set forth in SEQ ID NO:7 and (ii) a light chain variable region having the amino acid sequence set forth in SEQ ID NO:8.

In any of these embodiments, the functional activity of the MSLN binding antibody, or antigen binding portion thereof, or other binding agent, or conjugate, as described herein, may include specific binding to MSLN. Additional functional activities may include anti-cancer activity. Additionally, an anti-MSLN antibody, or antigen binding portion thereof, or other binding agent, or conjugate, as described herein, having functional activity may be interpreted as the polypeptide exhibits activity similar to, or better than, the activity of a reference antibody or antigen-binding portion thereof as described herein (for example, a MSLN binding antibody, or antigen binding portion thereof, comprising (i) a VH having the amino acid sequence set forth in SEQ ID NO:7 and/or a VL having the amino acid sequence set forth in SEQ ID NO:8; or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody or antigen-binding portion thereof as described herein (for example, the candidate polypeptide will exhibit greater activity relative to the reference antibody).

In some embodiments, the compositions and methods described herein relate to reduction of MSLN+ cells in a subject (for example, reducing the number of MSLN+ cells in a cancer or tumor) by an anti-MSLN antibody, antigen binding portion thereof, other binding agent or conjugate thereof in vivo. In some embodiments, the compositions and methods described herein relate to the treatment of MSLN+ cancer in a subject by administering an anti-MSLN antibody, antigen binding portion thereof, other binding agent or conjugate thereof.

TABLE 1
Sequences related to Anti-MSLN Antibodies

| Description | Sequence | SEQ ID NO: |
|---|---|---|
| Ab-1 HCDR1 | GGSISSSSYYWG | SEQ ID NO: 1 |
| Ab-1 HCDR2 | SVYYSGSAFYSPSLKS | SEQ ID NO: 2 |
| Ab-1 HCDR3 | RPYKSGLFDF | SEQ ID NO: 3 |
| Ab-1 LCDR1 | RASQSVSSNLA | SEQ ID NO: 4 |
| Ab-1 LCDR2 | GTSTRAT | SEQ ID NO: 5 |
| Ab-1 LCDR3 | QQYNTWPPEYT | SEQ ID NO: 6 |
| Ab-1 VH | QLQLQESGPGLVKPSETLSLTCTVSGGSISSSSYYWG | SEQ ID NO: 7 |
| | WIRQPPGKGLEWIGSVYYSGSAFYSPSLKSRLNIAV | |
| | DTSKKQFSLKLSSVTAADTAVYYCARRPYKSGLFD | |
| | FWGQGTLVTVSS | |
| Ab-1 VL | EIVMTQSPATLSVSPGERATLSCRASQSVSSNLAWY | SEQ ID NO: 8 |
| | LQKPGQAPRLLIYGTSTRATGIPARFSGSGSGTEFTL | |
| | TISSLQSEDFAVYYCQQYNTWPPEYTFGQGTKLEIK | |
| Heavy chain constant | ASTKGPSVFPLAPSSKSTSGGTAALGCLVKDYFPEP | SEQ ID NO: 9 |
| region | VTVSWNSGALTSGVHTFPAVLQSSGLYSLSSVVTVP | |
| | SSSLGTQTYICNVNHKPSNTKVDKRVEPKSCDKTHT | |
| | CPPCPAPEAAGGPSVFLFPPKPKDTLMISRTPEVTCV | |
| | VVDVSHEDPEVKFNWYVDGVEVHNAKTKPREEQY | |
| | NSTYRVVSVLTVLHQDWLNGKEYKCKVSNKALPA | |
| | PIEKTISKAKGQPREPQVYTLPPSREEMTKNQVSLTC | |
| | LVKGFYPSDIAVEWESNGQPENNYKTTPPVLDSDGS | |
| | FFLYSKLTVDKSRWQQGNVFSCSVMHEALHNHYT | |
| | QKSLSLSPGK | |
| Light chain constant | RTVAAPSVFIFPPSDEQLKSGTASVVCLLNNFYPREA | SEQ ID NO: 10 |
| region | KVQWKVDNALQSGNSQESVTEQDSKDSTYSLSSTL | |
| | TLSKADYEKHKVYACEVTHQGLSSPVTKSFNRGEC | |
| Ab-1 heavy chain | QLQLQESGPGLVKPSETLSLTCTVSGGSISSSSYYWG | SEQ ID NO: 11 |
| | WIRQPPGKGLEWIGSVYYSGSAFYSPSLKSRLNIAV | |
| | DTSKKQFSLKLSSVTAADTAVYYCARRPYKSGLFD | |
| | FWGQGTLVTVSSASTKGPSVFPLAPSSKSTSGGTAA | |
| | LGCLVKDYFPEPVTVSWNSGALTSGVHTFPAVLQS | |
| | SGLYSLSSVVTVPSSSLGTQTYICNVNHKPSNTKVD | |
| | KRVEPKSCDKTHTCPPCPAPEAAGGPSVFLFPPKPK | |
| | DTLMISRTPEVTCVVVDVSHEDPEVKFNWYVDGVE | |
| | VHNAKTKPREEQYNSTYRVVSVLTVLHQDWLNGK | |
| | EYKCKVSNKALPAPIEKTISKAKGQPREPQVYTLPPS | |
| | REEMTKNQVSLTCLVKGFYPSDIAVEWESNGQPEN | |
| | NYKTTPPVLDSDGSFFLYSKLTVDKSRWQQGNVFS | |
| | CSVMHEALHNHYTQKSLSLSPGK | |
| Ab-1 light chain | EIVMTQSPATLSVSPGERATLSCRASQSVSSNLAWY | SEQ ID NO: 12 |
| | LQKPGQAPRLLIYGTSTRATGIPARFSGSGSGTEFTL | |
| | TISSLQSEDFAVYYCQQYNTWPPEYTFGQGTKLEIK | |
| | RTVAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| Ab-1 heavy chain | caactgcagctgcaggagtcgggcccaggactggtgaagccttcggagaccct | SEQ ID NO: 13 |
| coding sequence | gtccctcacctgcactgtctctggtggctccatcagcagtagtagttactactgggg | |
| | ctggatccgccagccccccgggaagggtctagaatggattgggagtgtctattat | |
| | agtgggagcgccttctacagcccgtccctcaagagtcgactcaacatagctgtag | |
| | acacgtccaagaagcagttctccctgaagctgagctctgtgaccgccgcagaca | |
| | cggctgtttattattgtgcgagacgtccatataagagtggcctctttgacttctgggg | |
| | ccagggaaccctggtcaccgtctcctcagcgtcgaccaagggcccatccgtcttc | |
| | cccctggcaccctcctccaagagcacctctgggggcacagcggccctgggctg | |
| | cctggtcaaggactacttccccgaaccggtgacggtgtcctggaactcaggcgct | |
| | ctgaccagcggcgtgcacaccttcccggctgtcctacagtcctcaggactctactc | |
| | cctcagcagcgtggtgaccgtgccctccagcagcttgggcacccagacctacat | |
| | ctgcaacgtgaatcacaagcccagcaacaccaaggtggacaagagagttgagc | |
| | ccaaatcttgtgacaaaactcacacatgcccaccgtgcccagcacctgaagctgc | |
| | tgggggaccgtcagtcttcctcttccccccaaaacccaaggacaccctcatgatct | |
| | cccggacccctgaggtcacatgcgtggtggtggacgtgagccacgaagaccct | |
| | gaggtcaagttcaactggtacgtggacggcgtggaggtgcataatgccaagaca | |
| | aagccgcgggaggagcagtacaacagcacgtaccgtgtggtcagcgtcctcac | |
| | cgtcctgcaccaggactggctgaatggcaaggagtacaagtgcaaggtctccaa | |
| | caaagccctcccagcccccatcgagaaaaccatctccaaagccaaagggcagc | |
| | cccgagaaccacaggtctacaccctgcccccatcccgggaggagatgaccaag | |
| | aaccaggtcagcctgacctgcctggtcaaaggcttctatcccagcgacatcgccg | |
| | tggagtgggagagcaatgggcagccggagaacaactacaagaccacgcctcc | |
| | cgtgctggactccgacggctccttcttcctctatagcaagctcaccgtggacaaga | |
| | gcaggtggcagcaggggaacgtcttctcatgctccgtgatgcatgaggctctgca | |
| | caaccactacacgcagaagagcttaagcctgtctccgggtaaa | |
| Ab-1 light chain | gaaatagtgatgacgcagtctccagccaccctgtctgtgtctccaggggaaagag | SEQ ID NO: 14 |
| coding sequence | ccaccctctcctgcagggccagtcagagtgttagcagcaacttagcctggtacct | |
| | gcagaaacctggccaggctcccaggctcctcatctatggtacatccaccagggc | |
| | cactggtatcccagccaggttcagtggcagtgggtctgggacagagttcactctc | |
| | accatcagcagcctgcagtctgaagattttgcagtttattactgtcagcagtataata | |
| | cctggcctccggagtacacatttggccaggggaccaagctggagatcaaacgta | |
| | cggtggctgcaccatctgtcttcatcttcccgccatctgatgagcagttgaaatctg | |
| | gaactgcctctgttgtgtgcctgctgaataacttctatcccagagaggccaaagtac | |
| | agtggaaggtggataacgccctccaatcgggtaactcccaggagagtgtcacag | |
| | agcaggacagcaaggacagcacctacagcctcagcagcaccctgacgctgag | |
| | caaagcagactacgagaaacacaaagtctacgcctgcgaagtcacccatcagg | |
| | gcctgagctcgcccgtcacaaagagcttcaacaggggagagtgt | |

### II. Antibody Drug Conjugates

In some embodiments, an “antibody drug conjugate” (or ADC) or “conjugate”, may include a binding agent, at least one linker attached to the binding agent; and at least one cytotoxic agent attached to the at least one linker. In some embodiments, the binding agent may be an antibody or an antigen-binding portion thereof. In some embodiments, the conjugate may be an anti-MSLN antibody drug conjugate (or anti-MSLN conjugate). In some embodiments, an anti-MSLN antibody (for example, an Ab-1 antibody or variant thereof), or an antigen-binding portion thereof, may be included in the anti-MSLN antibody drug conjugate (or anti-MSLN conjugate). In some embodiments, the binding agent of the anti-MSLN antibody drug conjugate may include the anti-MSLN antibody (for example, an Ab-1 antibody or variant thereof), or an antigen-binding portion thereof. In some embodiments, the anti-MSLN antibody, or an antigen-binding portion thereof, may be attached to at least one linker, and at least one cytotoxic agent may be attached to each linker.

In some embodiments, the binding agent may be mono-specific.

In some embodiments, the binding agent may be bivalent.

In some embodiments, the binding agent may include a second binding domain and may be bi-specific.

The anti-MSLN conjugates contemplated for use in the compositions and methods herein may comprise at least one cytotoxic agent.

As used herein, a “cytotoxic agent” refers to a compound that exerts a cytotoxic or cytostatic effect on a cell, e.g., by preventing cell growth or replication. A “small molecule” or “compound” is an organic compound with a molecular weight of less than 1500, or 100, or 900, or 750, or 600, or 500 Daltons. A “small molecule drug” is a small molecule that has a therapeutic effect such as treating a disease or disorder. In some embodiments, a small molecule may not be a protein, a polysaccharide, or a nucleic acid.

In some embodiments, the cytotoxic agent may be an anti-mitotic agent or a topoisomerase I inhibitor.

In some embodiments, the cytotoxic agent may be a microtubule disrupting agent (for example, a tubulin disrupting agent) or a DNA modifying agent. In some embodiments, the the cytotoxic agent may be an anti-mitotic agent, for example, a microtubule disrupting agent or a tubulin disrupting agent.

In some embodiments, the anti-MSLN conjugate may include a cytotoxic agent that is a tubulin disrupting agent. Several different categories of tubulin disrupting agent are known, including, auristatins, tubulysins, colchicine, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine or monomethyl auristatin E) and MMAF (N-methylvaline-valine-dolaisoleuine-dolaproine-phenylalanine or monomethyl auristatin F) and AFP (see, for example, PCT Publication Nos. WO2004/010957 and WO2007/008603). PCT Publication No. WO 2015/057699 describes PEGylated auristatins including MMAE. Additional dolastatin derivatives contemplated for use are disclosed in U.S. Pat. No. 9,345,785, incorporated herein by reference.

Examples of tubulysins may include, but are not limited to, tubulysin D, tubulysin M, tubuphenylalanine and tubutyrosine. PCT Publication Nos. WO 2017/096311 and WO 2016/040684 describe tubulysin analogs including tubulysin M.

Examples of colchicines may include, but are not limited to, colchicine and CA-4.

Examples of vinca alkaloids may include, but are not limited to, vinblastine (VBL), vinorelbine (VRL), vincristine (VCR) and vindesine (VOS).

Examples of taxanes may include, but are not limited to, paclitaxel and docetaxel.

Examples of cryptophycins may include but are not limited to cryptophycin-1 and cryptophycin-52.

Examples of maytansinoids may include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Examples of maytansinoid drug moieties may also include those having modifications such as: C-9-SH (U.S. Pat. No. 4,424,219) (prepared by the reaction of maytansinol with H₂S or P2S5); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol). The cytotoxicity of the TA.1-maytansonoid conjugate that binds HER-2 (Chari et al., Cancer Research 52:127-131, 1992) was tested in vitro on the human breast cancer cell line SK-BR-3. The drug conjugate achieved a degree of cytotoxicity similar to the free maytansinoid drug, which could be increased by increasing the number of maytansinoid molecules per antibody molecule.

Examples of hemiasterlins may include, but are not limited to, hemiasterlin and HTl-286.

Other tubulin disrupting agents may include taccalonolide A, taccalonolide B, taccalonolide AF, taccalonolide AJ, taccalonolide Al-epoxide, discodermolide, epothilone A, epothilone B, and laulimalide.

In some embodiments, the cytotoxic agent may be a DNA modifying agent. In some embodiments, the DNA modifying agent may be an alkylating agent or topoisomerase inhibitor. In some embodiments, the DNA modifying agent may be a duocarmycin or analog thereof, calicheamicin, or pyrrolobenzodiazepine,

In some embodiments, the cytotoxic agent may be a topoisomerase inhibitor, such as a camptothecin or a camptothecin analog, or an anthracycline. In some embodiments, the cytotoxic agent may be a topoisomerase I inhibitor. Examples of camptothecins, or analogs thereof, may include irinotecan (also referred to as CPT-11), topotecan, 10-hydroxy-CPT, SN-38, exatecan and the exatecan analog DXd (see, for example, U.S. Patent Publication No. 2015/0297748). In some embodiments, the cytotoxic agent, for example, the camptothecin, or camptothecin analog, may be part of a drug-linker as disclosed in PCT Publication No. WO2023280227A2, incorporated herein by reference in its entirety. In some embodiments the anti-MSLN conjugate may have one of the following structures, where Ab is a MSLN binding agent as disclosed herein:

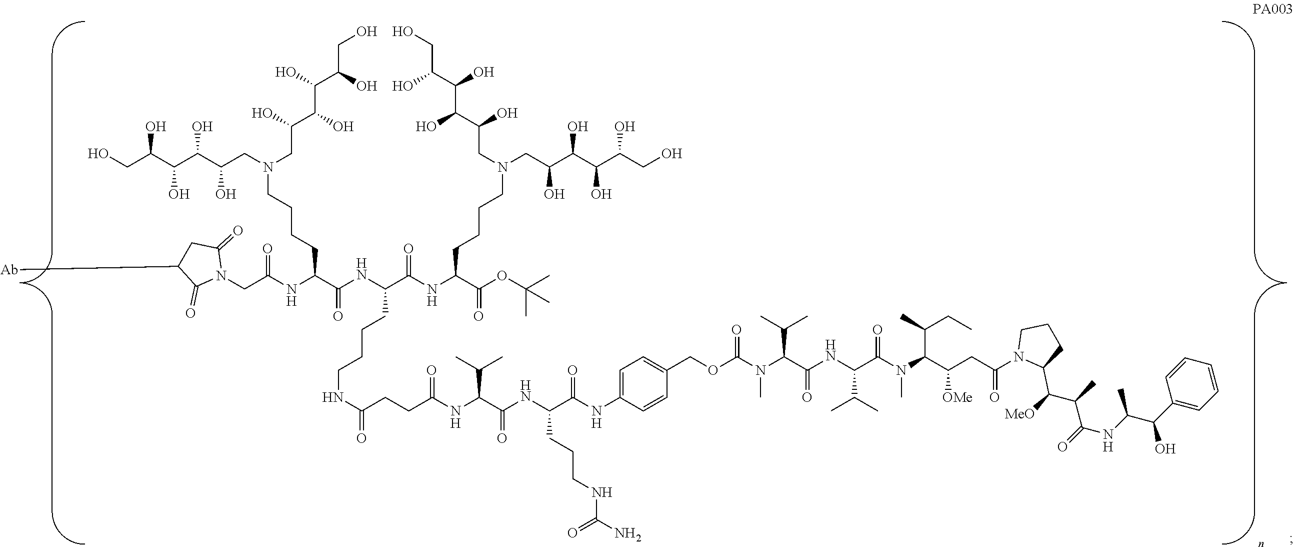

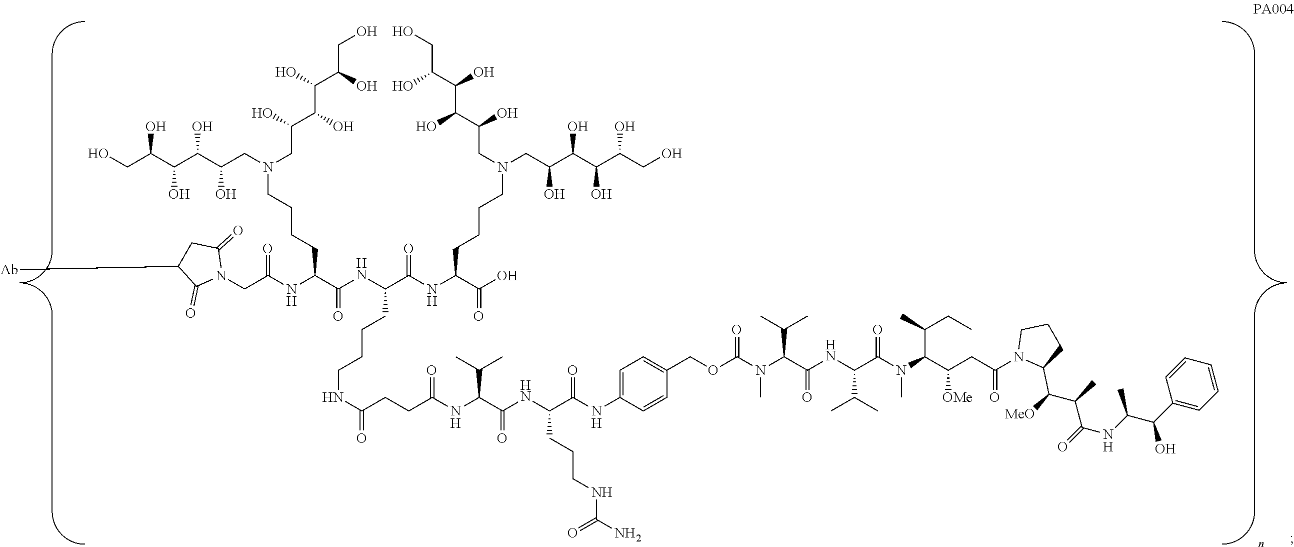

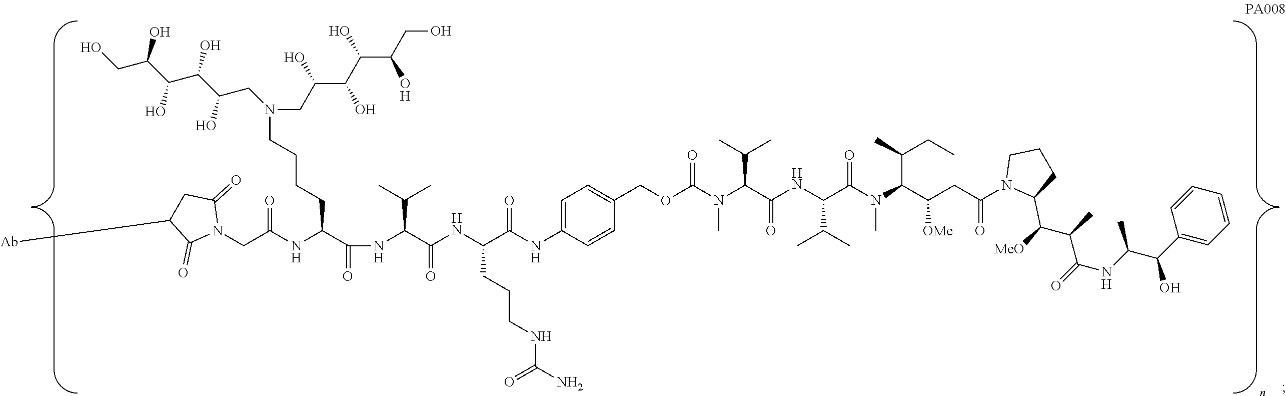

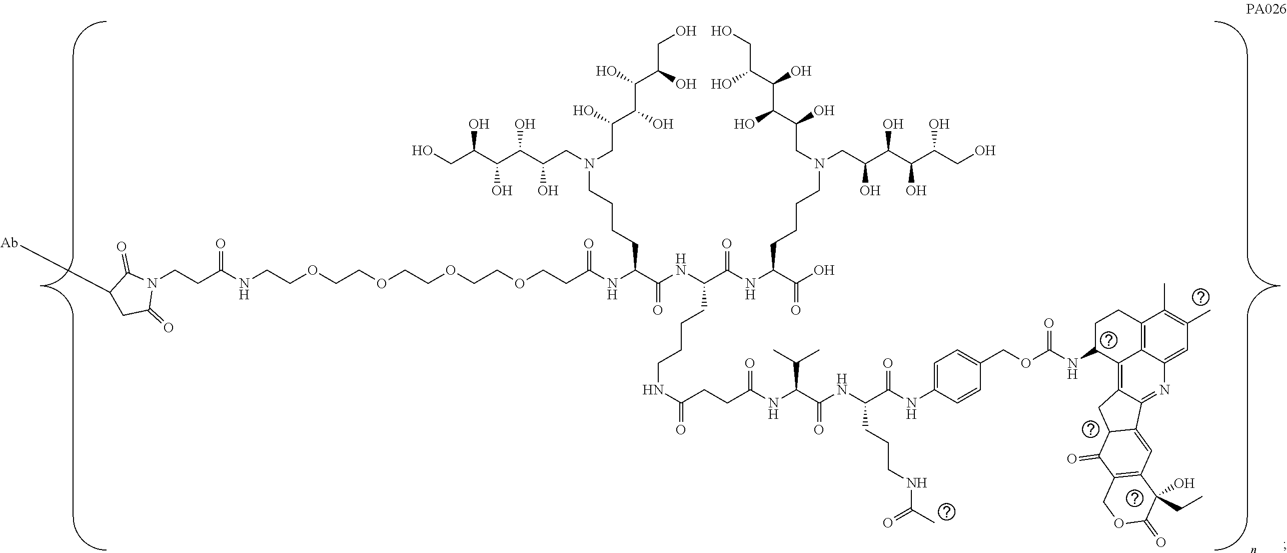

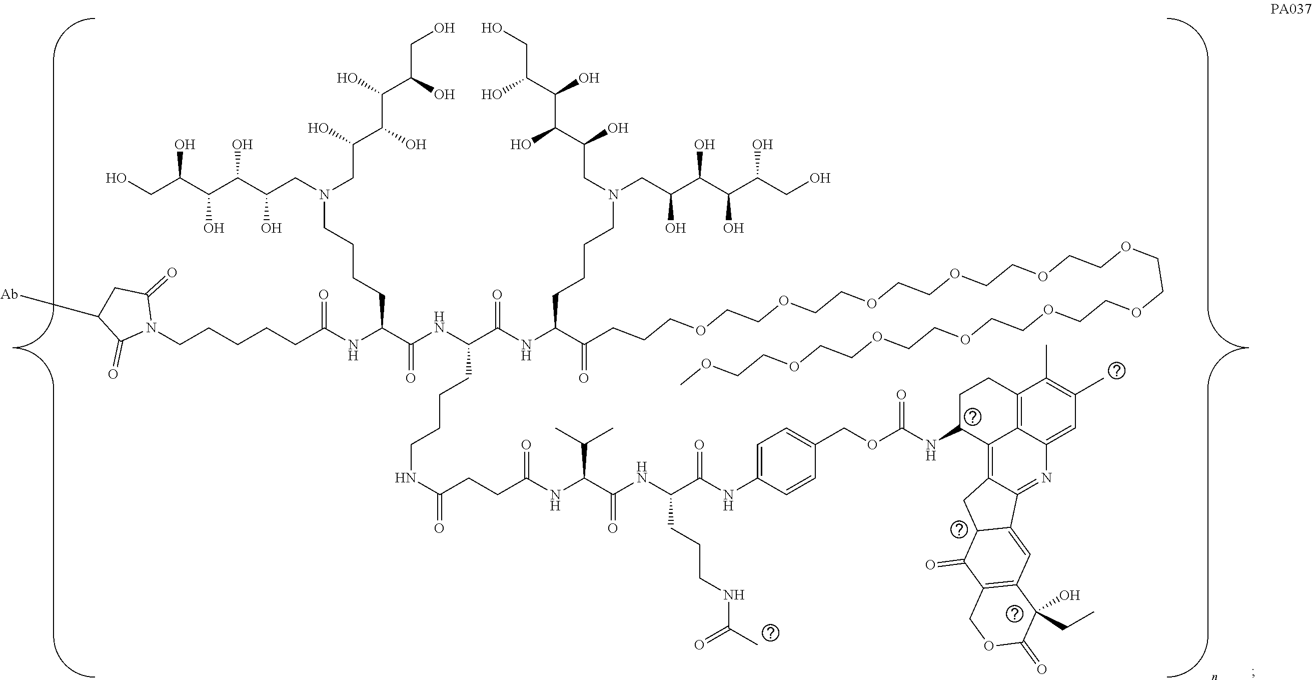

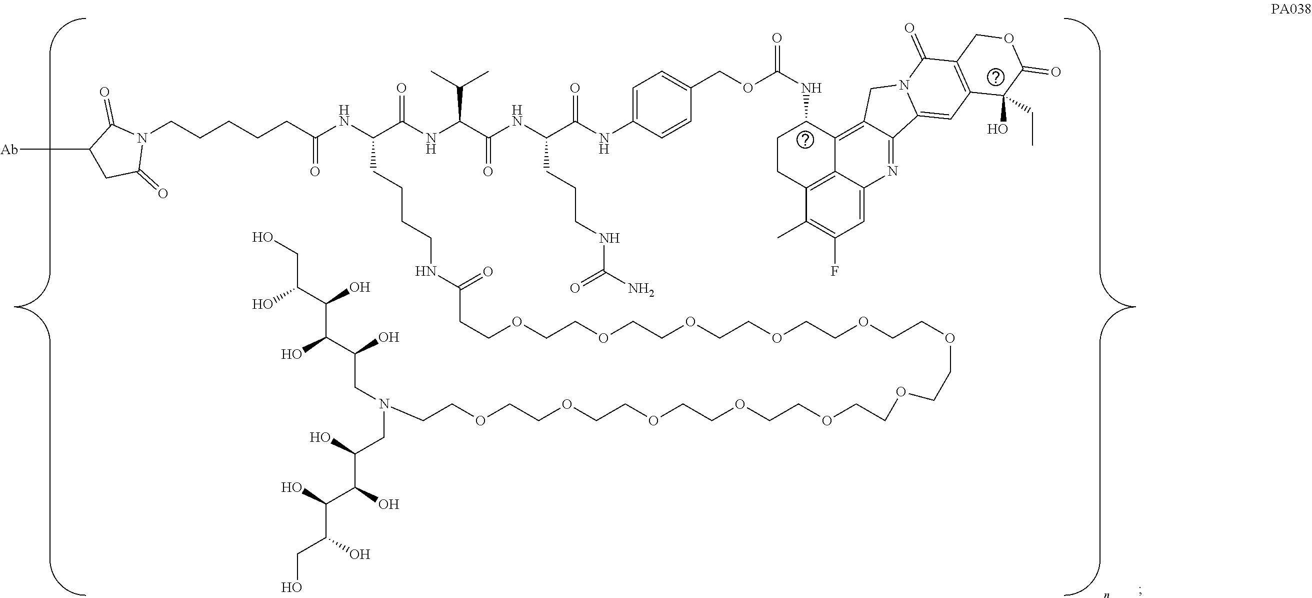

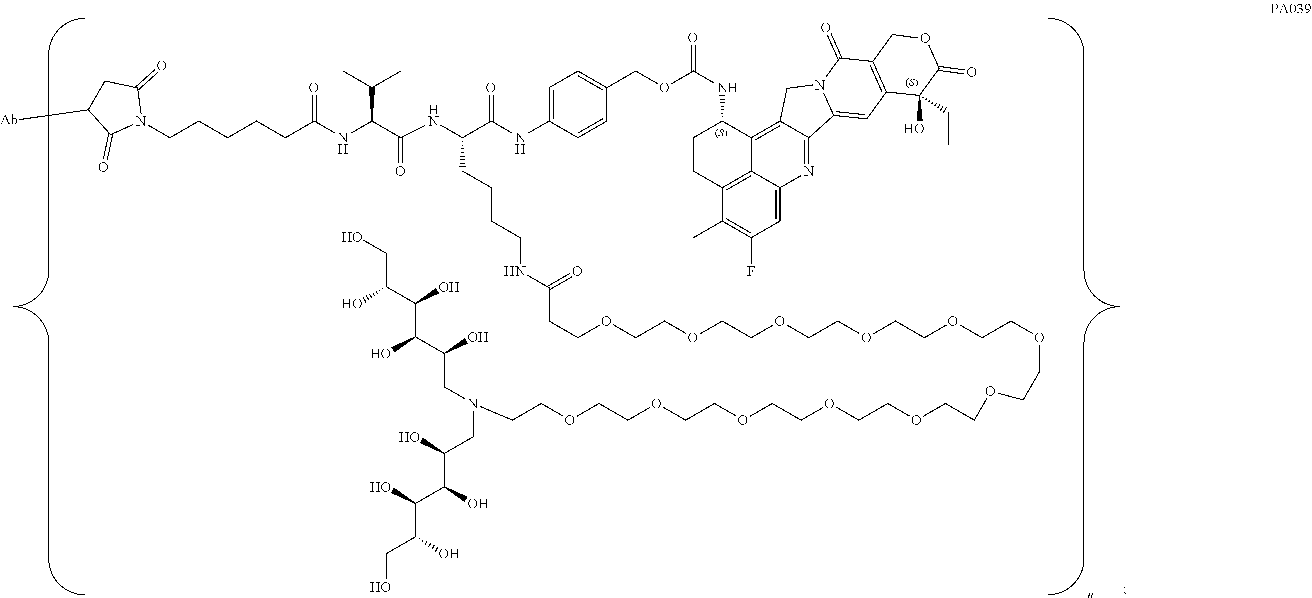

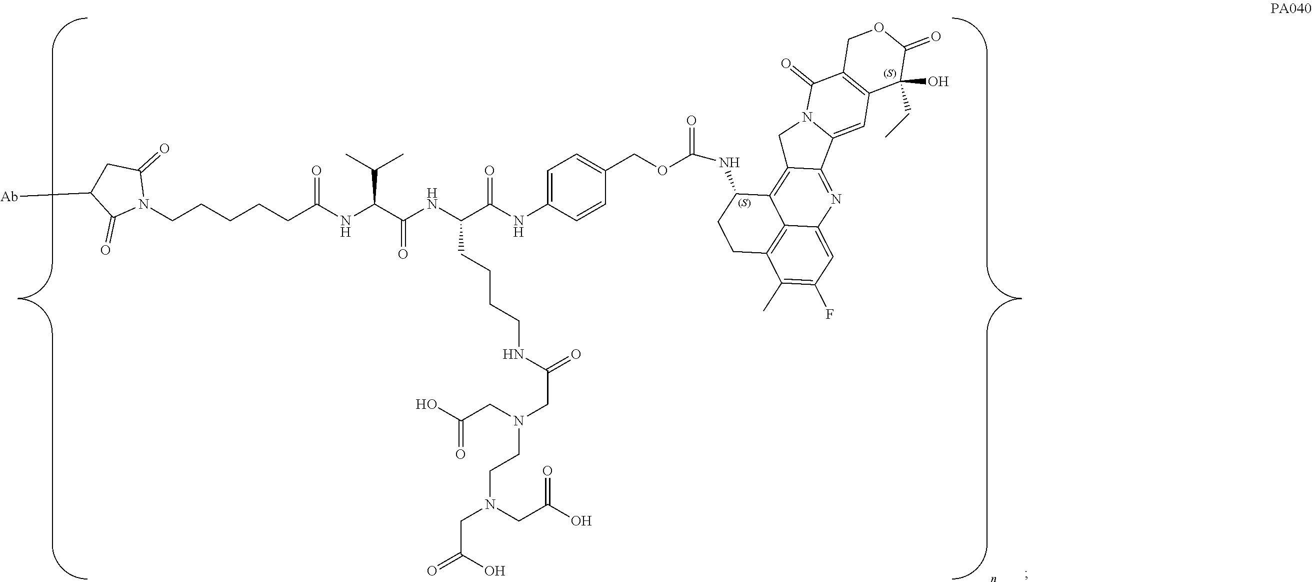

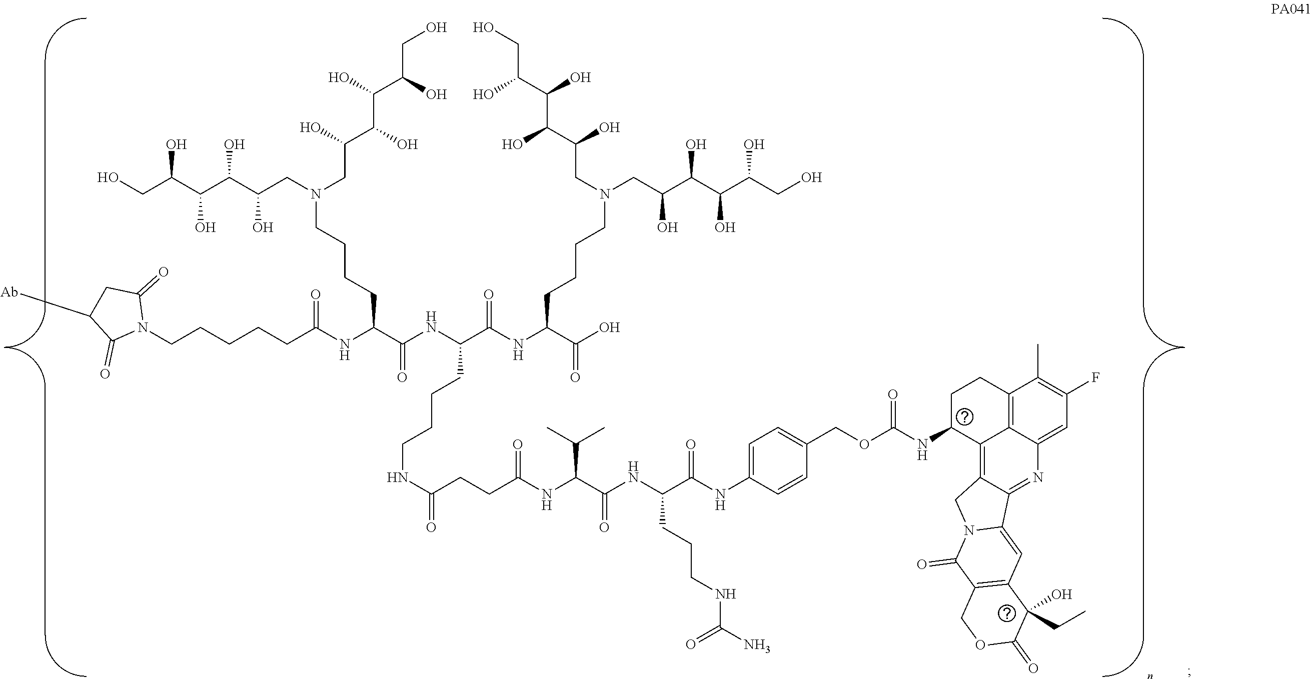

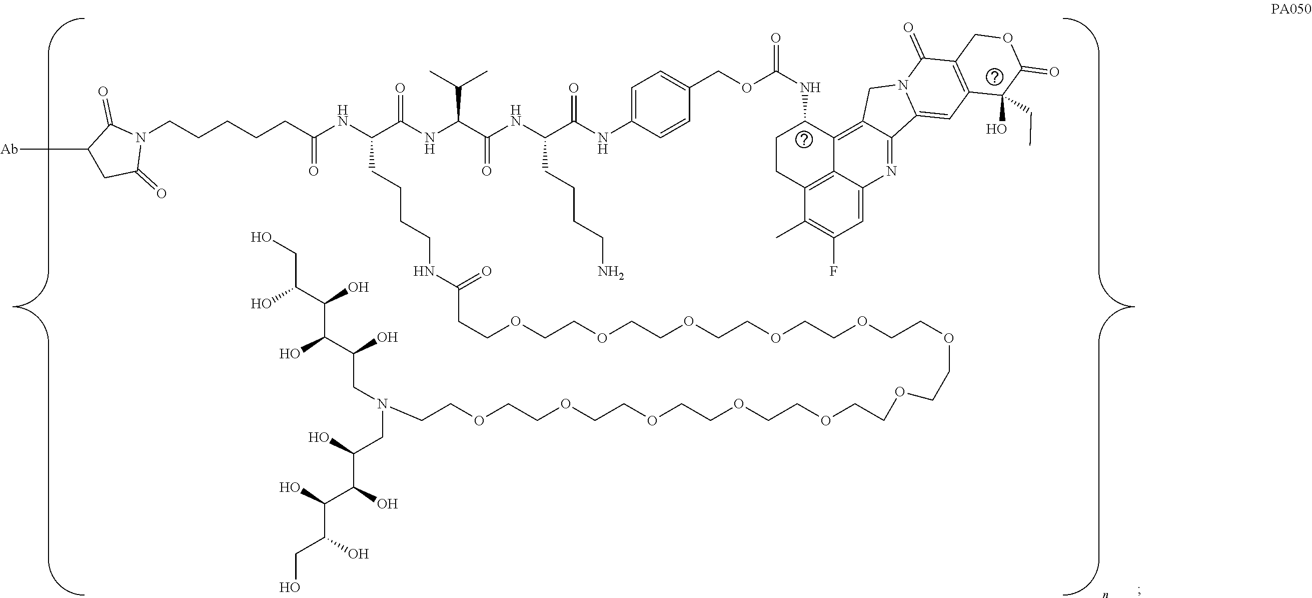

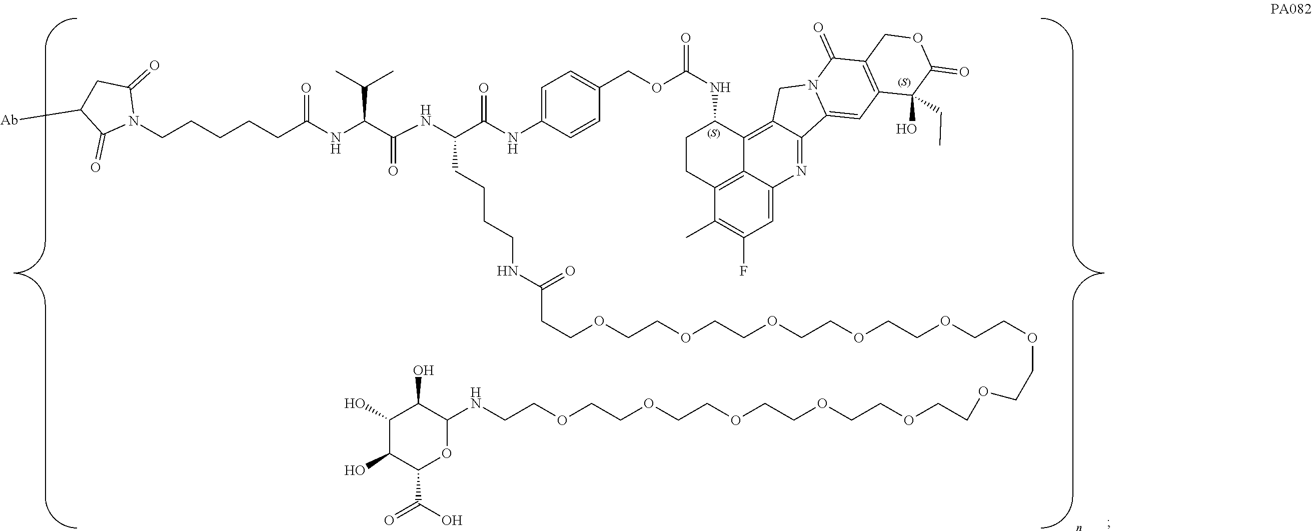

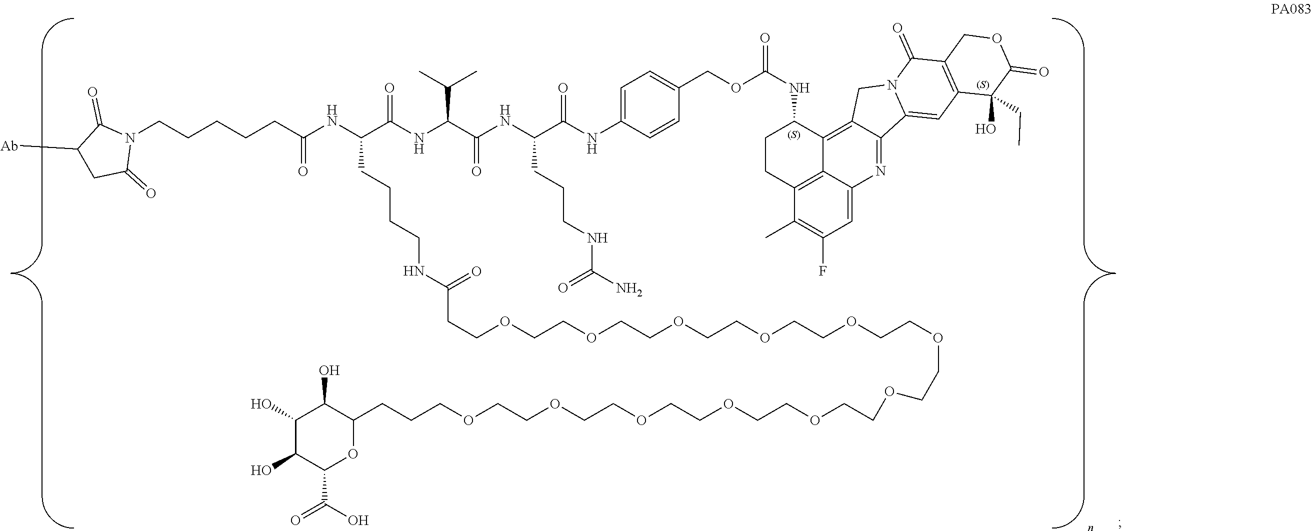

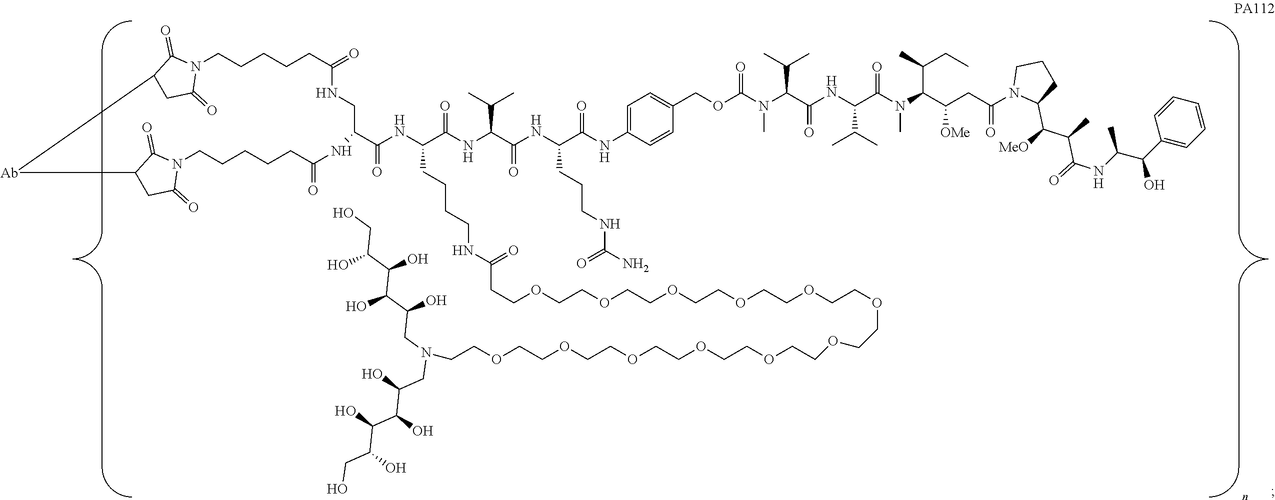

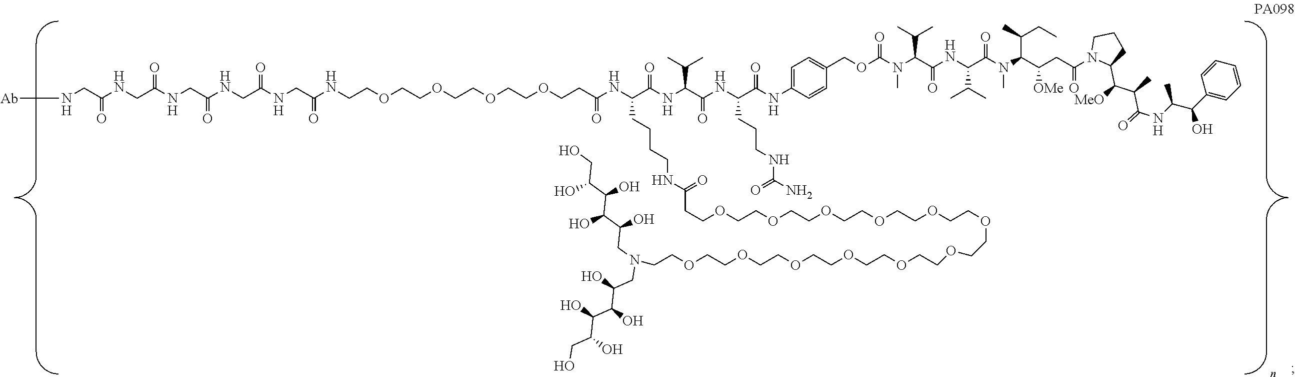

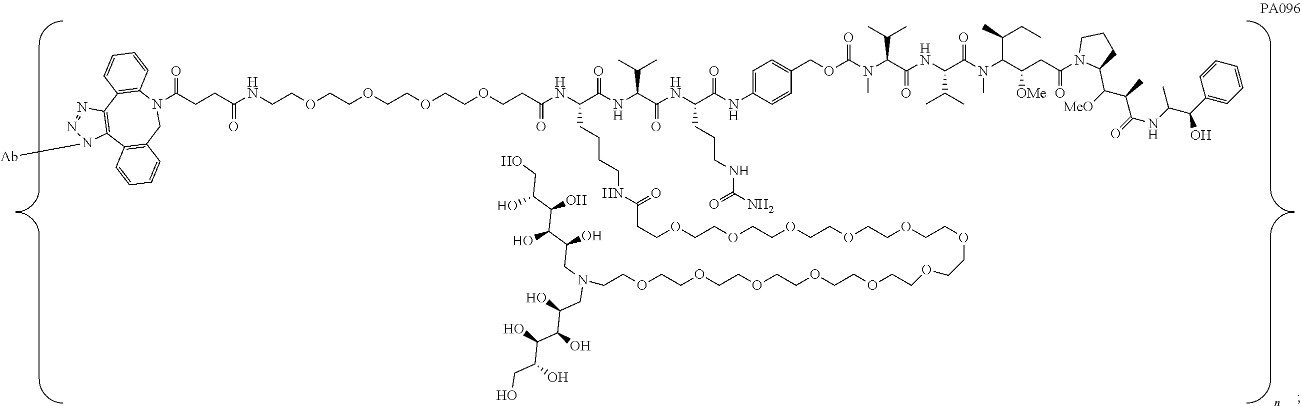

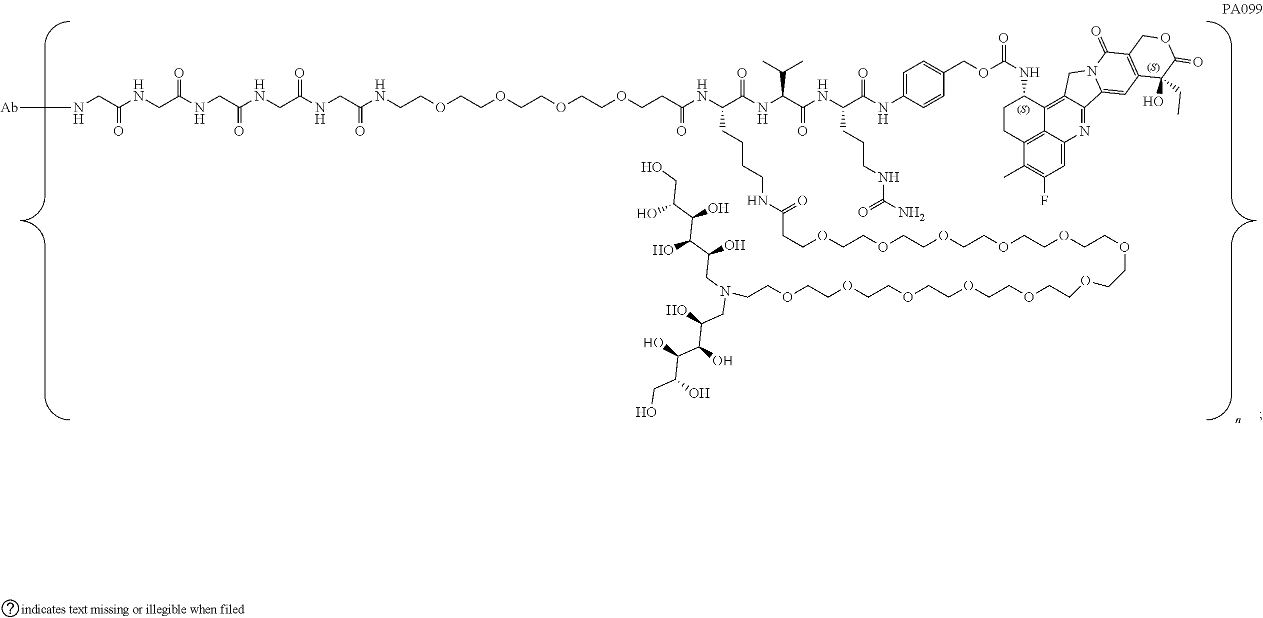

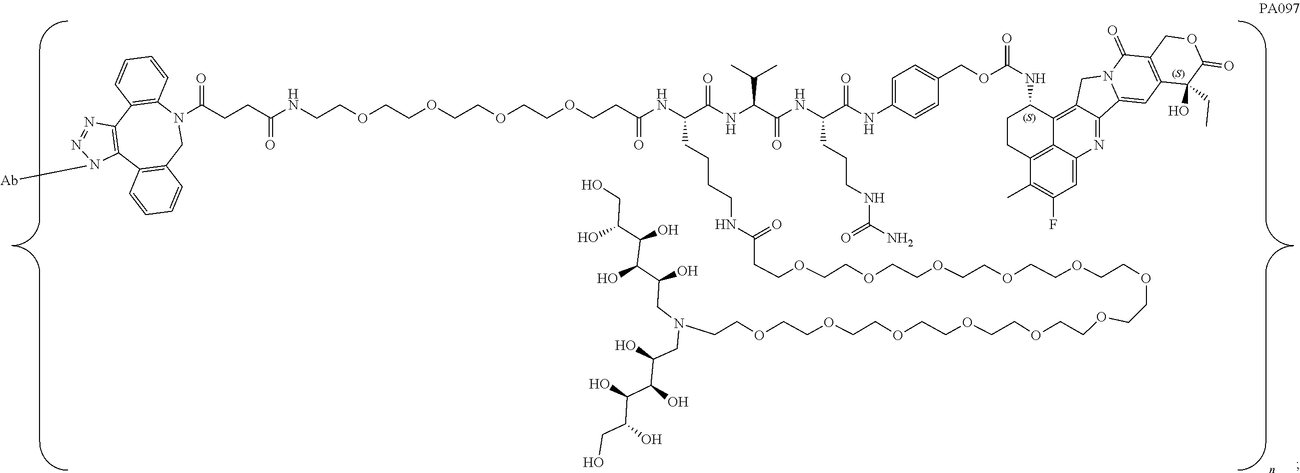

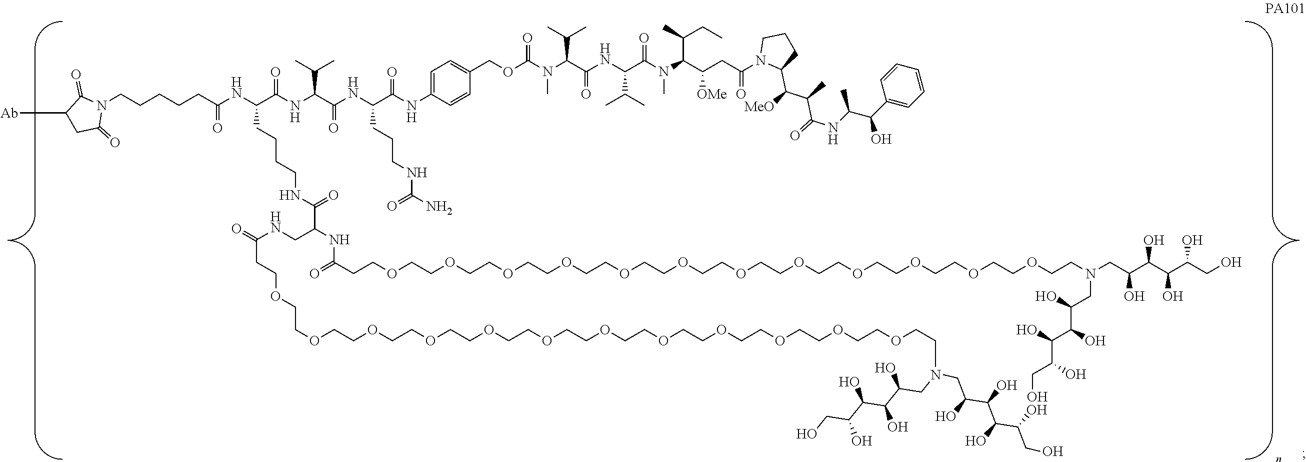

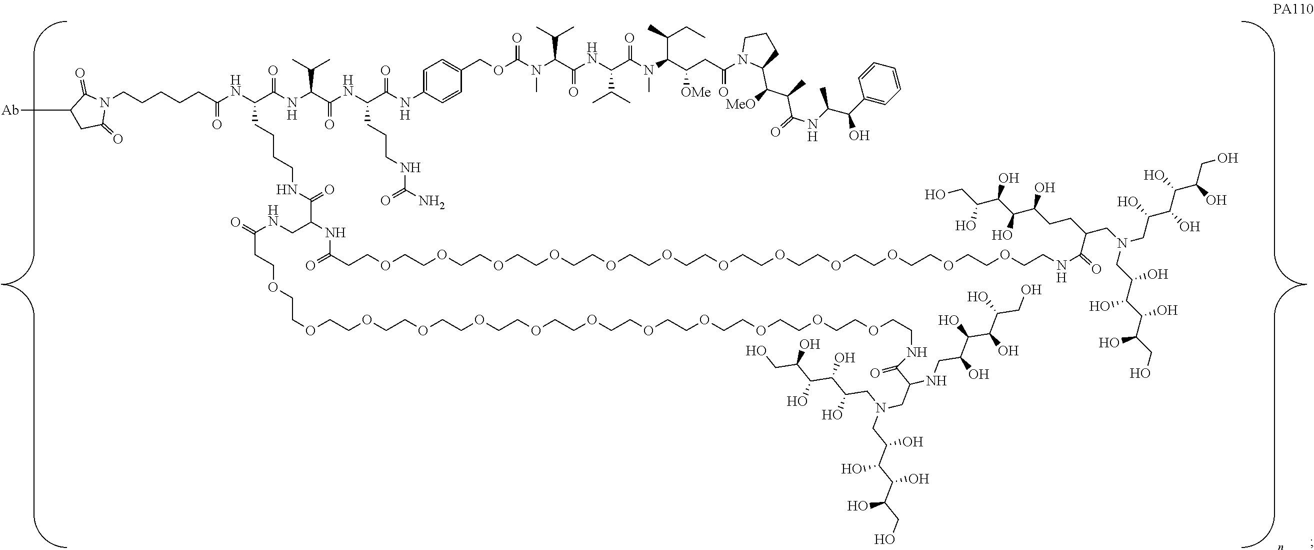

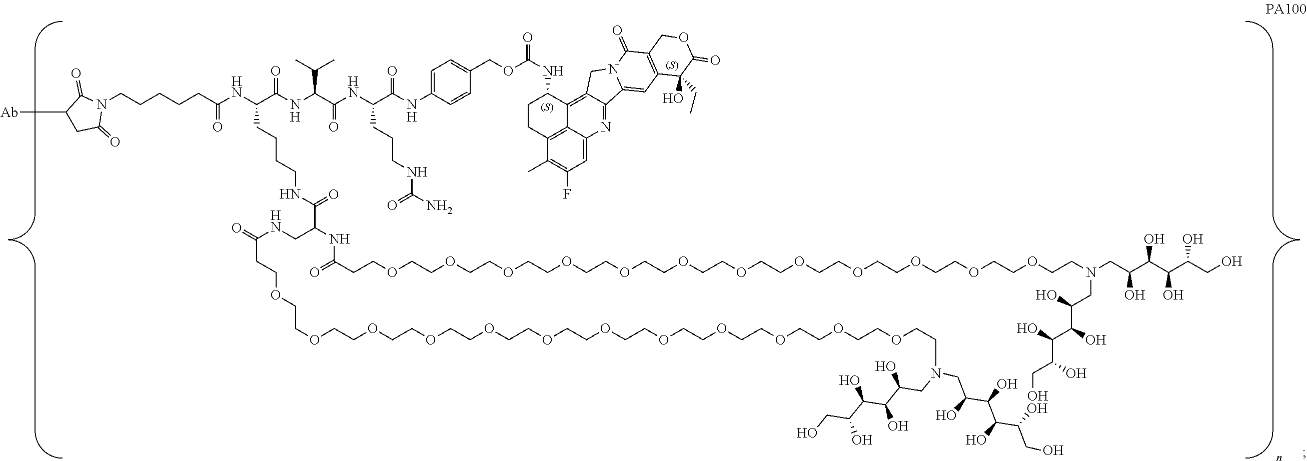

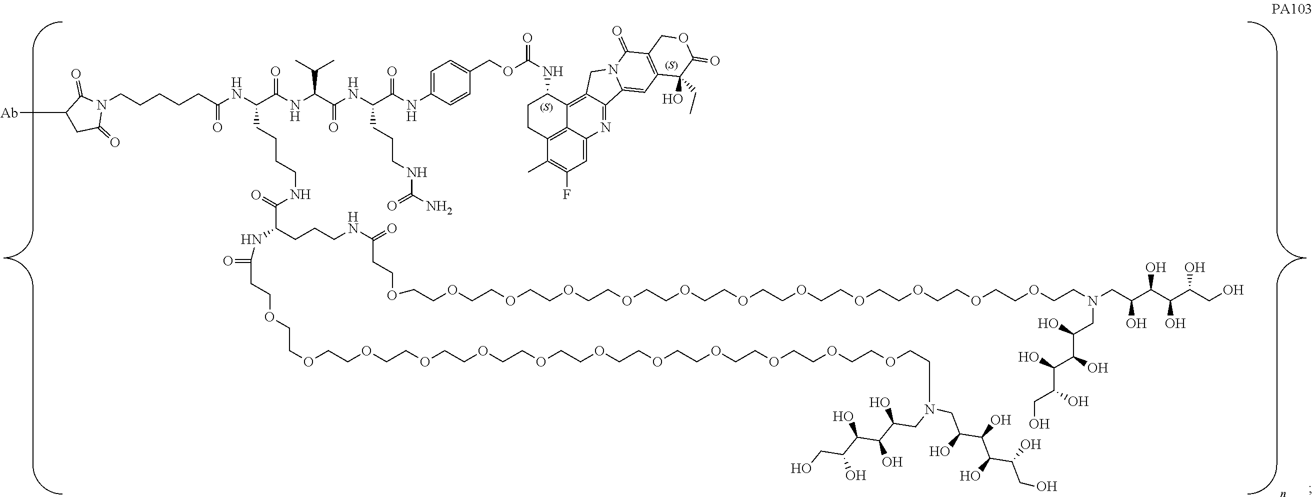

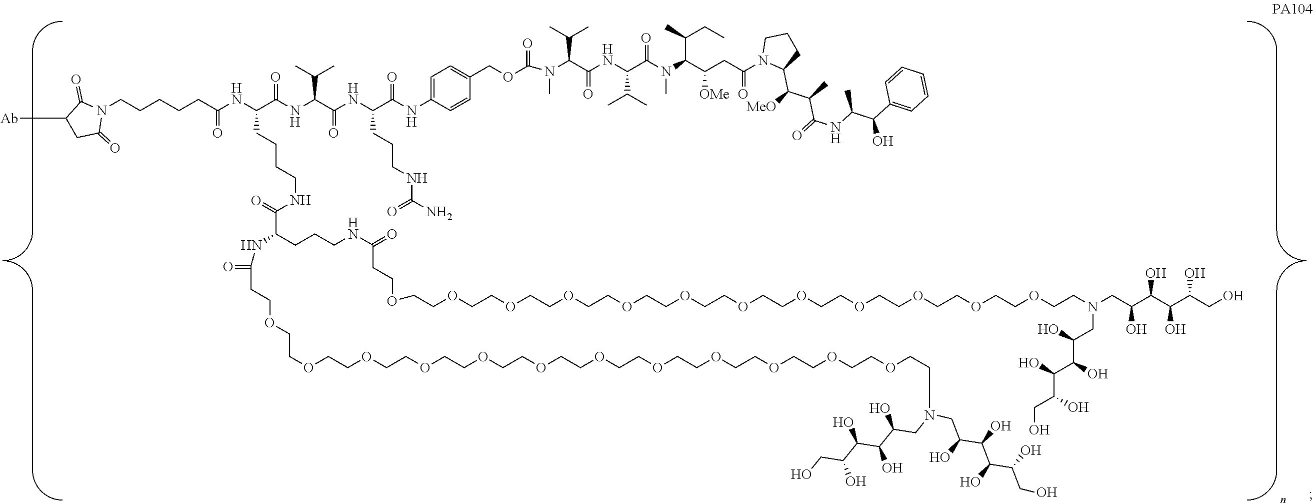

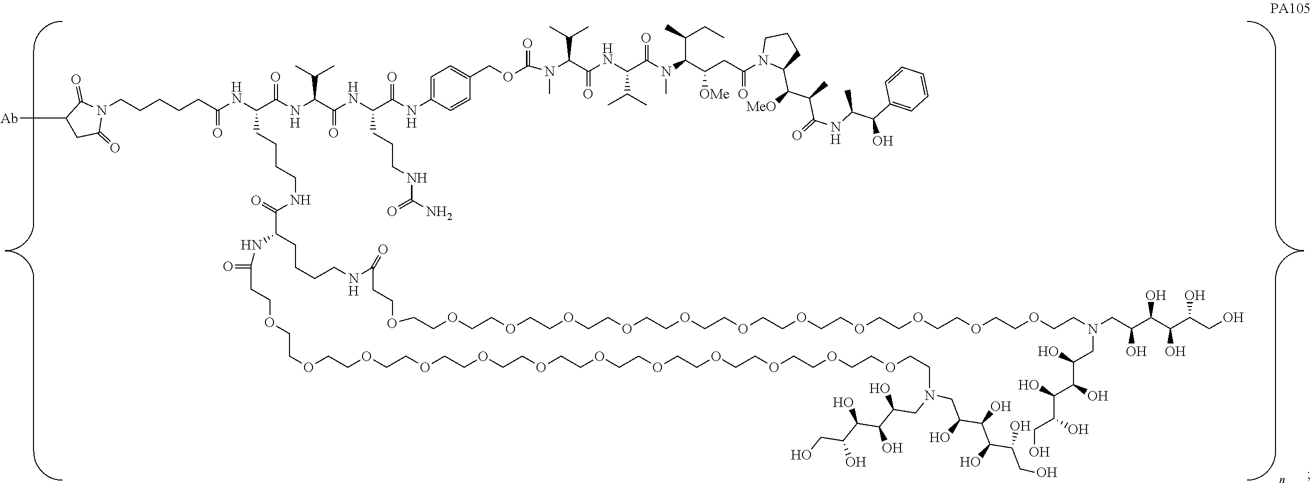

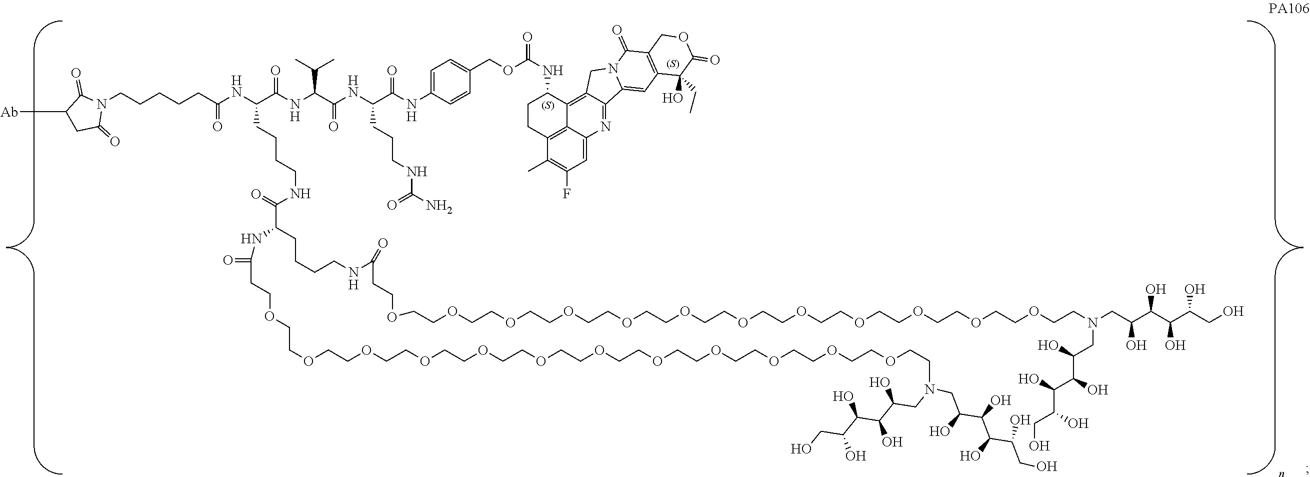

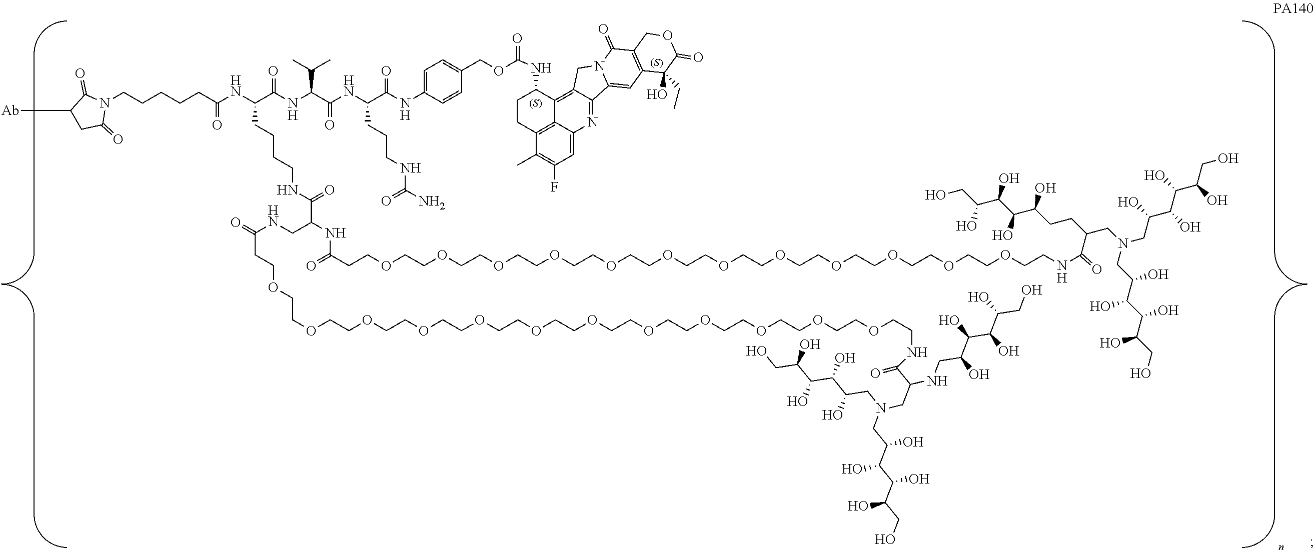

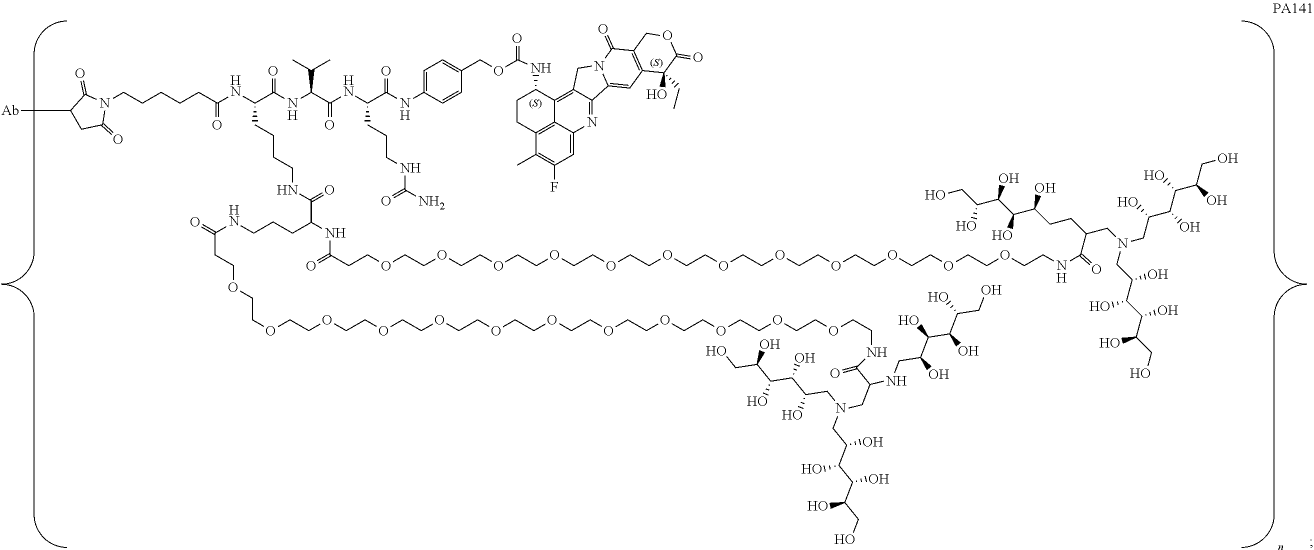

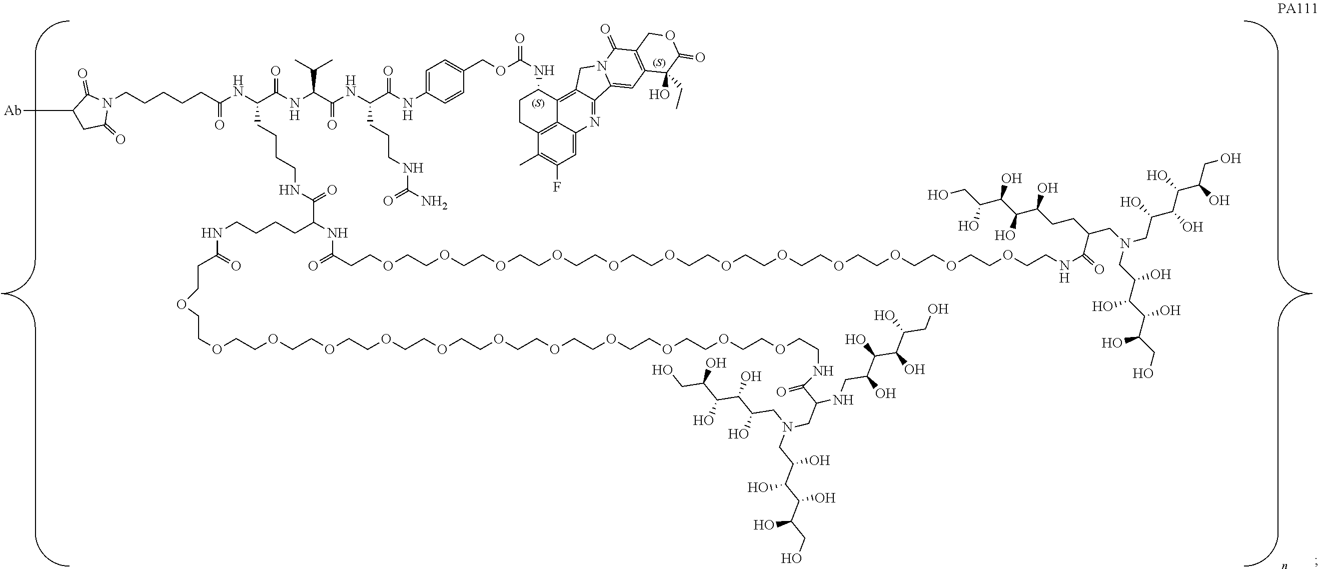

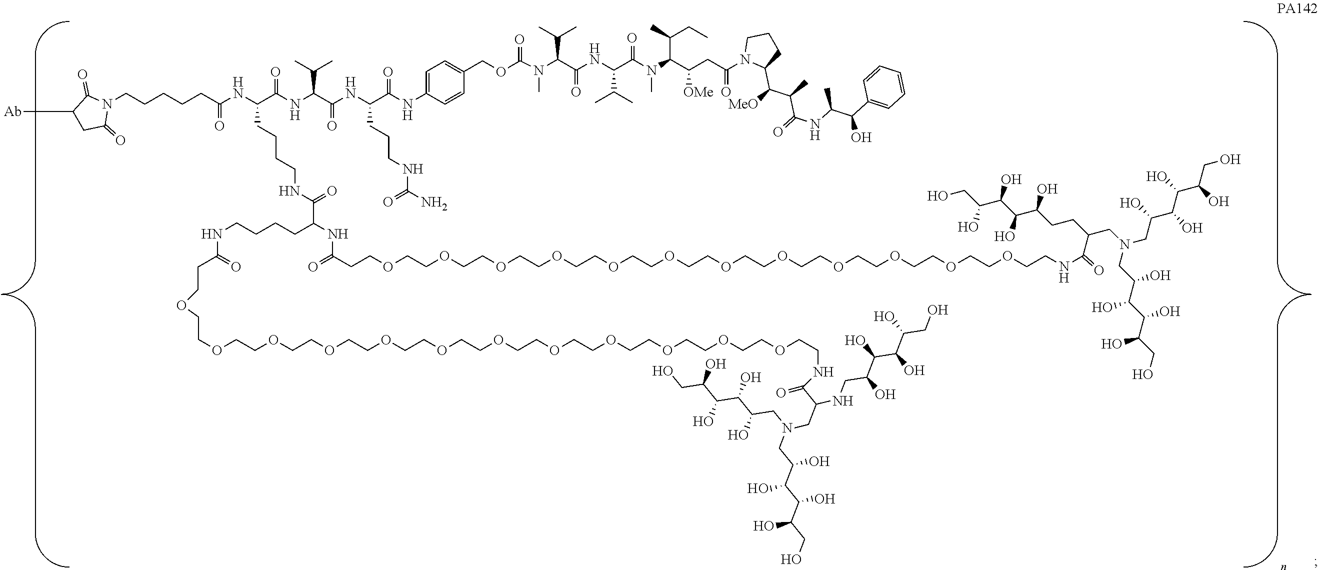

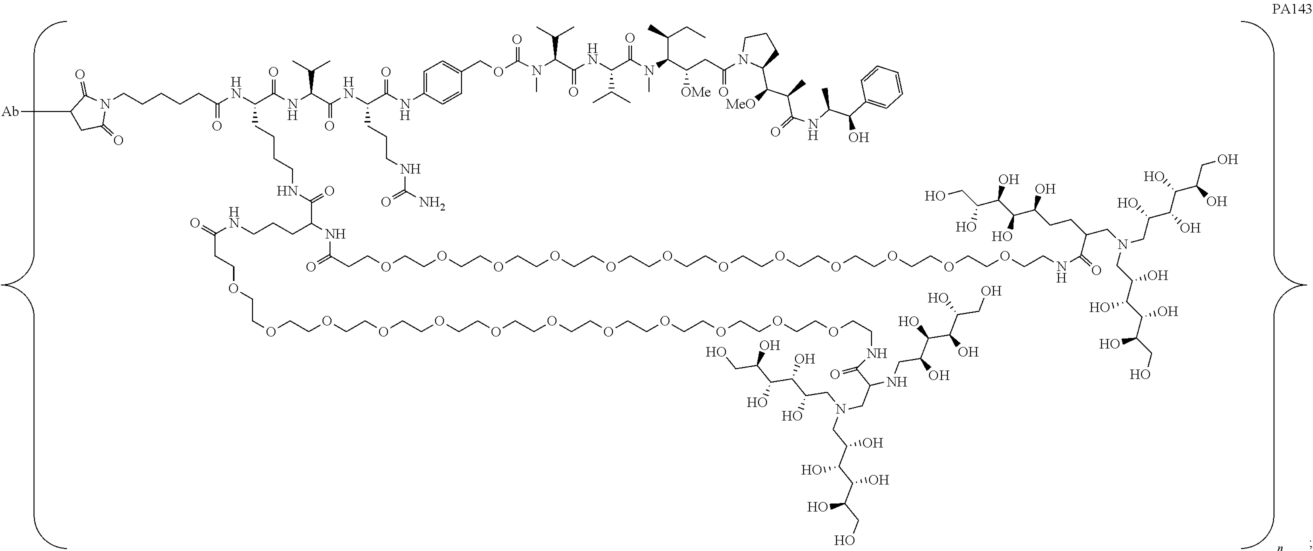

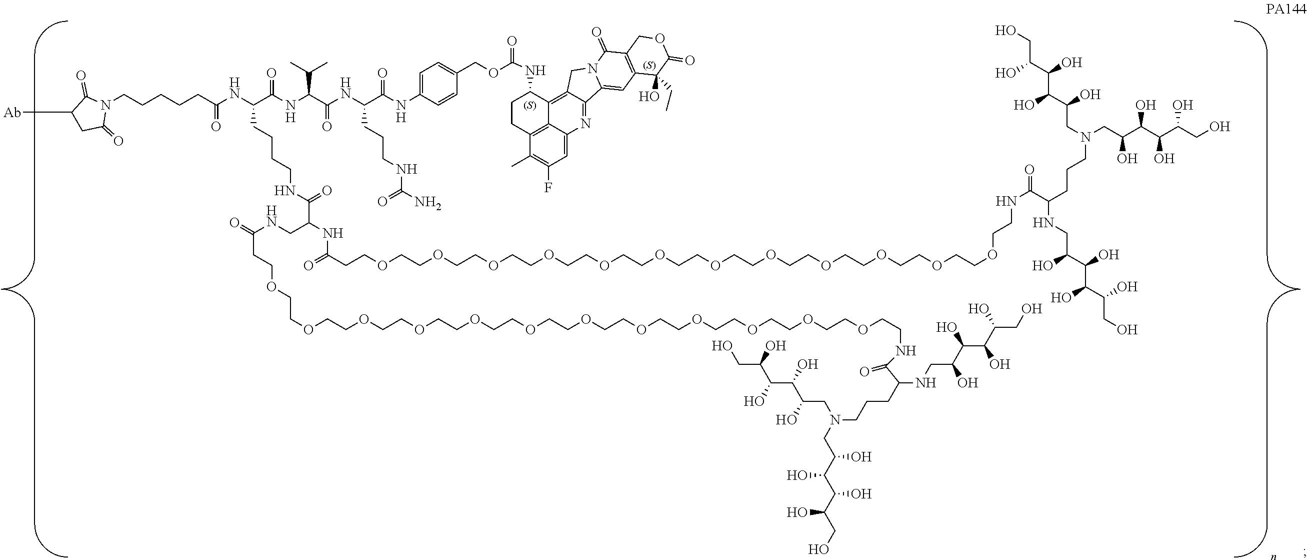

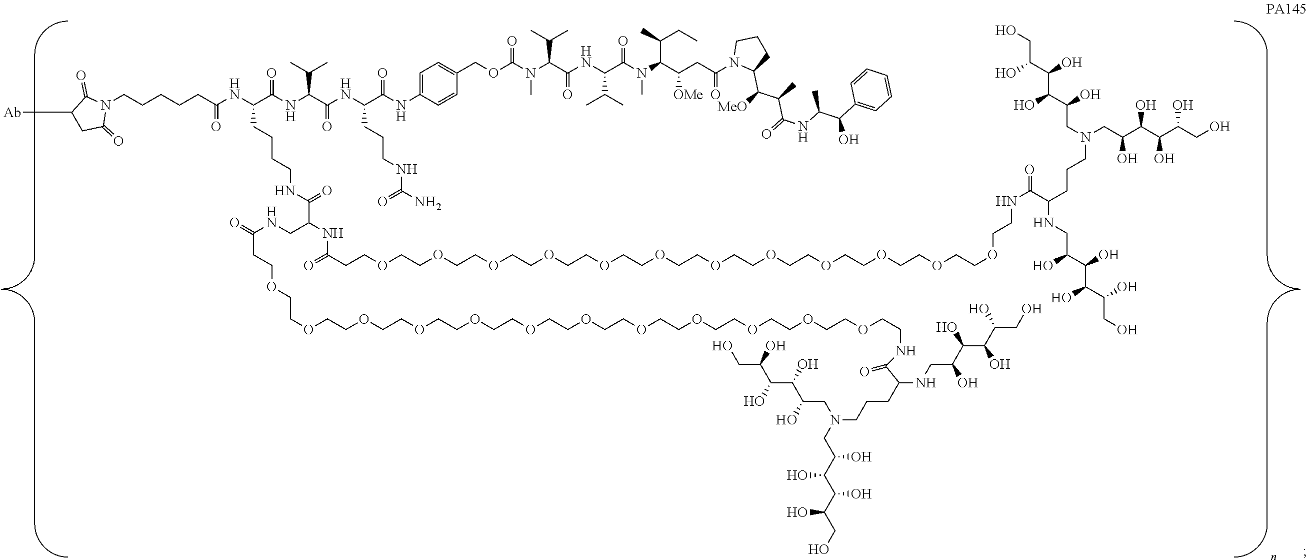

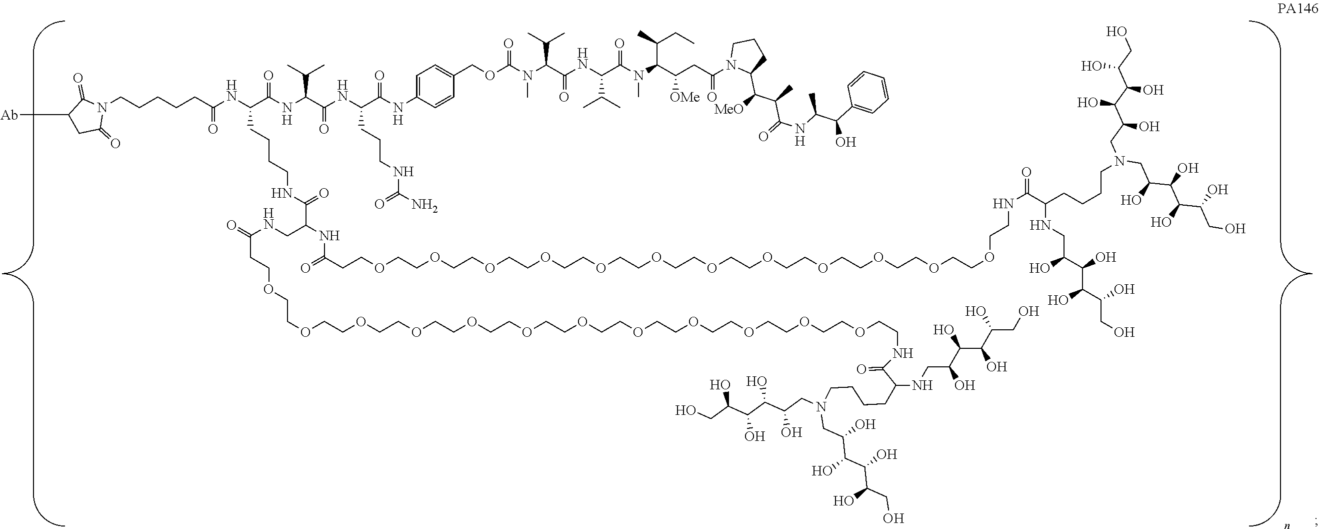

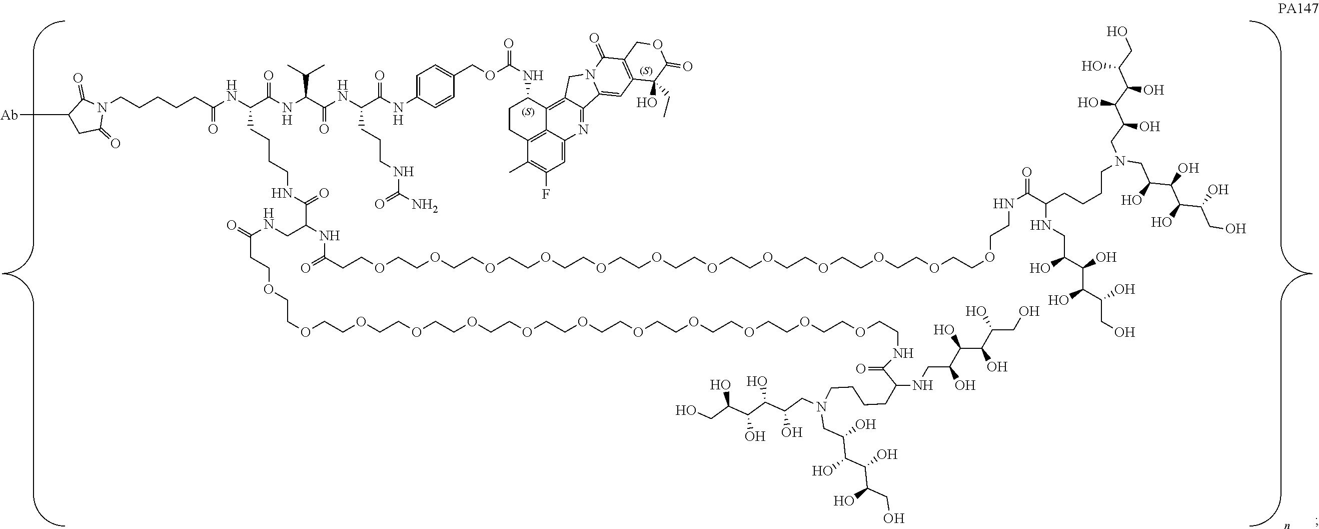

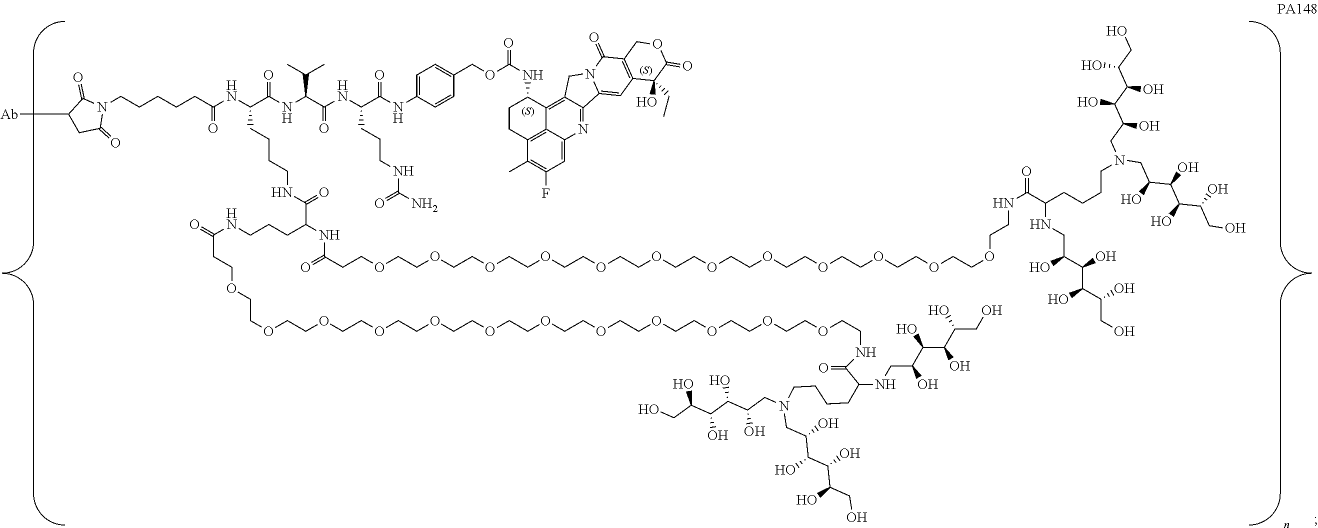

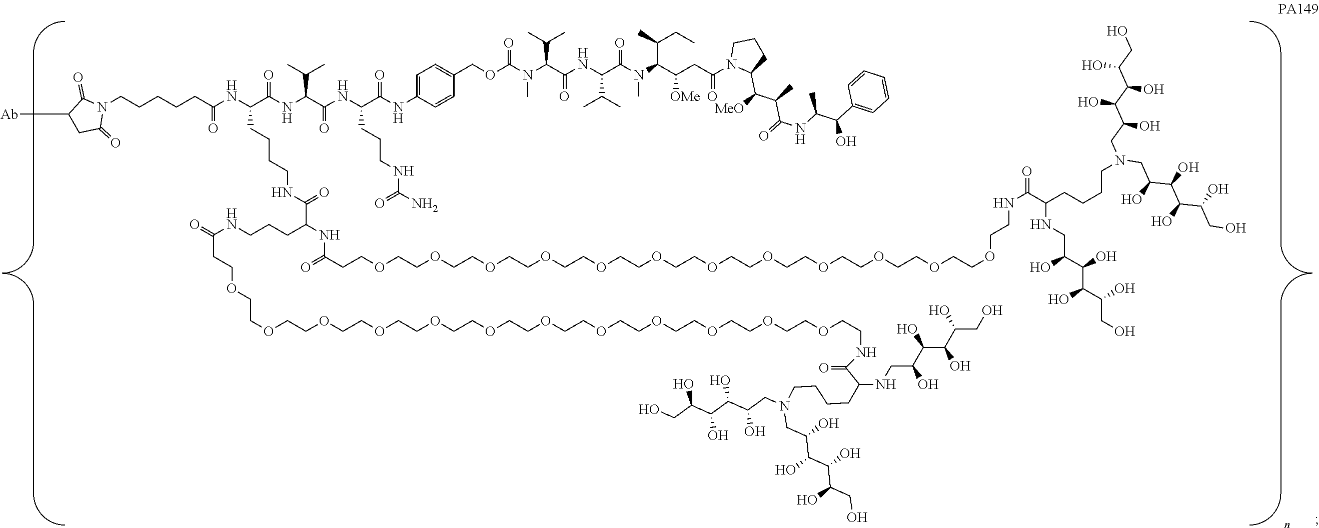

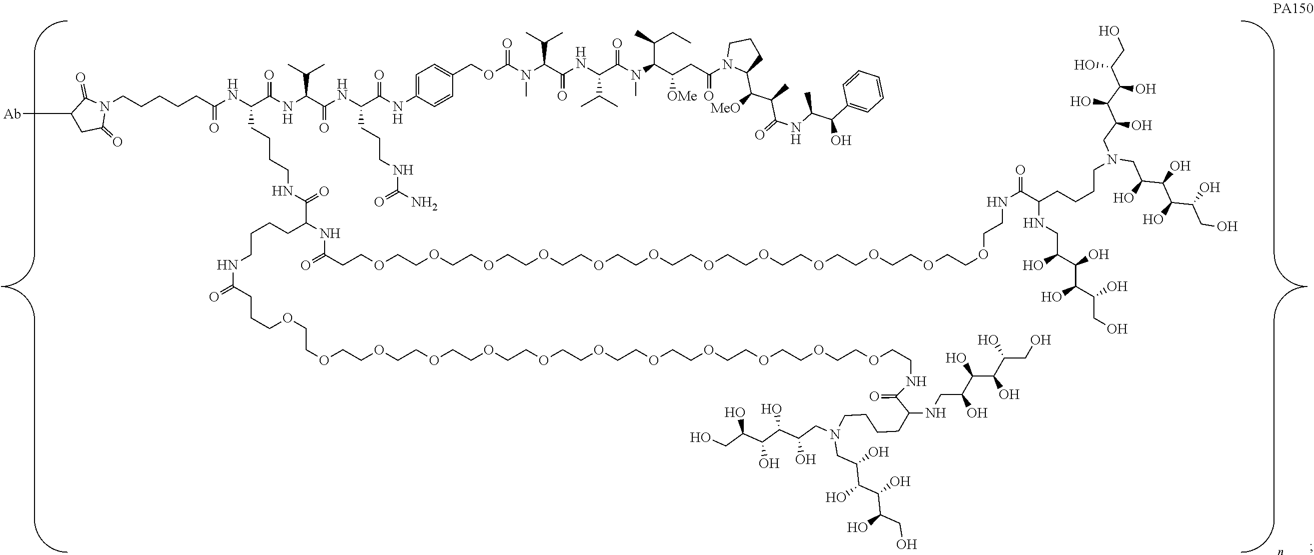

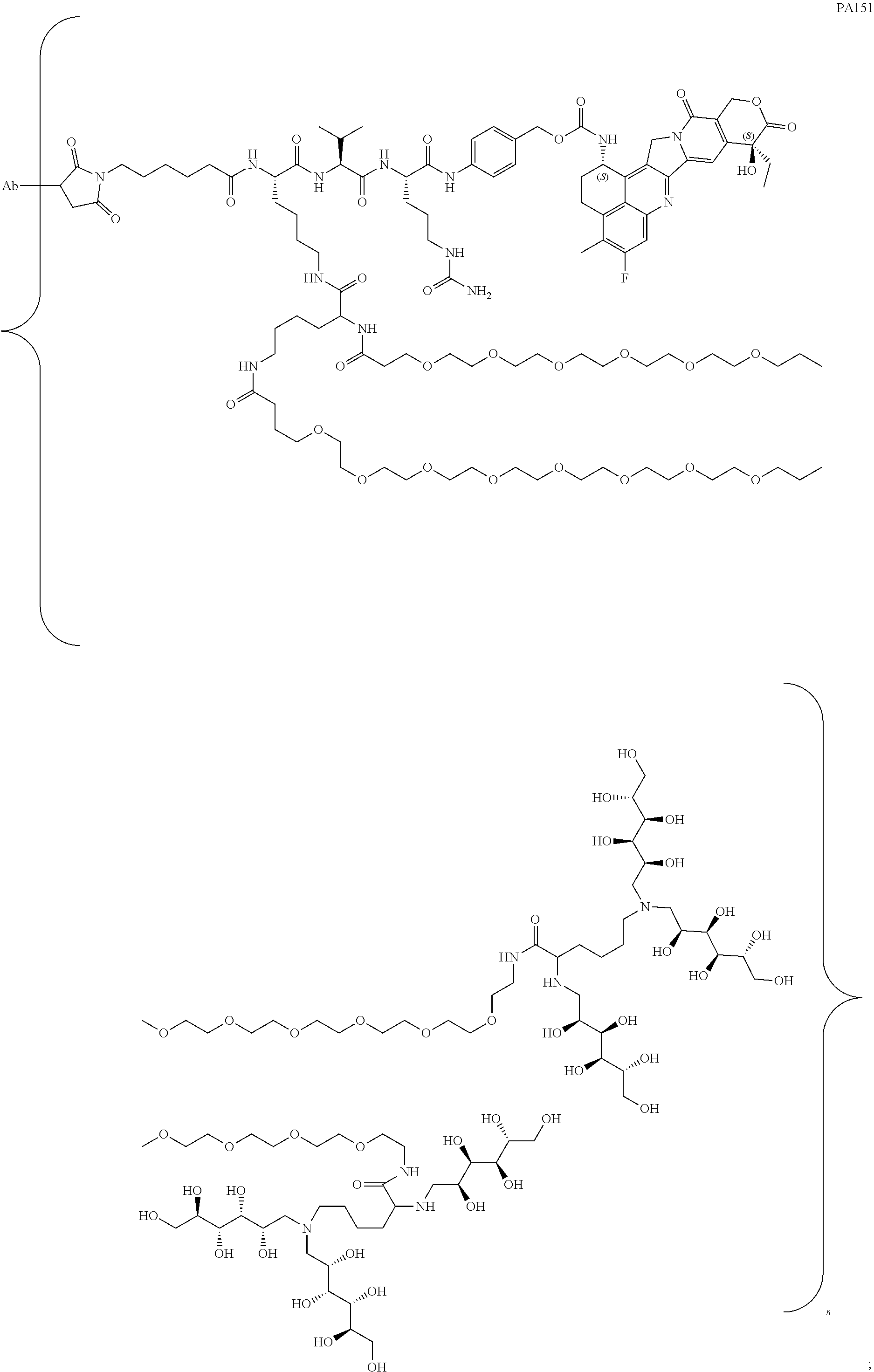

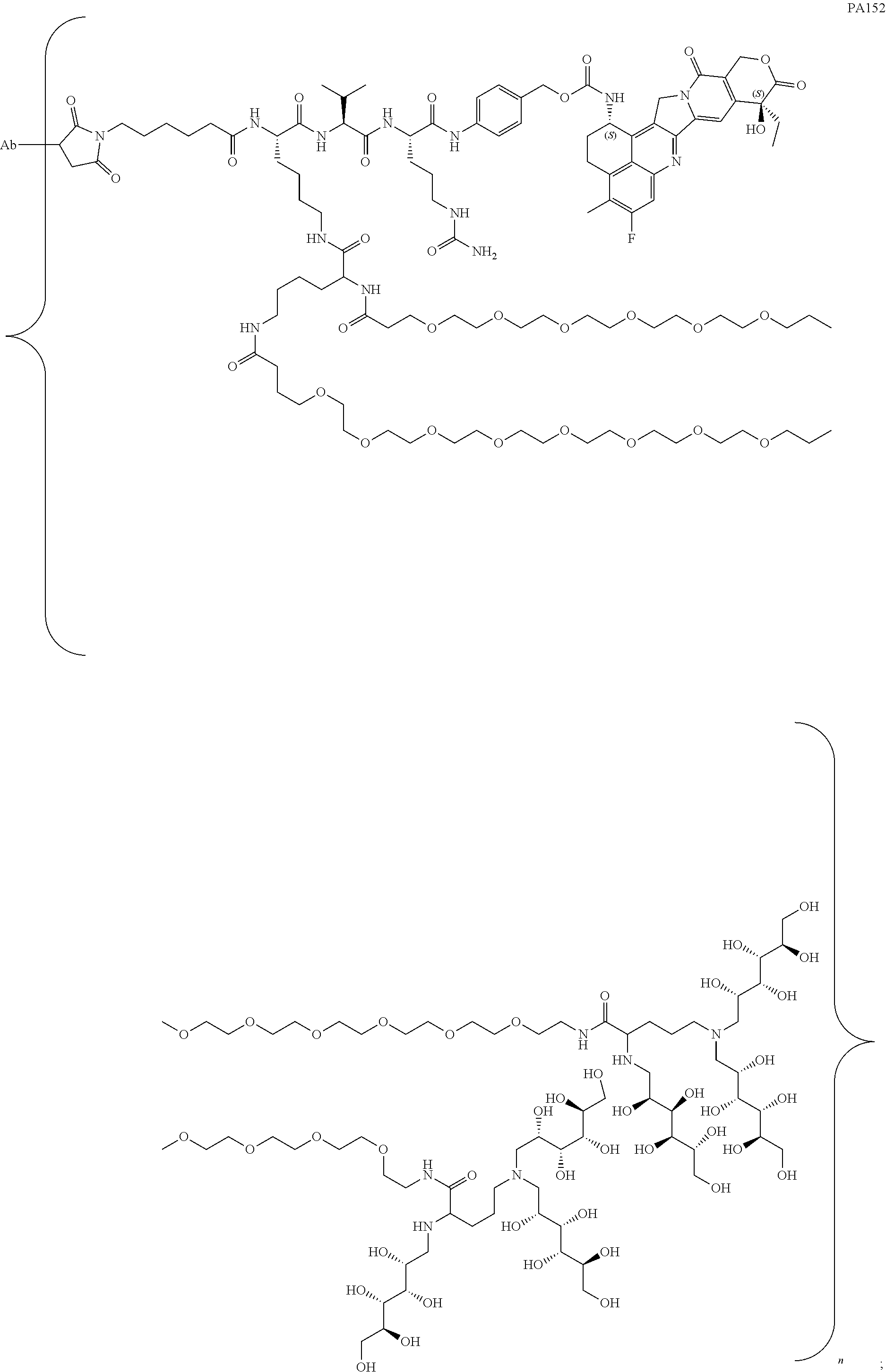

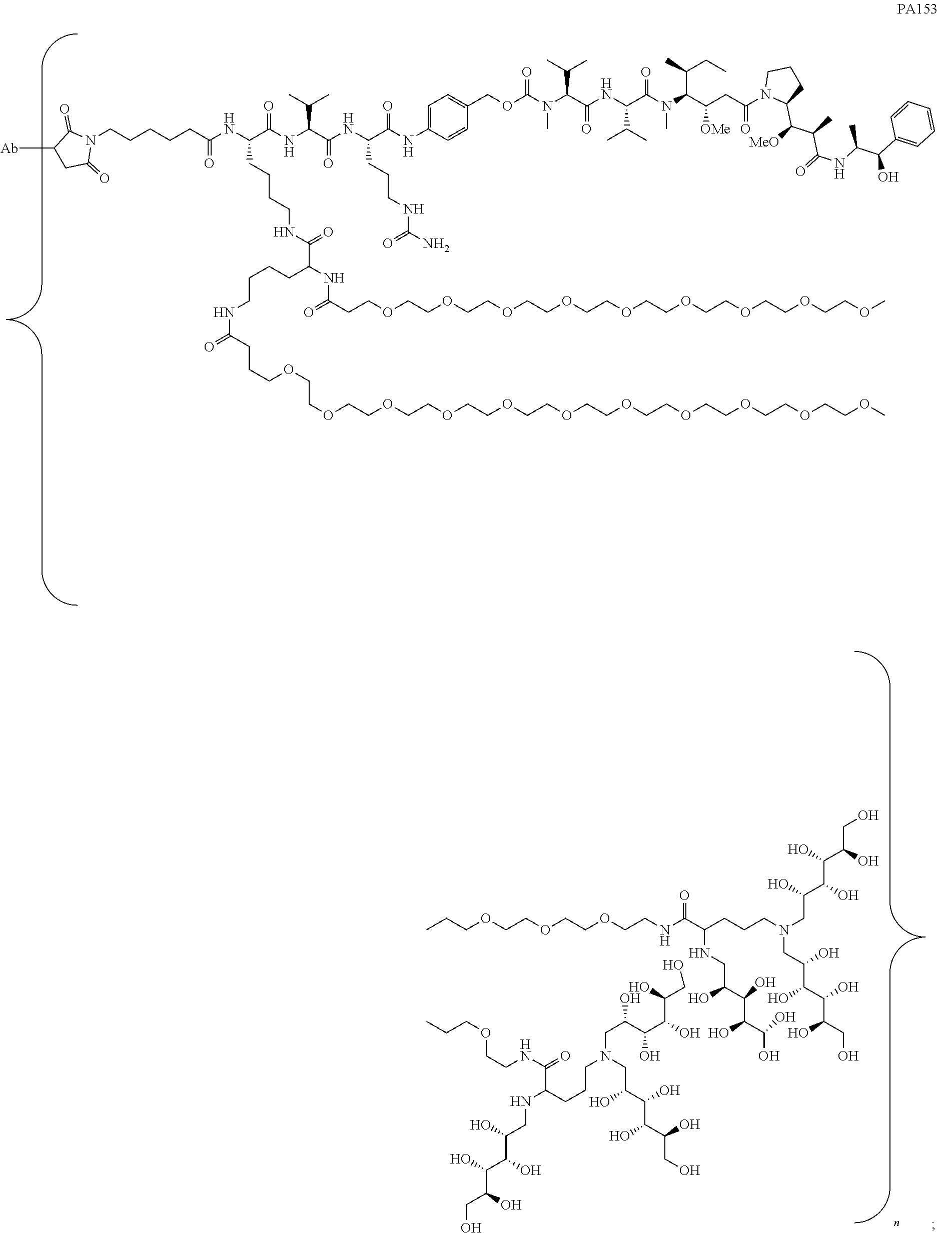

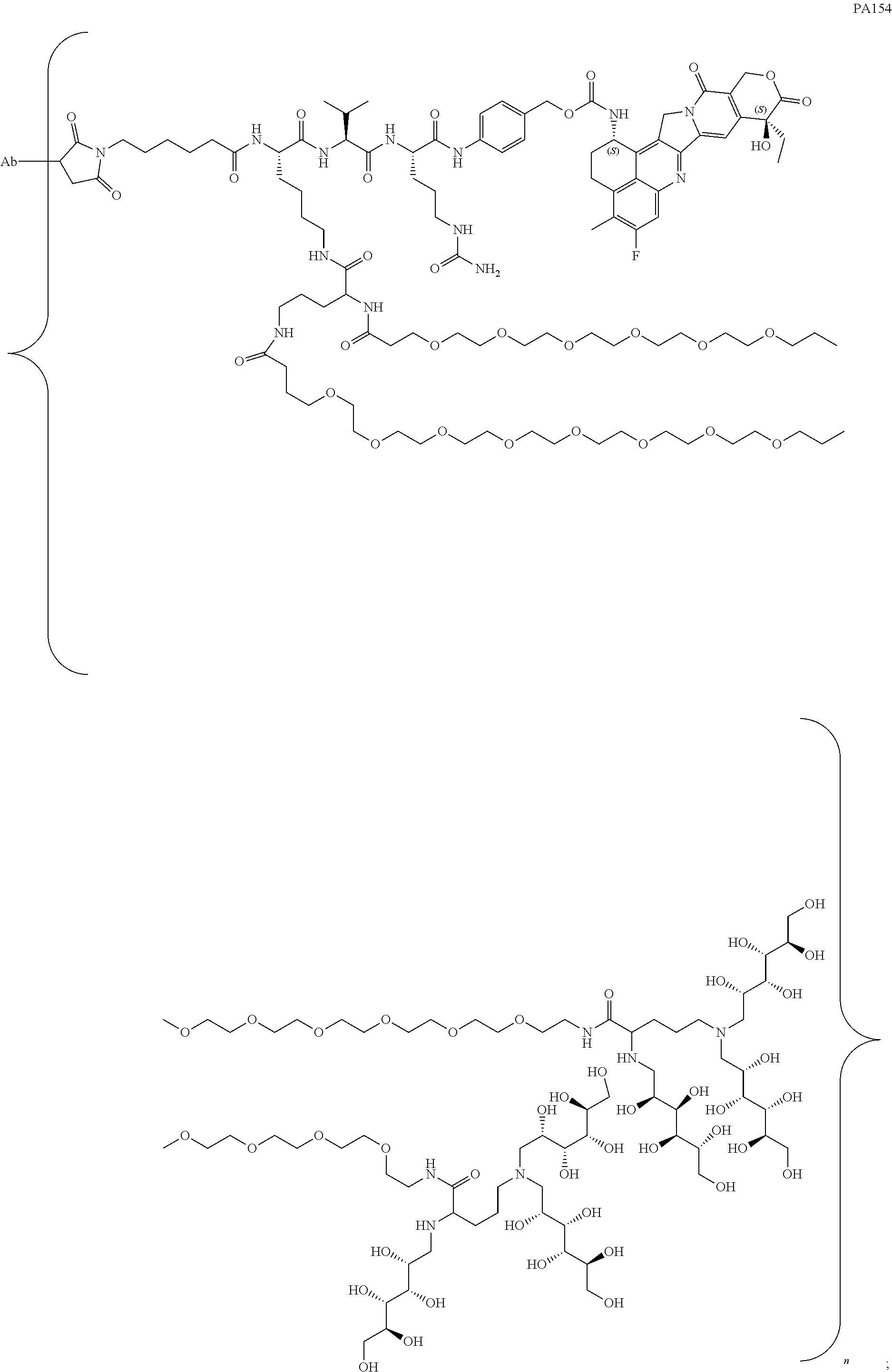

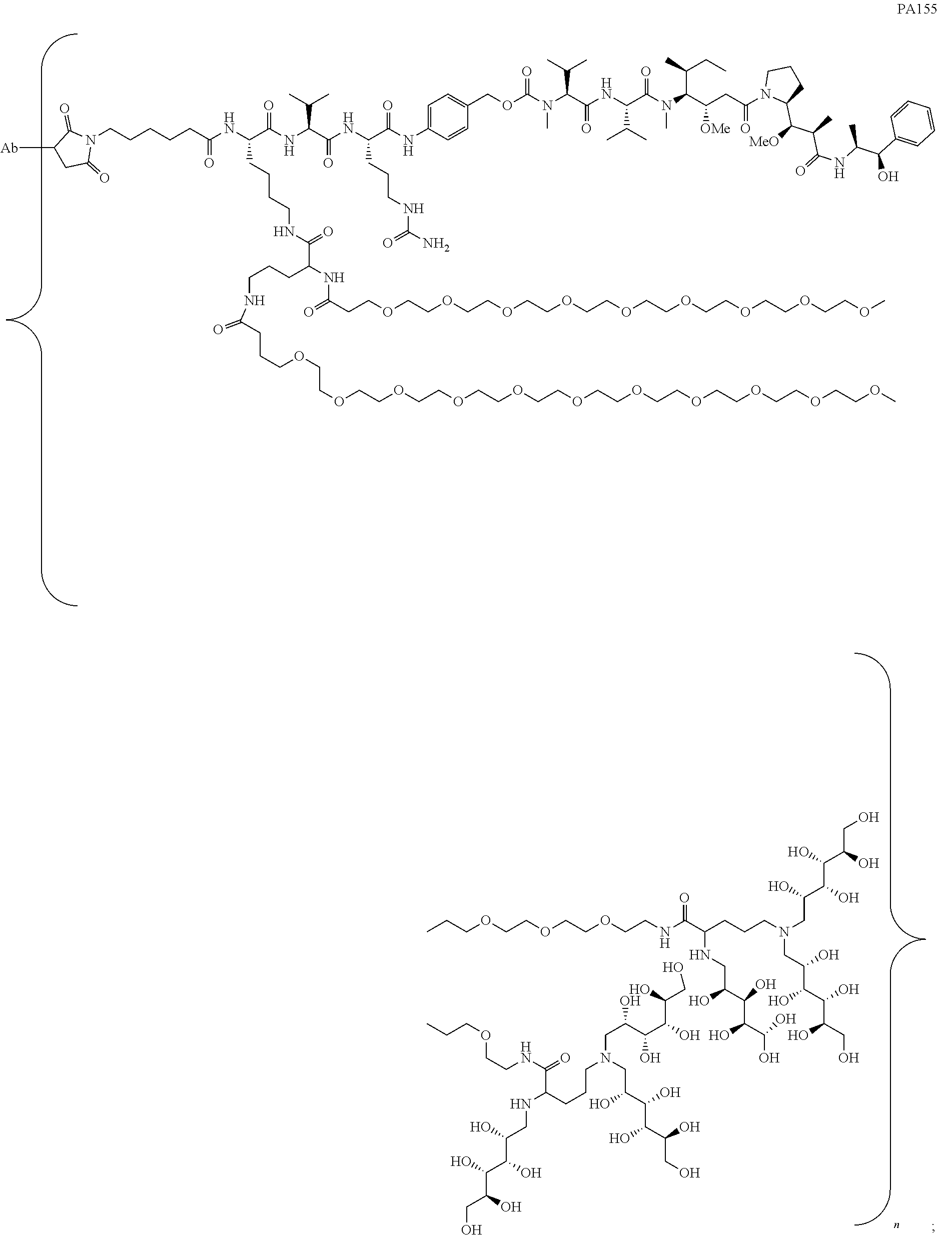

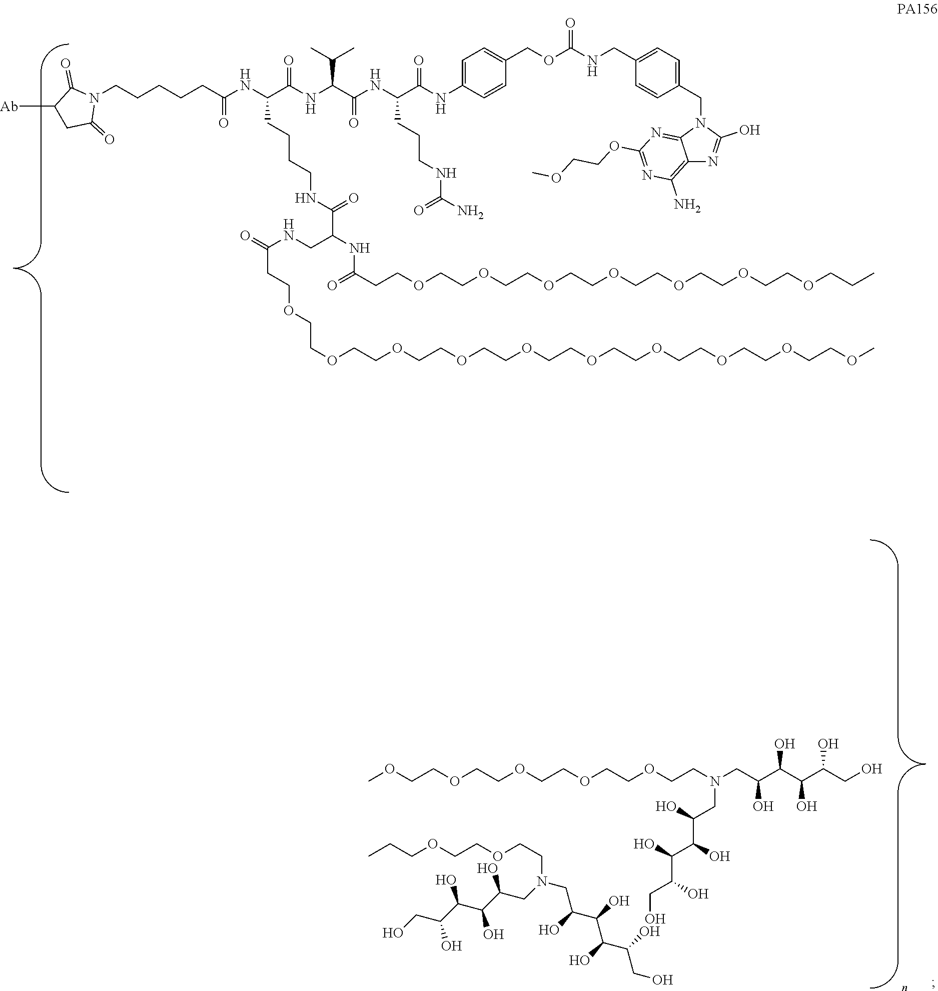

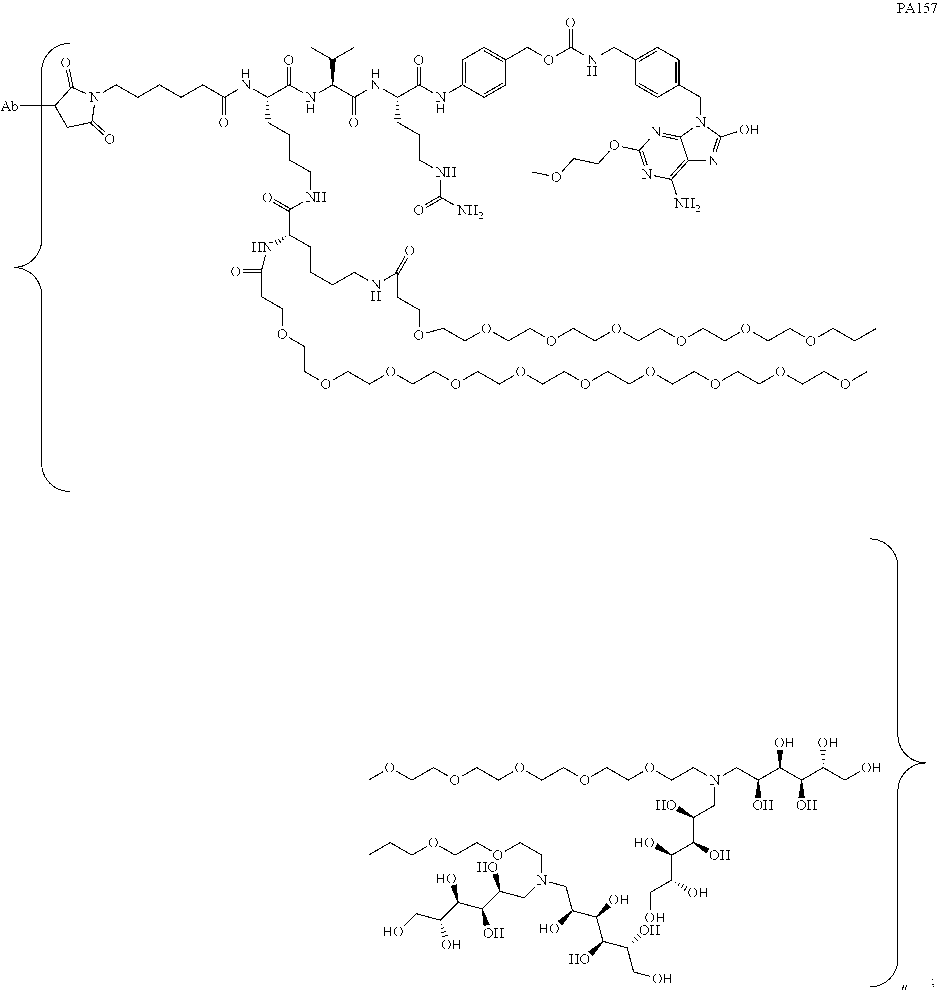

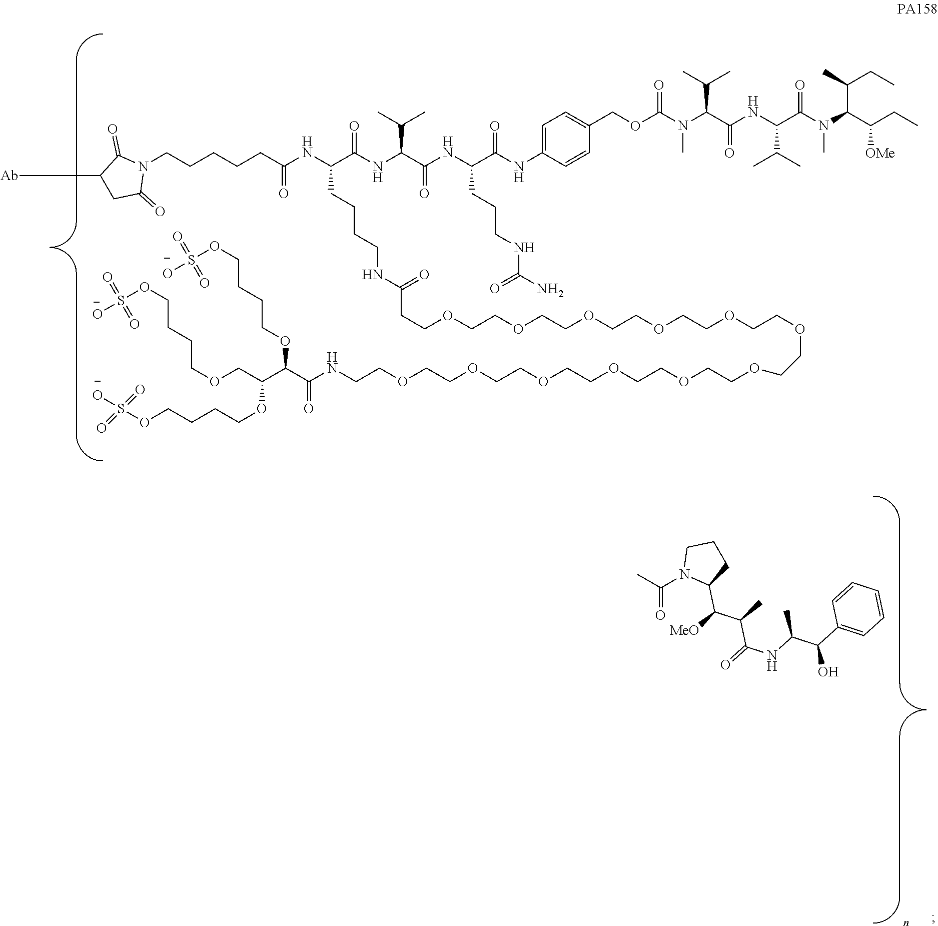

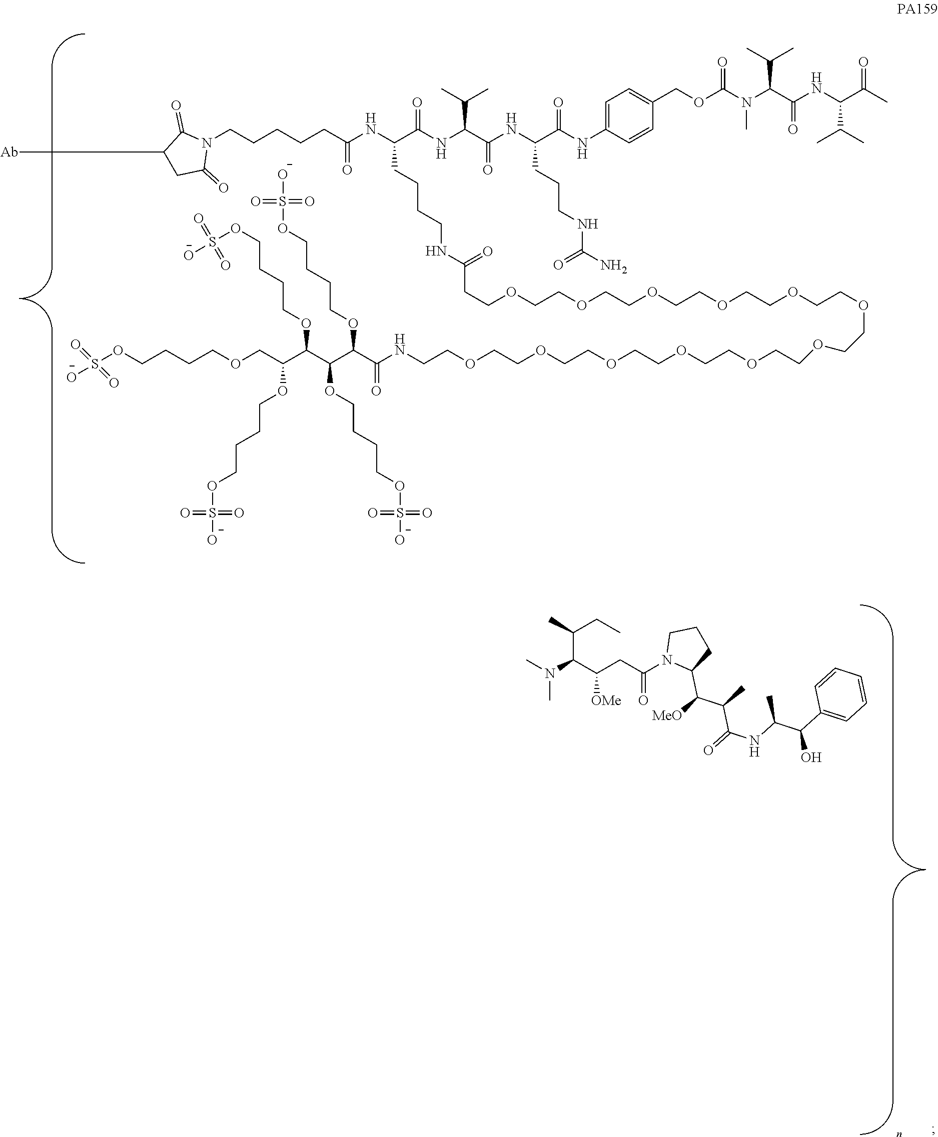

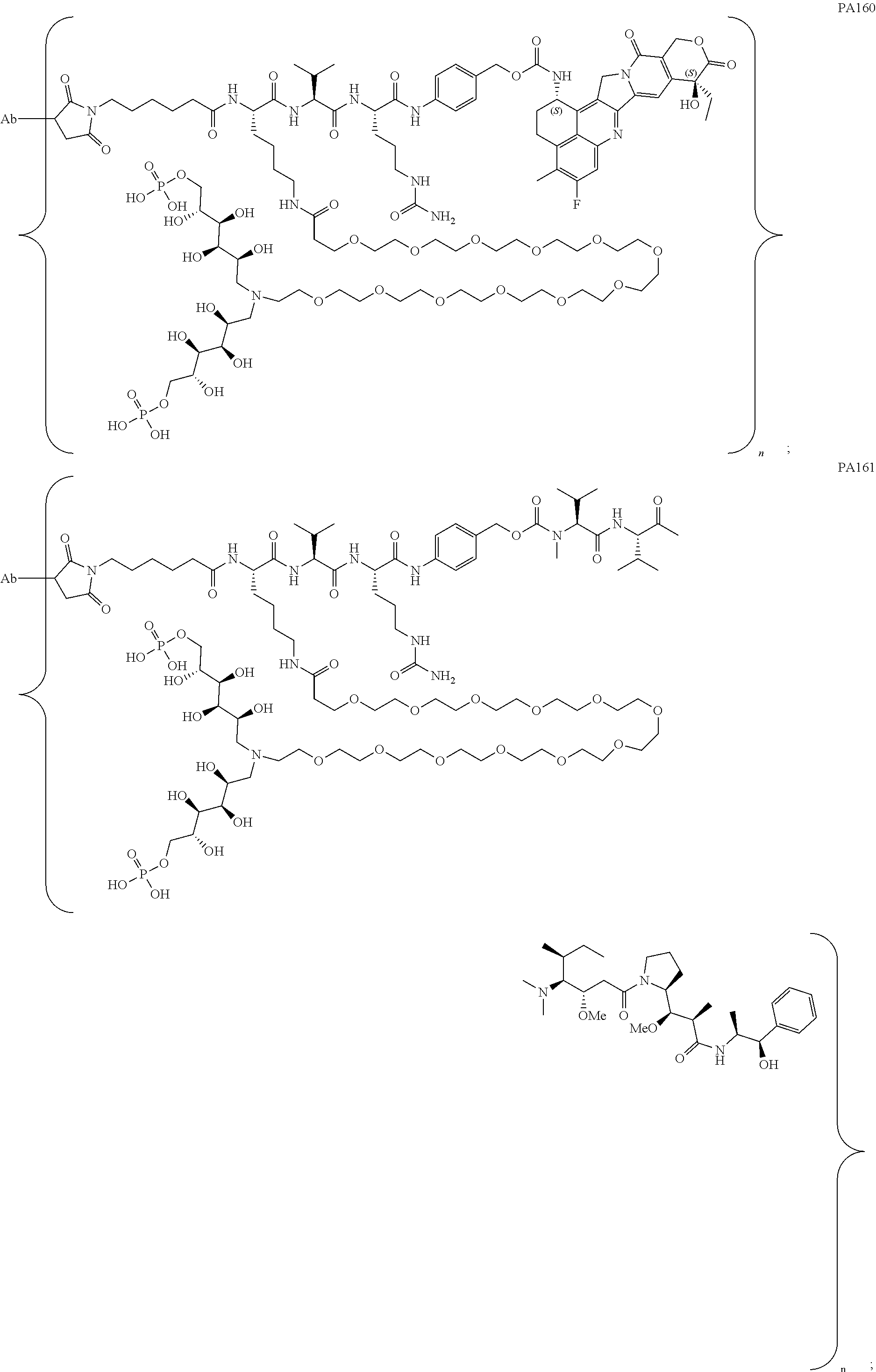

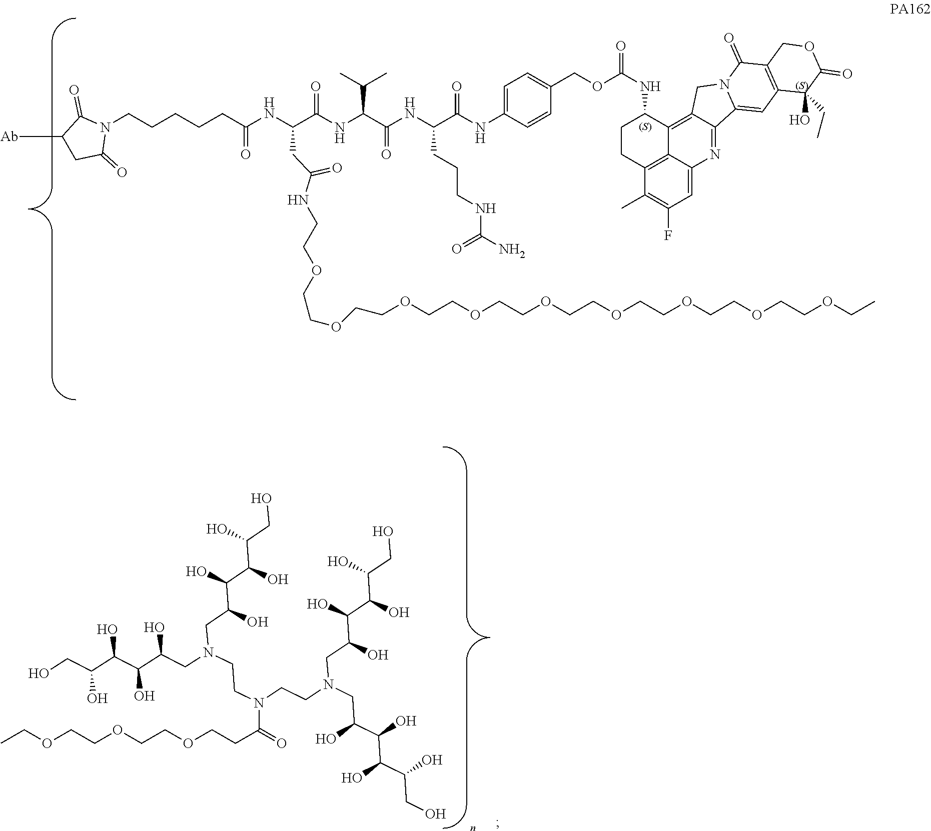

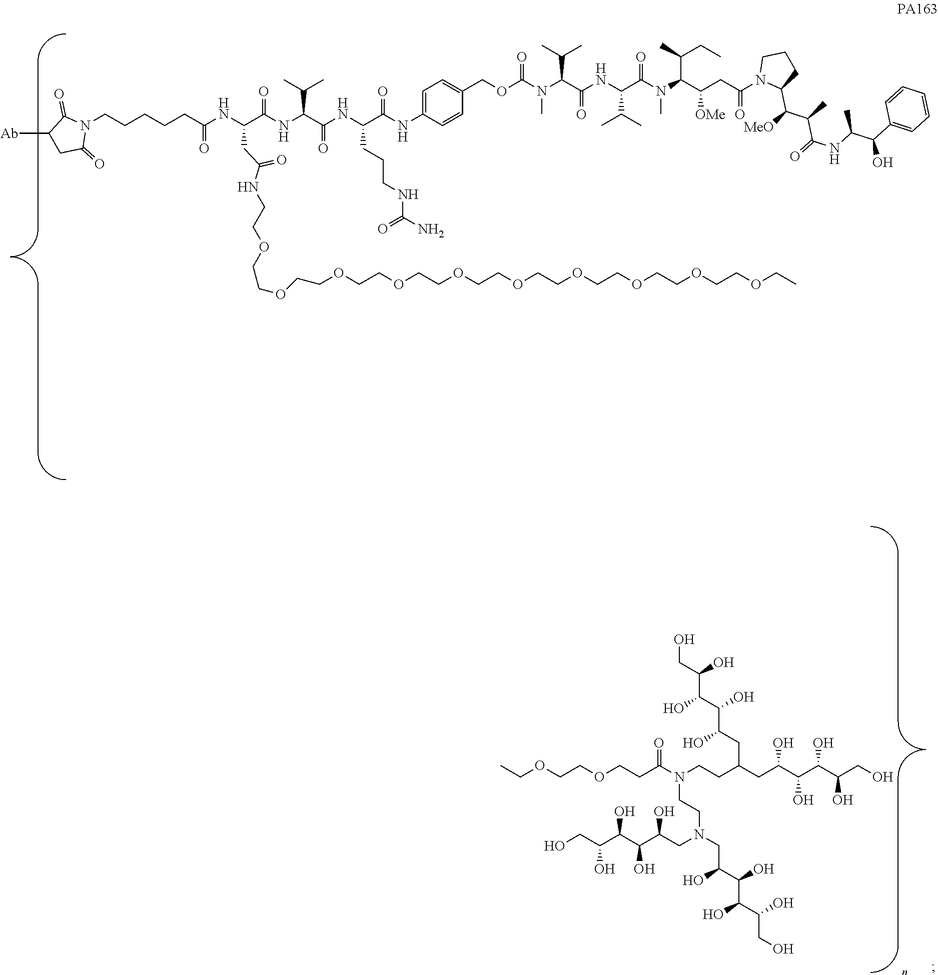

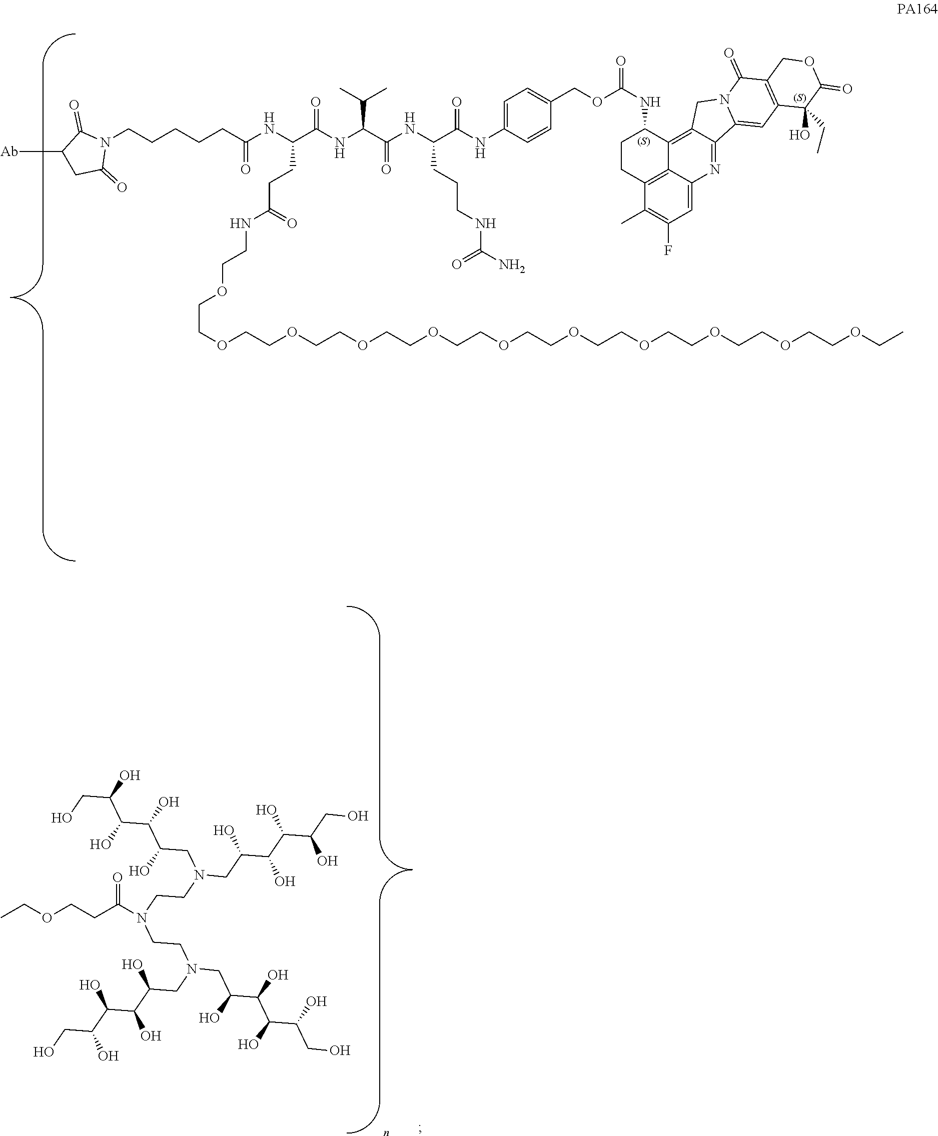

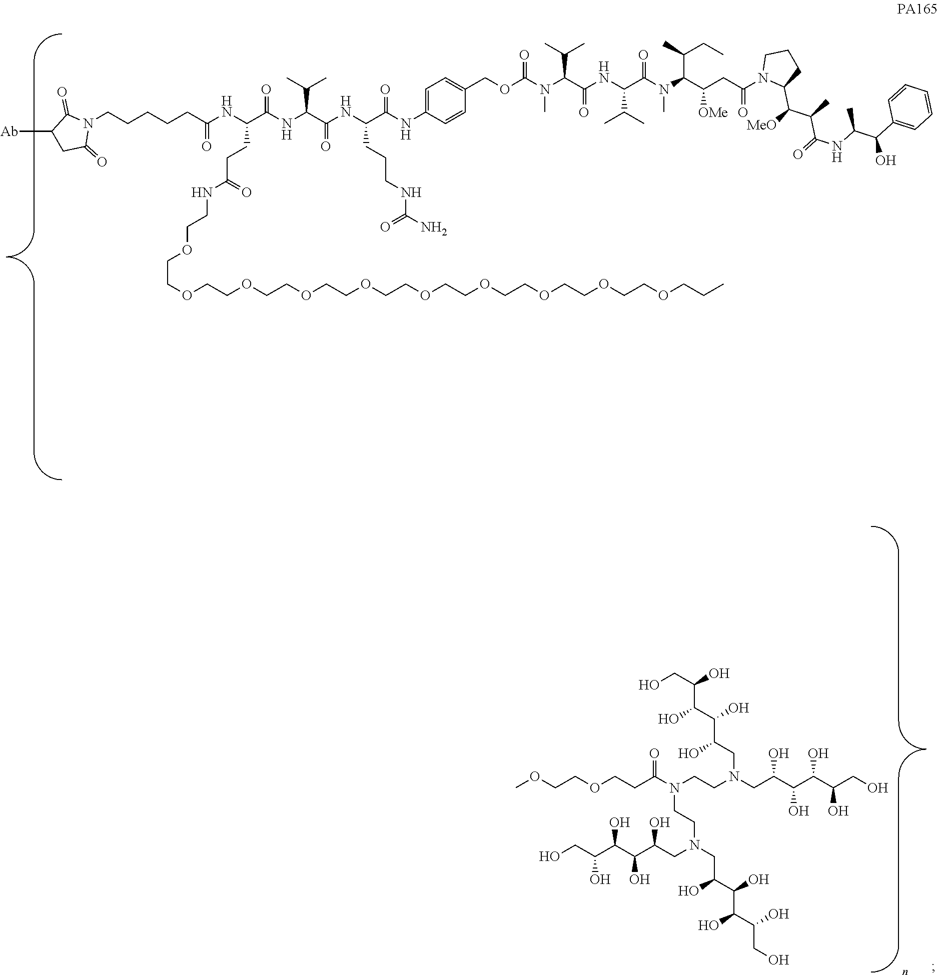

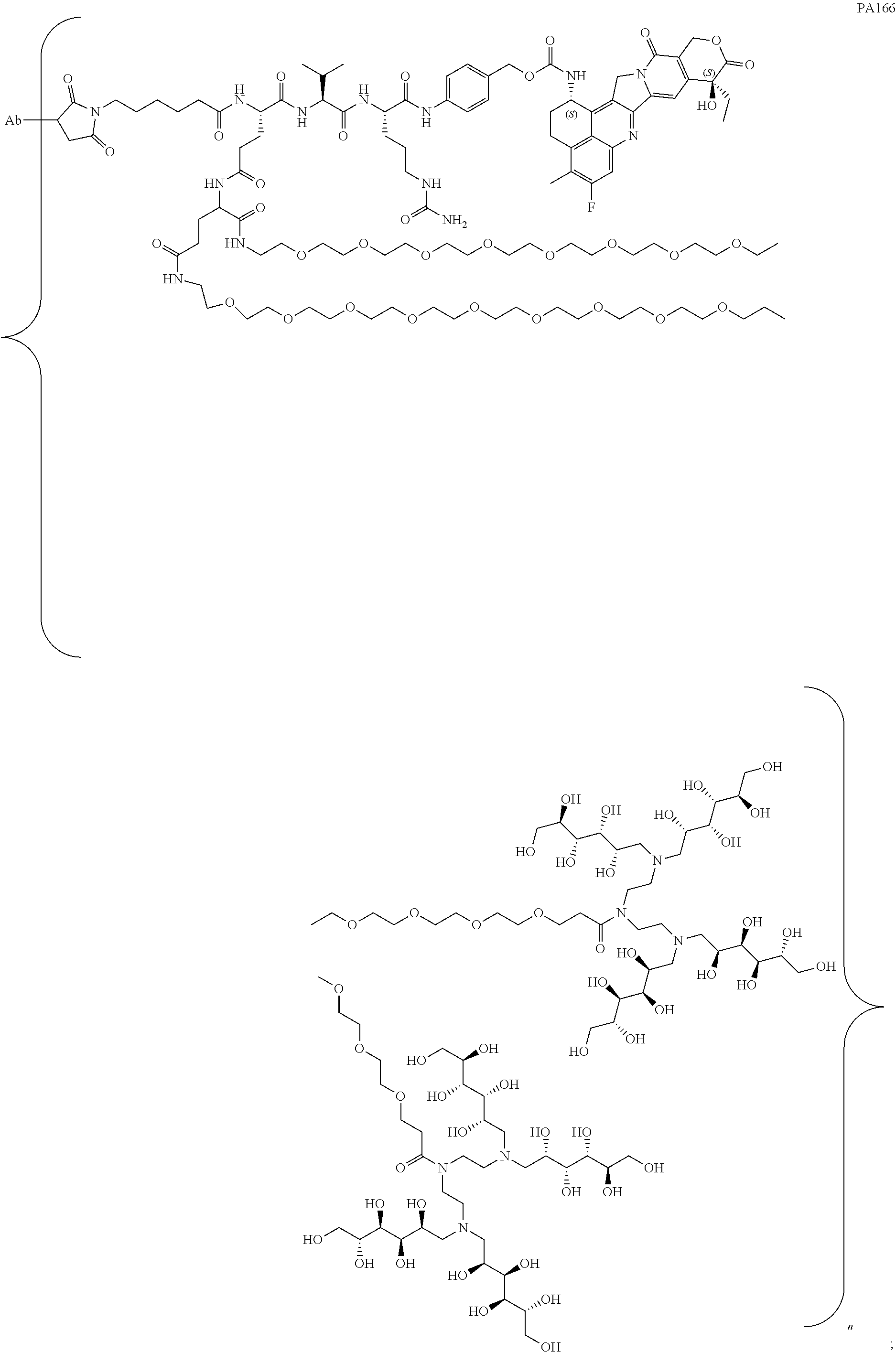

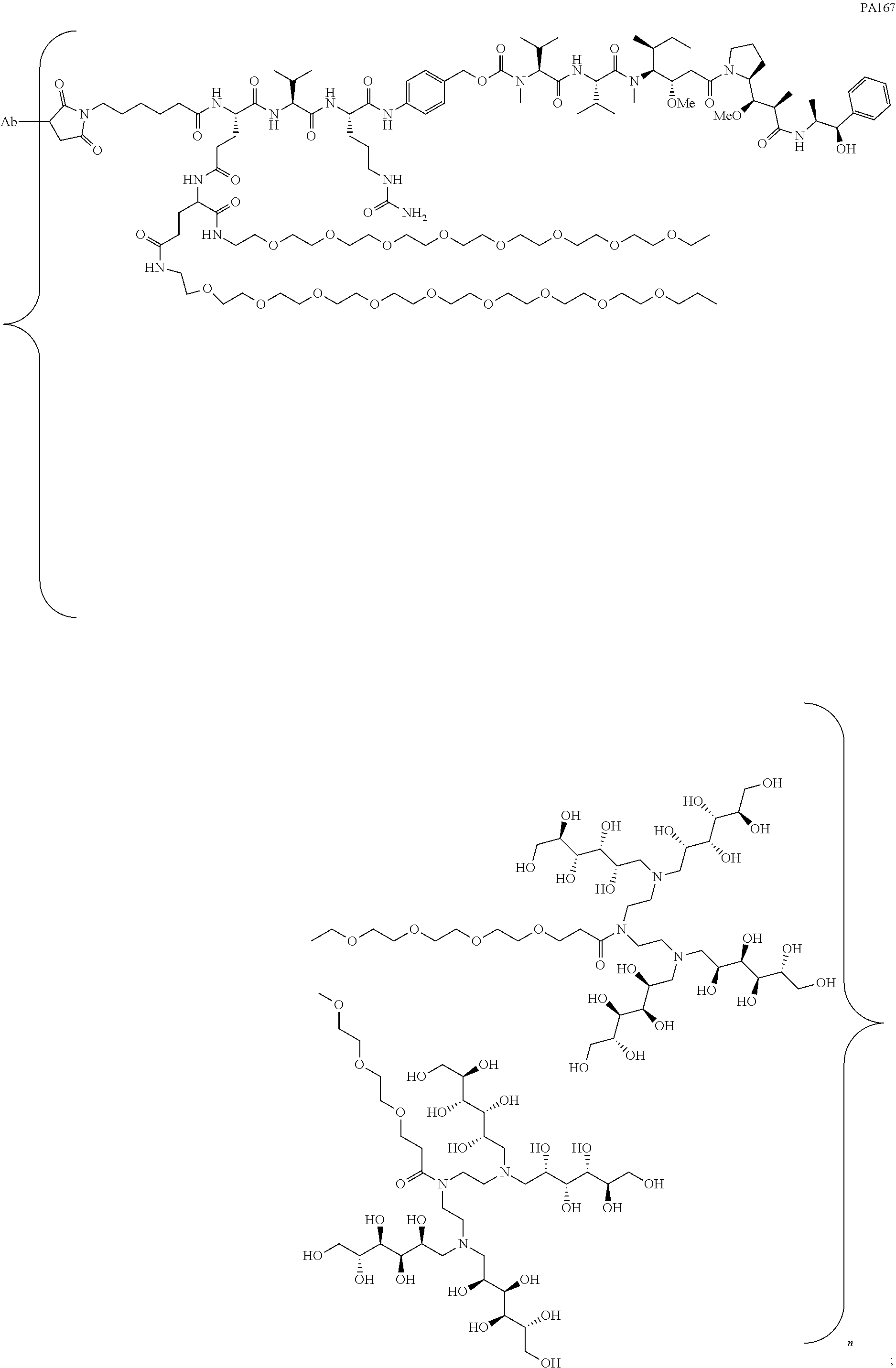

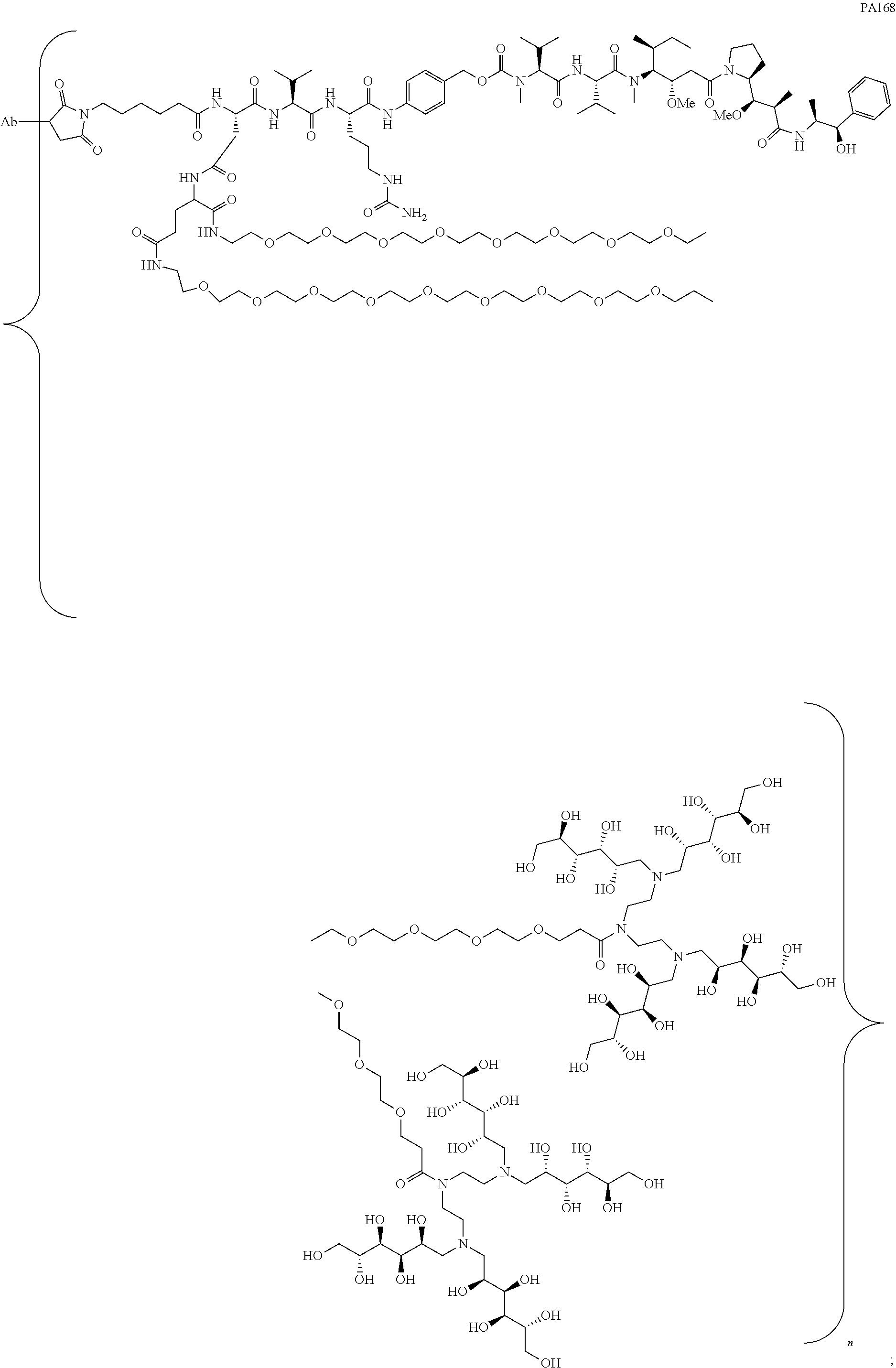

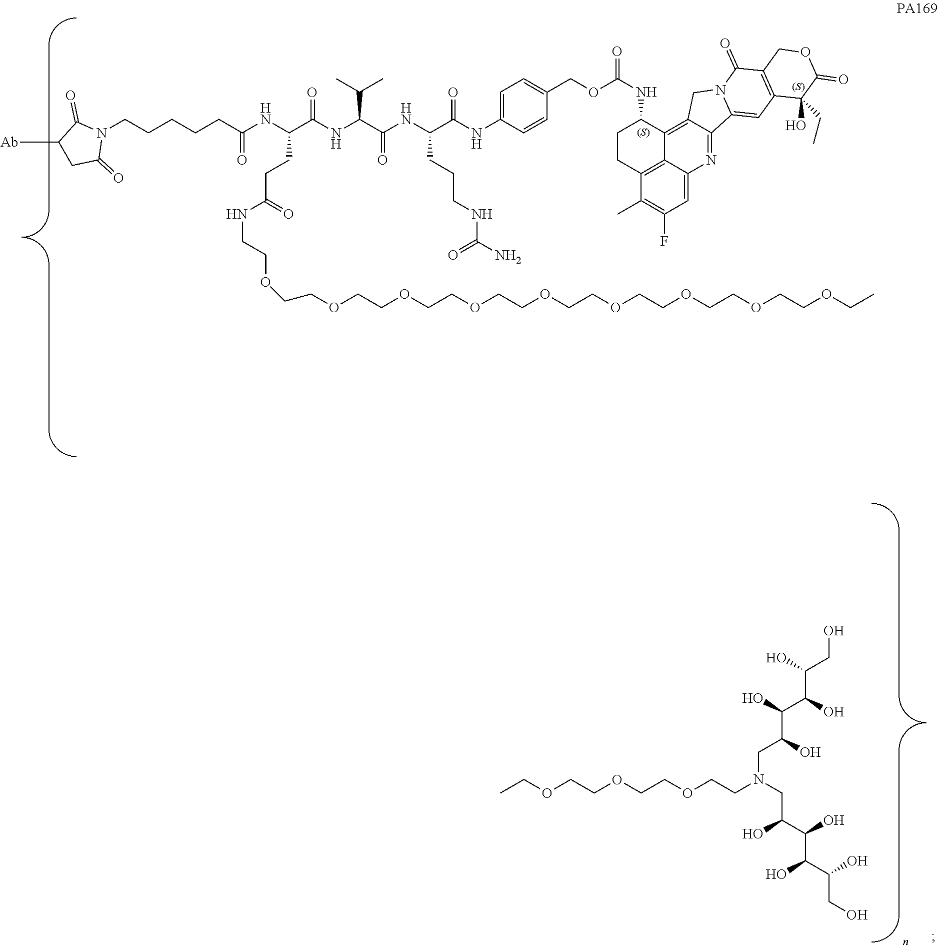

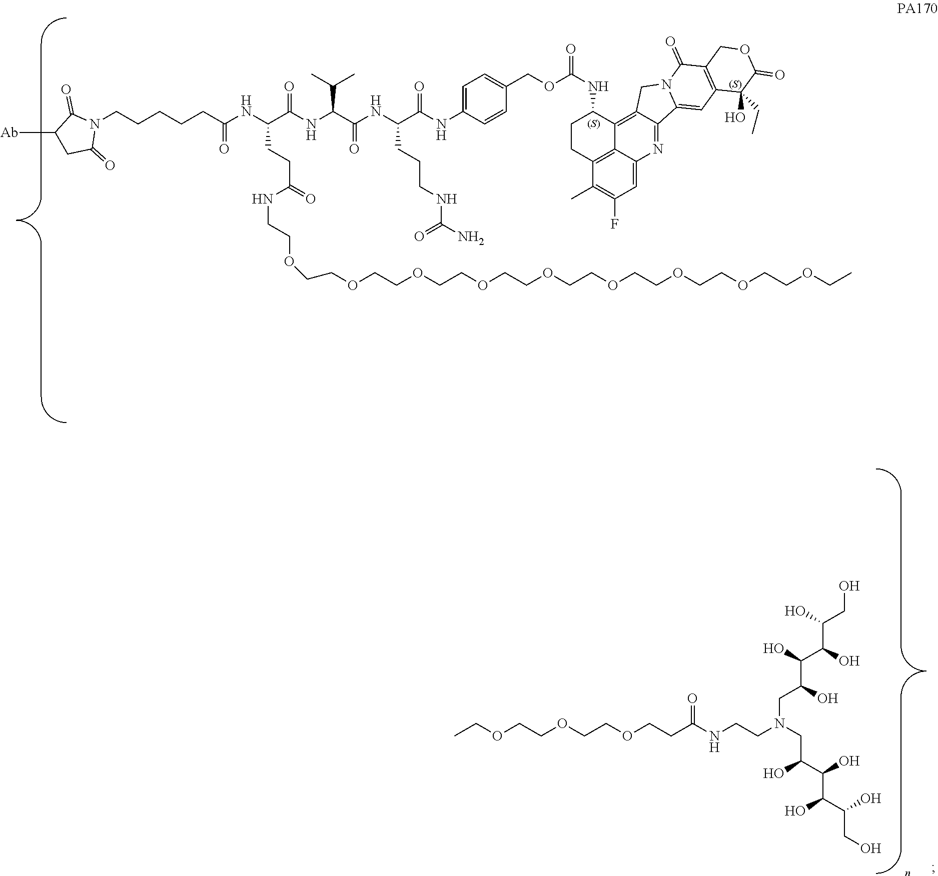

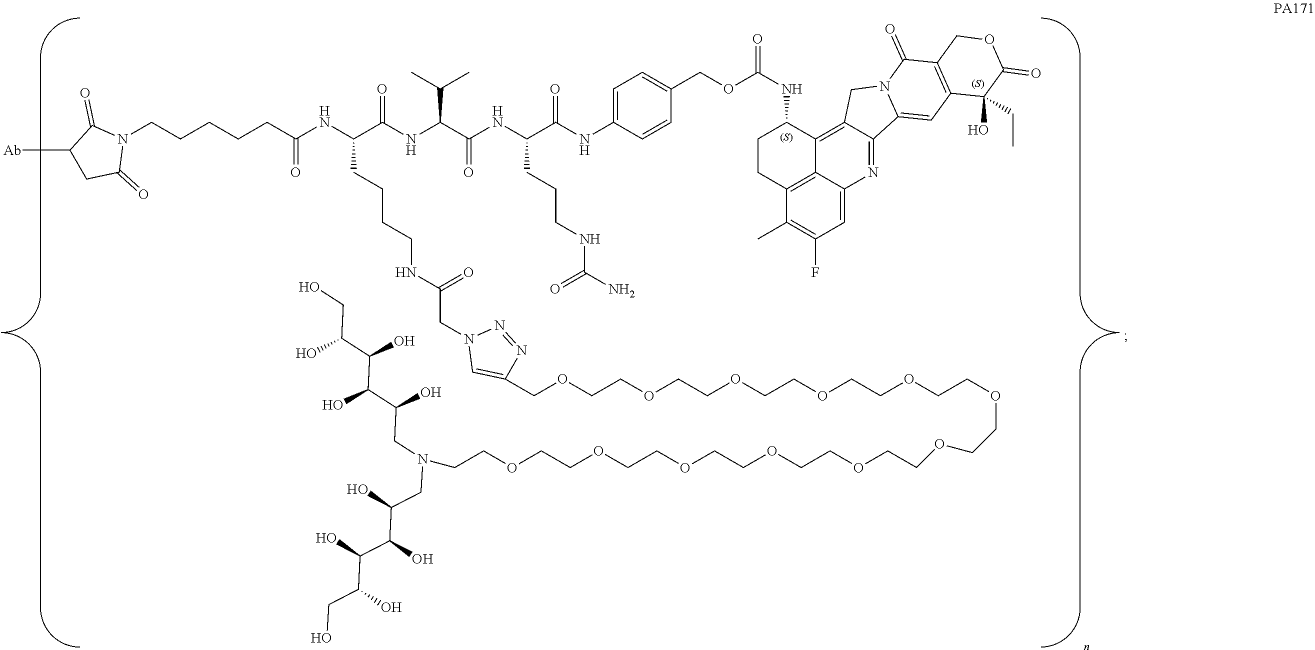

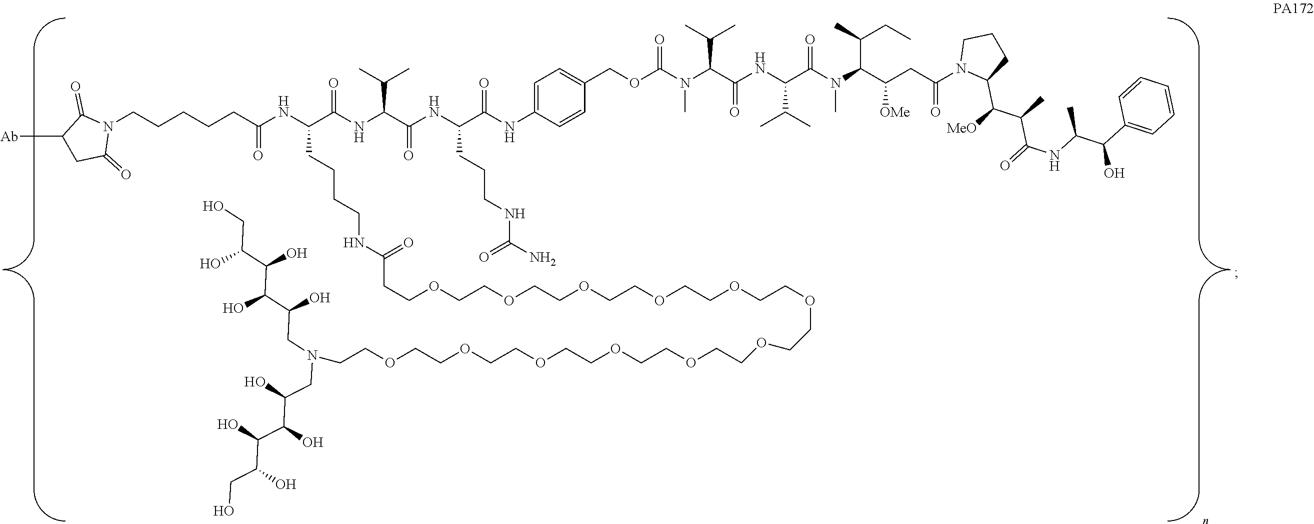

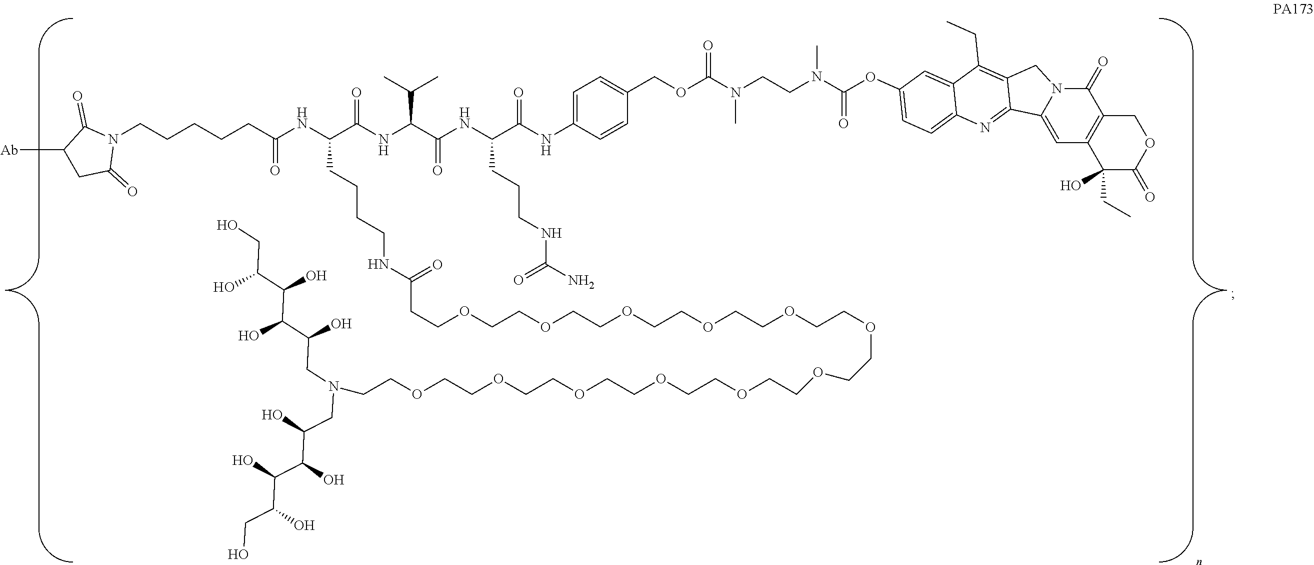

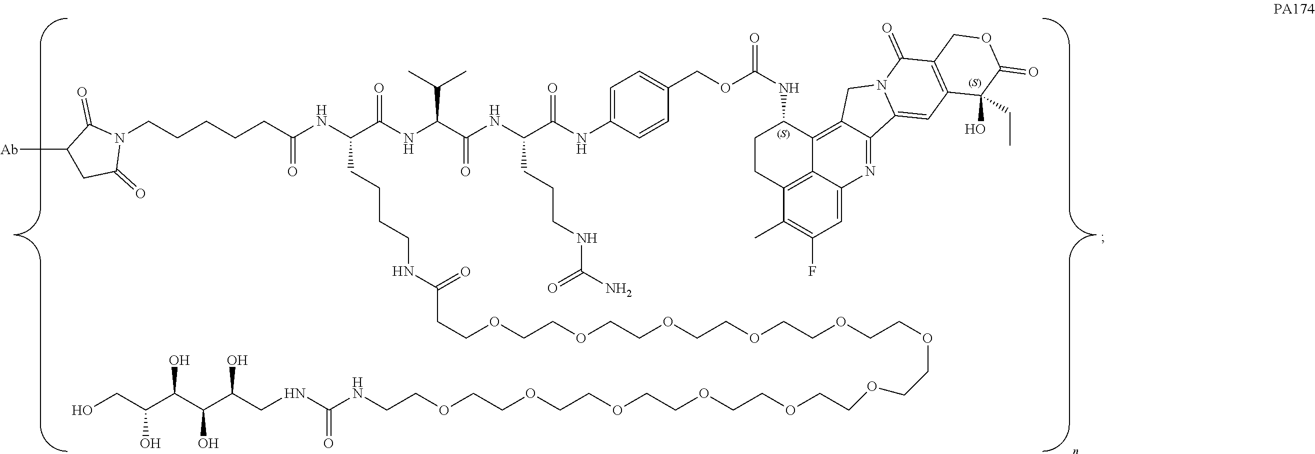

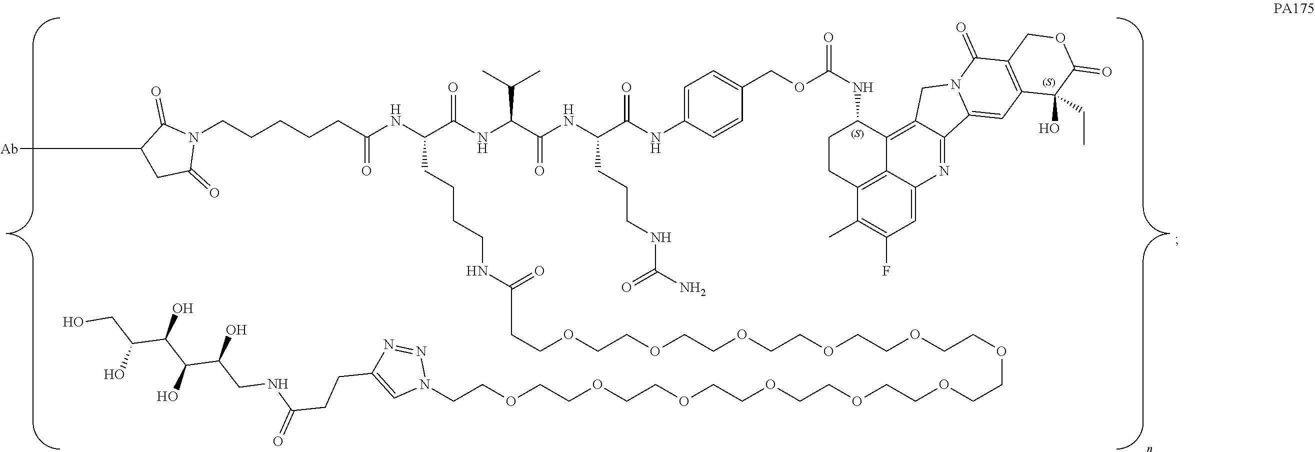

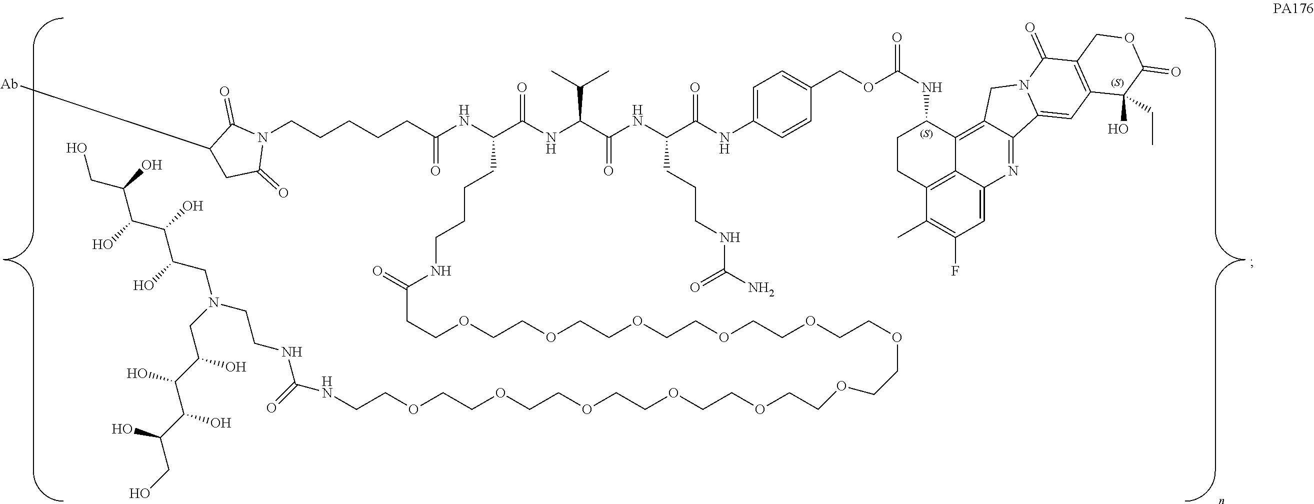

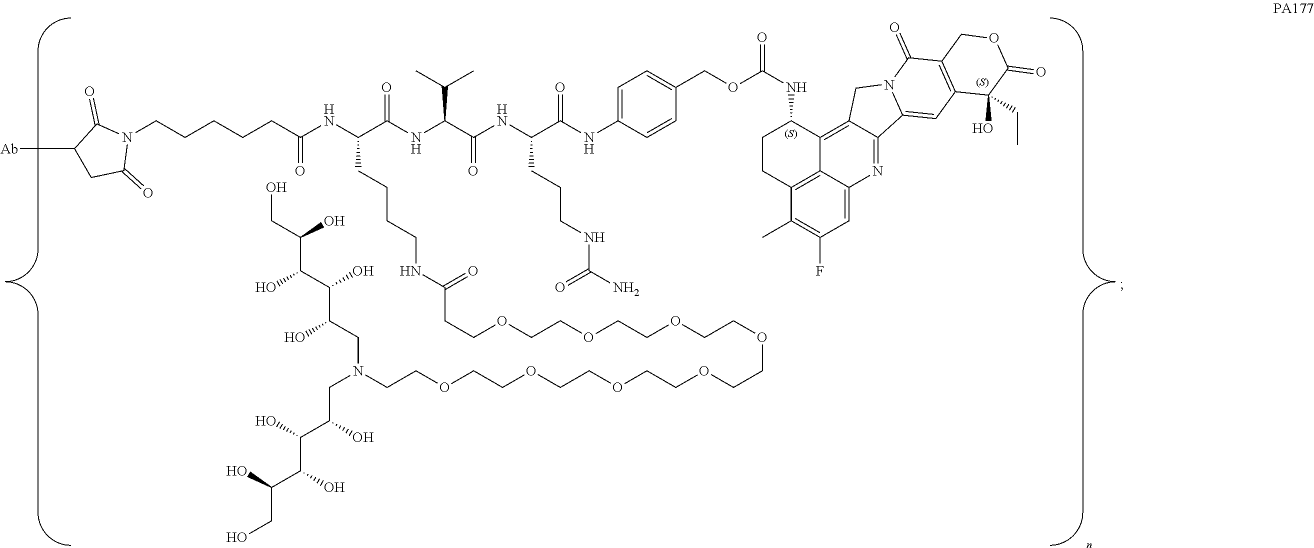

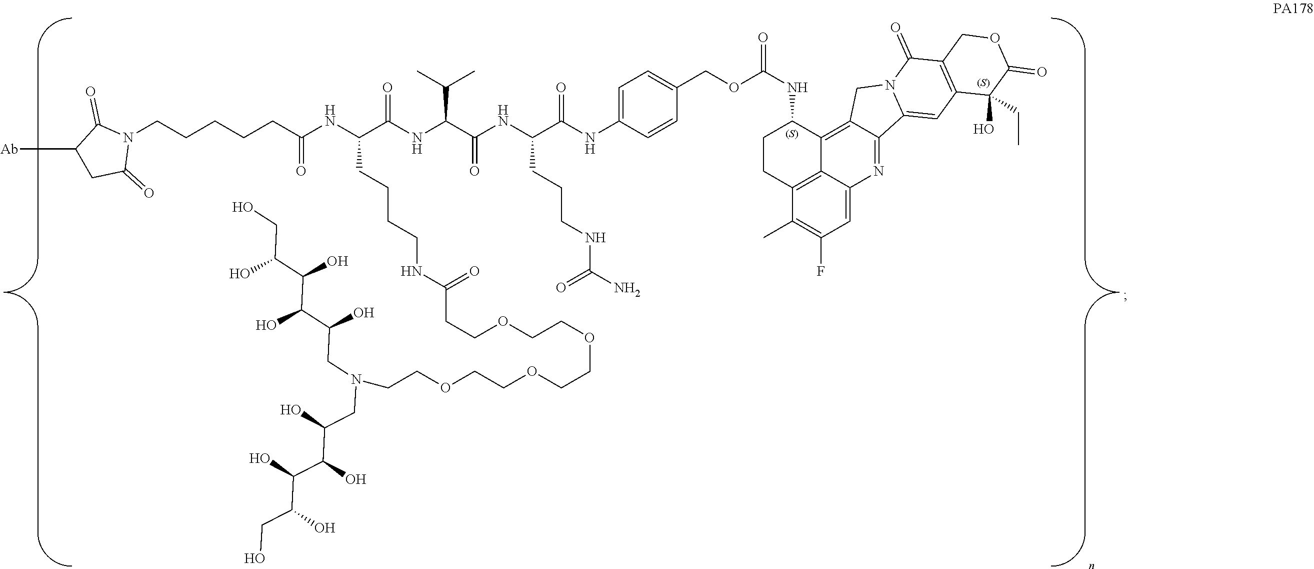

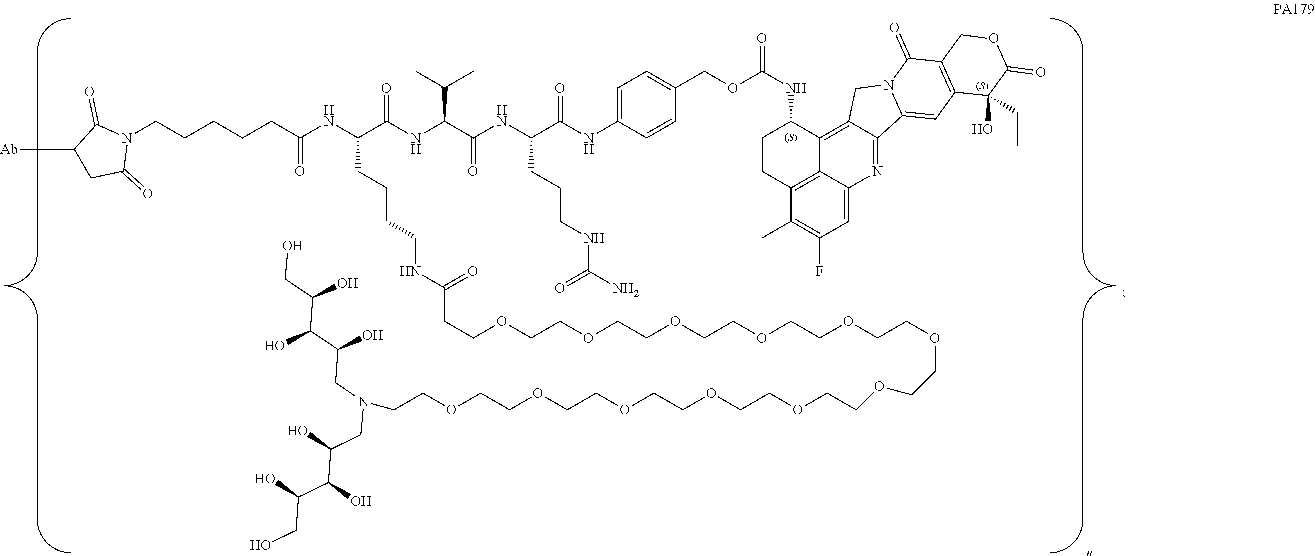

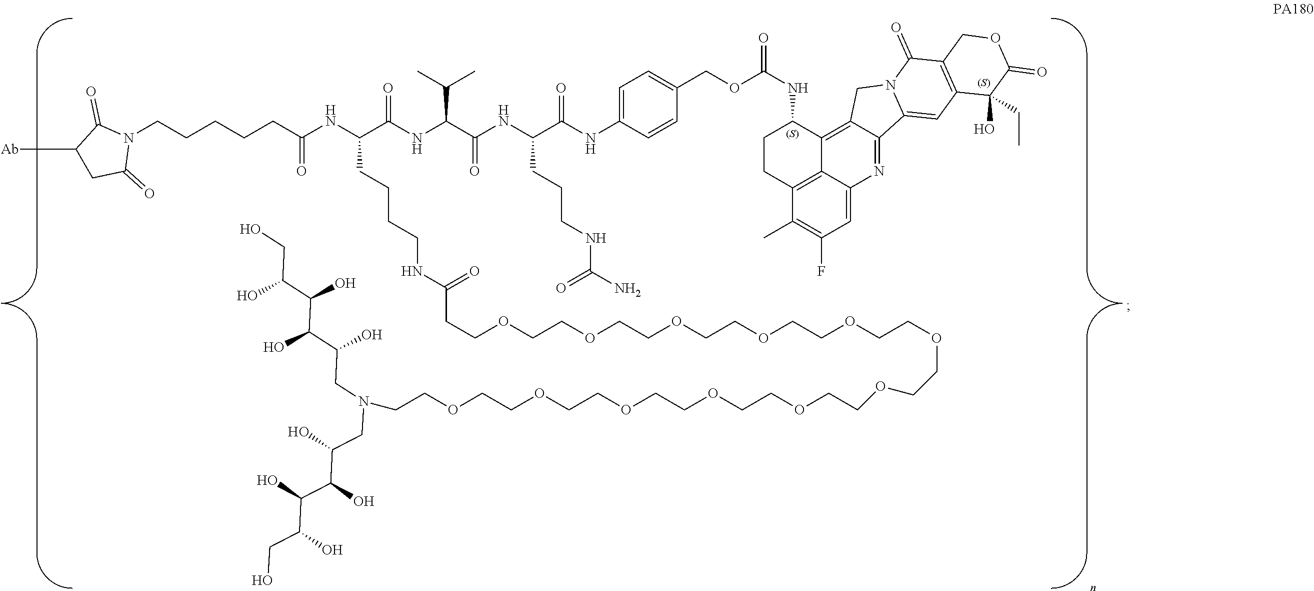

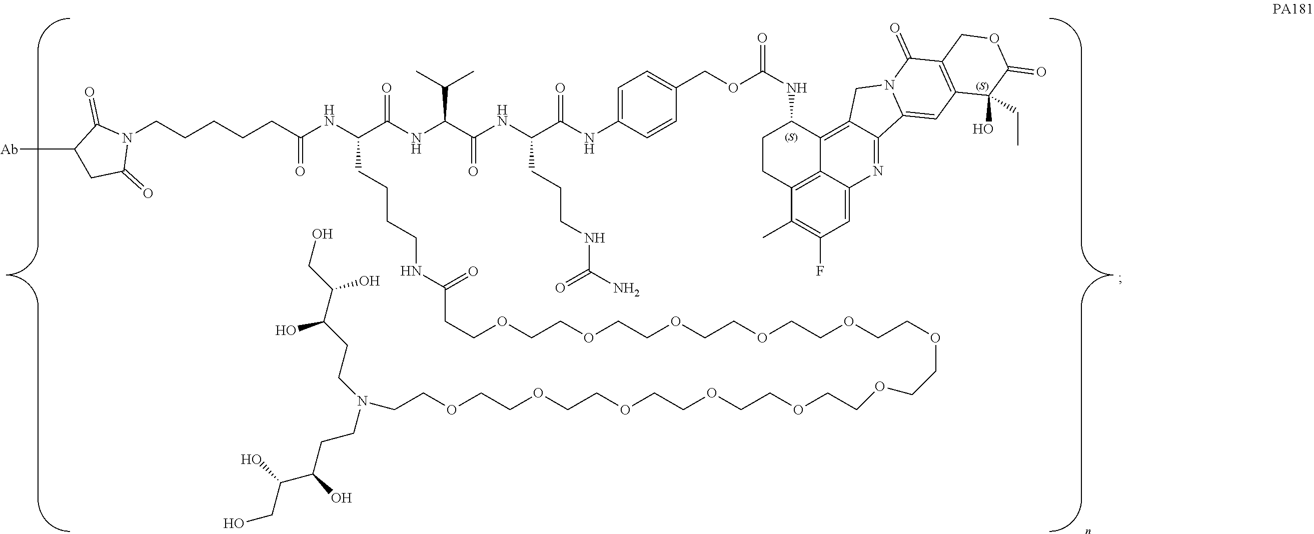

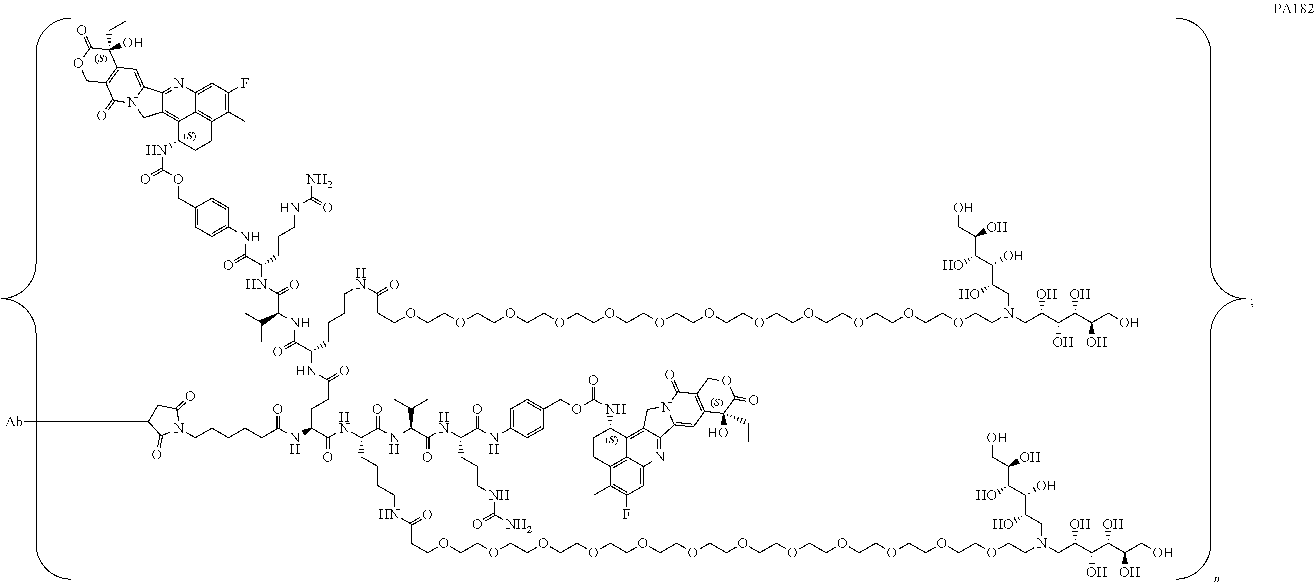

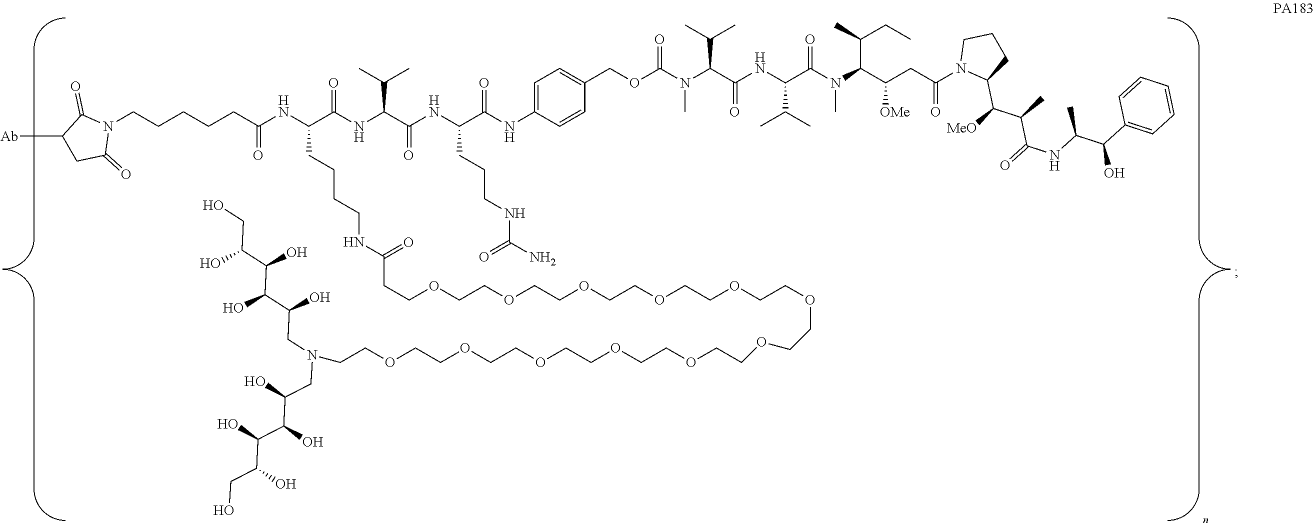

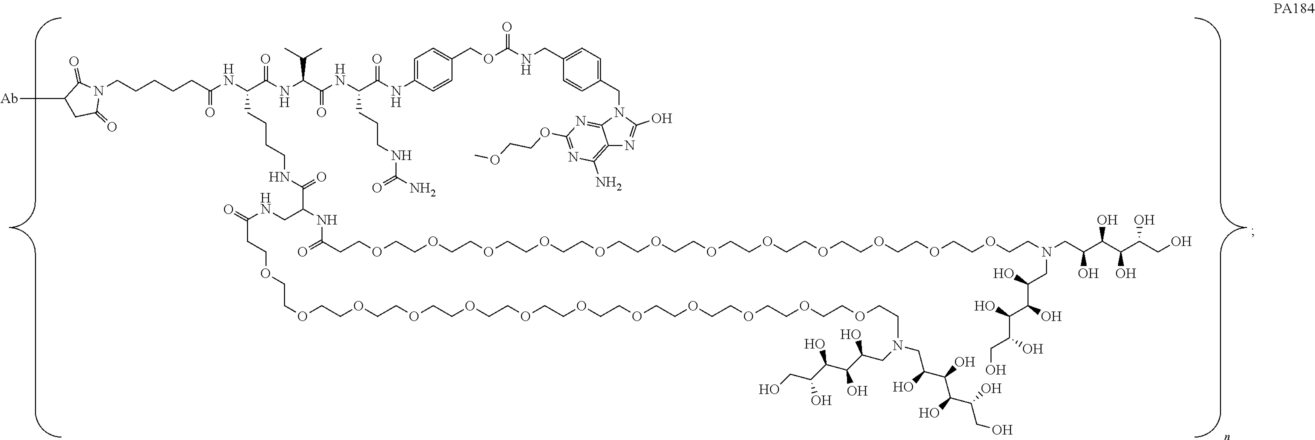

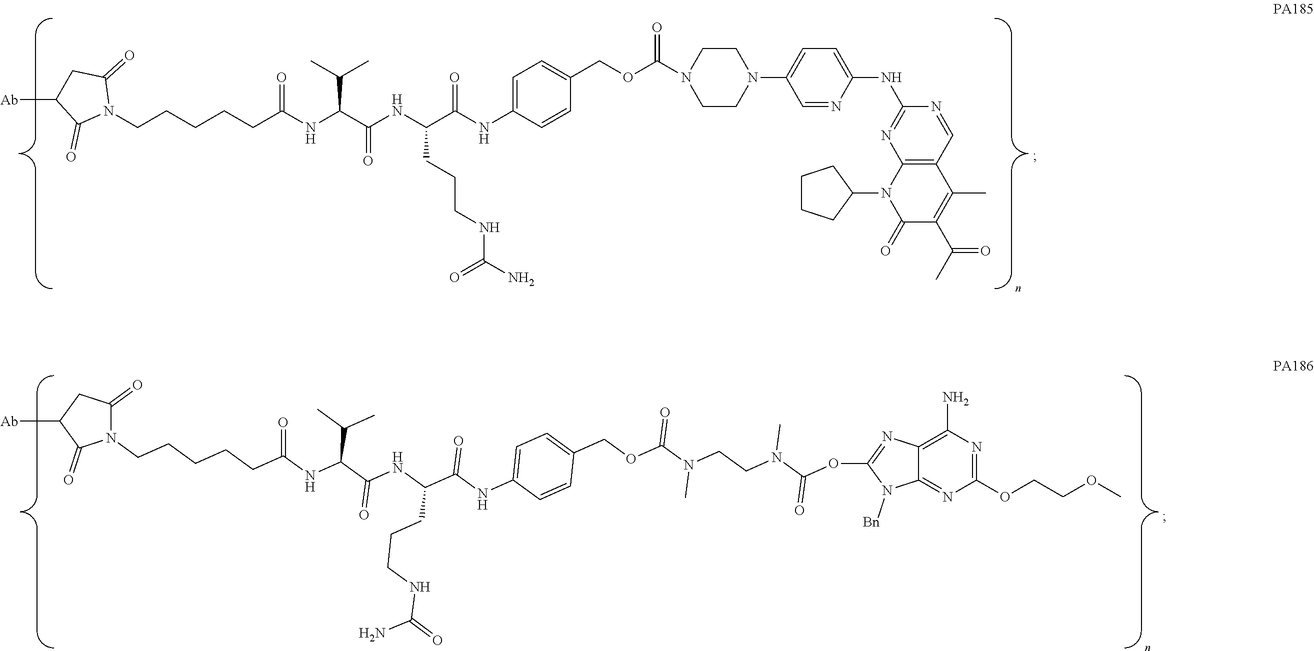

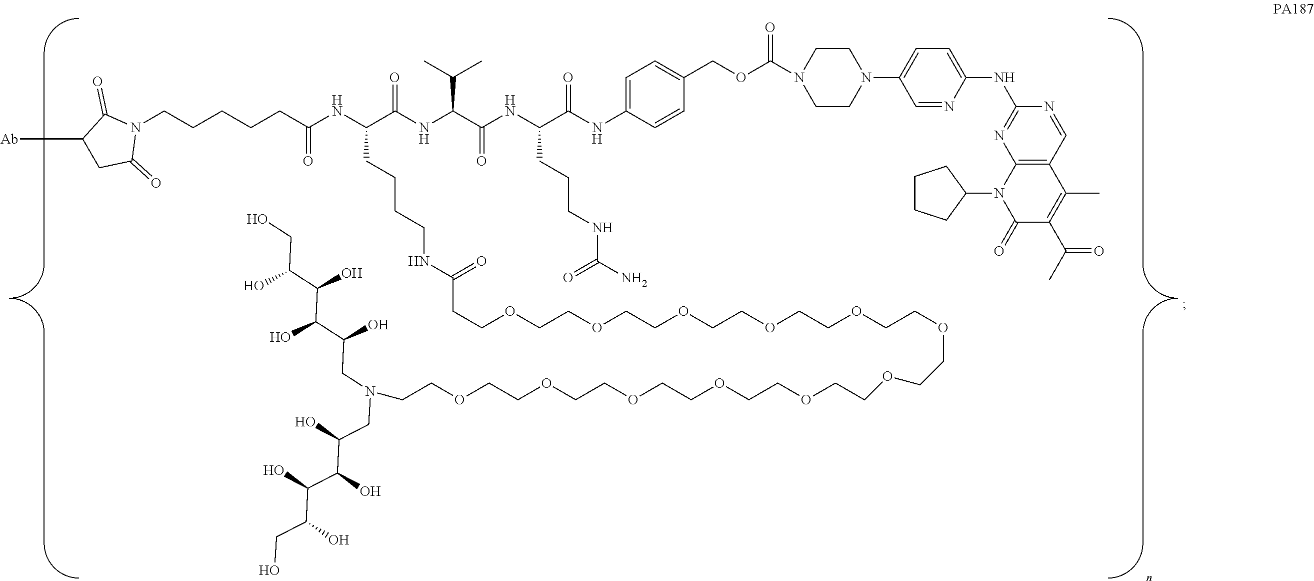

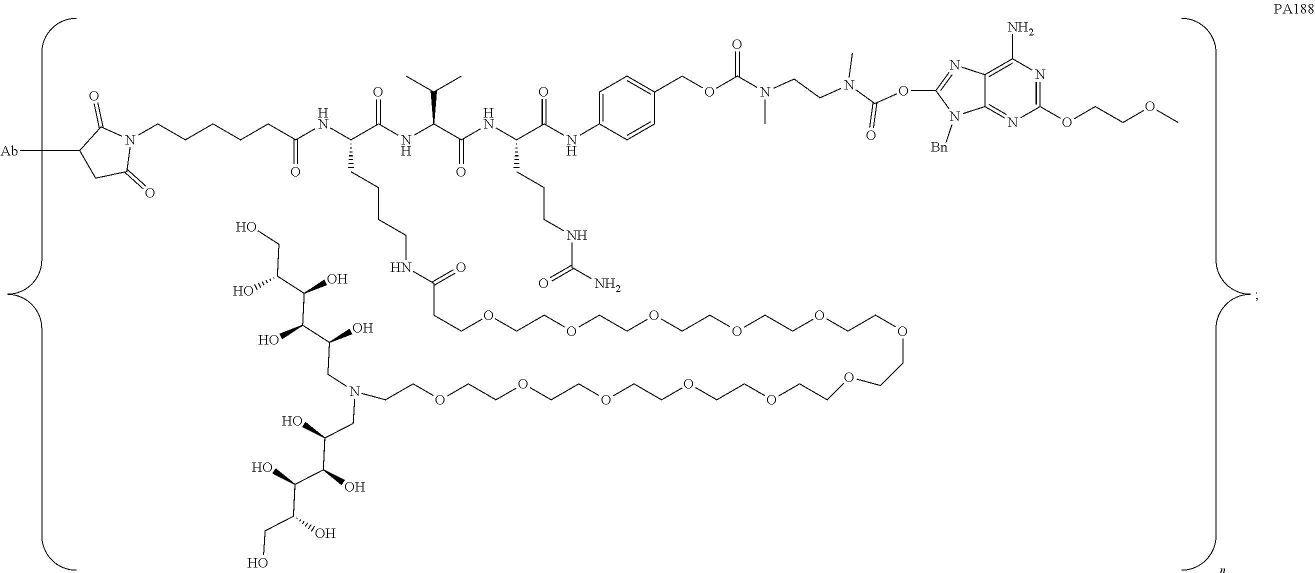

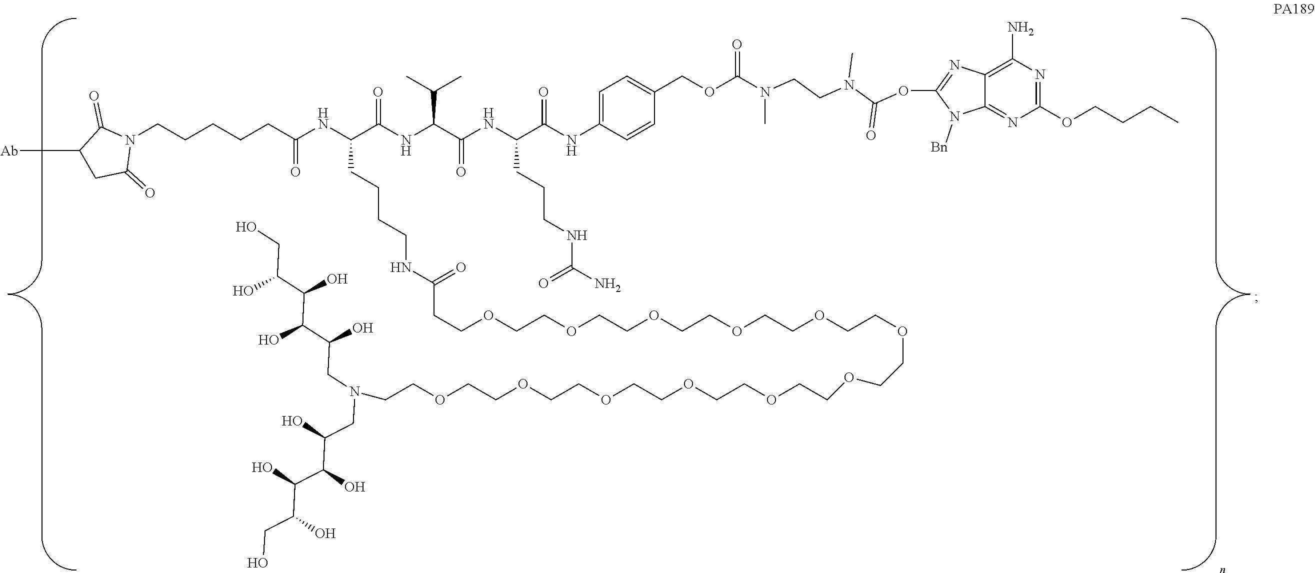

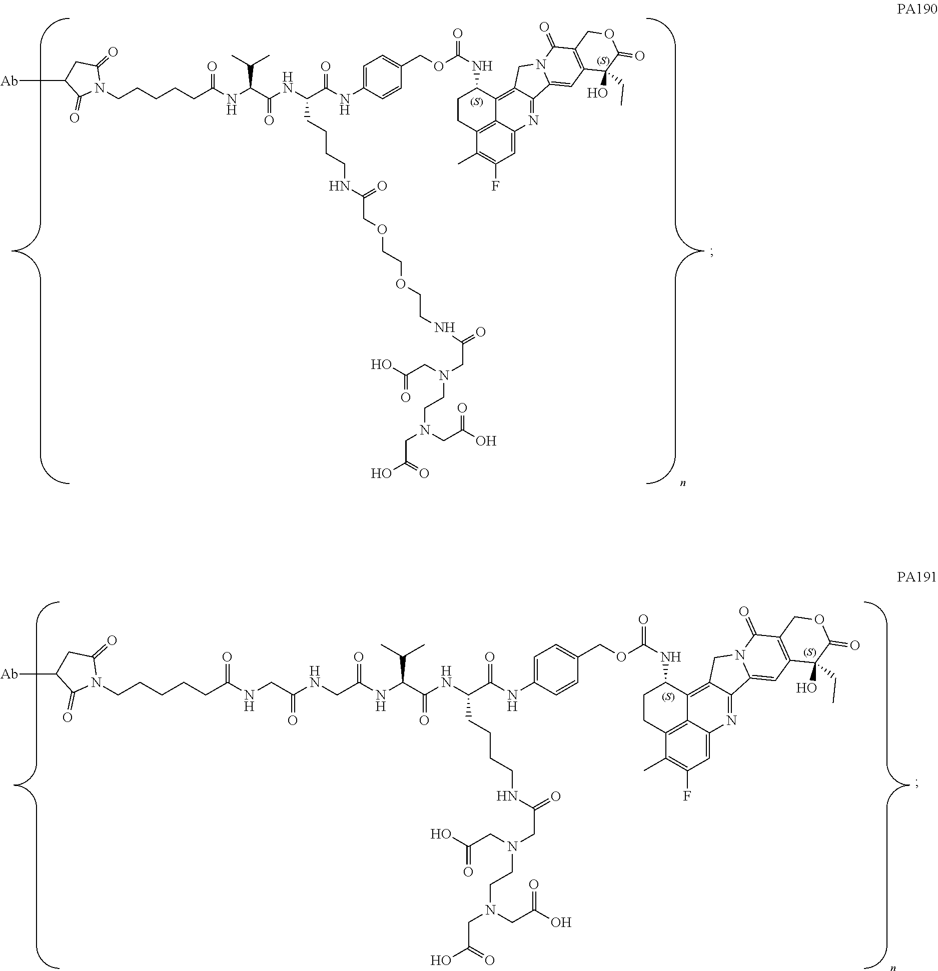

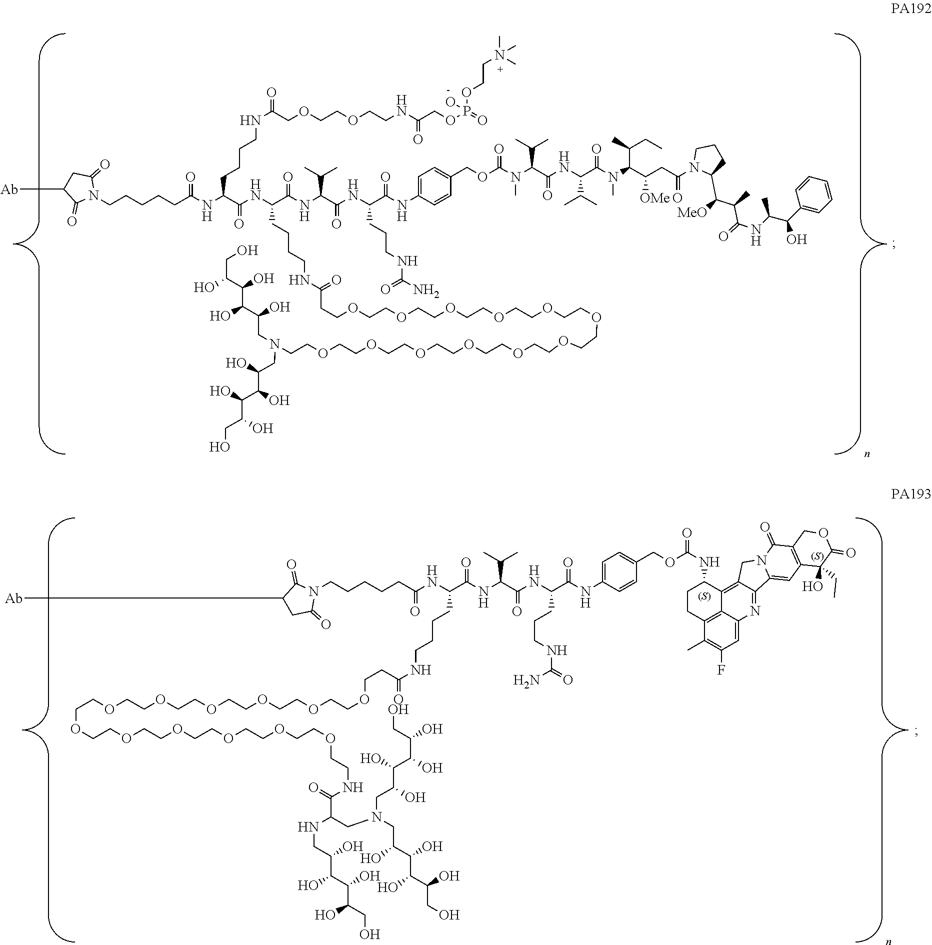

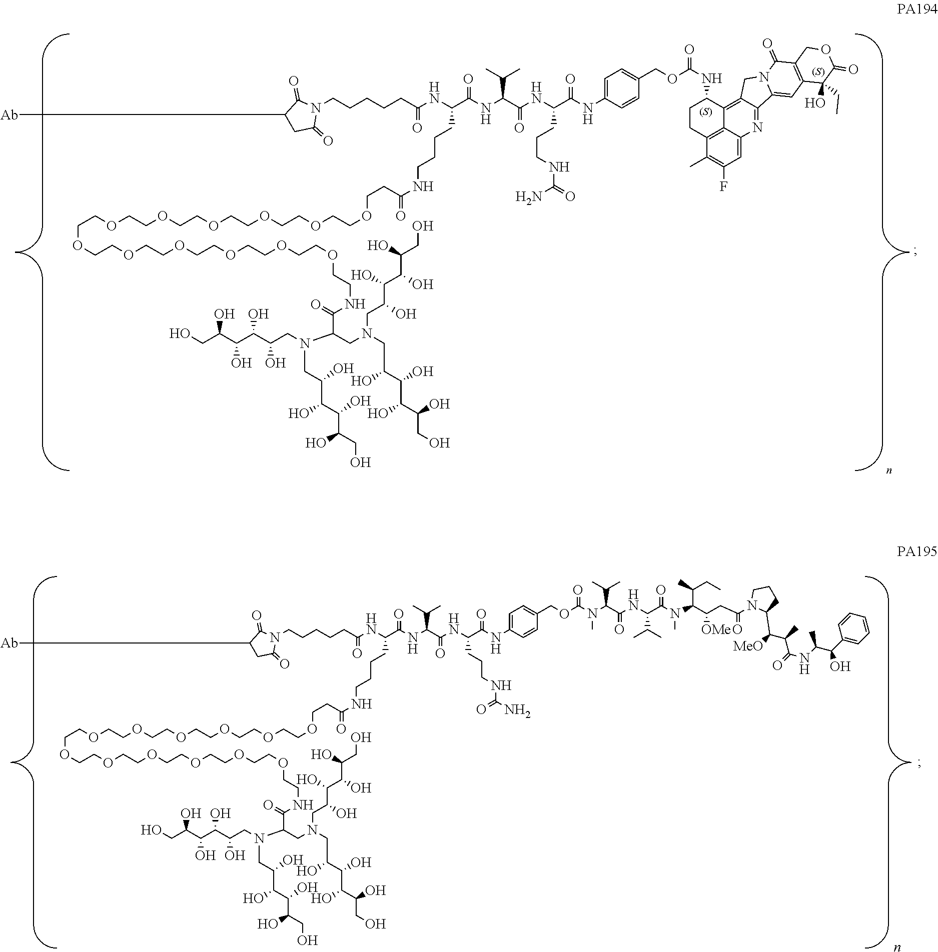

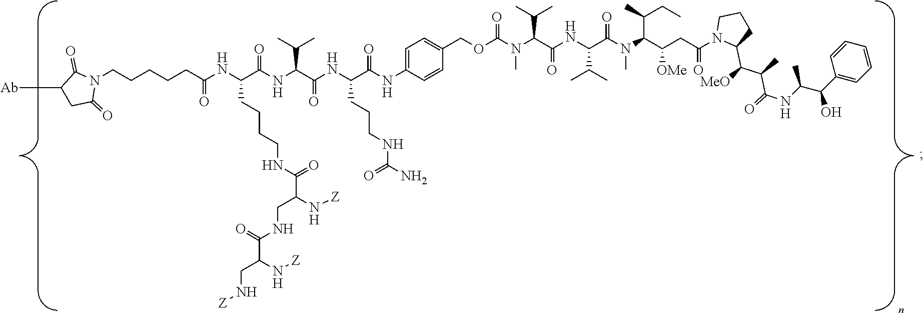

wherein each Z is attached at * and is individually selected from:

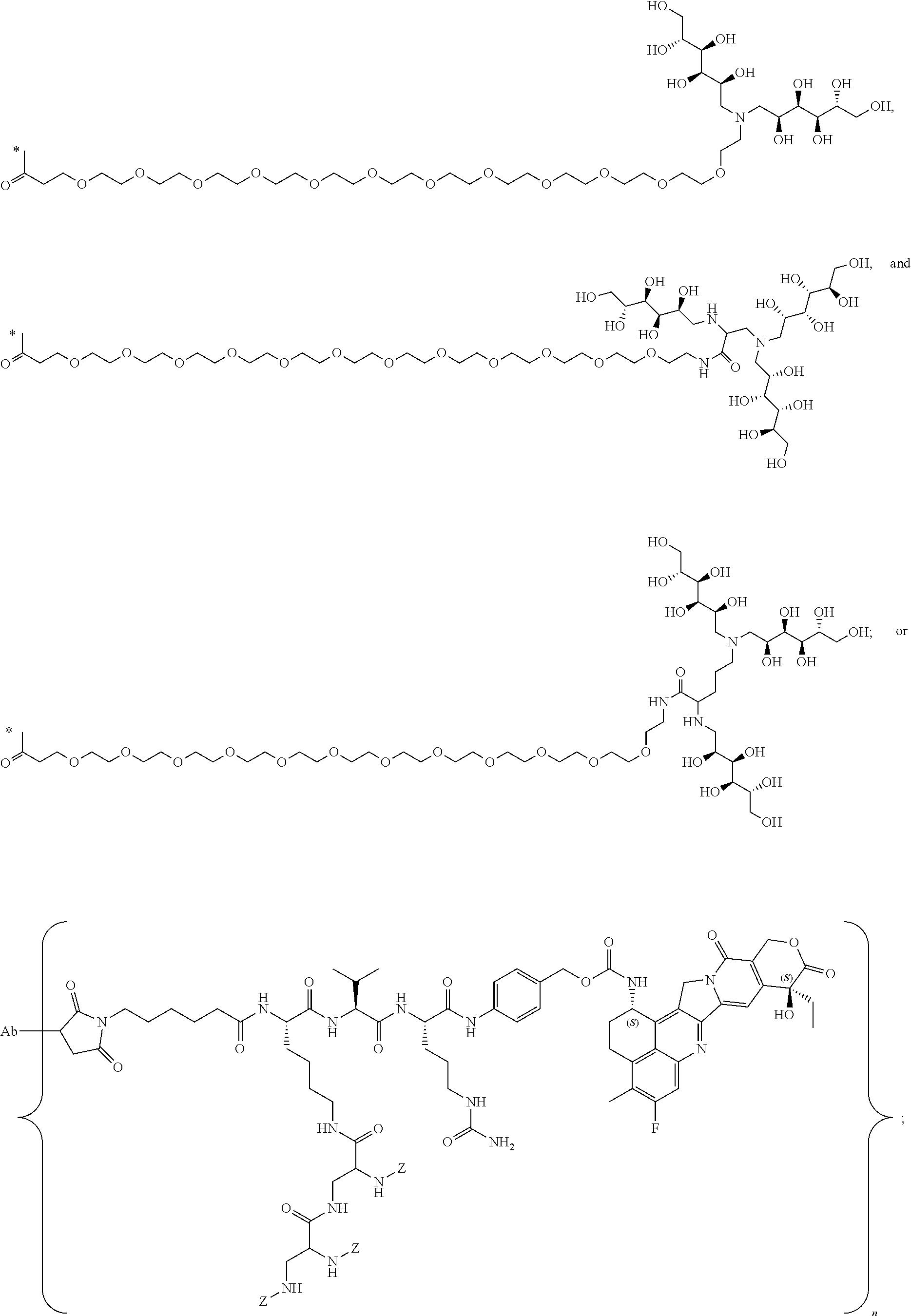

wherein each Z is attached at * and is individually selected from:

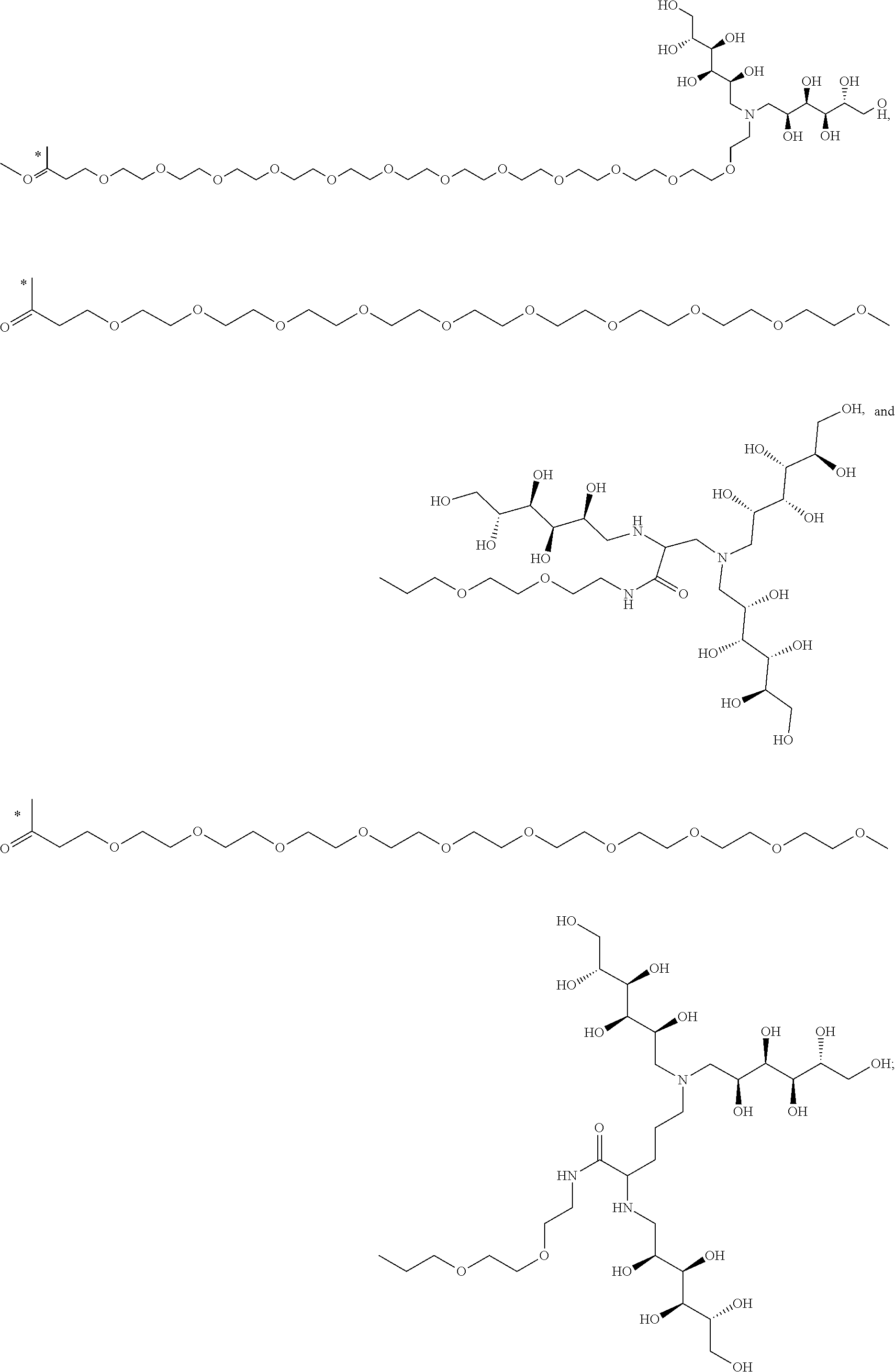

wherein n is pload.

The conjugates listed above may be synthesized according to the procedures disclosed in PCT Publication No. WO2023280227A2, incorporated herein by reference.

Examples of anthracyclines include doxorubicin, epirubicin, nemorubicin; PNU-159682 and derivatives thereof (see, for example, U.S. Pat. No. 10,960,083; Quintieri et al. Clin. Cancer Res. 11:1608-1617, 2005; and Stefan et al. Mol. Cancer Ther. 16:879-892, 2017).

In some embodiments, the cytotoxic agent may be a duocarmycin, including the synthetic analogues, KW-2189 and CBI-TMI.

In some embodiments, the cytotoxic agent may comprise or consists of an auristatin, a camptothecin, a duocarmycin, an anthracycline, a calicheamicin, any analogs thereof, or any combination thereof.

In some embodiments, the cytotoxic agent may comprise or consists of the auristatin.

In some embodiments, the cytotoxic agent may comprise or consists of MMAE.

In some embodiments, the cytotoxic agent may comprise or consists of a camptothecin, or a camptothecin analog/derivative.

In some embodiments, the cytotoxic agent may comprise or consist of exatecan, or an exatecan analog/derivative.

In some embodiments, the cytotoxic agent may comprise or consists of SN-38.

In some embodiments, wherein the cytotoxic agent may comprise or consist of DXd.

The anti-MSLN conjugates contemplated for use in the compositions and methods herein may include at least one linker. Each linker may be attached to at least one cytotoxic agent. Typically, the conjugate may include a linker between the anti-MSLN antibody, or antigen binding fragment thereof, and the cytotoxic agent. The linker may be an enzyme-cleavable linker, for example, a protease cleavable linker (see, for example, PCT Publication No. WO2004/010957), an acid-cleavable linker, a disulfide linker, self-stabilizing linker (see, for example, PCT Publication Nos. WO2018/031690 and WO2015/095755), a non-cleavable linker (see, for example, PCT Publication No. WO2007/008603), and/or a hydrophilic linker (see, for example, PCT Publication No. WO2015/123679). In various embodiments, the linker is cleavable under intracellular conditions, such that cleavage of the linker releases the cytotoxic agent from the antibody in the intracellular environment.

For example, in some embodiments, the linker is cleavable by a cleaving agent that is present in the intracellular environment (for example, within a lysosome or endosome). In some embodiments, the linker may be an enzyme-cleavable linker. The enzyme-cleavable linker may be, for example, a peptidyl linker that is cleaved by an intracellular peptidase or protease enzyme, including, but not limited to, a lysosomal or endosomal protease. Typically, a peptidyl linker is at least one amino acid long or at least two amino acids long. In some embodiments, the enzyme-cleavable linker may be cleaved by cathepsin B, cathepsin D and/or plasmin, all of which are known to hydrolyze dipeptide drug derivatives resulting in the release of active drug inside target cells (see, for example, Dubowchik and Walker, Pharm. Therapeutics 83:67-123, 1999). In some embodiments, the enzyme-cleavable linker may be a peptidyl linker that is cleavable by enzymes that are present in target antigen-expressing cells. For example, a peptidyl linker that is cleavable by the thiol-dependent protease cathepsin-B, which is highly expressed in cancerous tissue, may be used (for example, a Phe-Leu or a Gly-Phe-Leu-Gly linker (SEQ ID NO:23). Other such linkers are described, for example, in U.S. Pat. No. 6,214,345. In specific embodiments, the peptidyl linker cleavable by an intracellular protease is a Val-Cit linker or a Phe-Lys linker (see, for example, U.S. Pat. No. 6,214,345, which describes the synthesis of doxorubicin with the val-cit linker) or Gly-Gly-Phe-Gly linker (SEQ ID NO:24) (see, for example, U.S. Patent Publication 2015/0297748). One advantage of using intracellular proteolytic release of the cytotoxic agent is that the agent is typically attenuated when conjugated and the serum stabilities of the conjugates are typically high (see, for example, U.S. Pat. No. 9,345,785).

As used herein, the terms “intracellularly cleaved” and “intracellular cleavage” refer to a metabolic process or reaction inside a cell on an antibody drug conjugate, whereby the covalent attachment, for example, a linker, between a cytotoxic agent and a binding agent (for example, an anti-MSLN antibody, or an antigen-binding portion thereof), is broken, resulting in the free cytotoxic agent, or other metabolite of the conjugate, dissociated from the binding agent (for example, the anti-MSLN antibody or the antigen-binding portion thereof) inside the cell. The cleaved moieties of the conjugate are thus intracellular metabolites.

In some embodiments, a linker may be a cleavable linker that may be pH-sensitive, for example, sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) may be used (see, for example, U.S. Pat. Nos. 5,122,368; 5,824,805; and 5,622,929; Dubowchik and Walker, Pharm. Therapeutics 83:67-123, 1999; and Neville et al., Biol. Chem. 264:14653-14661, 1989.) Such linkers may be relatively stable under neutral pH conditions, such as those in the blood, but may be unstable at below pH 5.5 or 5.0, the approximate pH of the lysosome. In certain embodiments, a linker may be a hydrolyzable linker that may be a thioether linker (such as, for example, a thioether attached to the therapeutic agent via an acylhydrazone bond (see, for example, U.S. Pat. No. 5,622,929)).

In some embodiments, the linker may be cleavable under reducing conditions, for example, the linker may be a disulfide linker. A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)—(see, for example, Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987); and U.S. Pat. No. 4,880,935)).

In some embodiments, the linker may be a malonate linker (see, for example, Johnson et al., Anticancer Res. 15:1387-93, 1995), a maleimidobenzoyl linker (see, for example, Lau et al., Bioorg-Med-Chem. 3(10):1299-1304, 1995), or a 3′-N-amide analog (see, for example, Lau et al., Bioorg-Med-Chem. 3(10):1305-12, 1995). In some embodiments, the linker unit may not cleavable and the drug may be released by antibody degradation (see, for example, U.S. Patent Publication No. 2005/0238649).

In some embodiments, the linker may not be substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC) or ADC derivative, are cleaved when the ADC or ADC derivative is present in an extracellular environment (for example, in plasma). Whether a linker is not substantially sensitive to the extracellular environment can be determined, for example, by incubating independently with plasma both (a) the ADC or ADC derivative (the “ADC sample”) and (b) an equal molar amount of unconjugated antibody or therapeutic agent (the “control sample”) for a predetermined time period (for example, 2, 4, 8, 16, or 24 hours) and then comparing the amount of unconjugated antibody or therapeutic agent present in the ADC sample with that present in control sample, as measured, for example, by high performance liquid chromatography.

In some embodiments, the linker may promote cellular internalization. In certain embodiments, the linker may promote cellular internalization when conjugated to the cytotoxic agent (for example, in the milieu of the linker-therapeutic agent moiety of the ADC or ADC derivative as described herein). In yet other embodiments, the linker may promote cellular internalization when conjugated to both the cytotoxic agent and the anti-MSLN antibody or derivative thereof (i.e., in the milieu of the ADC or ADC derivative as described herein).

Examples of linkers that may be used with the present conjugates, compositions and methods are described in PCT Publication No. WO 2004010957, incorporated herein in its entirety. In some embodiments, the enzyme-cleavable linker comprises a thiol-reactive spacer and a dipeptide.

In some embodiments, an acid-cleavable linker may be a hydrazine linker or a quaternary ammonium linker (see, for example, PCT Publications WO2017/096311 and WO2016/040684.)

In some embodiments, the linker may be a self-stabilizing linker. Self-stabilizing linkers comprising a maleimide group are described in U.S. Pat. No. 9,504,756.

In some embodiments, the linker may include a T-moiety, for example, a T1000.

Linkers comprising a T-moiety are described in, for example, Weng et al., Cancer Discov 13 (4): 950-973, 2023 incorporated herein by reference in its entirety. In some embodiments, T1000 may be a hydrophilic self-immolative moiety. In some embodiments, use of T1000 as a linker may be useful to overcome the high hydrophobicity of exatecan (see, for example, Ogitani Y, et al. Bioorg. Med. Chem. Lett.: 26:5069-72, 2016). In some embodiments, a conjugate may be an antibody-T1000-exatecan conjugate, for example, an Ab-1-T1000-exatecan conjugate. In some embodiments, a conjugate may be an antibody-MC-VA-T1000-exatecan conjugate, for example, an Ab-1-MC-VA-T1000-exatecan conjugate. In some embodiments, a conjugate including a linker than includes T1000 may be expected to have a drug loading average (or DAR) of about 8.

In some embodiments the anti-MSLN conjugate may have one of the following structures, where Ab is a MSLN binding agent as disclosed herein, for example, an anti-MSLN antibody (for example, Ab-1, or an antigen-binding portion thereof):

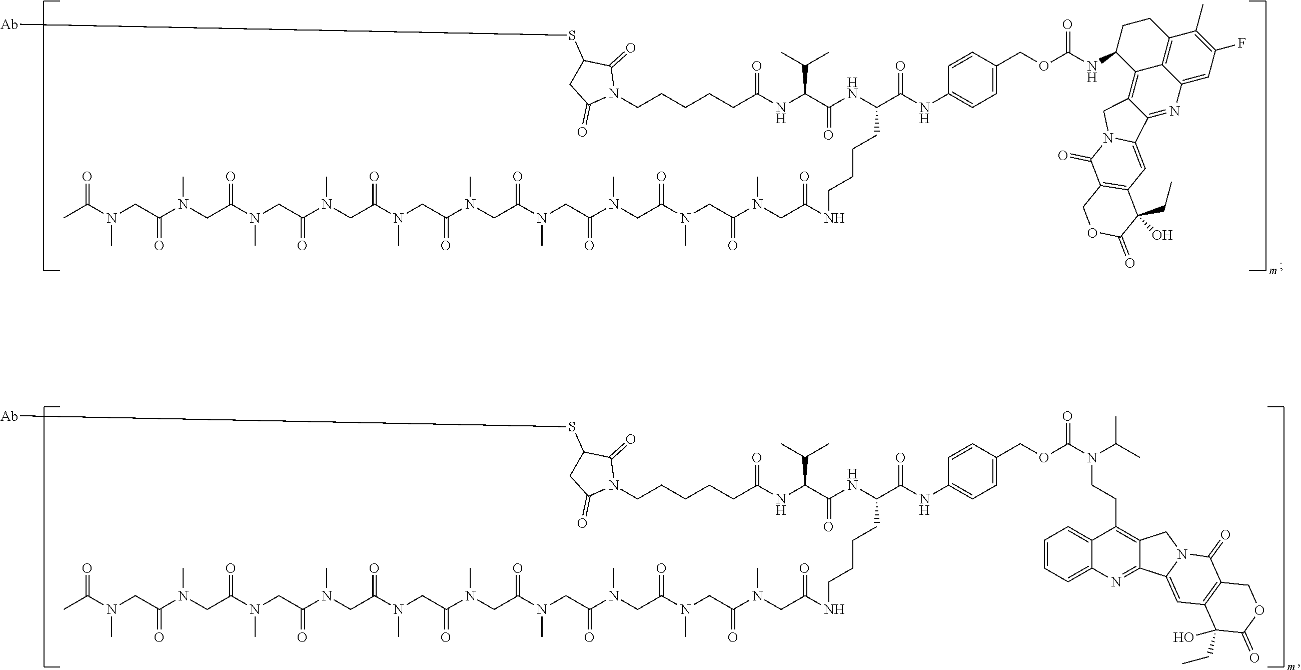

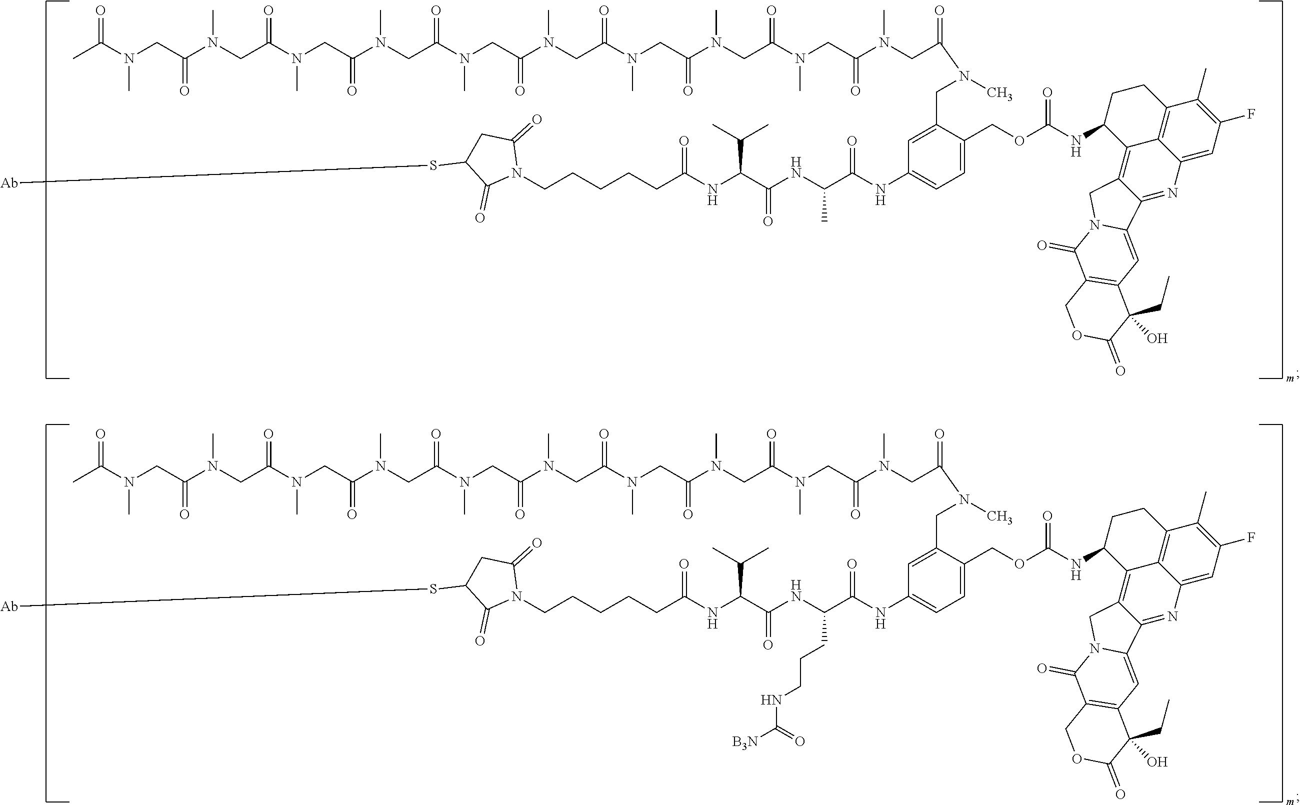

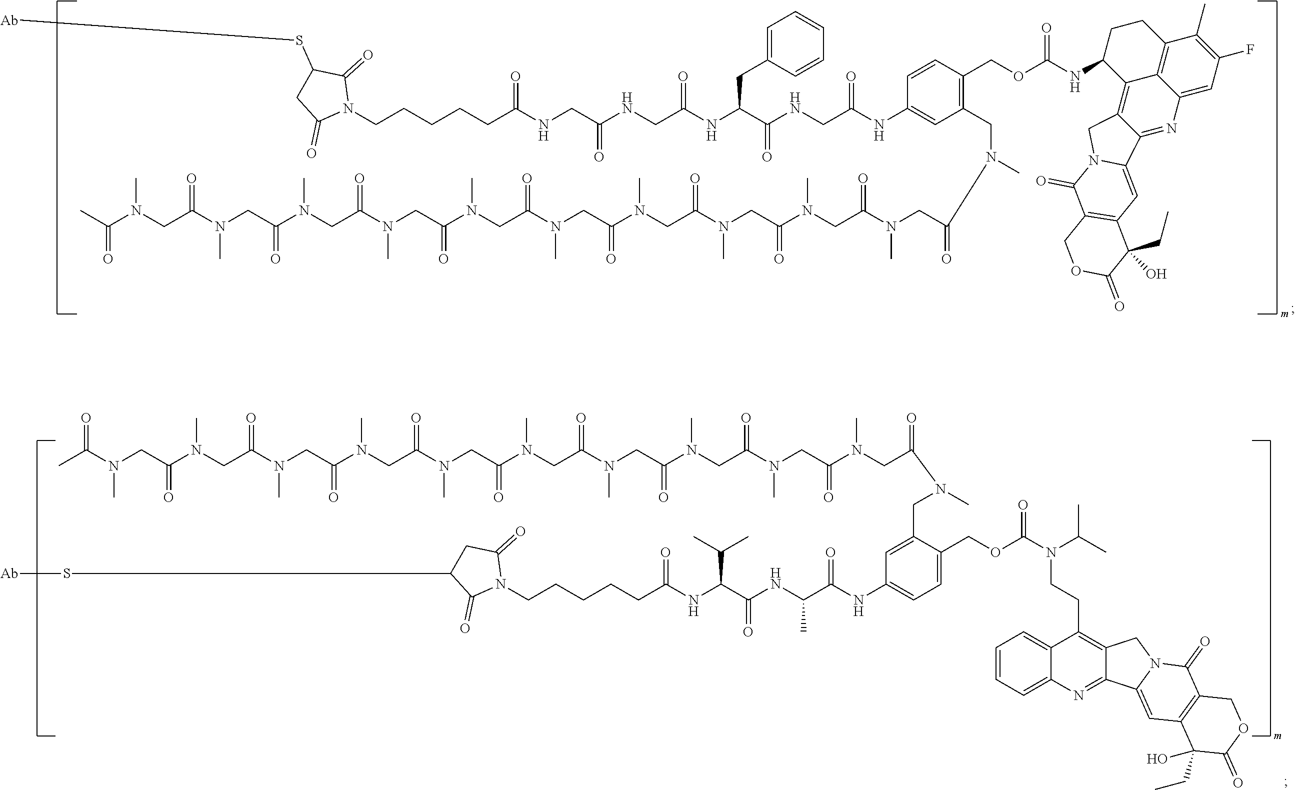

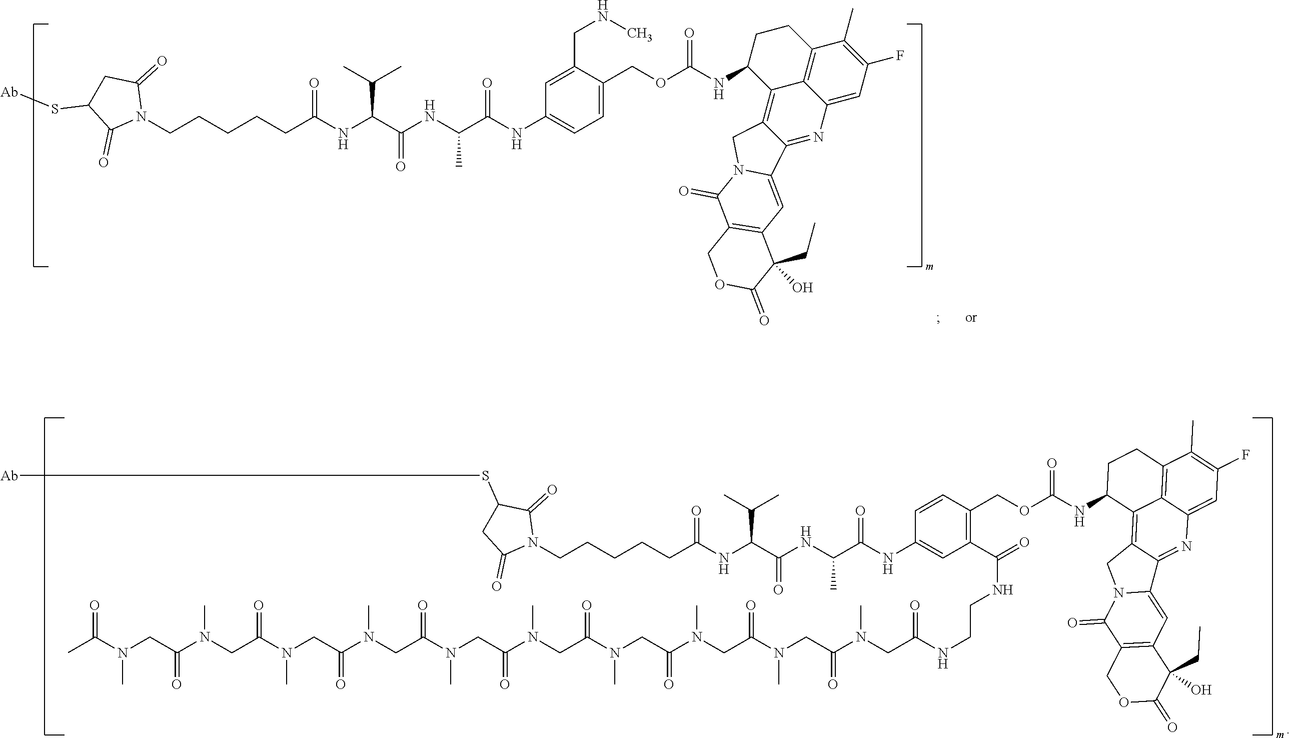

The conjugates listed above may be synthesized according to the procedures disclosed in PCT Publication No. WO2022/228495A1, incorporated herein by reference.

In some embodiments, a tubulin disrupting agent, such as an auristatin, may be conjugated to a linker by a C-terminal carboxyl group that forms an amide bond with a “Linker Unit” (LU) as described in U.S. Pat. No. 9,463,252, incorporated herein by reference. In various embodiments, the Linker Unit includes at least one amino acid. Example antibody drug conjugates (ADCs) of N,N-dialkylauristatins are disclosed in U.S. Pat. No. 8,992,932.

In some embodiments, the linker may further include a stretcher unit and/or an amino acid unit. Exemplary stretcher units and amino acid units are described in U.S. Pat. Nos. 9,345,785 and 9,078,931, each of which is herein incorporated by reference.

In various embodiments, provided herein are antibody drug conjugates comprising an anti-MSLN antibody, covalently linked to MMAE through an mc-val-cit-PAB linker (otherwise referred to as mc-VC-PAB) (see, for example, ADC-4). The anti-MSLN conjugates may be delivered to a subject as a pharmaceutical composition.

In some embodiments, the drug loading of an antibody drug conjugate may be represented by p, the average number of drug molecules (for example, cytotoxic agents) per antibody in a pharmaceutical composition. In some embodiments, the drug loading may be referred to as the average drug-to-antibody ratio, or DAR. For example, if p is about 4, the average drug loading taking into account all of the antibody present in the pharmaceutical composition is about 4. In some embodiments, P ranges from about 3 to about 5, more preferably from about 3.6 to about 4.4, even more preferably from about 3.8 to about 4.2. P can be about 3, about 4, or about 5. In some embodiments, P ranges from about 6 to about 8, more preferably from about 7.5 to about 8.4. P can be about 6, about 7, or about 8. The average number of drugs per antibody in preparation of conjugation reactions may be characterized by conventional means such as mass spectroscopy, ELISA assay, and HPLC. The quantitative distribution of antibody drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

In some embodiments, an average number of cytotoxic agents per binding agent (or DAR) (for example, an antibody) is from about 1 to about 8, about 2, about 4, about 6, about 8, about 10, about 12, about 14, about 16, about 3 to about 5, about 6 to about 8 or about 8 to about 16.

A Stretcher unit (A) is capable of linking an antibody (or an antigen-binding portion thereof) unit, to an amino acid unit (for example, a valine-citrulline peptide) via a sulfhydryl group of the antibody. Sulfhydryl groups may be generated, for example, by reduction of the interchain disulfide bonds of an anti-MSLN antibody, or an antigen-binding portion thereof. For example, a Stretcher unit may be linked to the antibody (or an antigen-binding portion thereof) via the sulfur atoms generated from reduction of the interchain disulfide bonds of the anti-MSLN antibody, or an antigen-binding portion thereof. In some embodiments, the Stretcher units are linked to the antibody (or an antigen-binding portion thereof) solely via the sulfur atoms generated from reduction of the interchain disulfide bonds of the antibody. In some embodiments, sulfhydryl groups can be generated by reaction of an amino group of a lysine moiety of an anti-MSLN antibody with 2-iminothiolane (Traut's reagent) or other sulfhydryl generating reagents. In certain embodiments, the anti-MSLN antibody, or an antigen-binding portion thereof, is a recombinant antibody and is engineered to carry one or more lysines. In certain other embodiments, the recombinant MSLN antibody, or an antigen-binding portion thereof, is engineered to carry additional sulfhydryl groups, e.g., additional cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945, each of which is incorporated herein by reference in its entirety.

Representative Stretcher units are described within the square brackets of Formulas Illa and lllb of U.S. Pat. No. 9,211,319, and are incorporated herein by reference.

In some embodiments, the anti-MSLN conjugates have the following formula:

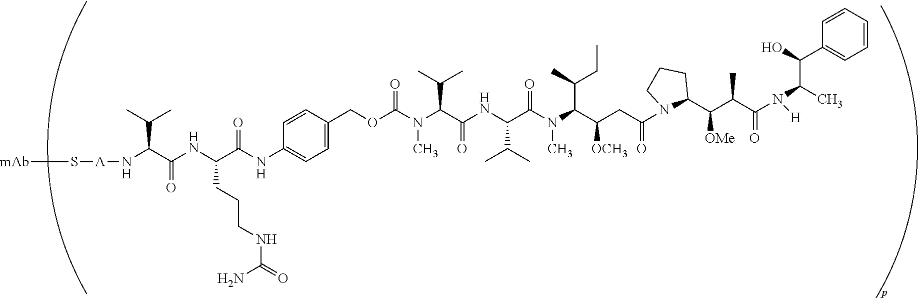

or a pharmaceutically acceptable salt thereof; wherein: mAb is an anti-MSLN antibody (for example, Ab-1, or an antigen-binding portion thereof), S is a sulfur atom of the antibody, and A- is a Stretcher unit. In some embodiments, p may be from about 3 to about 5, or from about 3 to about 8.

The abbreviation “MMAE” refers to monomethyl auristatin E.

The abbreviations “ve” and “val-cit” refer to the dipeptide valine-citrulline.

The abbreviation “PAB” refers to the self-immolative spacer:

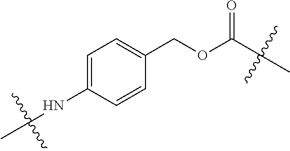 [SMILES: *Nc1ccc(COC(*)=O)cc1]

The abbreviation “MC” refers to the stretcher maleimidocaproyl:

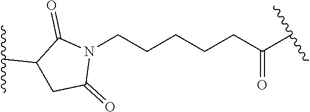 [SMILES: *C(=O)CCCCCN1C(=O)CC(*)C1=O]

In some embodiments, the antibody drug conjugate may include monomethyl auristatin E (MMAE) and a protease-cleavable linker. In some embodiments, the protease cleavable linker may include a thiol-reactive spacer and a dipeptide. In some embodiments, the protease cleavable linker consists of a thiol-reactive maleimidocaproyl spacer, a valine--citrulline dipeptide, and a p-amino-benzyloxycarbonyl or PAB spacer.

In some embodiments, the antibody drug conjugate may have the following general formula:

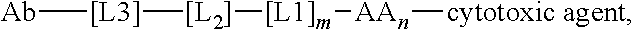

where Ab is an anti-MSLN antibody (for example, Ab-1, or an antigen-binding portion thereof); the cytotoxic agent may be a tubulin-disrupting agent or topoisomerase inhibitor; L3 is a component of a linker comprising an antibody-coupling moiety and one or more of acetylene (or azide) groups; L2 comprises a defined PEG (polyethylene glycol) azide (or acetylene) at one end, complementary to the acetylene (or azide) moiety in L3, and a reactive group such as carboxylic acid or hydroxyl group at the other end; L1 comprises a collapsible unit (for example, a self-immolative group(s)), or a peptidase-cleavable moiety optionally attached to a collapsible unit, or an acid-cleavable moiety; AA is an amino acid; m is an integer with values of 0 or 1, and n is an integer with values of 0, 1, 2, 3, or 4. In some embodiments, such linkers may be assembled using click chemistry (see, for example, U.S. Pat. Nos. 7,591,944 and 7,999,083).

In some embodiments, the cytotoxic agent is a camptothecin or a camptothecin (CPT) analog, such as irinotecan (also referred to as CPT-11), topotecan, 10-hydroxy-CPT, exatecan, an exatecan analog, DXd and SN-38. Representative structures are shown below.

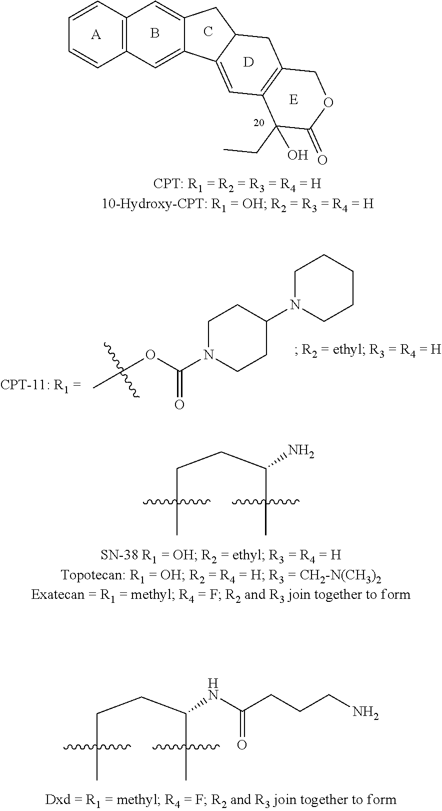

Referring to the conjugate formula Ab-[L3]-[L2]-[L1]ₘ-AAₙ-cytotoxic agent, in some embodiments, m is 0. In such embodiments, an ester moiety may first be formed between the carboxylic acid of an amino acid (AA) such as glycine, alanine, or sarcosine, or of a peptide such as glycylglycine, and a hydroxyl group of a cytotoxic agent. In this example, the N-terminus of the amino acid or polypeptide may be protected as a Boc or a Fmoc or a monomethoxytrityl (MMT) derivative, which is deprotected after formation of an ester bond with the hydroxyl group of the cytotoxic agent. Selective removal of amine-protecting group, in the presence of a BOC protecting group at a hydroxyl position of the cytotoxic agent containing an additional hydroxyl group(s) may be achieved using monomethoxytrityl (MMT) as the protecting group for the amino group of amino acid or polypeptide involved in ester formation, since ‘MMT’ is removable by mild acid treatment such as dichloroacetic acid that does not cleave a BOC group. After the amino group of the amino acid or polypeptide, forming an ester bond with hydroxyl of the cytotoxic agent, is demasked, the amino group may be reacted with the activated form of a COOH group on PEG moiety of L2 under standard amide-forming conditions. In some embodiments, L3 includes a thiol-reactive group which links to thiol groups of the antibody (for example, Ab-1), or an antigen-binding portion thereof. The thiol-reactive group is optionally a maleimide or vinylsulfone, or bromoacetamide, or iodoacetamide, which links to a thiol group of the antibody. In some embodiments, the reagent bearing a thiol-reactive group is generated from succinimidyl-4-(N maleimidomethyl)cyclohexane-1-carboxylate (SMCC) or from succinimidyl-(epsilon-maleimido)caproate, for example, with the thiol-reactive group being a maleimide group.

In another embodiments, m is 0, and AA comprises a peptide moiety, preferably a di, tri or tetrapeptide, that is cleavable by intracellular peptidase such as Cathepsin-B. Examples of cathepsin-B-cleavable peptides are: Phe-Lys, Val-Cit (see, for example, Dubowchick, 2002), Ala-Leu, Leu-Ala-Leu, and Ala-Leu-Ala-Leu (SEQ ID NO:25) (see, for example, Trouet et al., 1982).

In some embodiments, L1 may include an intracellularly-cleavable peptide, such as cathepsin-B-cleavable peptide, connected to the collapsible unit p-aminobenzyl alcohol (or p-amino-benzyloxycarbonyl) at the peptide's C-terminus, the benzyl alcohol portion of which is in turn directly attached to a hydroxyl group of the cytotoxic agent, in chloroformate form. In this embodiment, n is 0. Alternatively, when ‘n’ is non-zero, the benzyl alcohol portion of the p-amidobenzyl alcohol (or p-amino-benzyloxycarbonyl) moiety is attached to the N-terminus of the amino acid or peptide linking at the hydroxyl group of the cytotoxic agent through the activated form of p-amidobenzyl alcohol, namely PABOCOPNP where PNP is p-nitrophenyl. In some embodiments, the linker may include a thiol-reactive group which links to thiol groups of the antibody. The thiol-reactive group is optionally a maleimide or vinylsulfone, or bromoacetamide, or iodoacetamide, which links to thiol groups of the antibody, or antigen-binding portion thereof. In some embodiments, the component bearing a thiol-reactive group may be generated from succinimidyl-4-(N maleimidomethyl)cyclohexane-1-carboxylate (SMCC) or from succinimidyl-(epsilon-maleimido)caproate, for example, with the thiol-reactive group being a maleimide group.

In some embodiments, where the cytotoxic agent is a camptothecin or analog or derivative thereof having a 20-hydroxyl, L1 may be composed of an intracellularly-cleavable peptide, such as cathepsin-B-cleavable peptide, connected to the collapsible linker p-aminobenzyl alcohol (or p-amino-benzyloxycarbonyl) at the peptide's C-terminus, the benzyl alcohol portion of which is in turn may be directly attached to CPT-20-O-chloroformate. In this embodiment, n is 0. Alternatively, when ‘n’ is non-zero, the benzyl alcohol portion of the p-amidobenzyl alcohol moiety may be attached to the N-terminus of the amino acid or polypeptide linking at CPT's 20 position through the activated form of p-amidobenzyl alcohol, namely PABOCOPNP where PNP is p-nitrophenyl. In some embodiments, the linker may include a thiol-reactive group which links to thiol groups of an antibody, or an antigen-binding portion thereof. The thiol-reactive group may be a maleimide or vinylsulfone, or bromoacetamide, or iodoacetamide, which links to thiol groups of an antibody, or an antigen-binding portion thereof. In some embodiments, the component bearing a thiol-reactive group may be generated from succinimidyl-4-(N maleimidomethyl)cyclohexane-1-carboxylate (SMCC) or from succinimidyl-(epsilon-maleimido)caproate, for example, with the thiol-reactive group being a maleimide group.

In some embodiments, the L2 component of the conjugate may include a polyethylene glycol (PEG) spacer that may be of up to MW 5000 in size. In some embodiments, PEG is a defined PEG with (1-12 or 1-30) repeating monomeric units. In some embodiments, PEG is a defined PEG with 1-12 repeating monomeric units. The introduction of PEG may involve using heterobifunctionalized PEG derivatives which are available commercially. In the context of the present disclosure, the heterobifunctional PEG may include an azide or acetylene group. An example of a heterobifunctional defined PEG containing 8 repeating monomeric units, with ‘NHS’ being succinimidyl, is given below in the following formula:

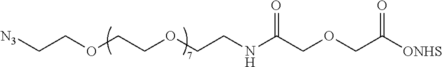

In some embodiments, L3 has a plurality of acetylene (or azide) groups, ranging from 2-40, but preferably 2-20, and more preferably 2-5, and a single antibody binding moiety.

An example conjugate, in which the cytotoxic agent is SN-38 (a CPT analog), prepared with a maleimide-containing SN-38-linker derivative, with the bonding to an antibody (for example, Ab-1, or an antigen-binding portion thereof) represented as a succinimide, is given below. Here, m=0, and the 20-O-AA ester bonding to SN-38 is glycinate; azide-acetylene coupling joining of L2 and L3 results in the triazole moiety as shown.

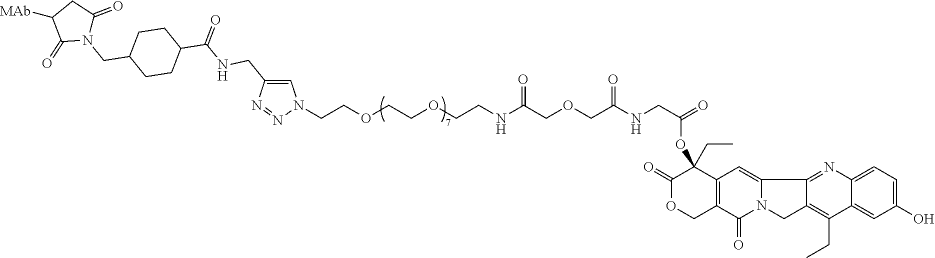

In another example conjugate, prepared with a maleimide-containing SN-38-linker derivative, with the bonding to an antibody (for example, Ab-1, or an antigen-binding portion thereof) represented as a succinimide, is shown below. Here, n=0 in the general formula 2; ‘L1’ contains a cathepsin-B-cleavable dipeptide attached to the collapsible p-aminobenzyl alcohol moiety, and the latter is attached to SN-38 as a carbonate bonding at the 20 position; azide-acetylene coupling joining the ‘L2’ and ‘L3’ parts results in the triazole moiety as shown.

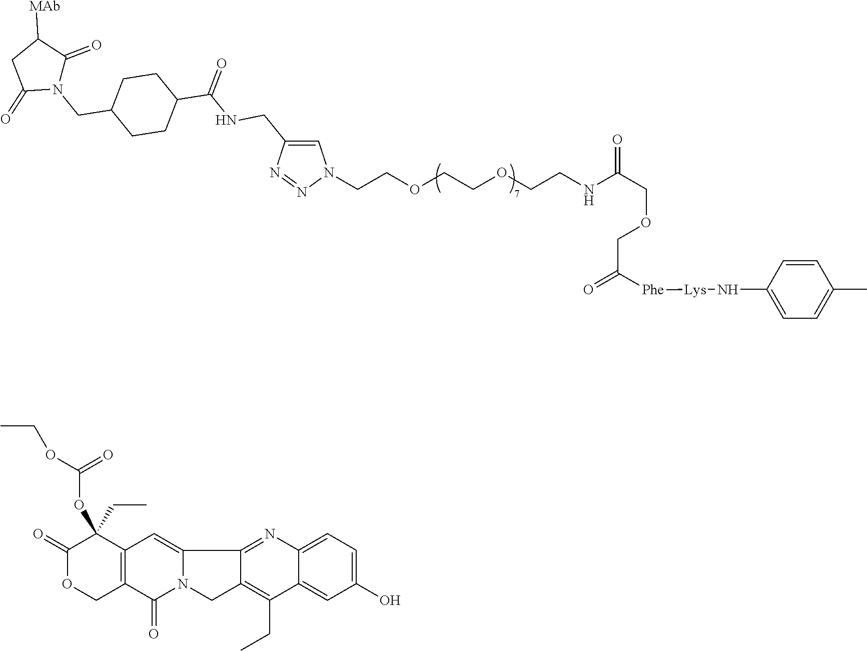

Another representative SN-38 conjugate, Mab-CL2-SN-38, prepared with a maleimide-containing SN-38-linker derivative, with the bonding to an antibody (for example, Ab-1, or an antigen-binding portion thereof) represented as a succinimide, is given below. Here, the 20-O-AA ester bonding to SN-38 is glycinate that is attached to L1 portion via a p-aminobenzyl alcohol moiety and a cathepsin-B-cleavable dipeptide; the latter is in turn attached to ‘L2’ via an amide bond, while ‘L2’ and ‘L3’ parts are coupled via azide-acetylene ‘click chemistry’.

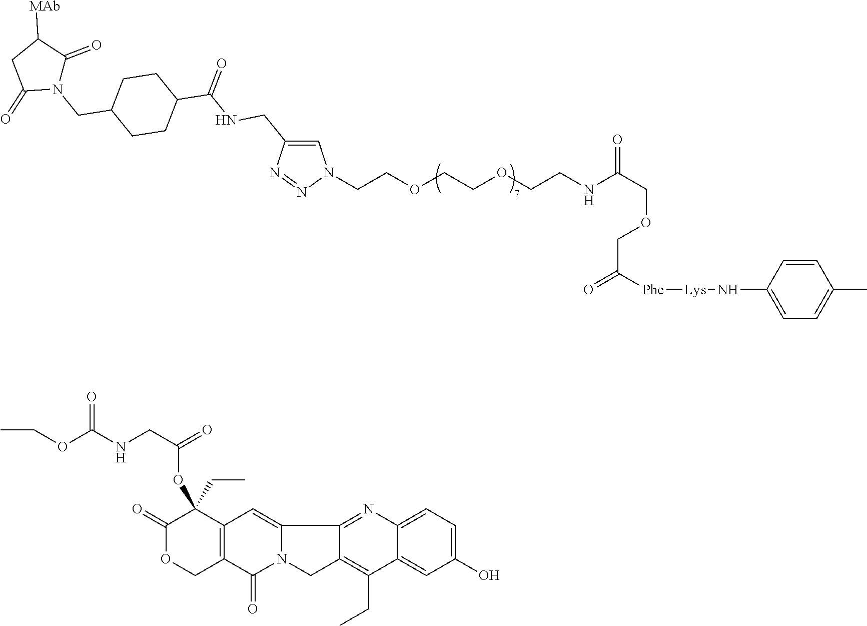

In another example below, ‘L1’ contains a single amino acid attached to the collapsible p-aminobenzyl alcohol moiety, where the p-aminobenzyl alcohol is substituted or unsubstituted (R), where m=1 and n=0 in the general conjugate formula, and the cytotoxic agent is exemplified with SN-38. The structure is represented below (referred to as MAb-CLX-SN-38; the antibody, for example, may be Ab-1, or an antigen-binding portion thereof). Single amino acid of AA can be selected from any one of the following L-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The substituent R on 4-aminobenzyl alcohol moiety is hydrogen or an alkyl group selected from C1-C10 alkyl groups.

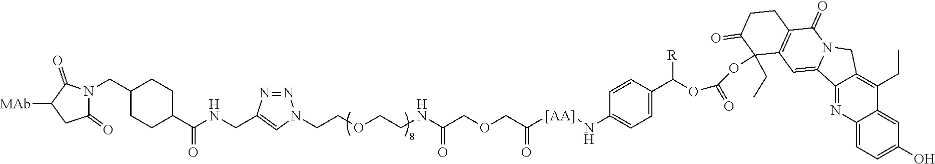

An embodiment of MAb-CLX-SN-38 (above), wherein the single amino acid AA is L-lysine and R=H, and the cytotoxic agent is exemplified by SN-38 (referred to as MAb-CL2A-SN-38; the antibody, for example, may be Ab-1, or an antigen-binding portion thereof; see also, for example, ADC-2) is shown below:

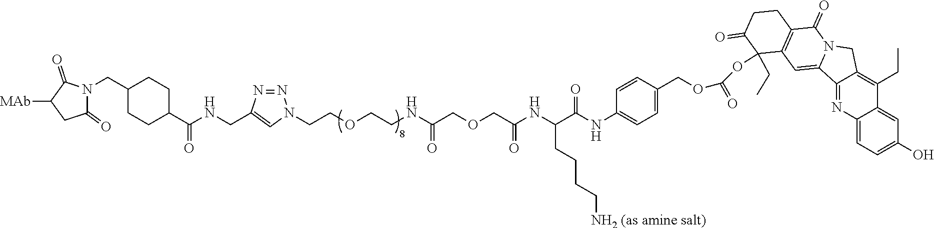

In other embodiments, a cytotoxic agent is attached to a linker comprising a Stretcher unit (Z) attached to an Amino Acid unit (AA) attached to a Spacer unit (Y), where the Stretcher unit is attached to the antibody (Ab or Mab; for example, Ab-1, or an antigen-binding portion thereof) and the Spacer unit is attached to an amino group of a cytotoxic agent. Such a linker has the following formula:

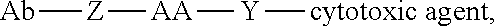

where Z is selected from -(Succinimid-3-yl-N)—(CH₂)ₙ²—C(═O)—, —CH₂—C(═O)—NH—(CH₂)n³-C(═O)—, —C(═O)-cyc.Hex(1,4)-CH₂—(N-ly-3-diminiccuS)-, or —C(═O)—(CH₂)n⁴-C(═O)—, wherein n² represents an integer of 2 to 8, n³ represents an integer of 1 to 8, and n⁴ represents an integer of 1 to 8; cyc.Hex(1,4) represents a 1,4-cyclohexylene group; and (N-ly-3-diminiccuS)- has a structure represented by the following formula:

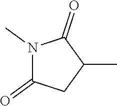

AA is a peptide of from 2 to 7 amino acids. The spacer unit Y is —NH—(CH₂)_{b}—(C═O)— or —NH—CH₂—O—CH₂—(C═O)—, where b is an integer from 1 to 5.

The abbreviation “CL2” refers to a cross-linker (CL) version 2 at C20 hydroxyl position (see, for example, Moon et al. J Med Chem 51:6916, 2008).

The abbreviation “CL2A” refers to a version of CL2 without Phe in the linker (see, for example, Cardillo et al. Clin Can Res 17: 3157, 2011).

In some embodiments, the cytotoxic agent is exatecan. In some embodiments, the amino acid unit (AA) is -Gly-Gly-Phe-Gly- (SEQ ID NO:24). In some embodiments, the spacer unit Y is —NH—CH₂—O—CH₂-(C═O)-.

In some embodiments, the linker-cytotoxic agent has the following structure:

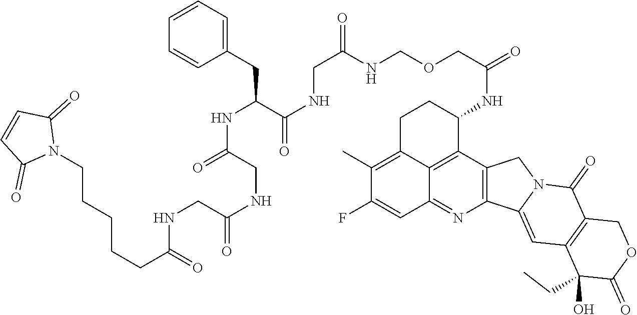 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

where the released cytotoxic agent is DXd (see U.S. Pat. No. 9,808,537).

In some embodiments, the linker-cytotoxic agent is a drug-linker disclosed in PCT Publication No. WO2023280227A2, incorporated herein by reference in its entirety). Drug-linkers may be synthesized according to the procedures disclosed in PCT Publication No. WO2023280227A2, incorporated herein by reference.

In some embodiments, the linker comprises or consists of mc-VC-PAB, CL2, CL2A, (Succinimid-3-yl-N)-(CH₂)ₙ²—C(═O)-Gly-Gly-Phe-Gly-NH—CH₂—OCH₂—(C═O)—, an enzyme-cleavable linker, or any combination thereof.

In some embodiments, the linker comprises or consists of mc-VC-PAB.

In some embodiments, the linker is attached to at least one molecule of MMAE.

In some embodiments, the linker comprises or consists of CL2A.

In some embodiments, the linker is attached to at least one molecule of SN-38.

In some embodiments, the linker comprises or consists of CL2.

In some embodiments, the linker is attached to at least one molecule of SN-38.

In some embodiments, the linker comprises or consists of (Succinimid-3-yl-N)-(CH₂)ₙ²—C(═O)-Gly-Gly-Phe-Gly-NH—CH₂—O—CH₂-(C═O)-.

In some embodiments, the linker is attached to at least one molecule of DXd.

In some embodiments, the linker comprises or consists of an enzyme-cleavable linker.

In some embodiments, the linker is attached to at least one molecule of exatecan.

In some embodiments, the conjugate is ADC-1 (Ab-1-GGFG-DXd).

In some embodiments, the conjugate is ADC-2 (Ab-1-CL2A-SN38).

In some embodiments, the conjugate is ADC-3 (Ab-1-enzyme-cleavable linker-exatecan).

In some embodiments, the conjugate is ADC-4 (Ab-1-mc-VC-PAB-MMAE).

### Attachment of Cytotoxic Agent-Linkers to Antibodies or Antigen-Binding Portions Thereof, or Other Binding Agents

Techniques for attaching cytotoxic agents to antibodies or antigen binding portions thereof, or other binding agents, via linkers are well-known in the art (see, for example, Alley et al., Current Opinion in Chemical Biology 14:1-9, 2010; and Senter, Cancer J., 14(3):154-169, 2008). In some embodiments, a linker is first attached to a cytotoxic agent(s) and then the linker-cytotoxic agent(s) is attached to the antibody or antigen binding portion thereof. In some embodiments, a linker is first attached to an antibody or antigen binding portion thereof, and then a cytotoxic agent(s) is attached to the linker. In the following discussion, the term linker-cytotoxic agent(s) is used to exemplify attachment of linkers or linker-cytotoxic agent(s) to antibodies or antigen binding portions thereof; the skilled artisan will appreciate that the selected attachment method can be selected according to linker and the cytotoxic agent. In some embodiments, a cytotoxic agent is attached to an antibody or antigen binding portion thereof via a linker in a manner that reduces its activity until it is released from the conjugate (for example, by hydrolysis, by proteolytic degradation or by a cleaving agent).

Generally, a conjugate may be prepared by several routes employing organic chemistry reactions, conditions, and reagents known to those skilled in the art, including: (1) reaction of a nucleophilic group of an antibody, or antigen binding portion thereof, with a bivalent linker reagent to form an antibody-linker intermediate via a covalent bond, followed by reaction with a cytotoxic agent; and (2) reaction of a nucleophilic group of a cytotoxic agent with a bivalent linker reagent, to form linker-cytotoxic agent(s), via a covalent bond, followed by reaction with a nucleophilic group of an antibody or antigen binding portion thereof. Exemplary methods for preparing conjugates via the latter route are described in U.S. Pat. No. 7,498,298, which is expressly incorporated herein by reference.

Nucleophilic groups on antibodies may include, but are not limited to: (i)N-terminal amine groups, (ii) side chain amine groups, for example, lysine, (iii) side chain thiol groups, for example, cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, for example, cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, for example, by reacting lysine residues with 2-iminothiolane (Traut's reagent), resulting in conversion of an amine into a thiol. Reactive thiol groups may also be introduced into an antibody by introducing one, two, three, four, or more cysteine residues (for example, by preparing variant antibodies comprising one or more non-native cysteine amino acid residues).

Conjugates of the disclosure may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In some embodiments, an antibody is modified to introduce electrophilic moieties that are capable of reacting with nucleophilic substituents on the linker reagent or drug. In some embodiments, the sugars of glycosylated antibodies may be oxidized, for example, with periodate oxidizing reagents, to form aldehyde or ketone groups which may react with the amine group of linker reagents or drug moieties. The resulting imine Schiff base groups may form a stable linkage, or may be reduced, for example, by borohydride reagents to form stable amine linkages. In some embodiments, reaction of the carbohydrate portion of a glycosylated antibody with either galactose oxidase or sodium meta-periodate may yield carbonyl (aldehyde and ketone) groups in the antibody, or antigen binding portion thereof, that can react with appropriate groups on the drug (see, for example, Hermanson, Bioconjugate Techniques). In another embodiment, antibodies containing N-terminal serine or threonine residues can react with sodium meta-periodate, resulting in production of an aldehyde in place of the first amino acid (see, for example, Geoghegan & Stroh, Bioconjugate Chem. 3:138-146, 1992; and U.S. Pat. No. 5,362,852). Such an aldehyde can be reacted with a cytotoxic agent or linker.

Exemplary nucleophilic groups on a cytotoxic agent may include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare a conjugate are described herein or are known to persons of ordinary skill in the art. Methods of using such cross-linker reagents to link two moieties, including a proteinaceous moiety and a chemical moiety, are known in the art. In some embodiments, a fusion protein comprising an antibody and a cytotoxic agent may be made, for example, by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate.

In yet another embodiment, an antibody may be conjugated to a “receptor” (such as streptavidin) for utilization in tumor pre-targeting wherein the antibody-receptor conjugate is administered to a subject, followed by removal of unbound conjugate from the circulation using a clearing agent and then administration of a “ligand” (e.g., avidin) which is conjugated to a cytotoxic agent (e.g., a drug or radionucleotide).

In some embodiments, a linker-cytotoxic agent(s) may be attached to interchain cysteine residues of an antibody, or antigen-binding fragment thereof (see, for example, PCT Publication Nos. WO2004/010957 and WO2005/081711). In such embodiments, the linker typically includes a maleimide group for attachment to the cysteine residues of an interchain disulfide. In some embodiments, the linker or linker-cytotoxic agent may be attached to cysteine residues of an antibody, or antigen binding portion thereof, as described in U.S. Pat. No. 7,585,491 or 8,080,250. The drug loading of the resulting conjugate typically ranges from 1 to 8.

In some embodiments, the linker or linker-cytotoxic agent may be attached to lysine or cysteine residues of an antibody, or antigen binding portion thereof, as described in PCT Publication Nos. WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences may be used for site-specific attachment of linkers or linker-cytotoxic agent(s) to antibodies or antigen binding portions thereof.

In some embodiments, a linker-cytotoxic agent(s) may be attached to an engineered cysteine residue at an Fc region residue other than an interchain disulfide. In some embodiments, a linker-cytotoxic agent(s) may be attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, for example, U.S. Patent Publication No. 2010/0158909; numbering of the Fc region is according to the EU index).

In some embodiments, a linker or linker-cytotoxic agent(s) may be attached to one or more introduced cysteine residues of an antibody or antigen binding portion thereof as described in PCT Publication Nos. WO2006/034488, WO2011/156328 and/or WO2016040856.

In some embodiments, an exemplary substitution for site specific conjugation using bacterial transglutaminase is N297S or N297Q of the Fc region. In some embodiments, a linker or linker-cytotoxic agent(s) may be attached to the glycan or modified glycan of an antibody, or antigen binding portion thereof, or a glycoengineered antibody, or antigen binding portion thereof (see, for example, PCT Publication Nos. WO2017/147542, WO2020/123425, WO2014/072482; WO2014//065661, WO2015/057066 and WO2016/022027.

### III. Pharmaceutical Formulations

Other aspects of the anti-MSLN antibodies (and antigen binding portions thereof), or other binding agents, or conjugates, relate to compositions comprising an active agent and/or active ingredients (for example, including an anti-MSLN conjugate, as described herein). In some embodiments, the composition is a pharmaceutical composition. As used herein, the term “pharmaceutical composition” refers to the active agent in combination with a pharmaceutically acceptable carrier, diluent, or excipient accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments, the term “pharmaceutically acceptable,” means that the vehicle, diluent, excipient and/or salts thereof, are chemically and/or physically is compatible with other ingredients in the formulation, and the physiologically compatible with the recipient.

In some embodiments, the present disclosure provides a pharmaceutical composition including an anti-MSLN conjugate.

In some embodiments, the present disclosure provides a pharmaceutical composition including a conjugate, wherein the conjugate includes a binding agent, at least one linker attached to the binding agent, and at least one cytotoxic agent attached to the at least one linker; and a pharmaceutically acceptable carrier.

In some embodiments, the pharmaceutical composition has an average number of cytotoxic agents per binding agent of from about 1 to about 8, about 2, about 4, about 6, about 8, about 10, about 12, about 14, about 16, about 3 to about 5, about 6 to about 8 or about 8 to about 16.

In some embodiments, the present disclosure provides a pharmaceutical composition including a conjugate, wherein the conjugate includes a binding agent including a heavy chain variable (VH) region and a light chain variable (VL) region, wherein the VH region comprises a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region includes a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6; at least one linker attached to the binding agent; and at least one cytotoxic agent attached to the at least one linker, and a pharmaceutically acceptable carrier.

As used herein, the term “a pharmaceutically acceptable carrier and/or excipient” refers to a carrier and/or excipient pharmacologically and/or physiologically compatible with a subject and an active agent, which is well known in the art (see, for example, Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminum adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

The preparation of a pharmacological composition that includes active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use may also be prepared. A preparation can also be emulsified or presented as a liposome composition. An anti-MSLN conjugate, may be mixed with excipients that are pharmaceutically acceptable and compatible with the active ingredient and in amounts suitable for use in the therapeutic methods described herein. Suitable excipients are, for example, water, saline, dextrose, glycerol, ethanol, or the like, and combinations thereof. In addition, if desired, a pharmaceutical composition can contain minor amounts of auxiliary substances such as wetting or emulsifying agents, pH buffering agents and the like which enhance or maintain the effectiveness of the active ingredient (for example, an anti-MSLN conjugate). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that include the active ingredients (for example, an anti-MSLN conjugate) and water, and may contain a buffer such as sodium phosphate at physiological pH value, physiological saline or both, such as phosphate-buffered saline. Still further, aqueous carriers may contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, the pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, mercapto sorbitol, butyl methyl anisole, butylated hydroxy toluene and/or propyl gallate. In some embodiments, the present disclosure provides a composition comprising one or more conjugates, and one or more anti-oxidants such as methionine. In some embodiments, wherein a conjugate is mixed with one or more anti-oxidants, such as methionine, the conjugate may be prevented from oxidation, to extend shelf life and/or provide increased activity.

In some embodiments, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions described herein may be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques.

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

In some embodiments, formulations for enteral administration may include hard or soft gelatin capsules, pills, tablets, including coated tablets, elixirs, suspensions, syrups or inhalations and controlled release forms thereof.

Formulations suitable for parenteral administration (for example, by injection), may include aqueous or non-aqueous, isotonic, pyrogen-free, sterile liquids (for example, solutions, suspensions), in which the active ingredient is dissolved, suspended, or otherwise provided (for example, in a liposome or other microparticulate). Such liquids may additionally contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (for example, half-life, clearance rate, and the like).

In some embodiments, pharmaceutical compositions of the disclosure may be formulated in a single dose unit or in a form comprising a plurality of dosage units. Methods of preparing such dosage forms are known, or will be apparent, to those skilled in this art (see, for example, Remington: The Science and Practice of Pharmacy, 20th Edition (Philadelphia College of Pharmacy and Science, 2000)).

In some embodiments, a pharmaceutical composition comprising an anti-MSLN conjugate, as described herein, may be a lyophilisate.

In some embodiments, a syringe comprising a therapeutically effective amount of an anti-MSLN conjugate, or a pharmaceutical composition described herein, is provided.

### IV. Therapeutic Uses of the Conjugates Comprising

In some embodiments, the anti-MSLN conjugates as described herein may be used in a method(s) comprising administering an anti-MSLN conjugate as described herein to a subject in need thereof.

In some embodiments, the method may include an anti-MSLN conjugate, as described herein, that includes a heavy chain variable (VH) region and a light chain variable (VL) region, wherein the VH region comprises a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region comprises a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6.

In some embodiments, the method may include an anti-MSLN conjugate, that includes (i) a heavy chain variable region having the amino acid sequence set forth in SEQ ID NO:7, and (ii) a light chain variable region having the amino acid sequence set forth in SEQ ID NO:8. In some embodiments, the anti-MSLN conjugate, comprises: (i) a heavy chain variable region having the amino acid sequence set forth in SEQ ID NO:7 and (ii) a light chain variable region having the amino acid sequence set forth in SEQ ID NO:8, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, the methods may include an anti-MSLN conjugate, that includes: (i) a heavy chain variable region having the amino acid sequence set forth in SEQ ID NO:7, and (ii) a light chain variable region having the amino acid sequence set forth in SEQ ID NO:8, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

An anti-MSLN conjugate may include an antibody or antigen binding portion of any of these embodiments. In some embodiments, the anti-MSLN conjugate may include an antibody or antigen binding portion thereof, wherein the antibody is Ab-1 (also referred to as W305044-1.100.1-p3-uIgG1KV320 and W305044 mAb).

The anti-MSLN conjugate, as disclosed herein, may be used in therapy of mesothelin-related diseases. For example, the anti-MSLN conjugate, can be used to elicit in vivo or in vitro one or more of the following biological activities: to inhibit the growth of and/or kill a cell (for example, tumor cells) expressing mesothelin; to mediate phagocytosis of a cell expressing mesothelin in the presence of human effector cells, or to block mesothelin ligand binding to mesothelin.

In some embodiments, anti-MSLN conjugate, as disclosed herein, may be used in vivo to treat, prevent or diagnose a variety of mesothelin-related diseases, such as a mesothelin-expressing cancer. Examples of mesothelin-related diseases include, among others, ovarian, pancreatic, stomach, lung, uterine, endometrial, bile duct, gastric/esophageal, colorectal, breast cancers, sarcoma, hematologic cancers including leukemia and lymphoma, such as chronic lymphocytic leukemia, acute myeloid leukemia, acute lymphoblastic leukemia, diffuse large B cell lymphoma, follicular lymphoma, Hodgkin lymphoma and Non-Hodgkin's lymphoma, and myeloma.

In view of the association of mesothelin with certain tumors, embodiments of the invention provide a method of inhibiting the growth of a mesothelin-expressing tumor cell, comprising contacting the tumor cell with an anti-MSLN conjugate, as disclosed herein, such that growth of the tumor cells is inhibited. In some embodiments, the mesothelin-expressing tumor cell is a mesothelioma cell, or a tumor cell associated with ovarian, pancreatic, stomach, lung, uterine, endometrial, bile duct, gastric/esophageal, colorectal, and breast cancers. In some other embodiments, the mesothelin-expressing tumor cell is a mesothelioma cell, a pancreatic tumor cell, an ovarian tumor cell, a stomach tumor cell, a lung tumor cell or an endometrial tumor cell. In some embodiments, the tumor cell is from a cancer selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas, cholangiocarcinoma, lung adenocarcinoma, triple-negative breast cancer, and resectable pancreatic adenocarcinoma. In some other embodiments, the tumor cell is from a hematologic cancer including leukemia and lymphoma, such as chronic lymphocytic leukemia, acute myeloid leukemia, acute lymphoblastic leukemia, diffuse large B cell lymphoma, follicular lymphoma, Hodgkin lymphoma and Non-Hodgkin's lymphoma, and myeloma.

In some embodiments, a subject may be in need of treatment for a cancer and/or a malignancy. In some embodiments, the subject may be in need of treatment for a MSLN+ cancer or a MSLN+ malignancy, such as for example, mesothelioma, lung adenocarcinoma, gastric cancer, triple negative breast cancer, pancreatic cancer, ovarian carcinoma, ovarian adenocarcinoma, extrahepatic bile duct cancer, uterine serous carcinoma, endometrial adenocarcinoma, colon carcinoma, soft tissue sarcomas, head and neck cancers, or cholangiocarcinoma. In some embodiments, the method may be for treating a subject having a MSLN+ cancer or malignancy. In some embodiments, the method may be for treating mesothelioma in a subject. In some embodiments, the method may be for treating lung adenocarcinoma in a subject. In some embodiments, the method may be for treating gastric cancer in a subject. In some embodiments, the method may be for treating triple negative breast cancer in a subject. In some embodiments, the method may be for treating pancreatic cancer in a subject. In some embodiments, the method may be for treating ovarian carcinoma in a subject. In some embodiments, the method may be for treating ovarian adenocarcinoma in a subject. In some embodiments, the method may be for treating extrahepatic bile duct cancer in a subject. In some embodiments, the method may be for treating uterine serous cancer in a subject. In some embodiments, the method may be for treating endometrial adenocarcinoma in a subject. In some embodiments, the method may be for treating colon carcinoma in a subject. In some embodiments, the method may be for treating soft tissue sarcomas in a subject. In some embodiments, the method may be for treating head and neck cancers in a subject. In some embodiments, the method may be for treating cholangiocarcinoma in a subject.

In some aspects, the present disclosure provides a method of treating a disorder or a disease in a mammal, which comprises administering to the subject (for example, a human) in need of treatment a therapeutically effective amount of the anti-MSLN conjugate, as disclosed herein, The disorder or disease may be a cancer.

MSLN is implicated in a variety of cancers, whether malignant or benign and whether primary or secondary, which may be treated or prevented with a method provided by the disclosure. The cancers may be solid cancers or hematologic malignancies. Examples of such cancers include lung cancers such as bronchogenic carcinoma (for example, non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, chondromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (for example, glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (for example, seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; hematological cancers such as acute lymphocytic (lymphoblastic) leukemia, acute myeloid (myelocytic, myelogenous, myeloblasts, myelomonocytic) leukemia, chronic lymphocytic leukemia (for example, Sezary syndrome and hairy cell leukemia), chronic myelocytic (myeloid, myelogenous, granulocytic) leukemia, Hodgkin's lymphoma, non-Hodgkin's lymphoma, B cell lymphoma, including low grade/follicular non-Hodgkin's lymphoma (NHL), small lymphocytic (SL) NHL, intermediate grade/follicular NHL, intermediate grade diffuse NHL, high grade immunoblastic NHL; high grade lymphoblastic NHL, high grade small non-cleaved cell NHL, bulky disease NHL, mantle cell lymphoma, AIDS-related lymphoma; and myeloproliferative disorders (including polycythemia vera, myelofibrosis, thrombocythemia, and chronic myelocytic leukemia).

In some embodiments, the anti-MSLN conjugate, as disclosed herein, can be used for the treatment of mesotheliomas, ovarian cancers, pancreatic cancers, stomach cancers, lung cancers or endometrial cancers. In some embodiments, the anti-MSLN conjugate, as disclosed herein, are used in the treatment of a cancer selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas. In some specific embodiments, the antibodies are used in the treatment of a cancer selected from leukemia and lymphoma, such as chronic lymphocytic leukemia, acute myeloid leukemia, acute lymphoblastic leukemia, diffuse large B cell lymphoma, follicular lymphoma, Hodgkin lymphoma and Non-Hodgkin's lymphoma, and myeloma.

The methods described herein include administering a therapeutically effective amount of an anti-MSLN conjugate to a subject having a MSLN+ cancer or malignancy. As used herein, the phrase “therapeutically effective amount”, “effective amount” or “effective dose” may refer to an amount of the anti-MSLN conjugate, as described herein, that provides a therapeutic benefit in the treatment of, management of or prevention of relapse of a cancer or malignancy, for example, an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, for example, that is it has become invasive, seeding tumor growth in tissues remote from the original tumor site. A “tumor” refers to an uncontrolled growth of cells which interferes with the normal functioning of the bodily organs and systems. A subject that has a cancer may be a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell carcinoma, biliary tract cancer, bladder cancer, bone cancer, brain and CNS cancer, breast cancer (for example, triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic carcinoma, hepatoma, intra-epithelial neoplasm, kidney or renal cancer (for example, clear cell cancer), larynx cancer, leukemia, liver cancer, lung cancer (for example, small-cell lung cancer, non-small cell lung cancer, adenocarcinoma of the lung, and squamous carcinoma of the lung), lymphoma including Hodgkin's and non-Hodgkin's lymphoma, melanoma, mesothelioma, myeloma, neuroblastoma, oral cavity cancer (for example, lip, tongue, mouth, and pharynx), ovarian cancer, pancreatic cancer, prostate cancer, retinoblastoma, rhabdomyosarcoma, cancer of the respiratory system, salivary gland carcinoma, sarcoma, skin cancer, squamous cell cancer, testicular cancer, thyroid cancer, uterine or endometrial cancer, uterine serious carcinoma, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the carcinoma is selected from a solid tumor, including but not limited to, mesothelioma, lung adenocarcinoma, gastric cancer, triple negative breast cancer, pancreatic cancer, ovarian adenocarcinoma, uterine serous carcinoma, acute myeloid leukemia, colorectal cancer, esophageal cancer, endometrial cancer, head and neck cancer, sarcomas, and cholangiocarcinoma.

In some embodiments, the cancer or malignancy may be MSLN-positive (MSLN+). As used herein, the terms “MSLN-positive” or “MSLN+” are used to describe a cancer cell, a cluster of cancer cells, a tumor mass, or a metastatic cell that express MSLN on the cell surface (for example, membrane-bound MSLN). Some non-limiting examples of MSLN-positive cancers include mesothelioma, lung adenocarcinoma, gastric cancer, triple negative breast cancer, pancreatic cancer, ovarian adenocarcinoma, uterine serous carcinoma, acute myeloid leukemia, colorectal cancer, esophageal cancer, endometrial cancer, head and neck cancer, sarcomas, and cholangiocarcinoma.

It is contemplated that the methods herein may reduce tumor size or tumor burden in the subject, and/or reduce metastasis in the subject. In various embodiments, tumor size in the subject may be decreased by about 25-50%, about 40-70% or about 50-90% or more. In various embodiments, the methods may reduce the tumor size by about 10%, about 20%, about 30% or more. In various embodiments, the methods may reduce the tumor size by about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In various embodiments, the methods may reduce the tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” may refer to a human or animal. Usually the animal is a vertebrate such as a primate, rodent, domestic animal or game animal. Primates include chimpanzees, cynomolgus monkeys, spider monkeys, and macaques, for example, Rhesus. Rodents include mice, rats, woodchucks, ferrets, rabbits and hamsters. Domestic and game animals include cows, horses, pigs, deer, bison, buffalo, feline species, for example, domestic cat, canine species, for example, dog, fox, wolf, avian species, for example, chicken, emu, ostrich, and fish, for example, trout, catfish and salmon. In some embodiments, the subject is a mammal, for example, a primate, for example, a human. The terms, “patient”, “individual” and “subject” are used interchangeably herein.

In some embodiments, the subject is a mammal. The mammal can be a human, non-human primate, mouse, rat, dog, cat, horse, or cow, but are not limited to these examples. Mammals other than humans may be advantageously used, for example, as subjects that represent animal models of, for example, various cancers. In addition, the methods described herein can be used to treat domesticated animals and/or pets. A subject can be male or female. In some embodiments, the subject is a human.

A subject may have been previously diagnosed with, or identified as suffering from, a MSLN+ cancer. In some embodiments, the subject may have not already undergone treatment for the MSLN+ cancer. In some embodiments, a subject may have not been previously diagnosed as having a MSLN+ cancer. A subject may exhibit one or more risk factors for a condition or one or more complications related to a MSLN+ cancer. In some embodiments, a subject may not exhibit risk factors. A “subject in need” of treatment for a MSLN+ cancer may be a subject having that condition or diagnosed as having that condition. In other embodiments, a subject “at risk of developing” a condition may refer to a subject diagnosed as being at risk for developing the condition (for example, a MSLN+ cancer).

The term “prevent,” “prevention” or “preventing,” as used herein, with reference to a certain disease condition in a mammal, refers to preventing or delaying the onset of the disease, or preventing the manifestation of clinical or subclinical symptoms thereof.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. In some embodiments, treatment is “effective” if the progression of a condition is reduced or halted. That is, “treatment” includes not just the improvement of symptoms or markers, but also may include a cessation or at least slowing of progress or worsening of symptoms that would be expected in the absence of treatment. For cancer, “treating” may refer to dampen or slow the tumor or malignant cell growth, proliferation, or metastasis, or some combination thereof. Beneficial or desired clinical results may include, but are not limited to, reduction in MSLN+ cancer cells in the subject, alleviation of one or more symptom(s), diminishment of extent of the deficit, stabilized (for example, not worsening) state of a cancer or malignancy, delay or slowing of tumor growth and/or metastasis, and an increased lifespan as compared to that expected in the absence of treatment. As used herein, the term “administering,” refers to providing a conjugate, as described herein, into a subject by a method or route which results in binding of the conjugate to MSLN+ cancer cells or malignant cells. Similarly, a pharmaceutical composition comprising a conjugate as described herein can be administered by any appropriate route which results in an effective treatment in the subject.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of a disease or condition, refers to a conjugate in an amount or concentration effective to treat the said disease or condition.

The dosage ranges for an anti-MSLN conjugate, as described herein, depend upon the potency, and encompass amounts large enough to produce the desired effect, for example, slowing of tumor growth or a reduction in tumor size. The dosage should not be so large as to cause unacceptable adverse side effects. Generally, the dosage will vary with the age, condition, and sex of the subject and may be determined by one of skill in the art. The dosage may also be adjusted by an individual physician in the event of any complication. In some embodiments, the dosage may range from about 0.1 mg/kg body weight to about 12 mg/kg body weight. In some embodiments, the dosage may range from 0.1 mg/kg body weight to 10 mg/kg body weight. In some embodiments, the dosage may range from about 0.5 mg/kg body weight to about 15 mg/kg body weight. In some embodiments, the dosage may range from 0.5 mg/kg body weight to 15 mg/kg body weight. In some embodiments, the dose may range from 0.5 mg/kg body weight to 5 mg/kg body weight. Alternatively, the dose range may be titrated to maintain serum levels between about 1 μg/mL and about 1000 μg/mL. For systemic administration, subjects may be administered a therapeutic amount, such as, for example, 0.1 mg/kg, 0.5 mg/kg, 1.0 mg/kg, 2.0 mg/kg, 2.5 mg/kg, 5 mg/kg, 10 mg/kg, 12 mg/kg body weight or more. In some embodiments, the conjugate may be administered in a dose of about 0.1 mg/kg body weight to about 12 mg/kg body weight.

Compatible formulations for parenteral administration (for example, intravenous injection) may include the anti-MSLN conjugate, as described herein in concentrations of from about g/ml to about 100 mg/ml, such as 20 g/ml, 40 g/ml, 60 g/ml, 80 g/ml, 100 g/ml, 200 g/ml, 300, g/ml, 400 g/ml, 500 g/ml, 600 g/ml, 700 g/ml, 800 g/ml, 900 g/ml, 1 mg/ml, 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml. It will be apparent to one of skill in the art that the dosage of the anti-MSLN conjugate, as described herein, may vary depending on the individual, the type of neoplastic condition, the stage of neoplastic condition, whether the neoplastic condition has begun to metastasize to other location in the individual, the past and concurrent treatments being used, and the dosage of therapeutic agents used in combination with the conjugate as disclosed herein.

Administration of the doses recited above may be repeated. In some embodiments, the doses recited above may be administered weekly, biweekly, every three weeks, every four weeks, or monthly for several weeks or months. The duration of treatment may depend upon the subject's clinical progress and responsiveness to treatment.

In some embodiments, the course of treatment involving the anti-MSLN conjugate, as described herein, will include multiple doses of the selected drug product over a period of weeks or months. In some embodiments, the anti-MSLN conjugate, as described herein, may be administered once every day, every two days, every four days, every week, every ten days, every two weeks, every three weeks, every four weeks, every month, every six weeks, every two months, every ten weeks or every three months. In this regard, it will be appreciated that the dosages may be altered or the interval may be adjusted based on patient response and clinical practices.

Frequency of administration may be determined and adjusted over the course of therapy and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate. To assess efficacy of the selected composition, a marker of the specific disease, disorder or condition can be followed. For cancer, these may include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (for example, PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In some embodiments, an anti-MSLN conjugate, as described herein, may be administered in various ranges. These include about 5 g/kg body weight to about 40 mg/kg body weight per dose; about 50 g/kg body weight to about 5 mg/kg body weight per dose; about 100 g/kg body weight to about 10 mg/kg body weight per dose. Other ranges include about 100 g/kg body weight to about 20 mg/kg body weight per dose and about 0.5 mg/kg body weight to about 20 mg/kg body weight per dose. In certain embodiments, the dosage is at least about 100 g/kg body weight, at least about 250 g/kg body weight, at least about 750 g/kg body weight, at least about 3 mg/kg body weight, at least about 5 mg/kg body weight, at least about 10 mg/kg body weight.

In some embodiments, a dose may be from about 0.1 mg/kg body weight to about 100 mg/kg body weight. In some embodiments, a dose may be from about 0.1 mg/kg body weight to about 25 mg/kg body weight. In some embodiments, a dose may be from about 0.1 mg/kg body weight to about 20 mg/kg body weight. In some embodiments, a dose may be from about 0.1 mg/kg body weight to about 15 mg/kg body weight. In some embodiments, a dose may be from about 0.1 mg/kg body weight to about 12 mg/kg body weight. In some embodiments, a dose may be from about 1 mg/kg body weight to about 100 mg/kg body weight. In some embodiments, a dose may be from about 1 mg/kg body weight to about 25 mg/kg body weight. In some embodiments, a dose may be from about 1 mg/kg body weight to about 20 mg/kg body weight. In some embodiments, a dose may be from about 1 mg/kg body weight to about 15 mg/kg body weight. In some embodiments, a dose may be from about 1 mg/kg body weight to about 12 mg/kg body weight. In some embodiments, a dose may be about 0.1 mg/kg body weight. In some embodiments, a dose may be about 0.5 mg/kg body weight. In some embodiments, a dose may be about 1 mg/kg body weight. In some embodiments, a dose may be about 2 mg/kg body weight. In some embodiments, a dose may be about 4 mg/kg body weight. In some embodiments, a dose may be about 5 mg/kg body weight. In some embodiments, a dose may be about 6 mg/kg body weight. In some embodiments, a dose may be about 8 mg/kg body weight. In some embodiments, a dose may be about 10 mg/kg body weight. In some embodiments, a dose may be about 12 mg/kg body weight.

In some embodiments, a dose may be from about 100 mg/m² body surface area to about 700 mg/m² body surface area. In some embodiments, a dose may be about 250 mg/m² body surface area. In some embodiments, a dose may be about 375 mg/m² body surface area. In some embodiments, a dose may be about 400 mg/m² body surface area. In some embodiments, the dose may be about 500 mg/m² body surface area.

In some embodiments, a dose may be administered intravenously. In some embodiments, a conjugate is administered intravenously. In some embodiments, a conjugate is to be administered intravenously. In some embodiments, an intravenous administration may be an infusion occurring over a period of from about 10 minutes to about 4 hours. In some embodiments, an intravenous administration may be an infusion occurring over a period of from about 30 minutes to about 90 minutes.

In some embodiments, a dose may be administered weekly. In some embodiments, a dose can be administered bi-weekly. In some embodiments, a dose may be administered daily. In some embodiments, a dose may be administered about every 2 weeks. In some embodiments, a dose can be administered about every 3 weeks. In some embodiments, a dose may be administered every three weeks. In some embodiments, a dose may be administered every four weeks. In some embodiments, a dose may be administered monthly. In some embodiments, a dose may be administered every two months. Determination of the frequency of administration may be made by persons skilled in the art, such as an attending physician based on considerations of the condition being treated, age of the subject being treated, severity of the condition being treated, general state of health of the subject being treated and the like.

In some embodiments, a total of from about 2 to about 10 doses are administered to a subject. In some embodiments, a single dose is administered. In some embodiments, a total of 2 doses are administered. In some embodiments, a total of 3 doses are administered. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing an anti-MSLN conjugate, may be administered in a unit dose. The term “unit dose” when used in reference to a pharmaceutical composition refers to physically discrete units suitable as unitary dosage for the subject, each unit containing a predetermined quantity of active material (for example, an anti-MSLN conjugate), calculated to produce the desired therapeutic effect in association with the required physiologically acceptable diluent, for example, carrier, or vehicle.

In some aspects, the disclosure also provides a method of enhancing (for example, stimulating) an immune response in a subject comprising administering an anti-MSLN conjugate of the disclosure to the subject such that an immune response in the subject is enhanced. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (for example, cell-mediated, such as cytotoxic T lymphocyte mediated) or a humoral response (for example, antibody mediated response), and may be a primary or secondary immune response. The enhancement of immune response can be assessed using a number of in vitro or in vivo measurements known to those skilled in the art, including, but not limited to, cytotoxic T lymphocyte assays, release of cytokines (for example IL-2 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the anti-MSLN conjugate, may be used to enhance the immune response of a human to a vaccine.

The anti-MSLN conjugate, may be used alone as a monotherapy, or may be used in combination with chemical therapies, radiotherapies and immune cell therapies.

In some embodiments, a method of treating a MSLN+ cancer is provided, including administering to a subject in need thereof a therapeutically effective amount of an anti-MSLN conjugate or a pharmaceutical composition thereof.

In some embodiments, the use of an anti-MSLN conjugate, or a pharmaceutical composition thereof, for the treatment of MSLN+ cancer in a subject is contemplated.

In some embodiments, an anti-MSLN conjugate, or a pharmaceutical composition thereof, may be used in the manufacture of a medicament for the treatment of MSLN+ cancer in a subject.

In some embodiments, an anti-MSLN conjugate or a pharmaceutical composition thereof, is for use in the treatment of MSLN+ cancer in a subject.

In some embodiments, the MSLN+ cancer is a carcinoma or a malignancy.

In some embodiments, the MSLN+ cancer is mesothelioma, lung adenocarcinoma, gastric cancer, triple negative breast cancer, pancreatic cancer, ovarian adenocarcinoma, uterine serous carcinoma, acute myeloid leukemia, colorectal cancer, esophageal cancer, endometrial cancer, head and neck cancer, sarcomas, or cholangiocarcinoma.

Combined Use with Chemotherapies

The anti-MSLN conjugate may be used in combination with (prior to, simultaneously with or following administration of the anti-MSLN conjugate as disclosed herein) an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents.

Examples of cytotoxic agents that may be used in a combination therapy include, but are not limited to, small molecule toxins or enzymatically active toxins of bacteria (for example, Diptheria toxin, Pseudomonas endotoxin and exotoxin, Staphylococcal enterotoxin A), fungal (for example, α-sarcin, restrictocin), plants (for example, abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (for example, cytotoxic RNases, such as extracellular pancreatic RNases; DNase I, including fragments and/or variants thereof).

In some embodiments, a “chemotherapeutic agent” may include a chemical compound that non-specifically decreases or inhibits the growth, proliferation, and/or survival of cancer cells (for example, cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (for example, TIC). Such agents are often administered, and are often most effective, in combination, for example, in regimens such as CHOP or FOLFIRI.

Examples of anti-cancer agents that may be used in combination with the anti-MSLN conjugate of the present disclosure include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a MSLN expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies, Immunotherapies, and/or Chemotherapies

The present disclosure also provides for the combination of the anti-MSLN conjugate with radiotherapy (for example, any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed anti-MSLN conjugate may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

In some embodiments, the anti-MSLN conjugate, or a pharmaceutical composition of any of these, is administered with an immunotherapy. As used herein, “immunotherapy” refers to therapeutic strategies designed to induce or augment the subject's own immune system to fight the cancer or malignancy. Examples of an immunotherapy include, but are not limited to, antibodies such as check point inhibitors.

In some embodiments, a method, use, conjugate for use, or pharmaceutical composition for use, as described herein, includes administering an immunotherapy to a subject, or the subject is to be administered an immunotherapy.

In some embodiments, a method, use, conjugate for use, or pharmaceutical composition for use, as described herein, includes administering a chemotherapy to a subject, or the subject is to be administered a chemotherapy.

In some embodiments, a subject is receiving, or has received, immunotherapy or chemotherapy.

In some embodiments, the immunotherapy includes administration of an immune checkpoint inhibitor. In some embodiments, the immune checkpoint inhibitor may be CTLA-4, PD-1, PD-L1, PL-L2, B7-H3, B7-H4, BMA, HVEM, TIM3, GAL9, LAG3, VISTA, KIR, 2B4, CD160, CGEN-15049, CHK1, CHK2, and A2aR. In some embodiments, the immune checkpoint inhibitors include agents that inhibit CTLA-4, PD-1, PD-L1, and the like. In some embodiments, the immune checkpoint inhibitor is an antibody that specifically binds to human PD-1, human PD-L1, or human CTLA4, or any combination thereof.

Suitable anti-CTLA-4 therapy agents, include, for example, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, ipilimumab, tremelimumab, anti-CTLA-4 adnectins, anti-CTLA-4 domain antibodies, single chain anti-CTLA-4 mAbs, heavy chain anti-CTLA-4 mAbs, light chain anti-CTLA-4 mAbs, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present disclosure include, for example, those disclosed in: WO 98/42752; U.S. Pat. Nos. 6,682,736 and 6,207,156; Hurwitz et al., Proc. Natl. Acad. Sci. USA, 95(17): 10067-10071, 1998; Camacho et al., J. Clin. Oncology, 22(145): Abstract No. 2505, 2004 (antibody CP-675206); Mokyr et al., Cancer Res, 58:5301-5304, 1998, U.S. Pat. Nos. 5,977,318, 6,682,736, 7,109,003, and 7,132,281.

Suitable anti-PD-1 and anti-PD-L1 therapy agents, include, for example, anti-PD-1 and anti-PD-L1 antibodies, human anti-PD-1 and anti-PD-L1 antibodies, mouse anti-PD-1 and anti-PD-L1 antibodies, mammalian anti-PD-1 and anti-PD-L1 antibodies, humanized anti-PD-1 and anti-PD-L1 antibodies, monoclonal anti-PD-1 and anti-PD-L1 antibodies, polyclonal anti-PD-1 and anti-PD-L1 antibodies, chimeric anti-PD-1 and anti-PD-L1 antibodies, anti-PD-1 adnectins and anti-PD-L1 adnectins, anti-PD-1 domain antibodies and anti-PD-L1 domain antibodies, single chain anti-PD-1 mAbs and single chain anti-PD-L1 mAbs, heavy chain anti-PD-1 mAbs and heavy chain anti-PD-L1 mAbs, and light chain anti-PD-1 mAbs and light chain anti-PD-L1 mAbs. In specific embodiments, anti-PD-1 therapy agents include nivolumab, pembrolizumab, pidilizumab, MEDI0680, and combinations thereof. In other specific embodiments, anti-PD-L1 therapy agents include atezolizumab, avelumab, BMS-936559, durvalumab (MEDI4736), MSB0010718C, and combinations thereof.

Suitable anti-PD-1 and anti-PD-L1 antibodies are also described in Topalian, et al., Immune Checkpoint Blockade: A Common Denominator Approach to Cancer Therapy, Cancer Cell 27: 450-61, 2015, incorporated herein by reference in its entirety.

In some embodiments, the immune checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, the immunotherapy includes a checkpoint inhibitor. In some embodiments, the checkpoint inhibitor is an antibody that specifically binds to human PD-1, human PD-L1, or human CTLA4. In some embodiments, the checkpoint inhibitor is pembrolizumab, nivolumab, cemiplimab or ipilimumab.

In some embodiments, the chemotherapy may include administration of at least one anti-cancer drug, for example, a chemotherapeutic agent or an alkylating agent. In some embodiments, the anti-cancer drug may be a cytotoxic chemotherapeutic agent. In some embodiments, chemotherapy may refer to specific antineoplastic chemical agents or drugs that are “selectively” destructive to malignant cells and tissues, for example alkylating agents, antimetabolites including thymidylate synthase inhibitors, anthracyclines, anti-microtubule agents including plant alkaloids, topoisomerase inhibitors, PARP inhibitors and other antitumor agents. Examples of alkylating agents, which may be employed in the method of the present disclosure include an alkylating agent nitrogen mustards, nitrosoureas, tetrazines, aziridines, platins and derivatives, and non-classical alkylating agents.

In some embodiments, the platin may be cisplatin, carboplatin, oxaliplatin, satraplatin, picoplatin, nedaplatin, triplatin or lipoplatin (a liposomal version of cisplatin.

In some embodiments, the nitrogen mustards may include mechlorethamine, cyclophosphamide, melphalan, chlorambucil, ifosfamide and busulfan.

In some embodiments, nitrosoureas may include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

In some embodiments, aziridines may include thiotepa, mytomycin and diaziquone (AZQ).

Examples of antimetabolites include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil and prodrugs thereof such as capecitabine [Xeloda®]), floxuridine, gemcitabine, cytarabine, decitabine, raltitrexed (tomudex) hydrochloride, cladribine and 6-azauracil.

Examples of anthracyclines include daunorubicin (Daunomycin), daunorubicin (liposomal), doxorubicin (Adriamycin), doxorubicin (liposomal), epirubicin, idarubicin, and valrubicin.

Examples of anti-microtubule agents include vinca alkaloids and taxanes. Vinca alkaloids include completely natural chemicals for example vincristine and vinblastine and also semi-synthetic vinca alkaloids, for example vinorelbine, vindesine, and vinflunine. Taxanes include paclitaxel, docetaxel, abraxane, carbazitaxel and derivatives of thereof. Derivatives of taxanes may include reformulations of taxanes like taxol, for example in a micelluar formulations, derivatives may also include chemical derivatives wherein synthetic chemistry is employed to modify a starting material which is a taxane.

Topoisomerase inhibitors may include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193. Type II poisons may include amsacrine, etoposide, etoposide phosphate, teniposide and doxorubicin and fluoroquinolones.

In some embodiments, provided is a method of improving treatment outcome in a subject receiving immunotherapy and/or a chemotherapy. The method generally includes administering an effective amount of an immunotherapy or chemotherapy to the subject having cancer; and administering a therapeutically effective amount of an anti-MSLN conjugate, or a pharmaceutical composition thereof to the subject, wherein the binding agent or anti-MSLN conjugate specifically binds to MSLN+ cancer cells; wherein the treatment outcome of the subject is improved, as compared to administration of the immunotherapy or chemotherapy alone. In some embodiments, the anti-MSLN conjugate comprises (i) a heavy chain variable region having the amino acid sequence set forth in SEQ ID NO:7, and (ii) a light chain variable region having the amino acid sequence set forth in SEQ ID NO:8, wherein the heavy and light chain framework regions are optionally modified with from 1 to 8 amino acid substitutions, deletions or insertions in the framework regions. In some embodiments, the anti-MSLN conjugate comprises (i) a heavy chain variable region having the amino acid sequence set forth in SEQ ID NO:7, and (ii) a light chain variable region having the amino acid sequence set forth in SEQ ID NO:8, wherein the anti-MSLN conjugate, specifically binds to MSLN+ cancer cells. In some embodiments, the binding agent is an antibody or an antigen-binding portion thereof. In some embodiments, the binding agent is a monoclonal antibody, a Fab, a Fab′, an F(ab′), an Fv, a disulfide linked Fv, a scFv, a scFab, a single domain antibody, a diabody, a bi-specific antibody, or a multi-specific antibody. In some embodiments, the binding agent or anti-MSLN conjugate is a conjugate of an anti-MSLN monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, the improved treatment outcome is an objective response selected from stable disease, a partial response or a complete response as determined by standard medical criteria for the cancer being treated. In some embodiments, the improved treatment outcome is reduced tumor burden. In some embodiments, the improved treatment outcome is progression-free survival or disease-free survival.

### Pharmaceutical Packs and Kits

Pharmaceutical packs and kits comprising one or more containers, comprising one or more doses of the anti-MSLN conjugate are also provided. In certain embodiments, a unit dosage is provided wherein the unit dosage contains a predetermined amount of a composition comprising, for example, the anti-MSLN conjugate, with or without one or more additional agents. For other embodiments, such a unit dosage is supplied in single-use prefilled syringe for injection. In still other embodiments, the composition contained in the unit dosage may include saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In some embodiments, the composition includes one or more substances that inhibit protein aggregation, including, but not limited to, sucrose and arginine. Any label on, or associated with, the container(s) may indicate that the enclosed composition is used for treating the neoplastic disease condition of choice.

The present disclosure also provides kits for producing single-dose or multi-dose administration units of the anti-MSLN conjugate and, optionally, one or more anti-cancer agents. The kit may include a container and a label or package insert on or associated with the container. Suitable containers may include, for example, bottles, vials, syringes, and the like. The containers may be formed from a variety of materials such as glass or plastic and contain a pharmaceutically effective amount of the disclosed anti-MSLN conjugate. In other embodiments, the container(s) may include a sterile access port (for example, the container may be an intravenous solution bag or a vial having a stopper pierceable by a hypodermic injection needle). Such kits will generally contain in a suitable container a pharmaceutically acceptable formulation of the anti-MSLN conjugate, and, optionally, one or more anti-cancer agents in the same or different containers. The kits may also contain other pharmaceutically acceptable formulations, either for diagnosis or combined therapy. For example, in addition to the anti-MSLN conjugate of the disclosure, such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed anti-MSLN conjugate, with or without additional components, or they may have distinct containers for each desired agent. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the anti-MSLN conjugate, and any optional components to a patient, for example, one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### V. Enumerated Embodiments

The present disclosure provides the following non-limiting enumerated Embodiments.

Embodiment 1. An anti-MSLN conjugate comprising: a binding agent comprising:
a heavy chain variable (VH) region and a light chain variable (VL) region, wherein the VH region comprises a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region comprises a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6;
at least one linker attached to the binding agent; and
at least one cytotoxic agent attached to the at least one linker.

Embodiment 2. The conjugate of Embodiment 1, wherein the VH comprises one, two, three, or four human framework regions and the VL comprises one, two, three, or four human framework regions, or wherein the VH comprises human framework regions 1, 2, and 4, and in framework region 3 comprises one or two substitutions mutations relative to a human framework region 3, and the VL comprises four human framework regions.

Embodiment 3: The conjugate of Embodiment 1 or 2, wherein framework region 3 comprises the mutation N70Q and/or S72A.

Embodiment 4: The conjugate any one of Embodiments 1-3, wherein the VH region comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:7 and/or the VL region comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:8.

Embodiment 5: The conjugate of any one of Embodiments 1-4, wherein the VH region comprises the amino acid sequence set forth in SEQ ID NO:7 and/or the VL region comprises the amino acid sequence set forth in SEQ ID NO:8.

Embodiment 6: The conjugate of Embodiment 5, wherein the VH region comprises the amino acid sequence set forth in SEQ ID NO:7 and the VL region comprises the amino acid sequence set forth in SEQ ID NO:8.

Embodiment 7: The conjugate of any one of Embodiments 1-6, wherein the binding agent is an antibody or an antigen-binding portion thereof.

Embodiment 8: The conjugate of Embodiment 7, wherein the antibody or an antigen-binding portion thereof is a monoclonal antibody, a Fab, a Fab′, an F(ab′), an Fv, a disulfide linked Fv, a scFv, a scFab, a single domain antibody, a diabody, a bi-specific antibody, or a multi-specific antibody.

Embodiment 9: The conjugate of Embodiment 8, wherein the bi-specific antibody or the multi-specific antibody is a Bispecific T cell Engager (BiTE); a DART; a Knobs-Into-Holes (KIH) assembly; a scFv-CH3-KIH assembly; a KIH Common Light-Chain antibody; a TandAb; a Triple Body; a TriBi Minibody; a Fab-scFv; a scFv-CH-CL-scFv; a F(ab′)2-scFv2; a tetravalent Hcab; an intrabody; a CrossMab; a Dual Action Fab (DAF) (two-in-one or four-in-one); a DutaMab; a DT-IgG, a charge paired antibody; a Fab-arm Exchange antibody, a SEEDbody; a Triomab; a LUZ-Y assembly, an Fcab; a κλ-body; an orthogonal Fabs antibody; a DVD-Ig; am IgG(H)-scFv; an scFv-(H)IgG; an IgG(L)-scFv; an scFv-(L)IgG; an IgG(L,H)-Fv; an IgG(H)-V; a V(H)-IgG; an IgG(L)-V; a V(L)-IgG; a KIH IgG-scFab; a 2scFv-IgG; a IgG-2scFv; a scFv4-Ig; a Zybody; a DVI-IgG (four-in-one), a FIT-Ig; a WuxiBody; or an In-Elbow-Insert Ig.

Embodiment 10: The conjugate of any one of the preceding Embodiments, wherein the heavy chain variable region is comprised in a heavy chain that further comprises a heavy chain constant region.

Embodiment 11: The conjugate of Embodiment 10, wherein heavy chain constant region is of the IgG isotype.

Embodiment 12: The conjugate of Embodiment 11, wherein the heavy chain constant region is an IgG1 constant region.

Embodiment 13: The conjugate of any one of Embodiments 10⁻¹², wherein the heavy chain constant region has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:9.

Embodiment 14: The conjugate of any one of Embodiments 10⁻¹³, wherein the heavy chain constant region has the amino acid sequence set forth in SEQ ID NO:9.

Embodiment 15: The conjugate of any one of Embodiments 10⁻¹⁴, wherein the heavy chain has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:11.

Embodiment 16: The conjugate of any one of Embodiments 10⁻¹⁵, wherein the heavy chain has the amino acid sequence set forth in SEQ ID NO: 11.

Embodiment 17: The conjugate of any one of the preceding Embodiments, wherein the light chain variable region is comprised in a light chain that further comprises a light chain constant region.

Embodiment 18: The conjugate of Embodiment 17, wherein the light chain constant region has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:10.

Embodiment 19: The conjugate of Embodiment 18, wherein the light chain constant region has the amino acid sequence set forth in SEQ ID NO:10.

Embodiment 20: The conjugate of any one of Embodiments 17-19, wherein the light chain has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:12.

Embodiment 21: The conjugate of any one of Embodiments 17-20, wherein the light chain has the amino acid sequence set forth in SEQ ID NO:12.

Embodiment 22: The conjugate of any one of the preceding Embodiments, wherein the binding agent comprises the amino acid sequence set forth in SEQ ID NO:11 and the amino acid sequence set forth in SEQ ID NO:12.

Embodiment 23: The conjugate of any one of the preceding Embodiments, wherein the binding agent comprises two heavy chains and two light chains, wherein each heavy chain comprises or consists of SEQ ID NO:11 and each light chain comprises or consists of SEQ ID NO:12.

Embodiment 24: The conjugate of any one of the preceding Embodiments, wherein the linker is attached to the binding agent by an interchain disulfide residue, an engineered cysteine, a glycan or modified glycan, an N-terminal residue of the binding agent, a polyhistidine residue attached to the binding agent, or any combination thereof.

Embodiment 25: The conjugate of any one of the preceding Embodiments, wherein the binding agent is mono-specific.

Embodiment 26: The conjugate of any one of the preceding Embodiments, wherein the binding agent is bivalent.

Embodiment 27: The conjugate of any one of the preceding Embodiments, wherein the binding agent comprises a second binding domain and the binding agent is bi-specific.

Embodiment 28: The conjugate of any one of the preceding Embodiments, wherein the cytotoxic agent is an anti-mitotic agent or a topoisomerase I inhibitor.

Embodiment 29: The conjugate of any one of the preceding Embodiments, wherein the cytotoxic agent comprises or consists of an auristatin, a camptothecin, a duocarmycin, an anthracycline, a calicheamicin, an exatecan or any analogs thereof, or any combination thereof.

Embodiment 30: The conjugate of Embodiment 29, wherein the cytotoxic agent comprises or consists of the auristatin.

Embodiment 31: The conjugate of Embodiment 30, wherein the cytotoxic agent comprises or consists of MMAE.

Embodiment 32: The conjugate of Embodiment 29, wherein the cytotoxic agent comprises or consists of the camptothecin or camptothecin derivative.

Embodiment 33: The conjugate of Embodiment 32, wherein the cytotoxic agent comprises or consists of exatecan or exatecan derivative.

Embodiment 34: The conjugate of Embodiment 32, wherein the cytotoxic agent comprises or consists of SN-38.

Embodiment 35: The conjugate of Embodiment 32, wherein the conjugate comprises or consists of an anthracycline.

Embodiment 36: The conjugate of Embodiment 32, wherein the cytotoxic agent comprises or consists of DXd.

Embodiment 37: The conjugate of any of the preceding Embodiments, wherein the linker comprises or consists of mc-VC-PAB, CL2, CL2A, (Succinimid-3-yl-N)-(CH2)n2-C(═O)-Gly-Gly-Phe-Gly-NH-CH2-OCH2-(C═O)—, an enzyme-cleavable linker, or any combination thereof.

Embodiment 38: The conjugate of Embodiment 37, wherein the linker comprises or consists of mc-VC-PAB.

Embodiment 39: The conjugate of Embodiment 37 or 38, wherein the linker is attached to at least one molecule of MMAE.

Embodiment 40: The conjugate of any one of Embodiments 37-39, wherein the conjugate is ADC-4 (Ab-1-mc-VC-PAB-MMAE).

Embodiment 41: The conjugate of Embodiment 37, wherein the linker comprises or consists of CL2A.

Embodiment 42: The conjugate of Embodiment 37 or 41, wherein the linker is attached to at least one molecule of SN-38.

Embodiment 43: The conjugate of any one of Embodiments 37, 41 or 42, wherein the conjugate is ADC-2 (Ab-1-CL2A-SN38).

Embodiment 44: The conjugate of Embodiment 37, wherein the linker comprises or consists of CL2.

Embodiment 45: The conjugate of Embodiment 44, wherein the linker is attached to at least one molecule of SN-38.

Embodiment 46: The conjugate of Embodiment 37, wherein the linker comprises or consists of (Succinimid-3-yl-N)-(CH2)n2-C(═O)-Gly-Gly-Phe-Gly-NH—CH2—O—CH2-(C═O)-.

Embodiment 47: The conjugate of Embodiment 46, wherein the linker is attached to at least one molecule of DXd.

Embodiment 48: The conjugate of any one of Embodiments 37, 46 or 47, wherein the conjugate is ADC-1 (Ab-1-GGFG-DXd).

Embodiment 49: The conjugate of Embodiment 37, wherein the linker comprises or consists of the enzyme-cleavable linker.

Embodiment 50: The conjugate of Embodiment 49, wherein the linker is attached to at least one molecule of exatecan.

Embodiment 51: The conjugate of any one of Embodiments 37, 49 or 50, wherein the conjugate is ADC-3 (Ab-1-enzyme-cleavable linker-exatecan).

Embodiment 52: A pharmaceutical composition comprising the conjugate of any of the preceding Embodiments and a pharmaceutically acceptable carrier.

Embodiment 53: The pharmaceutical composition of Embodiment 52, wherein an average number of cytotoxic agents per binding agent is from about 1 to about 8, about 2, about 4, about 6, about 8, about 10, about 12, about 14, about 16, about 3 to about 5, about 6 to about 8 or about 8 to about 16.

Embodiment 54: A method of treating a MSLN+ cancer, comprising administering to a subject in need thereof a therapeutically effective amount of the conjugate of any one of Embodiments 1-51 or the pharmaceutical composition of Embodiment 52 or 53.

Embodiment 55: Use of the conjugate of any one of Embodiments 1-51 or the pharmaceutical composition of Embodiment 52 or 53 for the treatment of MSLN+ cancer in a subject.

Embodiment 56: Use of the conjugate of any one of Embodiments 1-51 or the pharmaceutical composition of Embodiment 52 or 53 in the manufacture of a medicament for the treatment of MSLN+ cancer in a subject

Embodiment 57: The conjugate of any one of Embodiments 1-51 or the pharmaceutical composition of Embodiment 52 or 53 for use in the treatment of MSLN+ cancer in a subject.

Embodiment 58: The method, use, conjugate for use, or pharmaceutical composition for use, of any one of Embodiments 54-57, wherein the MSLN+ cancer is a carcinoma or a malignancy.

Embodiment 59: The method, use, conjugate for use, or pharmaceutical composition for use, of any one of Embodiments 54-58, wherein the MSLN+ cancer is mesothelioma, lung adenocarcinoma, gastric cancer, triple negative breast cancer, pancreatic cancer, ovarian adenocarcinoma, uterine serous carcinoma, acute myeloid leukemia, colorectal cancer, esophageal cancer, endometrial cancer, head and neck cancer, sarcomas, or cholangiocarcinoma.

Embodiment 60: The method, use, conjugate for use, or pharmaceutical composition for use, of any one of Embodiments 54-59, further comprising administering an immunotherapy to the subject, or wherein the subject is to be administered an immunotherapy.

Embodiment 61: The method, use, conjugate for use, or pharmaceutical composition for use, of any one of Embodiments 54-60, further comprising administering chemotherapy to the subject, or wherein the subject is to be administered chemotherapy.

Embodiment 62: The method, use, conjugate for use, or pharmaceutical composition for use, of any one of Embodiments 54-61, wherein the subject is receiving, or has received, immunotherapy or chemotherapy.

Embodiment 63: The method, use, conjugate for use, or pharmaceutical composition for use, of any one of Embodiments 60-62, wherein the immunotherapy comprises a checkpoint inhibitor.

Embodiment 64: The method, use, conjugate for use, or pharmaceutical composition for use, of Embodiment 63, wherein the checkpoint inhibitor is an antibody that specifically binds to human PD-1, human PD-L1, or human CTLA4, or any combination thereof.

Embodiment 65: The method, use, conjugate for use, or pharmaceutical composition for use, of Embodiment 64, wherein the checkpoint inhibitor is pembrolizumab, nivolumab, cemiplimab or ipilimumab.

Embodiment 66: The method, use, conjugate for use, or pharmaceutical composition for use, of any one of Embodiments 54-65, wherein the conjugate is administered intravenously.

Embodiment 67: The method, use, conjugate for use, or pharmaceutical composition for use, of any one of Embodiments 54-66, wherein the conjugate is administered in a dose of about 0.1 mg/kg body weight to about 12 mg/kg body weight.

Embodiment 68: A method of improving treatment outcome in a subject receiving immunotherapy and/or chemotherapy for a MSLN+ cancer, comprising:
administering an effective amount of an immunotherapy or chemotherapy to the subject; and
administering a therapeutically effective amount of the conjugate of any one of Embodiments 1-51 or the pharmaceutical composition of Embodiment 52 or 53 to the subject;
wherein the treatment outcome of the subject is improved, as compared to receiving the immunotherapy or chemotherapy alone.

Embodiment 69: Use of the conjugate of any one of Embodiments 1-51 or the pharmaceutical composition of Embodiment 52 or 53 for improving treatment outcome in a subject receiving immunotherapy and/or chemotherapy for a MSLN+ cancer, wherein the subject is to be administered:
an effective amount of an immunotherapy or chemotherapy; and
a therapeutically effective amount of the conjugate of any one of Embodiments 1-51 or the pharmaceutical composition of Embodiment 52 or 53;
wherein the treatment outcome of the subject is improved, as compared to receiving the immunotherapy or chemotherapy alone.

Embodiment 70: Use of the conjugate of any one of Embodiments 1-51 or the pharmaceutical composition of Embodiment 52 or 53 in the manufacture of a medicament for improving treatment outcome in a subject receiving immunotherapy and/or chemotherapy for a MSLN+ cancer, wherein the subject is to be administered:
an effective amount of an immunotherapy or chemotherapy; and
a therapeutically effective amount of the conjugate of any one of Embodiments 1-51 or the pharmaceutical composition of Embodiment 52 or 53;
wherein the treatment outcome of the subject is improved, as compared to receiving the immunotherapy or chemotherapy alone.

Embodiment 71: The conjugate of any one of Embodiments 1-51 or the pharmaceutical composition of Embodiment 52 or 53 for use in improving treatment outcome in a subject receiving immunotherapy and/or chemotherapy for a MSLN+ cancer, wherein the subject is to be administered:
an effective amount of an immunotherapy or chemotherapy; and
a therapeutically effective amount of the conjugate of any one of Embodiments 1-51 or the pharmaceutical composition of Embodiment 52 or 53;
wherein the treatment outcome of the subject is improved, as compared to receiving the immunotherapy or chemotherapy alone.

Embodiment 72: The method, use, conjugate for use, or pharmaceutical composition for use, of any one of Embodiments 68-71, wherein the improved treatment outcome is an objective response selected from stable disease, a partial response or a complete response.

Embodiment 73: The method, use, conjugate for use, or pharmaceutical composition for use, of any one of Embodiments 68-71, wherein the improved treatment outcome is reduced tumor burden.

Embodiment 74: The method, use, conjugate for use, or pharmaceutical composition for use, of any one of Embodiments 68-71, wherein the improved treatment outcome is progression-free survival or disease-free survival.

Embodiment 75: The method, use, conjugate for use, or pharmaceutical composition for use, of any one of Embodiments 68-74, wherein the immunotherapy is an immune checkpoint inhibitor.

Embodiment 76: The method, use, conjugate for use, or pharmaceutical composition for use, of Embodiment 75, wherein the immune checkpoint inhibitor comprises an antibody that specifically binds to human PD-1, human PD-L1, or CTLA4.

Embodiment 77: The method, use, conjugate for use, or pharmaceutical composition for use, of Embodiment 76, wherein the immune checkpoint inhibitor is pembrolizumab, nivolumab, cemiplimab or ipilimumab.

Embodiment 78: The method, use, conjugate for use, or pharmaceutical composition for use, of any one of Embodiments 68-77, wherein the conjugate is administered intravenously.

Embodiment 79: The method, use, conjugate for use, or pharmaceutical composition for use, of any one of Embodiments 68-78, wherein the conjugate is administered in a dose of about 0.1 mg/kg body weight to about 10 mg/kg body weight.

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Antigens, Benchmark Antibodies and Cell Lines

### 1.1 Generation of Antigens

The amino acid sequences encoding the extracellular domain of human MSLN (UniProt: KB-Q13421-3, residues 296 to 580) and cynomolgus monkey MSLN (UniProt: A0A2K5TW94, residues 296 to 598) were first codon optimized for mammalian expression and then synthesized by GENEWIZ (Su Zhou, CHINA). The DNA segment was then sub-cloned into the pcDNA3.3 expression vector with 6×His or human Fc at the C-terminal and expressed.

TABLE 2
Abbreviations of the antigens

| Material code | Material Information |
|---|---|
| W3xx044-hPro1.ECD.hFc | Human extracellular domain MSLN protein (hFc Tag), |
| | UniProt: KB-Q13421-3, residues 296 to 580 |
| W3xx044-hPro1.ECD.AVI.His | Human extracellular domain MSLN protein (his Tag), |
| | UniProt: KB-Q13421-3, residues 296 to 580 |
| W3XX044-hPro1.V3.ECD.hFc | Human MSLN Region II & Region III (hFc Tag), |
| | UniProt: KB-Q13421-3, residues 391-598 |
| W3xx044-hPro1.V4.ECD.hFc | Human MSLN region III protein (hFc Tag), UniProt: KB- |
| | Q13421-3, residues 487 to 598 |
| W3XX044-cPro1.ECD.AVI.His | Cynomolgus monkey extracellular domain MSLN protein |
| | (his Tag), UniProt: A0A2K5TW94, residues 296 to 598 |

### 1.2 Preparation of Benchmark Antibodies (BMKs)

Two anti-MSLN antibodies were used as controls. The amino acid sequences encoding the variable domains of one anti-MSLN antibody (sequences disclosed in WO2018/209304; named as Harpoon mAb herein), and a second anti-MSLN antibody (sequences disclosed in WO2017/021356; named as Amgen mAb herein) were first codon optimized for mammalian expression and synthesized by GENEWIZ (Sn Zhou, CHINA). The DNA segments were then sub-cloned into pcDNA3.4 expression vectors with constant region of human IgG1. The information of the benchmark antibodies is further provided in Table 3.

TABLE 3
Reference antibody information

| Antibody | Information |
|---|---|
| Harpoon mAb | The parental of MSLN binding arm of |
| (VHH-hinge- | HPN-536 CD3 × MSLN bi-specific |
| Fc, IgG1) | antibody |
| | Variable region sequence: |
| | QVQLVESGGGVVQAGGSLTLSCAASGSTFSIRAMRW |
| | YRQAPGTERDLVAVIYGSSTYYADAVKGRFTISRDN |
| | SKNTLYLQMNSLRAEDTAVYYCNADTIGTARDYWGQ |
| | GTLVTVSS (SEQ ID NO: 26) |
| Amgen mAb | The parental of MSLN binding arm of |
| (IgG1) | Amgen MSLN-BiTE bi-specific antibody |
| | VH sequence: |
| | QVQLVESGGGVVQPGRSLRLSCAASGFTFSDHYMSW |
| | IRQTPGKGLEWVSYISSSGSTIYYADSVKGRFTISR |
| | DNAKNSLYLQMNSLRAEDTAVYYCARDLGPSFDYWG |
| | QGSLVTVSS (SEQ ID NO: 27) |
| | VL sequence: |
| | DIQMTQSPSSVSASVGDRVTITCRASQGISSWLAWY |
| | QQKPGKAPKLLIDAASRLQSGVPSRFSGSGSGTDFT |
| | LTISSLQPEDFATYYCQQANSFPRTFGQGTKVEIK |
| | (SEQ ID NO: 28) |
| W332- | Non-MSLN binding isotype control; |
| 1.80.12- | negative control antibody |
| xIgG1KV320 | |

### 1.3 Cell Pool/Line Generation

Engineered cell lines stably expressing human, cynomolgus monkey or mouse MSLNs were constructed. Briefly, the CHO-K1 cells at 70-90% confluents were transfected with human MSLN, mouse MSLN or cynomolgus monkey MSLN full length plasmid using lipofectamine 2000 reagent. according to manufacturer's protocol. The transfected cells were cultured in an incubator at 37° C., 5% CO₂. Twenty-four hours later, blasticidin or Hygromycin B was used to select the stable pool. Then, the positive pool cells were subcloned by limited dilution. Single clone was picked and tested by FACS using anti-MSLN antibodies.

### Example 2

### Generation of Human Anti-MSLN Antibodies

### 2.1 Generation of Hybridoma Antibodies

OMT rats (Open Monoclonal Technology Company) were immunized with human MSLN and measured for serum titer. When the serum titer was sufficiently high (>=1:24,300), the animal with the highest titer were given a final boost. B cells from lymph nodes or spleen were fused with Sp2/0 myeloma cells following general electro-fusion procedures. The fused cells were re-suspended in DMEM medium supplemented with 20% FBS and 1×HAT, transferred into 96-well plates, and the resulting hybridoma supernatants screened. Twelve mAbs were selected and tested for binding with extracellular region, region II & region III of MSLN, and by FACS with and without soluble MSLN. Finally, a lead clone that selectively bound to MSLN region III and whose binding was not affected by soluble MSLN was chosen and its CDR sequences are shown in Table 1 above (SEQ ID NOs:1-6). The binding results of the clone are shown in Tables 4-5.

TABLE 4
Direct ELISA with 3 Antigens (Rat IgG mAbs with 100 nM, 10 nM and 1 nM)

| | Binding Domain | | | | | | | | |
|---|---|---|---|---|---|---|---|---|---|
| | | | | Human MSLN | | | Human MSLN | | |
| | Human MSLN ECD (296-580) | | | Region II & Region III | | | Region III (AA 487- | | |
| | Protein | | | (AA 391-598) | | | 598) | | |
| | Ags | | | | | | | | |
| | W3XX044-hPro1.V2.ECD.hFc | | | W3XX044-hPro1.V3.ECD.hFc | | | W3XX044-hPro1.V4.ECD.hFc | | |
| Abs (nM) | 100 | 10 | 1 | 100 | 10 | 1 | 100 | 10 | 1 |
| Harpoon mAb | 2.85 | 2.77 | 2.72 | 2.81 | 2.74 | 2.71 | 2.88 | 2.80 | 2.76 |
| Amgen mAb | 2.81 | 2.79 | 2.79 | 2.70 | 2.83 | 2.72 | 2.84 | 2.82 | 2.82 |
| W305044- | 0.63 | 0.30 | 0.15 | 0.93 | 0.87 | 0.42 | 0.44 | 0.18 | 0.08 |
| 1.100.1 | | | | | | | | | |

TABLE 5
MFI of FACS Data pre-incubated with and without soluble
MSLN (mAbs with 100 nM, 10 nM, 1 nM and 0.1 nM)

| | Without soluble MSLN | | | | With 0.5 μg/mL soluble MSLN | | | |
|---|---|---|---|---|---|---|---|---|
| Abs (nM) | 100 | 10 | 1 | 0.1 | 100 | 10 | 1 | 0.1 |
| Harpoon mAb | 17700 | 16900 | 16800 | 3574 | 17900 | 18900 | 521 | 487 |
| Amgen mAb | 17700 | 17100 | 9833 | 1354 | 18600 | 19200 | 317 | 77 |
| W305044-1.100.1 | 8123 | 7643 | 2806 | 440 | 8585 | 7271 | 1837 | 232 |

### 2.2 FACS Binding Assay of IgG Converted Fully Human Antibodies

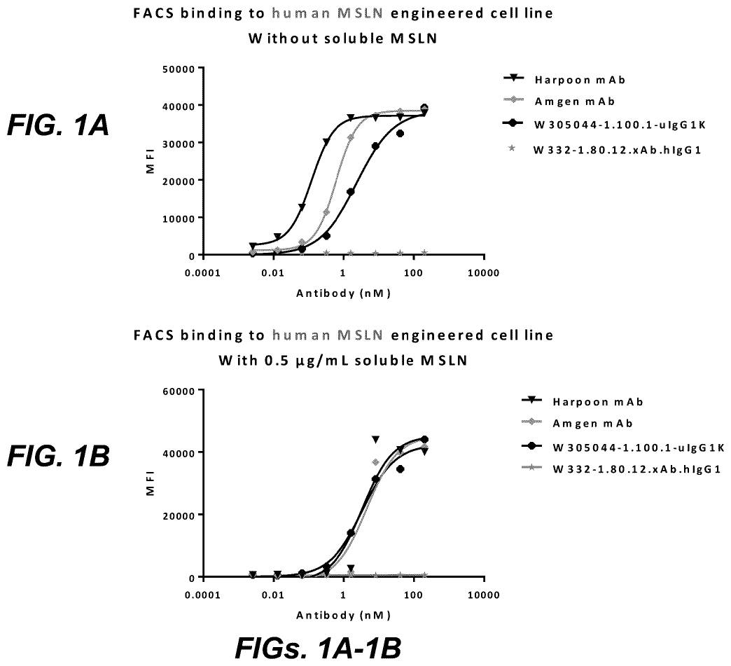

The rat mAbs were converted to human IgG1 to obtain fully human mAbs and their performance were tested. The human MSLN engineered cell line was used to test the impact of soluble MSLN on FACS binding of the mAbs. The results were shown in FIG. 1 and Table 6.

TABLE 6
The FACS binding EC50 and Max MFI of anti-MSLN mAbs to human
MSLN engineered cell with and without soluble MSLN

| | FACS | | | |
|---|---|---|---|---|
| | Without soluble | | With 0.5 μg/mL | |
| | MSLN | | soluble MSLN | |
| | EC50 | Max | EC50 | Max |
| Antibody | (nM) | MFI | (nM) | MFI |
| Harpoon mAb | 0.12 | 37800 | 3.36 | 43900 |
| Amgen mAb | 0.61 | 38800 | 4.53 | 41700 |
| W305044-1.100.1-uIgG1K | 2.36 | 39300 | 3.34 | 44000 |
| W332-1.80.12.xAb.hIgG1 | >100 | 516 | >100 | 619 |

### 2.3 Internalization Assay of IgG Converted Fully Human Antibodies

High content screening (HCS) internalization assay by Operetta was performed on the human MSLN engineered cell line to assess the internalization activity of the mAbs. Briefly, the a 96-well plate was coated with 2.5 g/cm² Poly-D-Lysine (PDL) at 37° C. for 2 hours (1:1000). Cells in the T75 flask were washed with PBS and detached by Versene. The cells were resuspended in an appropriate volume of culture medium to the concentration of 2×10⁵ cell/mL, aliquoted to each well of 96-well plate and incubated overnight. The next day, the medium was removed and serially diluted Abs in 1% BSA were added to the cell plates, and plates incubated at 4° C. for 2 hours. After incubation, the cells were washed and goat anti-human IgG PE (1:150 dilution in 1% BSA) added, and plates incubated for 1 hour at 4° C. in the dark. After washing the cells, 1% BSA was added to each well and the plates were incubated for 2 hours at 37° C. The cells were then quenched at 4° C. for 4 minutes. Then the plates were washed with PBS, Hoechst dye (1:2000 dilution in PBS) was added, and the cells incubated for 20 minutes at 25° C. The plates were washed and the cells were fixed with 4% PFA for 15 minutes at ambient temperature. The plates were read by Operetta Analysis System.

The internalization MFI of anti-MSLN mAbs by human MSLN engineered cells are shown in Table 7.

TABLE 7
The internalization activity of anti-MSLN
mAbs by human MSLN engineered cells

| | MFI | | | | |
|---|---|---|---|---|---|
| | 32 | 10 | 3.17 | 1 | 0.32 |
| Abs | nM | nM | nM | nM | nM |
| Harpoon mAb | 12308 | 10438 | 10403 | 9897 | 1789 |
| Amgen mAb | 10478 | 9033 | 9362 | 5491 | 1540 |
| W305044-1.100.1-uIgG1K | 10569 | 6911 | 3799 | 2407 | 260 |
| W332-1.80.12.xAb.hIgG1 | 0 | 0 | 0 | 1 | 0 |

### 2.4 ELISA Binding Assay of IgG Converted Fully Human Antibodies

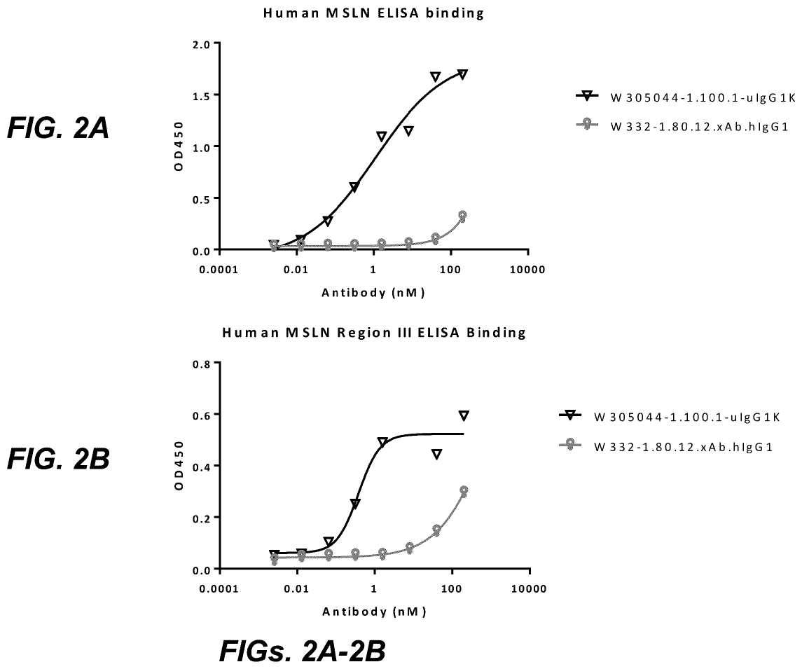

The ELISA binding of the IgG converted mAb was performed to determine the binding to region III of MSLN and ECD of MSLN. The results are shown in FIG. 2A-2B and Table 8. Compared to IgG converted mAbs of other clones, W305044-1.100.1-uIgG1K's binding to cell surface MSLN was minimally influenced by the presence of soluble MSLN protein (Table 5) and has the strongest binding to human MSLN Region III (Table 8, FIG. 2B).

TABLE 8
The ELISA binding EC50 and Max OD of the IgG converted
antibodies to human MSLN and MSLN region III proteins

| | Human MSLN | | Human MSLN | |
|---|---|---|---|---|
| | ECD | | Region III | |
| | EC50 | Max | EC50 | Max |
| Abs | (nM) | OD | (nM) | OD |
| W305044-1.100.1-uIgG1K | 1.12 | 1.694 | 0.38 | 0.593 |
| W332-1.80.12.xAb.hIgG1 | >200 | 0.311 | NA | 0.295 |

### 2.5 PTM Removal

For W305044-1.100.1-uIgG1K, there was one PTM site in the VH FRW3, so PTM removal was performed and the final lead selected. N70Q (FRW3) or S72A (FRW3) mutations were chosen for PTM removal. Two mAbs were constructed (W305044-1.100.1-p1-uIgG1V721 and W305044-1.100.1-p3-uIgG1V721). “IgG1V721” indicates the fused human IgG1 constant region has been engineered to comprise S298A/E333A/K334A/M252Y/S254T/T256E substitutions. The two mAbs were tested in the FACS with and without soluble MSLN and internalization assay to select the final lead.

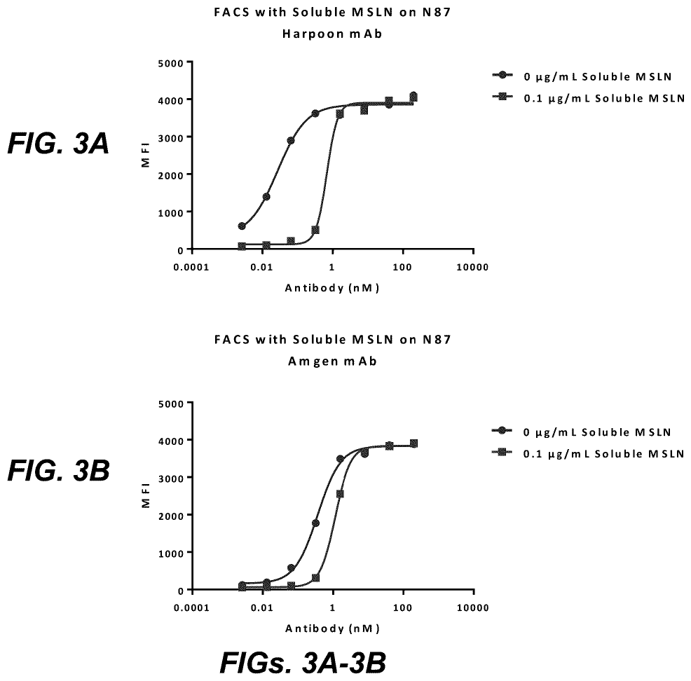

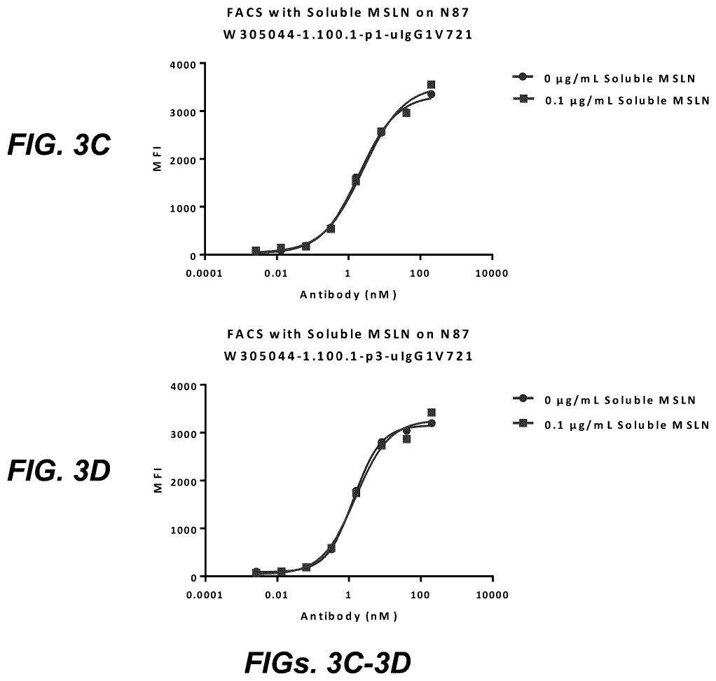

For FACS with and without soluble MSLN on NCI-N87 tumor cell (ATCC, CRL-5822TM), W305044-1.100.1-p1-uIgG1V721 and W305044-1.100.1-p3-uIgG1V721 showed comparable activity; both mAbs were minimally affected by soluble MSLN in contrast to the two reference mAbs (FIG. 3 and Table 9).

TABLE 9
The FACS binding EC50 and Max MFI of two PTM removal mAbs
to human MSLN engineered cell with and without soluble MSLN

| | Without soluble | | With 0.1 μg/mL | |
|---|---|---|---|---|
| | MSLN | | soluble MSLN | |
| | EC50 | Max | EC50 | Max |
| Abs | (nM) | MFI | (nM) | MFI |
| Harpoon mAb | 0.027 | 4102 | 0.68 | 4043 |
| Amgen mAb | 0.36 | 3881 | 1.16 | 3907 |
| W305044-1.100.1-p1- | 1.95 | 3353 | 2.55 | 3550 |
| uIgG1V721 | | | | |
| W305044-1.100.1-p3- | 1.35 | 3197 | 1.53 | 3425 |
| uIgG1V721 | | | | |
| Isotype control | NA | 118 | NA | 133 |

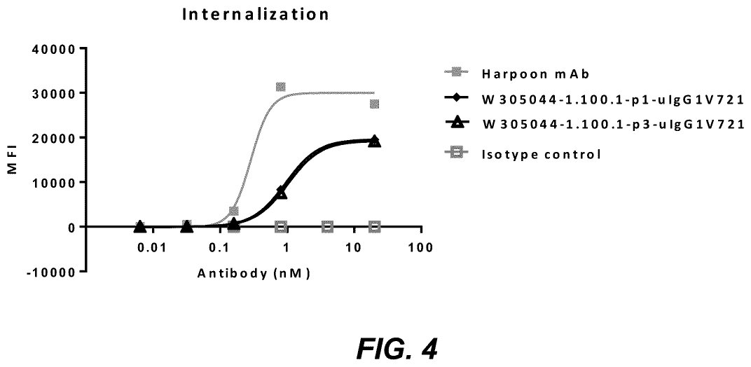

For the internalization assay, W305044-1.100.1-p1-uIgG1V721 and W305044-1.100.1-p3-uIgG1V721 showed comparable activity on human full-length MSLN expressing engineered cell (FIG. 4 and Table 10). Taking into consideration the developability profile, the minimal effect by soluble MSLN and the internalization activity, W305044-1.100.1-p3 was selected for further characterization.

TABLE 10
The internalization activity of PTM removal
mAbs to human MSLN engineered cell

Internalization

| Abs | EC50 (nM) | Max MFI |
|---|---|---|
| Harpoon mAb | 0.29 | 43225 |
| W305044-1.100.1-p1-uIgG1V721 | 0.95 | 32452 |
| W305044-1.100.1-p3-uIgG1V721 | 1.02 | 28615 |
| Isotype control | NA | 2 |

### Example 3

### In Vitro Characterization of W305044 Antibody (Ab-1)

### 3.1 SDS-PAGE

Nu PAGE Bis-Tris Mini Gels 4-12%, Nu PAGE MES SDS Running Buffer (20×), and the Simply Blue Safe Stain were used. The samples were mixed with the loading buffer, and heated at 75° C. for 10 minutes. Then the samples were loaded and PAGE was run at a constant voltage (200 V) for 35 minutes. The gel was rinsed and destained with water.

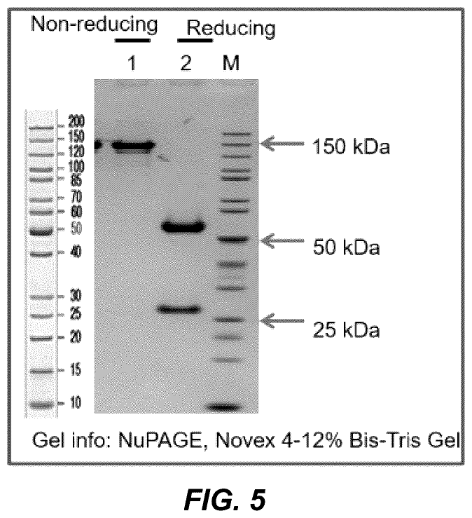

The lead mAb, W305044-1.100.1-p3-uIgG1KV320 (also known as Ab-1; abbreviated as “W305044” herein, “V320” indicates Leu234Ala/Leu235Ala substitutions in the human IgG1 constant region; sequences are set forth in Table 1) is shown in FIG. 5. Ab-1 has CDRs as set forth in SEQ ID NOs:1-6; a VH as set forth in SEQ ID NO:7; a VL as set forth in SEQ ID NO:8; a heavy chain constant region as set forth in SEQ ID NO:9; a light chain constant region as set forth in SEQ ID NO:10; a heavy chain as set forth in SEQ ID NO: 11 and a light chain as set forth in SEQ ID NO: 12. The expected bands were visible from the gel and appear in expected sizing (FIG. 5 and Table 11).

TABLE 11
The molecular mass of W305044 mAb (Ab-1)

| Lane | Protein name | MW (kDa) |
|---|---|---|
| 1 (Non- | W305044-1.100.1-p3-uIgG1KV320 (Ab-1) | 145 |
| reduced) | | |
| 2 | W305044-1.100.1-p3-uIgG1KV320 (Ab-1) | 50 + 25 |
| (Reduced) | | |

| | |
|---|---|
| M | PageRuler ™ Unstained Protein Ladder |

### 3.2 Size Exclusion Chromatography (SEC-HPLC)

SEC-HPLC assay was performed using Agilent 1260 Infinity HPLC. Briefly, 50 μL of antibody solution was injected on a TSKgel SuperSW3000 column using 50 mM sodium phosphate, 0.15 M NaCl, pH 7.0 as running buffer. The run time was 20 minutes. Peak retention times on the column were monitored at 280 nm. Data was analyzed using ChemStation software (V2.99.2.0).

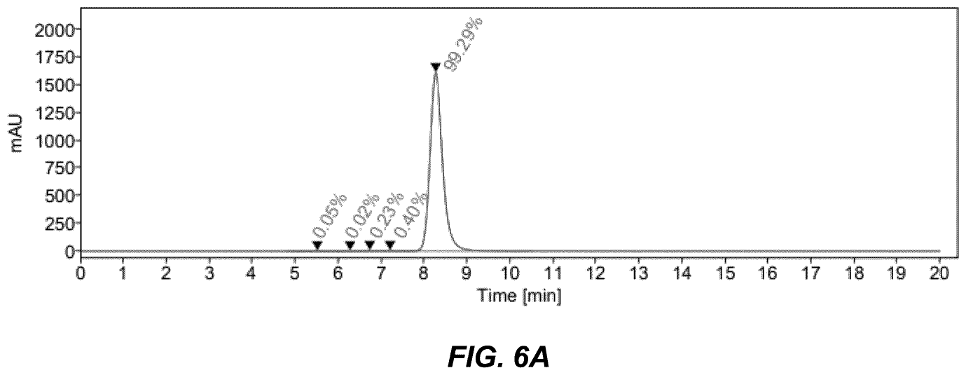

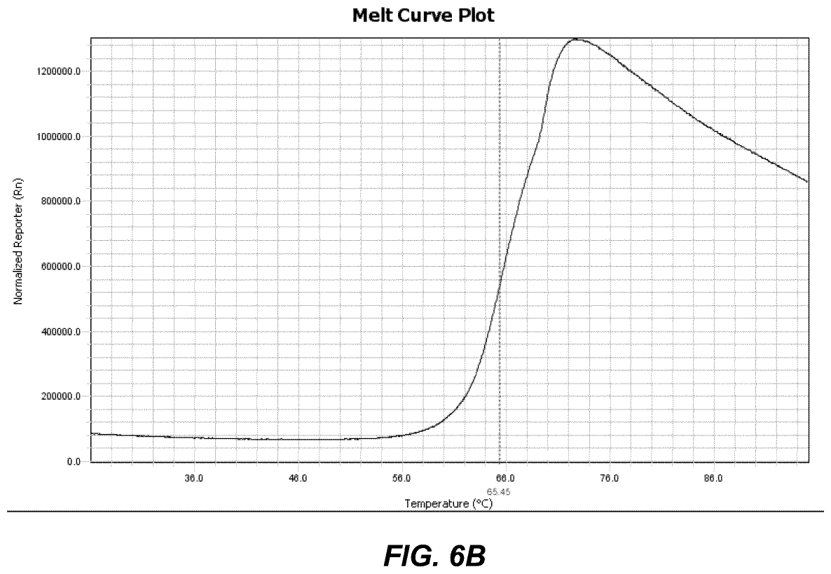

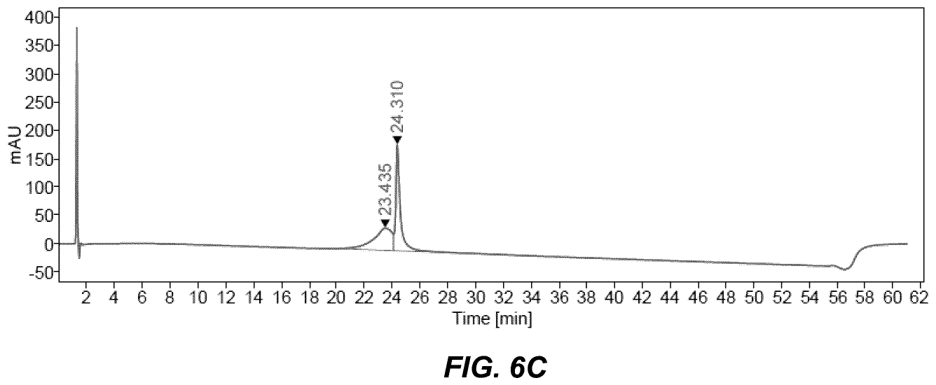

W305044 (also referred to as Ab-1) demonstrates good yield and purity. The purity of the antibody is above 95%. The retention time by SEC-HPLC of about 8.0 minutes indicates that the protein was monomer (FIG. 6A). The thermal stability of W305044 (also referred to as Ab-1) is good and Tm1 value of the antibody is 65.5° C. as determined by differential scanning fluorescence (DSF) (FIG. 6B and Table 12).

TABLE 12
The yield, purity, pI and thermal stability of W305044 mAb (Ab-1)

| | Yield in 293 (Transient | | | Tm1 | Tm2 |
|---|---|---|---|---|---|
| Protein Name | transfection) | pI | Buffer | (° C.) | (° C.) |
| W305044 (Ab-1) | 258.90 mg/L | 8.7 | PBS | 65.5 | 69.6 |

Retention time by HIC represents the hydrophobicity degree of the antibody, and long retention time indicates high potential hydrophobicity. The retention time of W305044 (Ab-1) by HIC is in the normal range (FIG. 6C and Table 13).

TABLE 13
The retention time of W305044 mAb (Ab-1)

| | | | Concentration | Retention Time |
|---|---|---|---|---|
| Antibody name | Buffer | Purity | (mg/mL) | (minutes) |
| W305044 (Ab-1) | PBS | 99.3% | 0.5 | 24.31 |

### 3.3 FACS Binding of Antibody to Cell Surface Human, Cynomolgus Monkey and Mouse MSLN Proteins

Binding of anti-MSLN antibodies to MSLN expressing cells was determined by flow cytometry (FACS). FACS can quantitatively analyze and identify specific molecules expressed on the surface of living cells. Unlabeled cells were used as a control to set the threshold before detection and then the percentage change of each group that exceeded the fluorescence intensity threshold was analyzed. CHO cells expressing human MSLN and five selected tumor cell lines including NCI-N87 (ATCC, CRL-5822™), OVCAR3 (ATCC, HTB-161™), SK-OV-3 cells (ATCC, HTB-77), HCC1806 (ATCC, CRL-2335™) and HT-29 (ATCC, HTB-38)(1×10⁵ cells/well) expressing different levels of human full-length MSLN were harvested using Versene (1×) or 0.25% Trypsin-EDTA (1×). The cells were incubated with serially diluted antibodies (starting at 200 nM, 4-fold dilution to 0.00019 nM) for 1 hour at 4° C. In some experiments, the serially diluted antibodies were preincubated with or without soluble human full-length MSLN at room temperature for 30 minutes. After washing the cells with 1×PBS/1% BSA, Alexa Fluor647-conjugated AffiniPure Goat Anti-Human IgG (1:500 dilution) was added and incubated for 30 minutes at 4° C. in the dark. After washing the cells, the mean fluorescence intensity (MFI) of the cells was measured by a flow cytometer and analyzed by FlowJo.

CHO cells expressing the full-length cynomolgus monkey MSLN and CHO cells expressing the full-length mouse MSLN were harvested using Versene (1×) or 0.25% Trypsin-EDTA (1×) and processed as described above.

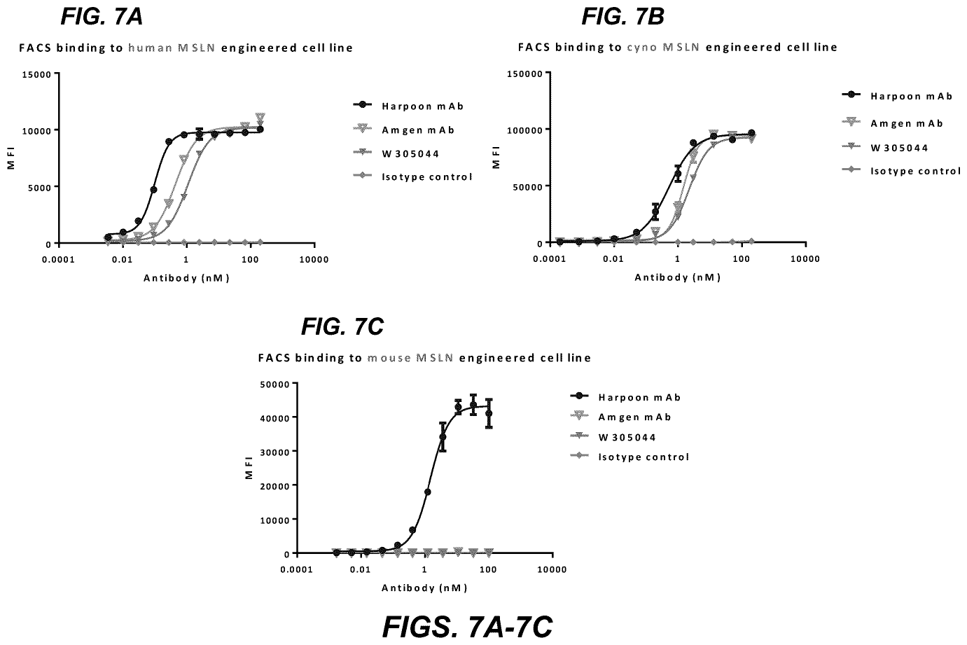

The binding results of W305044 (Ab-1) on three different engineered cells are shown in FIGS. 7A-7C. The EC50 and Max MFI are shown in Table 14. W305044 (Ab-1) specifically binds to engineered cells expressing human and cynomolgus monkey MSLN, while not binding to mouse MSLN. W305044 (Ab-1) specifically binds to the cell surface human and cynomolgus monkey MSLN with EC50 of 1.10 and 2.23 nM, which is comparable to the reference Amgen mAb. Without wishing to be bound by theory, it is believed that the three mAbs may bind to different epitopes of human MSLN; for example, Harpoon mAb may mainly bind to region I of MSLN, W305044 (Ab-1) may mainly bind to region III of MSLN, while Amgen mAb may bind to the region II of MSLN or the conformation epitope.

TABLE 14
The FACS binding EC50 and Max MFI of anti-MSLN mAbs

| | FACS | | | | | |
|---|---|---|---|---|---|---|
| | Human MSLN | | Cyno MSLN | | Mouse MSLN | |
| | EC50 | Max | EC50 | Max | EC50 | Max |
| Antibody | (nM) | MFI | (nM) | MFI | (nM) | MFI |
| Harpoon mAb | 0.099 | 10028 | 0.52 | 97200 | 1.51 | 45600 |
| Amgen mAb | 0.45 | 11050 | 1.47 | 96800 | NA | 662 |
| W305044 (Ab-1) | 1.10 | 10450 | 2.23 | 92800 | NA | 175 |

### 3.4 FACS Binding of Antibody to Five Selected Human Tumor Cell Lines

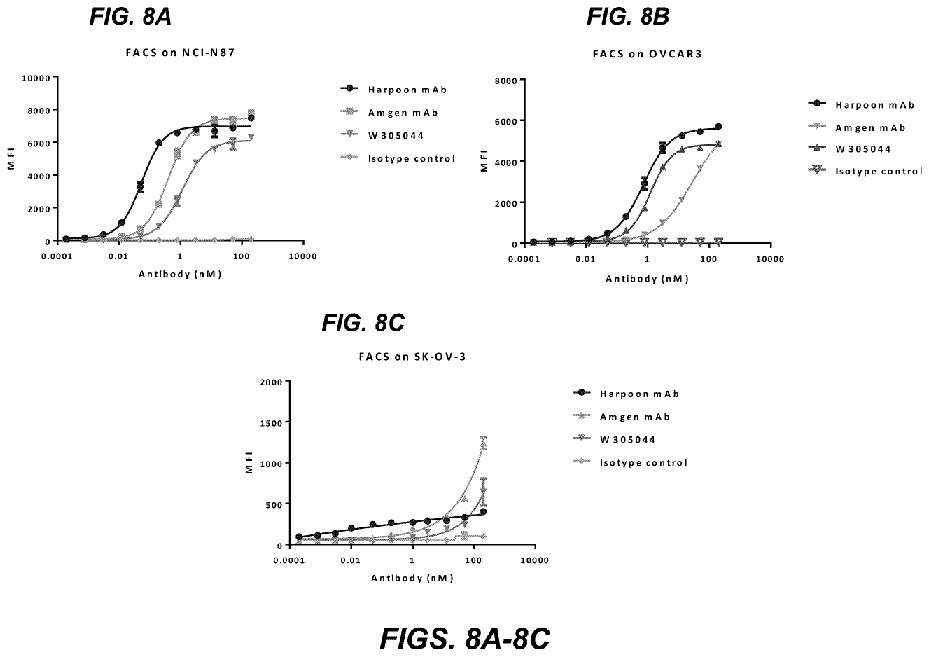

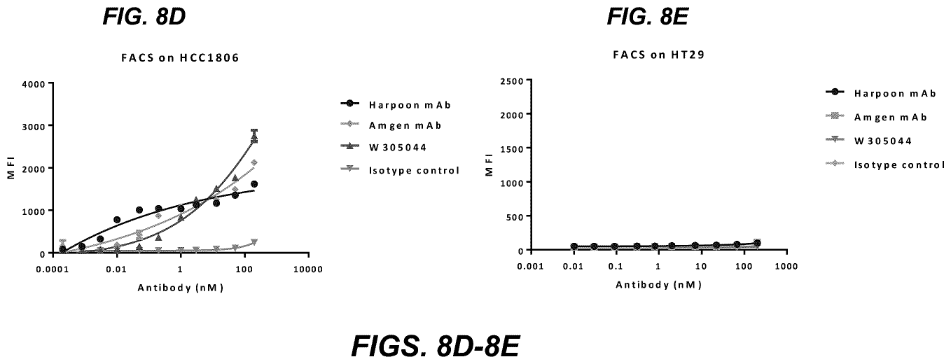

The binding results of W305044 (Ab-1) on five selected human tumor cell lines expressing different level of human full-length MSLN in section 3.3 are shown in FIGS. 8A-8E, including NC-N87 (high), OVCAR3 (medium), SK-OV-3 (low), HCC1806 (low) and HT-29 (negative). The EC50 and Max MFI are shown in Table 15. W305044 (Ab-1) specifically binds to the human tumor cell surface MSLN expressed on the antigen-positive cell lines, which is comparable to the reference Harpoon mAb and Amgen mAb. W305044, Harpoon and Amgen mAbs do not bind to the antigen-negative tumor cells HT-29.

TABLE 15
The FACS binding EC50 and Max MFI of anti-MSLN mAbs to tumor cell lines

| | FACS | | | | | | | | | |
|---|---|---|---|---|---|---|---|---|---|---|
| | NCI-N87 | | OVCAR3 | | SK-OV-3 | | HCC1806 | | HT29 | |
| | EC50 | Max | EC50 | Max | EC50 | Max | EC50 | Max | EC50 | Max |
| Antibody | (nM) | MFI | (nM) | MFI | (nM) | MFI | (nM) | MFI | (nM) | MFI |
| Harpoon mAb | 0.054 | 7509 | 0.71 | 5716 | NA | 419 | NA | 1664 | NA | 104 |
| Amgen mAb | 0.40 | 7883 | 27 | 4853 | NA | 1288 | NA | 2165 | NA | 135 |
| W305044 (Ab-1) | 1.14 | 6422 | 1.22 | 4885 | NA | 756 | NA | 2846 | NA | 46 |

### 3.5 ELISA Binding of Antibody to Human MSLN and MSLN Region III Proteins

ELISA plates were coated with human MSLN protein or human MSLN region III protein (2 μg/mL) in coating buffer and incubated at 4° C. overnight. The next day, the ELISA plates were washed with 1×PBST buffer. The ELISA plates were blocked with blocking buffer (2% BSA), incubated at room temperature for 1 hour. Serially diluted antibodies in 2% BSA (Ab1, starting at 200 nM, 5-fold dilution to 0.0001 nM) were added, and the plates incubated at room temperature for 2 hours. Biotin-labeled Harpoon mAb and Amgen mAb were used as positive controls, and the biotin-labeled W332-1.80.12.xAb.hIgG1 isotype control antibody was the negative control. Subsequently, the ELISA plates were washed and the plates processed with SA-HRP (Jackson ImmunoResearch, 1:5000, HRP conjugated) in 2% BSA. After washing, detection was done using TMB substrate. The plates were read on a microplate reader M5e at 450 nm and 540 nm.

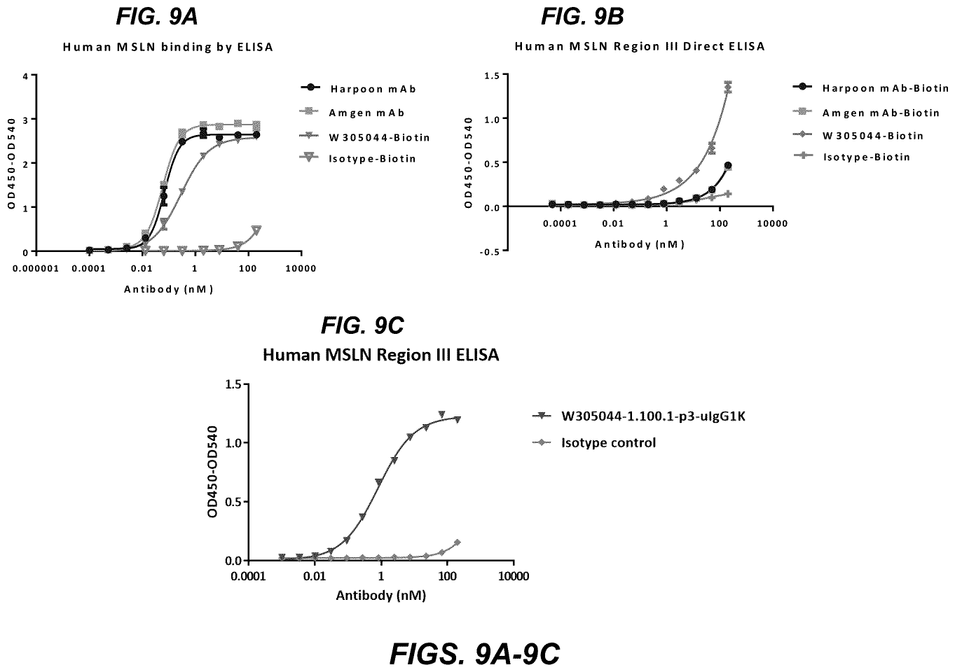

W305044 (Ab-1) showed good binding to recombinant human MSLN, which is comparable to the reference Harpoon mAb and Amgen mAb (FIG. 9A). Furthermore, the binding region of lead W305044 (Ab-1) involves MSLN region III which is different from Harpoon mAb and Amgen mAb (FIGS. 9B and 9C). The EC50 and Max OD are shown in Table 16 and Table 17.

TABLE 16
The ELISA binding EC50 and Max OD of anti-MSLN
mAbs to human MSLN and MSLN region III proteins

| | Human MSLN ECD | | Human MSLN Region III | |
|---|---|---|---|---|
| | EC50 | Max | EC50 | Max |
| Abs | (nM) | OD | (nM) | OD |
| Harpoon mAb-Biotin | 0.068 | 2.68 | NA | 0.46 |
| Amgen mAb-Biotin | 0.058 | 2.90 | NA | 0.44 |
| W305044-Biotin | 0.28 | 2.59 | NA | 1.35 |
| Isotype-Biotin | NA | 0.48 | NA | 0.14 |

TABLE 17
The ELISA binding EC50 and Max OD of the IgG converted
antibodies to human MSLN region III proteins

Human MSLN Region III

| Abs | EC50 (nM) | Max OD |
|---|---|---|
| W305044-1.100.1-p3-uIgG1K | 0.79 | 1.24 |
| W332-1.80.12.xAb.hIgG1 | NA | 0.16 |

### 3.6 Fab-ZAP Internalization Assay

CHO cells expressing human full-length MSLN were harvested by using Versene (lx, Gibco). The cell density was adjusted to 1000 cells/well. The plates were kept in a cell incubator set to 37° C., 5% CO₂ overnight. The next day, the test mAbs and Fab-ZAP (Advanced Targeting Systems, Cat. No: IT-51-100) (Ab: Fab-ZAP=1:3) were mixed and incubated at 37° C. for 30 minutes. The serially diluted mAbs and Fab-ZAP mixture in culture medium (starting at 5 nM, 6-fold dilution to 0.000018 nM) were added to the cell plates and incubated at 37° C. for 5 days. Subsequently, CTG reagent (CellTiter-Glo Chemiluminescent Cell Viability Assay Kit, Promega) was added to each well and the plates were detected by EnVision.

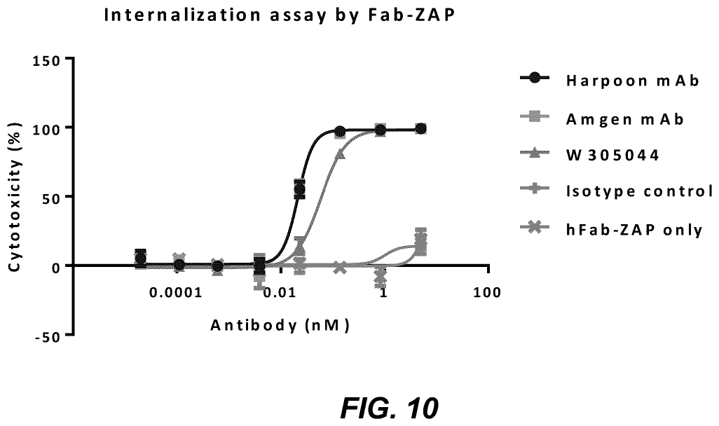

W305044 (Ab-1) mediated good target internalization and cytotoxic activity in CHO cells expressing human MSLN by Fab-ZAP assay, which is comparable to the reference Harpoon mAb and Amgen mAb (FIG. 10). The IC50 and Max cytotoxicity % are shown in Table 18.

TABLE 18
The IC50 and Max cytotoxicity % of Fab-
ZAP internalization of anti-MSLN mAbs

Internalization

| Abs | IC50 (nM) | Max cytotoxicity % |
|---|---|---|
| Harpoon mAb | 0.021 | 99 |
| Amgen mAb | 0.021 | 99 |
| W305044 (Ab-1) | 0.059 | 99 |
| Isotype control | NA | 18 |
| hFab-ZAP only | NA | 24 |

3.7 FACS Binding to Human MSLN Engineered Cells with and without Soluble MSLN

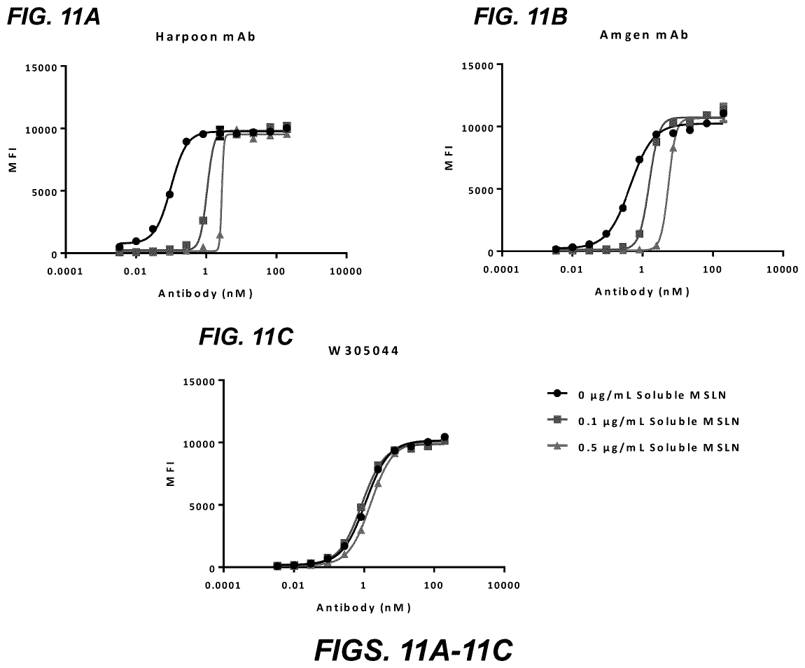

FACS binding of W305044 (Ab-1) to engineered CHO cells expressing human MSLN is minimally affected by soluble MSLN, in contrast to Harpoon and Amgen mAbs (FIG. 11). The EC50 and Max MFI values are shown in Table 19.

Compared to Harpoon and Amgen mAbs, the binding of W305044 (Ab-1) to human MSLN engineered cells was much less affected by soluble MSLN, as indicated by the minimal changes in EC50 values.

TABLE 19
The FACS binding EC50 and Max MFI of anti-
MSLN mAbs to human MSLN engineered cells
incubated with and without soluble MSLN

| | 0 μg/mL | | 0.1 μg/mL | | 0.5 μg/mL | |
|---|---|---|---|---|---|---|
| | soluble MSLN | | soluble MSLN | | soluble MSLN | |
| | EC50 | Max | EC50 | Max | EC50 | Max |
| Abs | (nM) | MFI | (nM) | MFI | (nM) | MFI |
| Harpoon mAb | 0.099 | 10028 | 1.06 | 10067 | 3.73 | 9920 |
| Amgen mAb | 0.45 | 11050 | 1.55 | 11350 | 5.45 | 11100 |
| W305044 (Ab-1) | 1.10 | 10450 | 0.85 | 10150 | 1.53 | 10250 |

3.8 FACS Binding to NCI-N87 Human Tumor Cells Incubated with and without Soluble MSLN

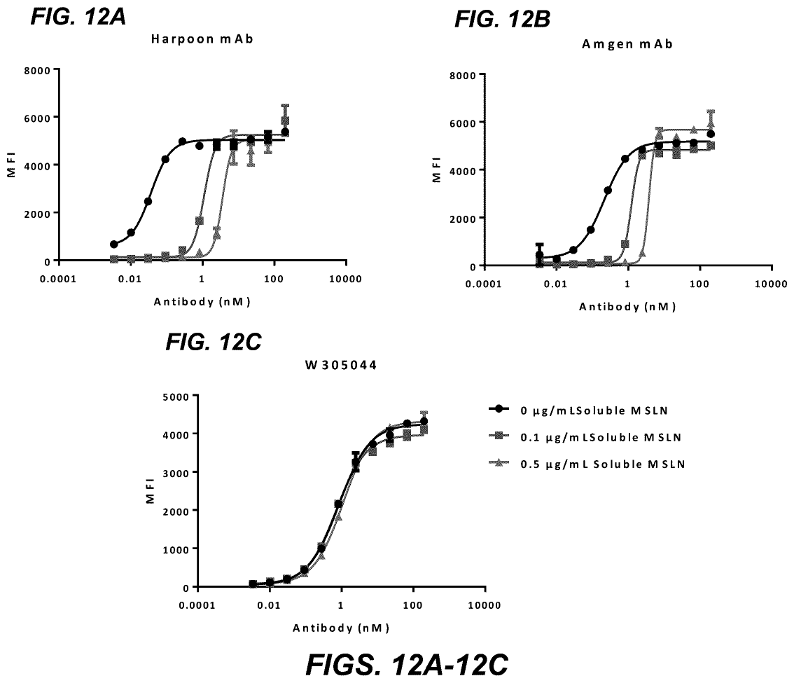

FACS binding of W305044 (Ab-1) to NCI-N87 human tumor cells that naturally express human MSLN is minimally affected by soluble MSLN, in contrast to Harpoon and Amgen mAbs (FIG. 12). The EC50 and Max MFI values are shown in Table 20.

Compared to Harpoon and Amgen mAbs, the binding of W305044 (Ab-1) to NCI-N87 human tumor cells was much less affected by soluble MSLN, as indicated by the minimal changes in EC50 values.

TABLE 20
The FACS binding EC50 and Max MFI of anti-MSLN mAbs to NCI-N87
human tumor cells incubated with and without soluble MSLN

| | 0 μg/mL | | 0.1 μg/mL | | 0.5 μg/mL | |
|---|---|---|---|---|---|---|
| | soluble MSLN | | soluble MSLN | | soluble MSLN | |
| | EC50 | Max | EC50 | Max | EC50 | Max |
| Abs | (nM) | MFI | (nM) | MFI | (nM) | MFI |
| Harpoon mAb | 0.037 | 5421 | 1.10 | 6287 | 3.57 | 5585 |
| Amgen mAb | 0.22 | 5544 | 1.22 | 5078 | 3.86 | 6298 |
| W305044 (Ab-1) | 0.85 | 4333 | 0.72 | 4114 | 1.11 | 4497 |

3.9 FACS Binding to OVCAR3 Human Tumor Cells Incubated with and without Soluble MSLN

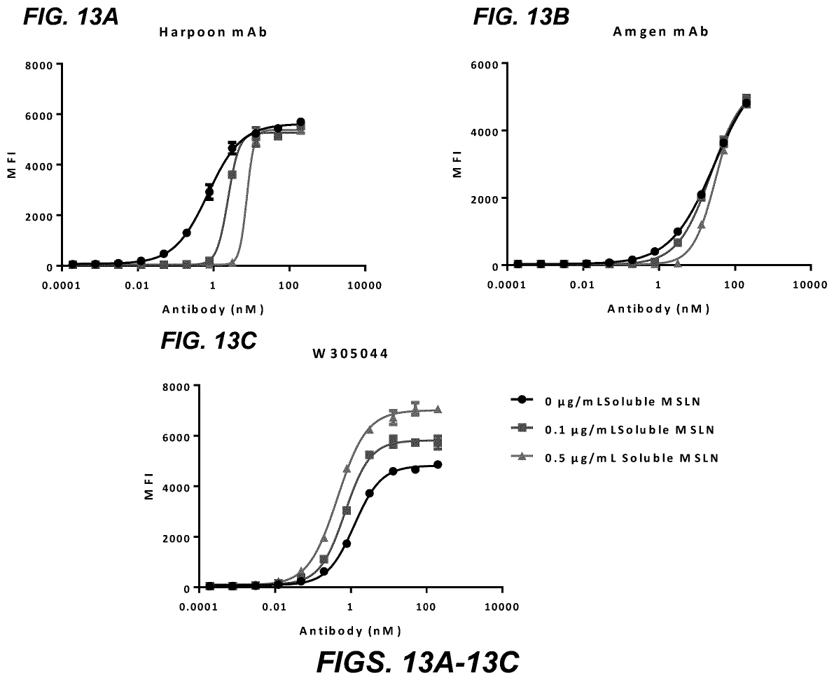

FACS binding of W305044 (Ab-1) to OVCAR3 human tumor cells naturally expressing human MSLN is not diminished by soluble MSLN, in contrast to Harpoon and Amgen mAbs (FIG. 13). The EC50 and Max MFI values are shown in Table 21.

Compared to Harpoon and Amgen mAbs, the binding of W305044 (Ab-1) to OVCAR3 human tumor cells was not decreased by soluble MSLN, as indicated by the EC50 values.

TABLE 21
The FACS binding EC50 and Max MFI of anti-
MSLN mAbs to OVCAR3 human tumor cells incubated
with and without soluble MSLN

| | 0 μg/mL | | 0.1 μg/mL | | 0.5 μg/mL | |
|---|---|---|---|---|---|---|
| | soluble MSLN | | soluble MSLN | | soluble MSLN | |
| | EC50 | Max | EC50 | Max | EC50 | Max |
| Abs | (nM) | MFI | (nM) | MFI | (nM) | MFI |
| Harpoon mAb | 0.71 | 5716 | 2.40 | 5628 | 7.50 | 5529 |
| Amgen mAb | 27 | 4853 | 23 | 4994 | 30 | 4922 |
| W305044 (Ab-1) | 1.22 | 4885 | 0.70 | 5914 | 0.44 | 7242 |

### 3.10 Affinity to MSLN (SPR)

The affinity of anti-MSLN mAbs to MSLN after IgG conversion was tested by SPR analysis. The activator was prepared by mixing 400 mM EDC and 100 mM NHS (GE) immediately prior to injection. The CM5 sensor chip was activated for 420 s with the mixture at a flow rate of 10 L/minutes. 30 g/mL of anti-human Fc IgG in 10 mM NaAc was then injected to chip for 420 s at a flow rate of 10 μL/minutes. The chip was deactivated by 1 M ethanolamine-HCl at a flow rate of 10 μL/minutes for 420 s. The analysis temperature is 25° C. The department temperature was 12° C. Ligand in running buffer 1×HBS-EP+ were captured onto chip via anti-human Fc IgG at a flow rate of 10 μL/minute. The series concentrations of analyte and running buffer were injected orderly to chip at a flow rate of 30 μL/minutes for an association phase and a dissociation phase.

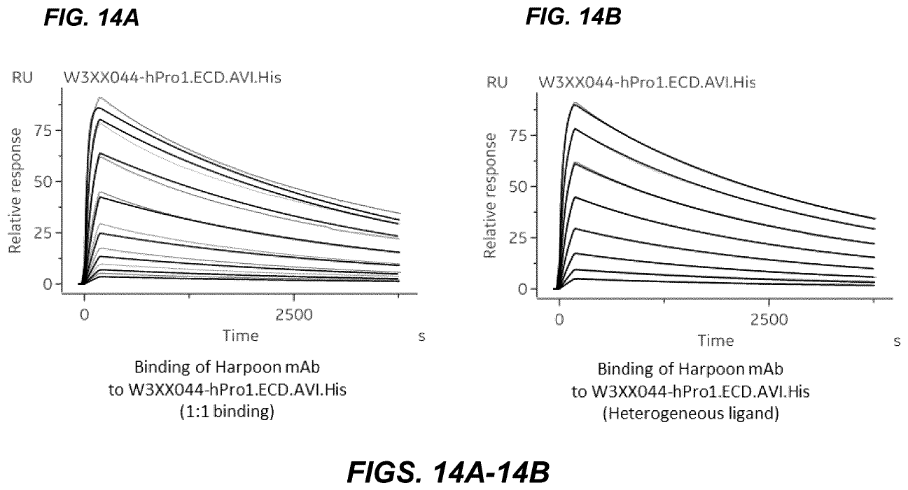

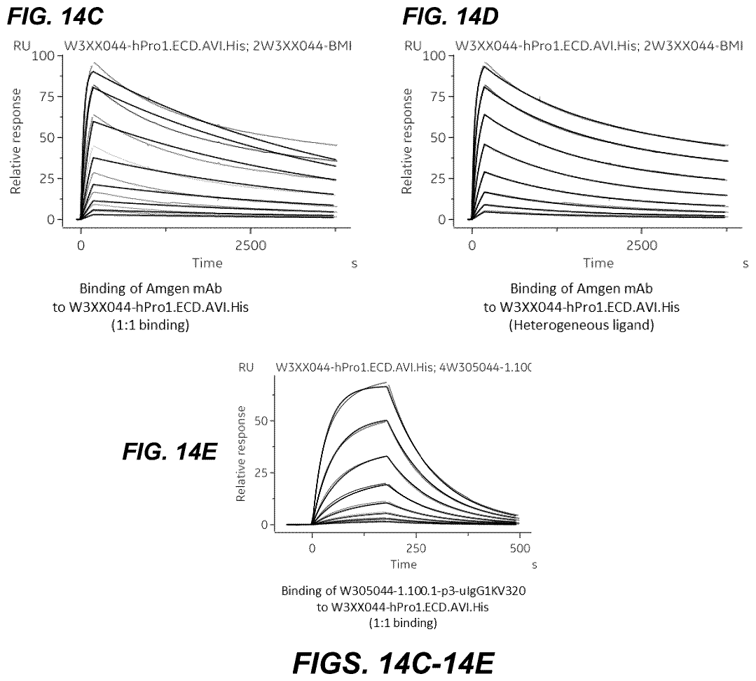

The affinity of W305044 (Ab-1) to soluble MSLN is weaker than that of Harpoon and Amgen mAbs (FIG. 14). SPR affinity data of anti-MSLN mAbs to MSLN is shown in Table 22. As expected, the weak affinity seen here is related to membrane proximal binding epitope and agrees with the minimal effect on target binding by soluble MSLN observed in the FACS assay.

TABLE 22
The SPR affinity data of anti-MSLN mAbs to MSLN

| Analyte | Ligand | kₐ₁ (1/Ms) | k_{d1} (1/s) | K_{D1} (M) | kₐ₂ (1/Ms) | k_{d2} (1/s) | K_{D2} (M) | Comment |
|---|---|---|---|---|---|---|---|---|
| Human | Harpoon mAb | 6.14E+05 | 2.83E−04 | 4.61E−10 | NA | | | 1:1 binding |
| MSLN | | 3.45E+05 | 2.52E−04 | 7.30E−10 | 2.58E+06 | 4.51E−04 | 1.75E−10 | Heterogeneous |
| | | | | | | | | ligand |
| | Amgen mAb | 1.20E+05 | 2.53E−04 | 2.12E−09 | NA | | | 1:1 binding |
| | | 3.85E+05 | 7.51E−04 | 1.95E−09 | 7.23E+04 | 7.80E−05 | 1.08E−09 | Heterogeneous |
| | | | | | | | | ligand |
| | W305044 | 2.71E+04 | 8.76E−03 | 3.23E−07 | NA | | | 1:1 binding |
| | (Ab-1) | | | | | | | |

3.11 Affinity of Anti-MSLN mAbs to Human Tumor Cell Surface MSLN (FACS)

The affinity of anti-MSLN mAbs to human tumor cell surface MSLN were tested by FACS analysis. The NCI-N87 and OVCAR3 cells were seeded in 96-well U-bottom plates at a density of 5×10⁴ cells/well. Antibodies to be tested were serially diluted in 1×PBS/1% BSA and incubated with cells at 4° C. for 1 hour. The plates were centrifuged and supernatant was discarded. The cells were then incubated with Alexa647 conjugated goat anti-human IgG Fc at 4° C. in the dark for 30 minutes. After washing the cells were re-suspended in 1×PBS/1% BSA, and fluorescence intensity was measured by flow cytometry and analyzed by FlowJo. The fluorescence intensity was converted to bound molecules/cell based on the quantitative beads standard curve by Quantum™ MESF Kits. KD was calculated by Graphpad Prism.

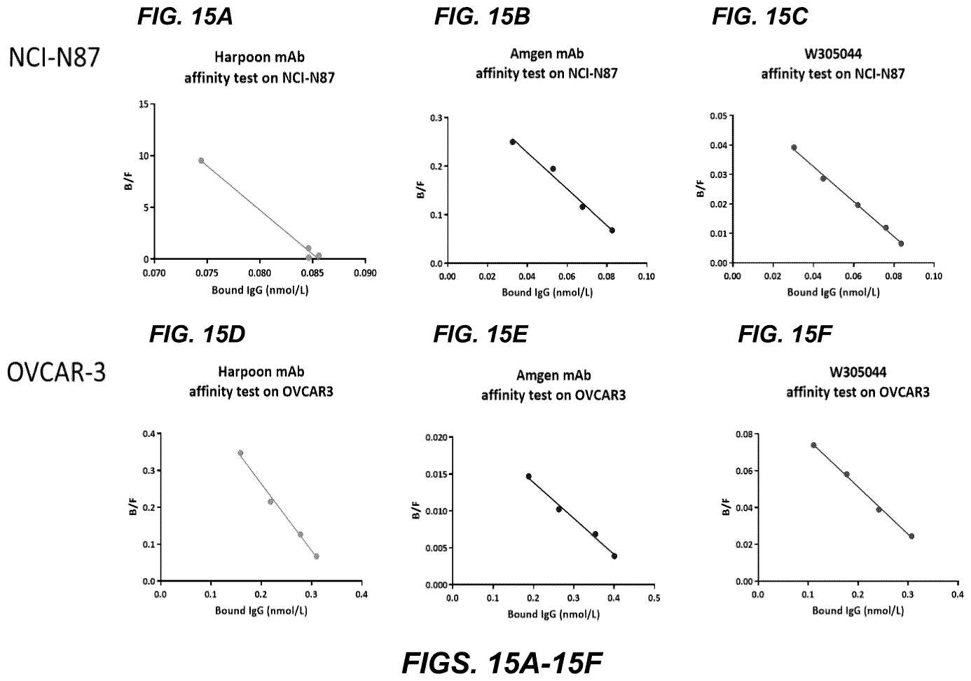

W305044 (Ab-1) showed good low nM affinity for cell surface MSLN (FIG. 15). FACS affinity data of anti-MSLN mAbs to MSLN is shown in Table 23.

TABLE 23
The FACS affinity data of anti-MSLN mAbs to human tumor cell surface MSLN

| | OVCAR3 | | | NCI-N87 | | |
|---|---|---|---|---|---|---|
| Sample | Bmax (M) | KD (M) | r² | Bmax (M) | KD (M) | r² |
| Harpoon mAb | 3.45E−10 | 5.51E−10 | 0.99 | 8.56E−11 | 1.17E−12 | 0.99 |
| Amgen mAb | 4.84E−10 | 2.05E−08 | 0.99 | 1.01E−10 | 2.67E−10 | 0.98 |
| W305044 (Ab-1) | 3.99E−10 | 3.89E−09 | 1 | 9.50E−11 | 1.68E−09 | 1 |

### 3.12 The Radius of W305044 mAb (Ab-1) Measurement by DLS

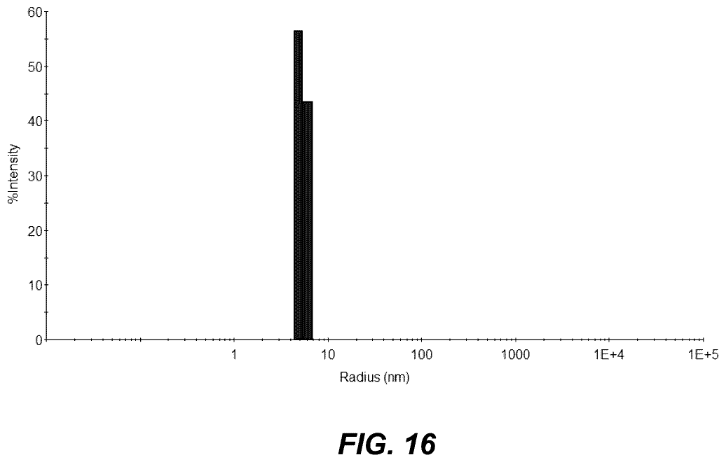

A high PD value for mAb suggests that size distribution contains several oligomeric forms of protein. To measure the real radius of the mAb, the sample was filtered by 0.1 μm filter. The radius of W305044 mAb (Ab-1) was 5.4 nm and the average PD was 1.5% in the normal range (FIG. 16 and Table 24). The W305044 mAb (Ab-1) contains very little oligomeric form protein.

TABLE 24
The radius of W305044 mAb measurement by DLS

| | Conc. | | | | Radius | |
|---|---|---|---|---|---|---|
| Sample | (mg/ | | Purity | | average | % PD |
| Name | mL) | Buffer | (%) | Treatment | (nm) | average |
| W305044 | 1 | PBS | / | Filtration, | 5.4 | 1.5 |
| (Ab-1) | | | | 12000 | | |
| | | | | rpm/3 | | |
| | | | | minutes | | |

Note:
PD: polydispersity; Normal: PD < 15%; PD > 15% indicates size distribution contains several oligomeric forms of protein.

### 3.13 Determination of Diffusion Interaction Parameter (kD) by DLS

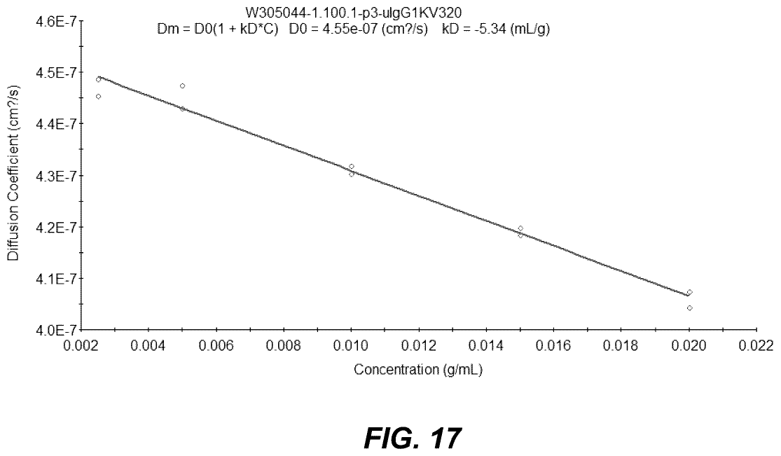

A mAb with high kD value and monodisperse size probably has low aggregation propensity. The diffusion interaction parameter kD value of the W305044 mAb (Ab-1) was −5.34 mL/g in the normal range (FIG. 17 and Table 25). The W305044 mAb (Ab-1) has low aggregation propensity.

TABLE 25
Determination of diffusion interaction parameter (kD) of W305044 mAb (Ab-1)

| | kD by DLS | | | | | Original | Purity | |
|---|---|---|---|---|---|---|---|---|
| | kD | | | | | Purity | (%) at 15 | |
| Sample ID | (mL/g) | R\hat{ }2 | Size Distribution | Lot No. | Buffer | (%) | mg/mL | Comment |
| W305044 | −5.34 | 0.9828 | Monodisperse size | 6/20/2022 | PBS | 99.29 | 98.71 | Good |
| (Ab-1) | | | | | | | | |

Note:
Good, >−10 mL/g;
Acceptable, 10~−20 mL/g;
Potential risk, <−20 mL/g

### Example 4

### Antibody Drug Conjugates and In Vivo Studies

### 4.1 Conjugation and Purification of ADCs

Ab-1-vcMMAE (Ab-1-mc-vc-PAB-MMAE) and control (hIgG-mc-vc-PAB-MMAE) were prepared by stochastic conjugation at room temperature in sodium borate buffer, pH 7.4. Ab-1 has CDRs as set forth in SEQ ID NOs:1-6; a VH as set forth in SEQ ID NO:7; a VL as set forth in SEQ ID NO:8; a heavy chain constant region as set forth in SEQ ID NO:9; a light chain constant region as set forth in SEQ ID NO:10; a heavy chain as set forth in SEQ ID NO: 11 and a light chain as set forth in SEQ ID NO: 12, as shown in Table 1. Briefly, Ab-1 or hIgG was reduced with TCEP (tris, 2-carboxyethyl phosphine) prior to incubation with drug linker, mc-vc-pab-MMAE, at 10:1 payload: antibody ratio. Excess drug linker was removed by dialysis. Size exclusion HPLC confirmed conjugate purity (99% monomer, <1% aggregate). Drug loading as assessed by LC-MS was an average of 4.

Ab-1-Dxd (Ab-1-MC-GGFG-Dxd) and control (hIgG MC-GGFG-Dxd) were prepared at room temperature in PBS buffer, pH 7.4. Briefly, Ab-1 or control antibody was reduced with TCEP prior to incubation with drug linker, MC-GGFG-Dxd, at 10:1 ratio. The reaction was stopped with N-ethylmaleimide. Excess drug linker was removed by dialysis. Size exclusion HPLC confirmed conjugate purity (98% monomer, 2% aggregate). Drug loading as assessed by LC-MS was an average of 8.

Ab-1-Exatecan would be prepared at room temperature in PBS buffer, pH 7.0. Briefly, Ab-1 antibody would be reduced with TCEP prior to incubation with drug linker at 10:1 ratio. The reaction would be stopped with N-ethylmaleimide. Excess drug linker would be removed by dialysis. Size exclusion HPLC would confirm conjugate purity >95% monomer. Drug loading as assessed by LC-MS would show an average loading of 8.

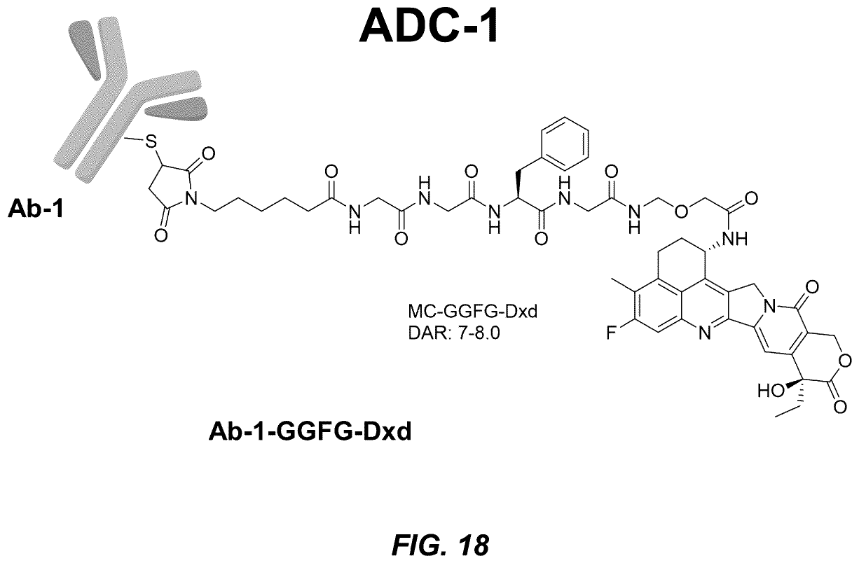

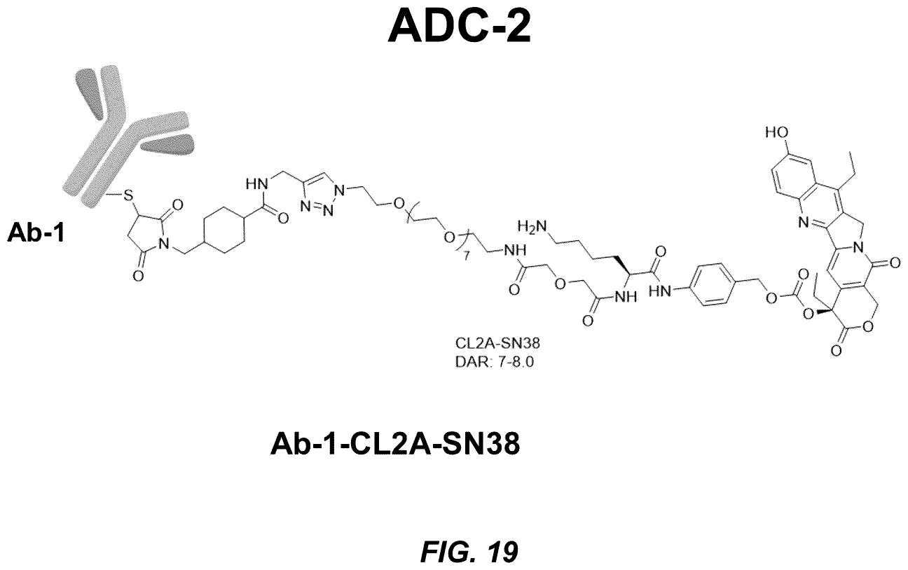

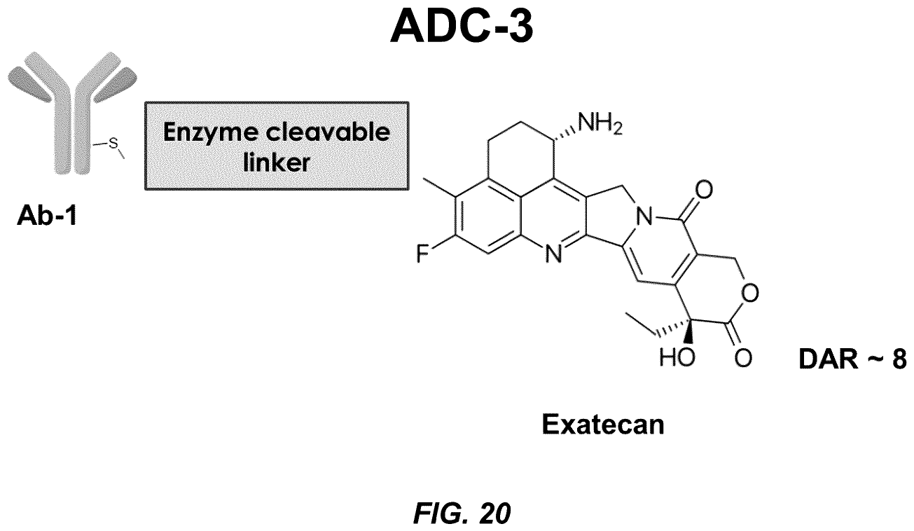

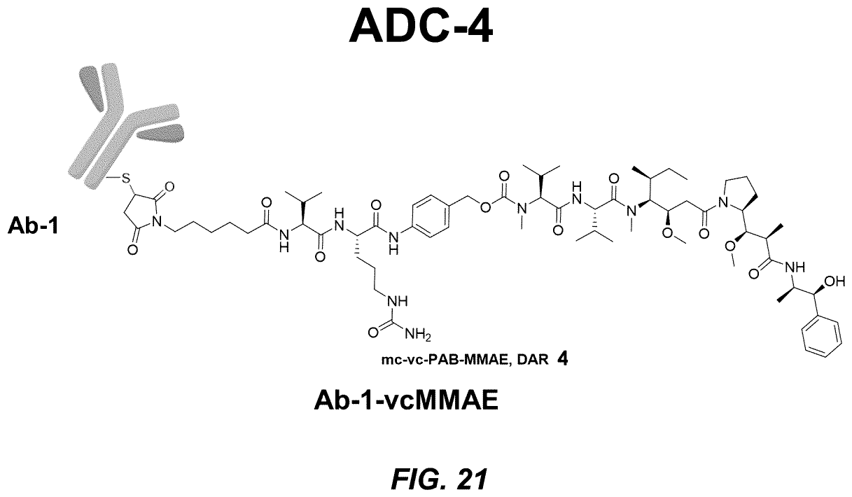

FIGS. 18-21 demonstrate the structures of Ab-1 ADCs with topoisomerase I inhibitor payloads (deruxtecan (Dxd) (FIG. 18; ADC-1), SN38 (FIG. 19; ADC-2) and exatecan (FIG. 20; ADC-3)), and auristatin payload (MMAE) (FIG. 21; ADC-4).

### 4.2 Flow Cytometry

Cells were rinsed with PBS and harvested with trypsin prior to incubation with FACs buffer (PBS+1% BSA) containing various concentrations of test antibody or ADCs for 1 h at 4° C. In some experiments, the mAb was pre-incubated with or without full-length soluble MSLN for 0.5 h at room temperature. Cells were rinsed and washed with FACs buffer and detection was performed with anti-hIgG-AF647 (Jackson Immuno Research). Cells were analyzed on a FACs Canto II (BD Biosciences).

### 4.3 Mouse Xenograft Study

All animal experiments were conducted according to IACUC (Institutional Animal Care and Use) approved protocols following AAALAC (Association for Assessment and Accreditation of Laboratory Animal Care) guidelines. For the gastric carcinoma model, 5 million NCI-N87 cells in RPMI/Matrigel (70:30) were implanted into the right flank of Balb/c nude mice. For the ovarian carcinoma model, ten million OVCAR3 cells in RPMI/Matrigel (1:1) were implanted into the right flank of CB17/SCID mice. Tumor growth was monitored twice weekly with caliper measurements and tumor volume calculated with the formula (V=0.5a×b² where a=longest and b=shortest diameter). Mice were treated intravenously with test compounds when tumors reached ˜200 to 300 mm3. Tumor growth, body weights, and general health of the mice were monitored for several weeks after the final dose of the test agent. Data was graphed with Prism (GraphPad, La Jolla, CA).

### Example 5

### Comparison of Binding of Ab-1 and Competitor mAb to Target-Expressing Cell Lines in the Absence or Presence of Soluble MSLN

In this Example, a comparison of FACs binding of Ab-1 and competitor mAb to target-expressing cell lines in the absence or presence of soluble MSLN was performed. Ab-1 has CDRs as set forth in SEQ ID NOs:1-6; a VH as set forth in SEQ ID NO:7; a VL as set forth in SEQ ID NO:8; a heavy chain constant region as set forth in SEQ ID NO:9; a light chain constant region as set forth in SEQ ID NO:10; a heavy chain as set forth in SEQ ID NO: 11 and a light chain as set forth in SEQ ID NO: 12, as shown in Table 1. Antibodies were pre-incubated with 0.1 or 0.5 μg/ml full-length soluble MSLN prior to addition to NCI-N87 gastric carcinoma or CHO cells engineered to express human MSLN.

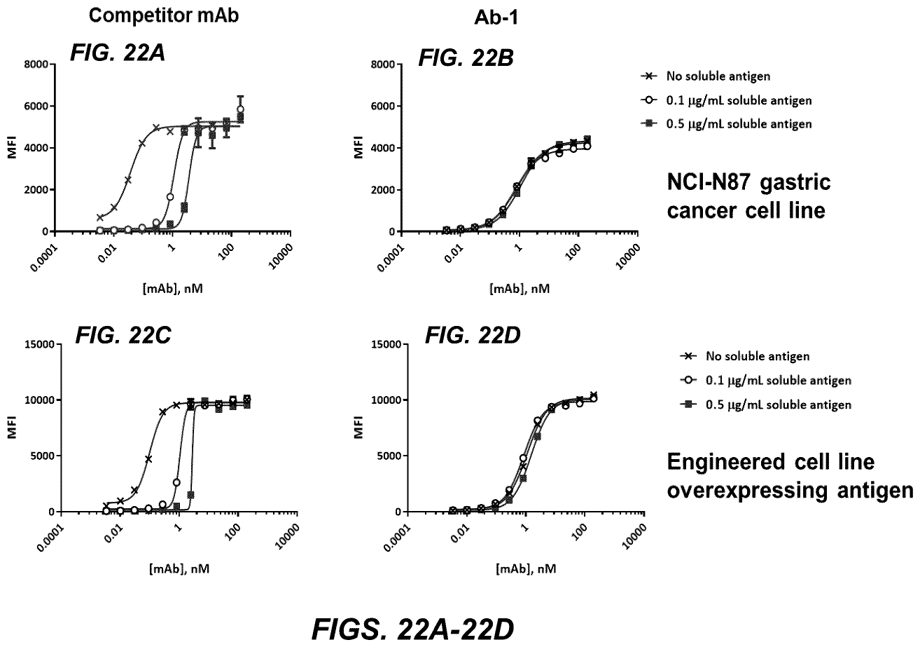

FIG. 22 shows that the competitor mAb alone bound with low nM to both cell lines. However, the binding of the competitor mAb to these cells was significantly reduced in the presence of soluble MSLN, as depicted by the shift in the binding curves. In comparison, minimal difference was observed in the binding of Ab-1 mAb to NCI-N87 gastric carcinoma or the engineered MSLN-CHO cells in the presence of soluble MSLN. These results demonstrate that the binding of Ab-1 was not impacted by the presence of soluble MSLN.

### Example 6

### Binding of Ab-1 and ADCs to Target-Positive and Target-Negative Cell Lines

In this Example, the binding of Ab-1 and ADCs (Ab-1-vcMMAE (ADC-4) and Ab-1-Dxd (ADC-1) to target-positive and target-negative cell lines was determined by flow cytometry. Ab-1 has CDRs as set forth in SEQ ID NOs:1-6; a VH as set forth in SEQ ID NO:7; a VL as set forth in SEQ ID NO:8; a heavy chain constant region as set forth in SEQ ID NO:9; a light chain constant region as set forth in SEQ ID NO:10; a heavy chain as set forth in SEQ ID NO: 11 and a light chain as set forth in SEQ ID NO: 12, as shown in Table 1.

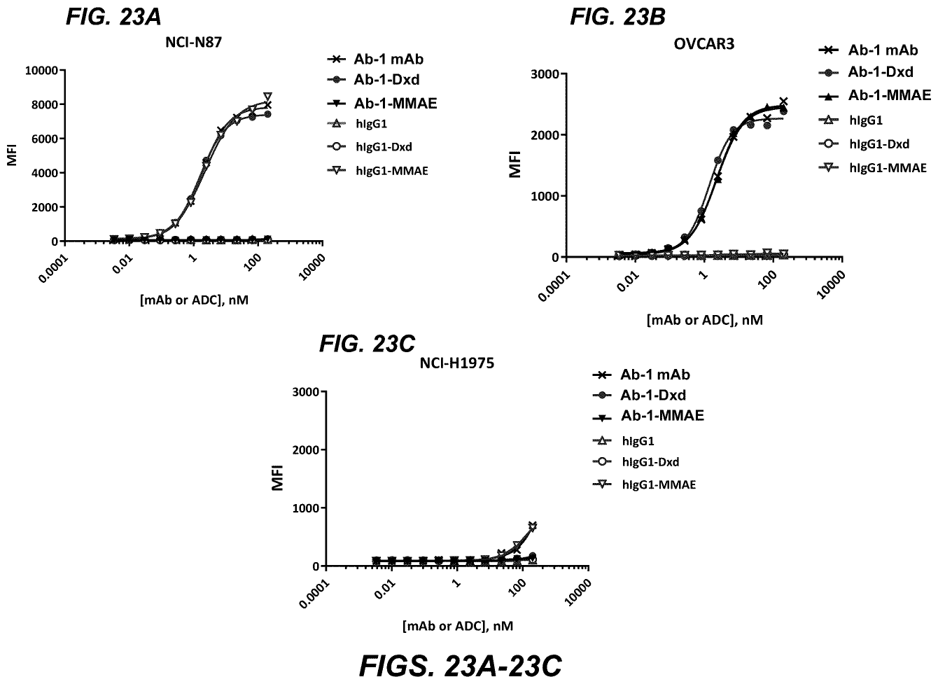

FIG. 23 shows that the mAb and ADCs bound specifically to target-positive NCI-N87 and OVCAR3 carcinoma cells, but not to the target-negative NCI-H1975 lung carcinoma cells. Furthermore, binding to NCI-N87 and OVCAR3 cells was dose-dependent, with K_{d} values of approximately 2 nM. An ADC of Ab-1-exatecan would be expected to show a similar binding pattern as Ab-1-vcMMAE and Ab-1-Dxd. These results demonstrate that Ab-1, and ADCs including Ab-1, specifically bind to cells that naturally express MSLN protein. Furthermore, the binding affinity of Ab-1 and ADCs including Ab-1 appear similar.

### Example 7

### Antitumor Effects of ADCs Including Ab-1

In this Example, the antitumor effects of ADCs including Ab-1 were measured in the NCI-N87 gastric carcinoma xenograft model and the OVCAR3 ovarian carcinoma xenograft model. Ab-1 has CDRs as set forth in SEQ ID NOs:1-6; a VH as set forth in SEQ ID NO:7; a VL as set forth in SEQ ID NO:8; a heavy chain constant region as set forth in SEQ ID NO:9; a light chain constant region as set forth in SEQ ID NO:10; a heavy chain as set forth in SEQ ID NO: 11 and a light chain as set forth in SEQ ID NO: 12, as shown in Table 1.

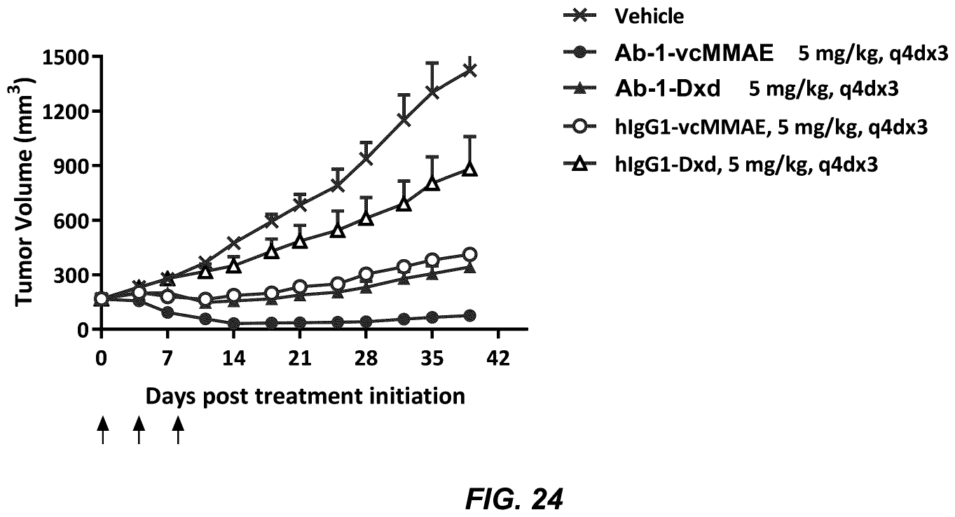

FIG. 24 shows the antitumor effects of Ab-1-vcMMAE (ADC-4) and Ab-1-Dxd (ADC-1) ADCs in the NCI-N87 gastric carcinoma xenograft model. Tumor-bearing mice were given 5 mg/kg ADCs intravenously once every 4 days for 3 doses (arrows). Ab-1-vcMMAE or Ab-1-Dxd significantly reduced tumor burden compared to Vehicle or corresponding control hIgG ADCs (p<0.005). It would be expected that the ADC Ab-1-exatecan would have better antitumor activity when compared to Ab-1-Dxd.

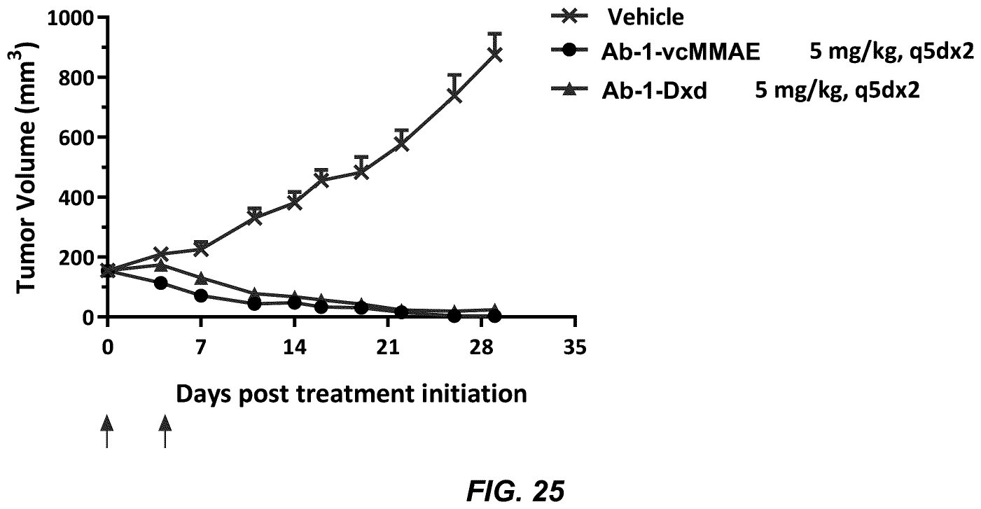

Significant anti-tumor effects of Ab-1-vcMMAE and Ab-1-Dxd ADCs were also observed in the OVCAR3 ovarian carcinoma xenograft model. FIG. 25 shows the antitumor effects of Ab-1-vcMMAE (ADC-4) and Ab-1-Dxd (ADC-1) ADCs in the OVCAR3 ovarian carcinoma xenograft model. Mice implanted with OVCAR3 tumor cells were treated with 5 mg/kg Ab-1-vcMMAE or Ab-1-Dxd once every 5 days for two doses (arrows). Two doses of either ADC significantly reduced tumor burden and yielded complete remissions in this mouse model. It would be expected that an ADC including AB-1 bearing SN38 would demonstrate comparable antitumor activity to ADC-1 whereas an ADC including Ab-1-exatecan would show complete tumor regression in this model.

The results in FIGS. 24 and 25 demonstrate that ADCs including Ab-1 are effective at slowing tumor growth and/or reducing tumor size in mouse models.

While specific embodiments have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An anti-MSLN conjugate comprising:
a. a binding agent comprising a heavy chain variable (VH) region and a light chain variable (VL) region, wherein the VH region comprises a complementarity determining region HCDR1 sequence having the amino acid sequence set forth in SEQ ID NO:1, a HCDR2 having the amino acid sequence set forth in SEQ ID NO:2, and a HCDR3 having the amino acid sequence set forth in SEQ ID NO:3; and wherein the VL region comprises a LCDR1 sequence having the amino acid sequence set forth in SEQ ID NO:4, a LCDR2 having the amino acid sequence set forth in SEQ ID NO:5, and a LCDR3 having the amino acid sequence set forth in SEQ ID NO:6;
b. at least one linker attached to the binding agent; and
c. at least one cytotoxic agent attached to the at least one linker.

2. The conjugate of claim 1, wherein the VH comprises one, two, three, or four human framework regions and the VL comprises one, two, three, or four human framework regions, or wherein the VH comprises human framework regions 1, 2, and 4, and in framework region 3 comprises one or two substitutions mutations relative to a human framework region 3, and the VL comprises four human framework regions.

3. The conjugate of claim 1 or 2, wherein framework region 3 comprises the mutation N70Q and/or S72A.

4. The conjugate any one of claims 1-3, wherein the VH region comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:7 and/or the VL region comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:8.

5. The conjugate of any one of claims 1-4, wherein the VH region comprises the amino acid sequence set forth in SEQ ID NO:7 and/or the VL region comprises the amino acid sequence set forth in SEQ ID NO:8.

6. The conjugate of claim 5, wherein the VH region comprises the amino acid sequence set forth in SEQ ID NO:7 and the VL region comprises the amino acid sequence set forth in SEQ ID NO:8.

7. The conjugate of any one of claims 1-6, wherein the binding agent is an antibody or an antigen-binding portion thereof.

8. The conjugate of claim 7, wherein the antibody or an antigen-binding portion thereof is a monoclonal antibody, a Fab, a Fab′, an F(ab′), an Fv, a disulfide linked Fv, a scFv, a scFab, a single domain antibody, a diabody, a bi-specific antibody, or a multi-specific antibody.

9. The conjugate of claim 8, wherein the bi-specific antibody or the multi-specific antibody is a Bispecific T cell Engager (BiTE); a DART; a Knobs-Into-Holes (KIH) assembly; a scFv-CH3-KIH assembly; a KIH Common Light-Chain antibody; a TandAb; a Triple Body; a TriBi Minibody; a Fab-scFv; a scFv-CH-CL-scFv; a F(ab′)2-scFv2; a tetravalent Hcab; an intrabody; a CrossMab; a Dual Action Fab (DAF) (two-in-one or four-in-one); a DutaMab; a DT-IgG, a charge paired antibody; a Fab-arm Exchange antibody, a SEEDbody; a Triomab; a LUZ-Y assembly, an Fcab; a κλ-body; an orthogonal Fabs antibody; a DVD-Ig; am IgG(H)-scFv; an scFv-(H)IgG; an IgG(L)-scFv; an scFv-(L)IgG; an IgG(L,H)-Fv; an IgG(H)-V; a V(H)-IgG; an IgG(L)-V; a V(L)-IgG; a KIH IgG-scFab; a 2scFv-IgG; a IgG-2scFv; a scFv4-Ig; a Zybody; a DVI-IgG (four-in-one), a FIT-Ig; a WuxiBody; or an In-Elbow-Insert Ig.

10. The conjugate of any one of the preceding claims, wherein the heavy chain variable region is comprised in a heavy chain that further comprises a heavy chain constant region.

11. The conjugate of claim 10, wherein heavy chain constant region is of the IgG isotype.

12. The conjugate of claim 11, wherein the heavy chain constant region is an IgG1 constant region.

13. The conjugate of any one of claims 10-12, wherein the heavy chain constant region has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:9.

14. The conjugate of any one of claims 10-13, wherein the heavy chain constant region has the amino acid sequence set forth in SEQ ID NO:9.

15. The conjugate of any one of claims 10-14, wherein the heavy chain has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO:11.

16. The conjugate of any one of claims 10-15, wherein the heavy chain has the amino acid sequence set forth in SEQ ID NO: 11.

17. The conjugate of any one of the preceding claims, wherein the light chain variable region is comprised in a light chain that further comprises a light chain constant region.

18. The conjugate of claim 17, wherein the light chain constant region has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 10.

19. The conjugate of claim 18, wherein the light chain constant region has the amino acid sequence set forth in SEQ ID NO:10.

20. The conjugate of any one of claims 17-19, wherein the light chain has an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 12.

21. The conjugate of any one of claims 17-20, wherein the light chain has the amino acid sequence set forth in SEQ ID NO:12.

22. The conjugate of any one of the preceding claims, wherein the binding agent comprises the amino acid sequence set forth in SEQ ID NO:11 and the amino acid sequence set forth in SEQ ID NO:12.

23. The conjugate of any one of the preceding claims, wherein the binding agent comprises two heavy chains and two light chains, wherein each heavy chain comprises or consists of SEQ ID NO:11 and each light chain comprises or consists of SEQ ID NO:12.

24. The conjugate of any one of the preceding claims, wherein the linker is attached to the binding agent by an interchain disulfide residue, an engineered cysteine, a glycan or modified glycan, an N-terminal residue of the binding agent, a polyhistidine residue attached to the binding agent, or any combination thereof.

25. The conjugate of any one of the preceding claims, wherein the binding agent is mono-specific.

26. The conjugate of any one of the preceding claims, wherein the binding agent is bivalent.

27. The conjugate of any one of the preceding claims, wherein the binding agent comprises a second binding domain and the binding agent is bi-specific.

28. The conjugate of any one of the preceding claims, wherein the cytotoxic agent is an anti-mitotic agent or a topoisomerase I inhibitor.

29. The conjugate of any one of the preceding claims, wherein the cytotoxic agent comprises or consists of an auristatin, a camptothecin, a duocarmycin, an anthracycline, a calicheamicin, an exatecan or any analogs thereof, or any combination thereof.

30. The conjugate of claim 29, wherein the cytotoxic agent comprises or consists of the auristatin.

31. The conjugate of claim 30, wherein the cytotoxic agent comprises or consists of MMAE.

32. The conjugate of claim 29, wherein the cytotoxic agent comprises or consists of the camptothecin or camptothecin derivative.

33. The conjugate of claim 32, wherein the cytotoxic agent comprises or consists of exatecan or exatecan derivative.

34. The conjugate of claim 32, wherein the cytotoxic agent comprises or consists of SN-38.

35. The conjugate of claim 32, wherein the conjugate comprises or consists of an anthracycline.

36. The conjugate of claim 32, wherein the cytotoxic agent comprises or consists of DXd.

37. The conjugate of any of the preceding claims, wherein the linker comprises or consists of mc-VC-PAB, CL2, CL2A, (Succinimid-3-yl-N)—(CH₂)ₙ²—C(═O)-Gly-Gly-Phe-Gly-NH—CH₂—OCH₂—(C═O)—, an enzyme-cleavable linker, or any combination thereof.

38. The conjugate of claim 37, wherein the linker comprises or consists of mc-VC-PAB.

39. The conjugate of claim 37 or 38, wherein the linker is attached to at least one molecule of MMAE.

40. The conjugate of any one of claims 37-39, wherein the conjugate is ADC-4 (Ab-1-mc-VC-PAB-MMAE).

41. The conjugate of claim 37, wherein the linker comprises or consists of CL2A.

42. The conjugate of claim 37 or 41, wherein the linker is attached to at least one molecule of SN-38.

43. The conjugate of any one of claim 37,41 or 42, wherein the conjugate is ADC-2 (Ab-1-CL2A-SN38).

44. The conjugate of claim 37, wherein the linker comprises or consists of CL2.

45. The conjugate of claim 44, wherein the linker is attached to at least one molecule of SN-38.

46. The conjugate of claim 37, wherein the linker comprises or consists of (Succinimid-3-yl-N)—(CH₂)ₙ²—C(═O)-Gly-Gly-Phe-Gly-NH—CH₂—O—CH₂—(C═O)—.

47. The conjugate of claim 46, wherein the linker is attached to at least one molecule of DXd.

48. The conjugate of any one of claims 37, 46 or 47, wherein the conjugate is ADC-1 (Ab-1-GGFG-DXd).

49. The conjugate of claim 37, wherein the linker comprises or consists of the enzyme-cleavable linker.

50. The conjugate of claim 49, wherein the linker is attached to at least one molecule of exatecan.

51. The conjugate of any one of claims 37, 49 or 50, wherein the conjugate is ADC-3 (Ab-1-enzyme-cleavable linker-exatecan).

52. A pharmaceutical composition comprising the conjugate of any of the preceding claims and a pharmaceutically acceptable carrier.

53. The pharmaceutical composition of claim 52, wherein an average number of cytotoxic agents per binding agent is from about 1 to about 8, about 2, about 4, about 6, about 8, about 10, about 12, about 14, about 16, about 3 to about 5, about 6 to about 8 or about 8 to about 16.

54. A method of treating a MSLN+ cancer, comprising administering to a subject in need thereof a therapeutically effective amount of the conjugate of any one of claims 1-51 or the pharmaceutical composition of claim 52 or 53.

55. Use of the conjugate of any one of claims 1-51 or the pharmaceutical composition of claim 52 or 53 for the treatment of MSLN+ cancer in a subject.

56. Use of the conjugate of any one of claims 1-51 or the pharmaceutical composition of claim 52 or 53 in the manufacture of a medicament for the treatment of MSLN+ cancer in a subject

57. The conjugate of any one of claims 1-51 or the pharmaceutical composition of claim 52 or 53 for use in the treatment of MSLN+ cancer in a subject.

58. The method, use, conjugate for use, or pharmaceutical composition for use, of any one of claims 54-57, wherein the MSLN+ cancer is a carcinoma or a malignancy.

59. The method, use, conjugate for use, or pharmaceutical composition for use, of any one of claims 54-58, wherein the MSLN+ cancer is mesothelioma, lung adenocarcinoma, gastric cancer, triple negative breast cancer, pancreatic cancer, ovarian adenocarcinoma, uterine serous carcinoma, acute myeloid leukemia, colorectal cancer, esophageal cancer, endometrial cancer, head and neck cancer, sarcomas, or cholangiocarcinoma.

60. The method, use, conjugate for use, or pharmaceutical composition for use, of any one of claims 54-59, further comprising administering an immunotherapy to the subject, or wherein the subject is to be administered an immunotherapy.

61. The method, use, conjugate for use, or pharmaceutical composition for use, of any one of claims 54-60, further comprising administering chemotherapy to the subject, or wherein the subject is to be administered chemotherapy.

62. The method, use, conjugate for use, or pharmaceutical composition for use, of any one of claims 54-61, wherein the subject is receiving, or has received, immunotherapy or chemotherapy.

63. The method, use, conjugate for use, or pharmaceutical composition for use, of any one of claims 60-62, wherein the immunotherapy comprises a checkpoint inhibitor.

64. The method, use, conjugate for use, or pharmaceutical composition for use, of claim 63, wherein the checkpoint inhibitor is an antibody that specifically binds to human PD-1, human PD-L1, or human CTLA4, or any combination thereof.

65. The method, use, conjugate for use, or pharmaceutical composition for use, of claim 64, wherein the checkpoint inhibitor is pembrolizumab, nivolumab, cemiplimab or ipilimumab.

66. The method, use, conjugate for use, or pharmaceutical composition for use, of any one of claims 54-65, wherein the conjugate is administered intravenously.

67. The method, use, conjugate for use, or pharmaceutical composition for use, of any one of claims 54-66, wherein the conjugate is administered in a dose of about 0.1 mg/kg body weight to about 12 mg/kg body weight.

68. A method of improving treatment outcome in a subject receiving immunotherapy and/or chemotherapy for a MSLN+ cancer, comprising:
a. administering an effective amount of an immunotherapy or chemotherapy to the subject; and
b. administering a therapeutically effective amount of the conjugate of any one of claims 1-51 or the pharmaceutical composition of claim 52 or 53 to the subject;
wherein the treatment outcome of the subject is improved, as compared to receiving the immunotherapy or chemotherapy alone.

69. Use of the conjugate of any one of claims 1-51 or the pharmaceutical composition of claim 52 or 53 for improving treatment outcome in a subject receiving immunotherapy and/or chemotherapy for a MSLN+ cancer, wherein the subject is to be administered:
a. an effective amount of an immunotherapy or chemotherapy; and
b. a therapeutically effective amount of the conjugate of any one of claims 1-51 or the pharmaceutical composition of claim 52 or 53;
wherein the treatment outcome of the subject is improved, as compared to receiving the immunotherapy or chemotherapy alone.

70. Use of the conjugate of any one of claims 1-51 or the pharmaceutical composition of claim 52 or 53 in the manufacture of a medicament for improving treatment outcome in a subject receiving immunotherapy and/or chemotherapy for a MSLN+ cancer, wherein the subject is to be administered:
a. an effective amount of an immunotherapy or chemotherapy; and
b. a therapeutically effective amount of the conjugate of any one of claims 1-51 or the pharmaceutical composition of claim 52 or 53;
wherein the treatment outcome of the subject is improved, as compared to receiving the immunotherapy or chemotherapy alone.

71. The conjugate of any one of claims 1-51 or the pharmaceutical composition of claim 52 or 53 for use in improving treatment outcome in a subject receiving immunotherapy and/or chemotherapy for a MSLN+ cancer, wherein the subject is to be administered:
a. an effective amount of an immunotherapy or chemotherapy; and
b. a therapeutically effective amount of the conjugate of any one of claims 1-51 or the pharmaceutical composition of claim 52 or 53;
wherein the treatment outcome of the subject is improved, as compared to receiving the immunotherapy or chemotherapy alone.

72. The method, use, conjugate for use, or pharmaceutical composition for use, of any one of claims 68-71, wherein the improved treatment outcome is an objective response selected from stable disease, a partial response or a complete response.

73. The method, use, conjugate for use, or pharmaceutical composition for use, of any one of claims 68-71, wherein the improved treatment outcome is reduced tumor burden.

74. The method, use, conjugate for use, or pharmaceutical composition for use, of any one of claims 68-71, wherein the improved treatment outcome is progression-free survival or disease-free survival.

75. The method, use, conjugate for use, or pharmaceutical composition for use, of any one of claims 68-74, wherein the immunotherapy is an immune checkpoint inhibitor.

76. The method, use, conjugate for use, or pharmaceutical composition for use, of claim 75, wherein the immune checkpoint inhibitor comprises an antibody that specifically binds to human PD-1, human PD-L1, or CTLA4.

77. The method, use, conjugate for use, or pharmaceutical composition for use, of claim 76, wherein the immune checkpoint inhibitor is pembrolizumab, nivolumab, cemiplimab or ipilimumab.

78. The method, use, conjugate for use, or pharmaceutical composition for use, of any one of claims 68-77, wherein the conjugate is administered intravenously.

79. The method, use, conjugate for use, or pharmaceutical composition for use, of any one of claims 68-78, wherein the conjugate is administered in a dose of about 0.1 mg/kg body weight to about 10 mg/kg body weight.


SEQUENCE LISTING (28 sequences)

SEQ ID NO: 1 - protein, 12 aa, synthetic construct
  source 1..12: note = Ab-1 HCDR1
GGSISSSSYYWG

SEQ ID NO: 2 - protein, 16 aa, synthetic construct
  source 1..16: note = Ab-1 HCDR2
SVYYSGSAFYSPSLKS

SEQ ID NO: 3 - protein, 10 aa, synthetic construct
  source 1..10: note = Ab-1 HCDR3
RPYKSGLFDF

SEQ ID NO: 4 - protein, 11 aa, synthetic construct
  source 1..11: note = Ab-1 LCDR1
RASQSVSSNLA

SEQ ID NO: 5 - protein, 7 aa, synthetic construct
  source 1..7: note = Ab-1 LCDR2
GTSTRAT

SEQ ID NO: 6 - protein, 11 aa, synthetic construct
  source 1..11: note = Ab-1 LCDR3
QQYNTWPPEYT

SEQ ID NO: 7 - protein, 120 aa, synthetic construct
  source 1..120: note = Ab-1 VH
QLQLQESGPGLVKPSETLSLTCTVSGGSISSSSYYWGWIRQPPGKGLEWIGSVYYSGSAFYSPSLKSRLNIAVDTSKKQFSLKLSSVTAADTAVYYCARRPYKSGLFDFWGQGTLVTVSS

SEQ ID NO: 8 - protein, 109 aa, synthetic construct
  source 1..109: note = Ab-1 VL
EIVMTQSPATLSVSPGERATLSCRASQSVSSNLAWYLQKPGQAPRLLIYGTSTRATGIPARFSGSGSGTEFTLTISSLQSEDFAVYYCQQYNTWPPEYTFGQGTKLEIK

SEQ ID NO: 9 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 450 aa, synthetic construct
  source 1..450: note = Ab-1 heavy chain
QLQLQESGPGLVKPSETLSLTCTVSGGSISSSSYYWGWIRQPPGKGLEWIGSVYYSGSAFYSPSLKSRLNIAVDTSKKQFSLKLSSVTAADTAVYYCARRPYKSGLFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, synthetic construct
  source 1..216: note = Ab-1 light chain
EIVMTQSPATLSVSPGERATLSCRASQSVSSNLAWYLQKPGQAPRLLIYGTSTRATGIPARFSGSGSGTEFTLTISSLQSEDFAVYYCQQYNTWPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1350 bp, synthetic construct
  source 1..1350: mol_type = other DNA; note = Ab-1 heavy chain coding sequence
caactgcagctgcaggagtcgggcccaggactggtgaagccttcggagaccctgtccctcacctgcactgtctctggtggctccatcagcagtagtagttactactggggctggatccgccagccccccgggaagggtctagaatggattgggagtgtctattatagtgggagcgccttctacagcccgtccctcaagagtcgactcaacatagctgtagacacgtccaagaagcagttctccctgaagctgagctctgtgaccgccgcagacacggctgtttattattgtgcgagacgtccatataagagtggcctctttgacttctggggccagggaaccctggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagctgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 14 - DNA, 648 bp, synthetic construct
  source 1..648: mol_type = other DNA; note = Ab-1 light chain coding sequence
gaaatagtgatgacgcagtctccagccaccctgtctgtgtctccaggggaaagagccaccctctcctgcagggccagtcagagtgttagcagcaacttagcctggtacctgcagaaacctggccaggctcccaggctcctcatctatggtacatccaccagggccactggtatcccagccaggttcagtggcagtgggtctgggacagagttcactctcaccatcagcagcctgcagtctgaagattttgcagtttattactgtcagcagtataatacctggcctccggagtacacatttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 622 aa, Homo sapiens
  source 1..622: note = NCBI Ref Seq. NP_001170826
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - protein, 622 aa, Homo sapiens; identical to SEQ ID NO: 15
  source 1..622: note = NCBI Ref Seq. NP_005814

SEQ ID NO: 17 - protein, 630 aa, Homo sapiens
  source 1..630: note = NCBI Ref Seq. NP_037536
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 18 - protein, 5 aa, synthetic construct
  source 1..5: note = Gly4Ser synthetic linker
GGGGS

SEQ ID NO: 19 - protein, 10 aa, synthetic construct
  source 1..10: note = (Gly4Ser)2 synthetic linker
GGGGSGGGGS

SEQ ID NO: 20 - protein, 15 aa, synthetic construct
  source 1..15: note = (Gly4Ser)3 synthetic linker
GGGGSGGGGSGGGGS

SEQ ID NO: 21 - protein, 20 aa, synthetic construct
  source 1..20: note = (Gly4Ser)4 synthetic linker
GGGGSGGGGSGGGGSGGGGS

SEQ ID NO: 22 - protein, 25 aa, synthetic construct
  source 1..25: note = (Gly4Ser)5 synthetic linker
GGGGSGGGGSGGGGSGGGGSGGGGS

SEQ ID NO: 23 - protein, 4 aa, synthetic construct
  source 1..4: note = Gly-Phe-Leu-Gly linker
GFLG

SEQ ID NO: 24 - protein, 4 aa, synthetic construct
  source 1..4: note = Gly-Gly-Phe-Gly linker
GGFG

SEQ ID NO: 25 - protein, 4 aa, synthetic construct
  source 1..4: note = cathepsin-B-cleavable peptides
ALAL

SEQ ID NO: 26 - protein, 116 aa, synthetic construct
  source 1..116: note = Harpoon mAb (VHH-hinge-Fc, IgG1) - variable region sequence
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 27 - protein, 117 aa, synthetic construct
  source 1..117: note = Amgen mAb (IgG1) - VH sequence
QVQLVESGGGVVQPGRSLRLSCAASGFTFSDHYMSWIRQTPGKGLEWVSYISSSGSTIYYADSVKGRFTISRDNAKNSLYLQMNSLRAEDTAVYYCARDLGPSFDYWGQGSLVTVSS

SEQ ID NO: 28 - protein, 107 aa, synthetic construct
  source 1..107: note = Amgen mAb (IgG1) - VL sequence
DIQMTQSPSSVSASVGDRVTITCRASQGISSWLAWYQQKPGKAPKLLIDAASRLQSGVPSRFSGSGSGTDFTLTISSLQPEDFATYYCQQANSFPRTFGQGTKVEIK